(19) United States
(12) Patent Application Publication
(10) Pub. No.: US 2023/0265386 A1
(43) Pub. Date: Aug. 24, 2023

(54) METHODS AND COMPOSITIONS FOR MODULATING ARGININE LEVELS IN IMMUNE CELLS

(71) Applicant: Sky Perfect International Limited, Central, Hong Kong (CN)

(72) Inventor: Kwok Ming Cheung, Hong Kong (CN)

(21) Appl. No.: 17/800,958

(22) PCT Filed: Feb. 20, 2021
(86) PCT No.: PCT/CN2021/077002

§ 371 Date: Aug. 19, 2022

Related U.S. Application Data
(60) Provisional application No. 62/979,805, filed on Feb. 21, 2020.

(51) Int. Cl. C12N 5/0783; C12N 15/63; A61K 35/17; A61P 35/00; C07K 16/28

(52) U.S. Cl. CPC C12N 5/0636; A61K 35/17; A61P 35/00; C07K 16/2827; C12N 15/63; C12N 2500/32; C12N 2510/00

(57) ABSTRACT

Disclosed herein are genetically modified T-cells and CAR-T cells that have an increased ability to process the essential amino acid arginine, for example, by overexpressing amino acid transporters, particularly arginine transporters. Such genetically modified T-cells and CAR-T cells can better survive the often hostile tumor microenvironment because of their increased ability to process arginine. The methods and compositions described here are used to augment the amount of arginine available to a T-cell. The methods and compositions described herein are also used to augment the amount of arginine available to a CAR-T-cell, thus providing a CAR-T cell that can be effective in the treatment of solid tumors by surviving the tumor microenvironment.

### CROSS-REFERENCE TO RELATED APPLICATION

This application claims priority to and the benefit of, and incorporates by reference herein in its entirety, U.S. Provisional Pat. Application Number 62/979,805, which was filed on Feb. 21, 2020.

### SEQUENCE LISTING

[0001.1] The instant application contains a Sequence Listing which has been submitted electronically in ASCII format and is hereby incorporated by reference in its entirety. Said ASCII copy, created on Feb. 18, 2021, is named SKP-001WO_SL.txt and is 312,657 bytes in size.

### FIELD OF THE INVENTION

The invention is directed to compositions and methods for modulating arginine levels in immune cells to, for example, prolong cell survival in a tumor microenvironment.

### BACKGROUND

Chimeric Antigen Receptor (CAR) T-cell therapy has emerged as a major breakthrough in cancer treatment. In CAR-T therapy, patient T-cells are harvested and genetically engineered to produce CARs that bind specific, pre-selected antigens, e.g., transmembrane receptors on cancer cells. CAR-T cells are reintroduced into the patient’s body, allowing them to attack pre-determined targets, e.g., cancer cells. Upon binding of the CAR receptor with its target antigen, the CAR-T cell becomes activated and launches an immune response against the cell displaying the target antigen. CAR-T cell therapy has induced successful patient responses and, in some cases, remission in patients who have previously failed to respond to standard treatments. For example, in some forms of leukemia, CAR-T therapy has demonstrated remission rates as high as 94%.

Existing CAR-T cell therapies are currently approved for use in hematological cancers only. Currently there are two FDA approved CAR-T cell therapies on the market, Kymriah (tisagenlecleucel) and Yescarta (axicabtagene ciloleucel), which are used to treat hematological malignancies at a few specialized research hospitals. These treatments have resulted in complete and long-lasting remissions in several subjects, including in those with cancers previously resistant to standard treatment regimens.

Previous CAR development has focused on targeting B-lymphocyte antigen CD19, a transmembrane protein which recruits cytoplasmic signaling proteins to the membrane and decreases the threshold for B cell receptor signaling pathways. Due to these necessary functions, CD19 is ubiquitous on all B cells and is used as a biomarker for malignancies that arise from B cells, notably B cell lymphomas, acute lymphoblastic leukemias, and chronic lymphocytic leukemias. Other domains that have been targeted for CAR-T therapies are CD22 – a sugar binding transmembrane protein found on the surface of mature B cells, CD123 – an interleukin-3 transmitter expressed across acute myeloid leukemia subtypes, and B-cell maturation antigen – a cell-surface receptor of the tumor necrosis factor receptor superfamily which recognizes B-cell activating factor relevant in a variety of leukemias, lymphomas, and multiple myelomas.

The tumor microenvironment (TME) of solid tumors is hostile to all effector T-cells, including engineered CAR-T cells. Immunosuppressive signals and shortage of essential nutrients within the TME result in T-cell exhaustion. Thus, the ability of CAR-T cells to penetrate and be functional in the TME has remained limited.

Thus, there is a need for CAR-T cells and pharmaceutical compositions comprising CAR-T cells that are resistant to the challenges of the TME and are able to function in cancer cell destruction within the TME. There is also a need for methods of effectively treating cancer with CAR-T cells and pharmaceutical compositions comprising CAR-T cells that are effective in inducing patient responsiveness and remission where such patients are refractory to other forms of cancer treatment or other methods of treatment with CAR-T cells. Furthermore, there is a need for methods of treating cancer by administering superior CAR-T cells that are effective to destroy cancer cells within the TME without undergoing exhaustion.

### SUMMARY

The disclosure is directed, at least in part, to T-cells expressing an amino acid transporter protein, for example, an arginine transporter protein, and a CAR that specifically binds a cell surface antigen on a target cell. Such CAR-T cells are useful for the treatment of malignancies such as cancer. Genetically modified T-cells and expression vectors described herein may have enhanced robustness and/or survival in a tumor microenvironment and resource-depleted, for example, arginine-depleted, microenvironments, as compared to, for example, T-cell populations not subject to genetic modification. The described genetically modified T-cells and expression vectors are useful for treating cancers and other diseases that require targeting of T-cells to a specific cell population.

In another aspect, the disclosure is directed, to genetically modified T-cells expressing an amino acid transporter, for example, an arginine transporter. The amino acid transporter can be the product of a recombinant amino acid transporter nucleotide sequence. T-cells described herein that are genetically modified to express or overexpress an amino acid transporter may have enhanced robustness and/or survival in a tumor microenvironment and resource-depleted, for example, arginine-depleted, microenvironments, as compared to, for example, T-cell populations not genetically modified to express or overexpress the amino acid transporter. The described genetically modified T-cells and expression vectors are useful for treating cancers and other diseases that require targeting of T-cells to a specific cell population or which require enhanced robustness of T-cells in order to survive in a biological environment depleted of one or more amino acids, for example, arginine.

In one aspect, disclosed herein is a genetically modified T-cell that is genetically modified to express an arginine transporter and a chimeric antigen receptor (CAR). In some embodiments, the CAR has at least one antigen-specific targeting region that specifically binds a cell surface antigen present on a target cell population, a transmembrane domain, and an intracellular signaling domain. In some embodiments, the CAR has at least an antigen-specific targeting region that specifically binds a cell surface antigen present on a target cell population, a transmembrane domain, at least one co-stimulatory domain, and an intracellular signaling domain.

Also described herein are expression vectors that include nucleotide sequences encoding a CAR and/or an amino acid transporter, for example, an arginine transporter. In some embodiments, transcription of expression vectors described herein results in producing a ribonucleic acid (RNA), for example a messenger RNA (mRNA) sequence encoding a CAR and/or an amino acid transporter, for example, an arginine transporter nucleotide sequence. In some embodiments, expression vectors described herein are capable of expressing a CAR and/or an amino acid transporter, for example, an arginine transporter. In some embodiments described herein is an expression vector comprising an isolated nucleic acid sequence encoding an antigen-specific targeting region, a transmembrane domain, optionally, at least one co-stimulatory domain, an intracellular signaling domain, and an arginine transporter. In some embodiments described herein is an expression vector comprising an isolated nucleic acid sequence encoding an antigen-specific targeting region, a transmembrane domain, optionally, at least one co-stimulatory domain, and an intracellular signaling domain. In some embodiments described herein is an expression vector comprising an isolated nucleic acid sequence encoding an arginine transporter. In some embodiments described herein is an expression vector comprising an isolated nucleic acid sequence encoding an antigen-specific targeting region, a transmembrane domain, optionally at least one co-stimulatory domain, an intracellular signaling domain, and an amino acid transporter. In some embodiments described herein is an expression vector comprising an isolated nucleic acid sequence encoding an amino acid transporter.

Also described herein are expression vectors that include nucleotide sequences encoding an amino acid transporter, for example, an arginine transporter. In some embodiments, transcription of expression vectors described herein results in producing a ribonucleic acid (RNA), for example a messenger RNA (mRNA) sequence encoding an amino acid transporter, for example, an arginine transporter nucleotide sequence. In some embodiments, expression vectors described herein are capable of expressing an amino acid transporter, for example, an arginine transporter. In some embodiments described herein is an expression vector comprising an isolated nucleic acid sequence encoding an arginine transporter. In some embodiments described herein is an expression vector comprising an isolated nucleic acid sequence encoding an arginine transporter. In some embodiments described herein is an expression vector comprising an isolated nucleic acid sequence encoding an amino acid transporter. In some embodiments described herein is an expression vector comprising an isolated nucleic acid sequence encoding an amino acid transporter. In embodiments described herein, a nucleic acid sequence can be, for example, a ribonucleic acid (RNA) sequence, a deoxyribonucleic acid (DNA) sequence, or a mixed DNA and RNA sequence.

In some embodiments, an expression vector described herein comprises a nucleotide sequence encoding a CAR and an amino acid transporter, wherein the CAR and the amino acid transporter nucleotide sequences are transcribed into separate mRNA transcripts. In some embodiments, an expression vector described herein comprises a nucleotide sequence encoding a CAR and an amino acid transporter, wherein the CAR and the amino acid transporter nucleotide sequences are transcribed together into a single mRNA transcript. In embodiments wherein the CAR and the amino acid transporter nucleotide sequences are transcribed together into a single mRNA transcript, the expression vector nucleotide sequence encoding the CAR and the amino acid transporter mRNA transcript can include an internal ribosome entry sequence (IRES). In some embodiments, the IRES is disposed between the portion of the nucleotide sequence encoding the CAR and the portion of the nucleotide sequence encoding the amino acid transporter. Thus, in embodiments, a CAR nucleotide sequence and an amino acid transporter nucleotide sequence are separated by an IRES sequence. In embodiments wherein the CAR and the amino acid transporter nucleotide sequences are transcribed together into a single mRNA transcript, the expression vector nucleotide sequence encoding the CAR and the amino acid transporter mRNA transcript may include a 2A self-cleavage sequence disposed between the portion of the nucleotide sequence encoding the CAR and the portion of the nucleotide sequence encoding the amino acid transporter. Thus, in some embodiments, a CAR nucleotide sequence and an amino acid transporter nucleotide sequence are separated by a 2A self-cleavage sequence. In some embodiments, a peptide translated from an mRNA that includes a CAR nucleotide sequence, a 2A self-cleavage sequence, and an amino acid transporter nucleotide sequence, is cleaved after translation at the 2A self-cleavage site.

Also described herein is a genetically modified T-cell modified to express a CAR encoded by an expression vector. Also described herein is a genetically modified T-cell modified to express a CAR and an amino acid transporter, for example, an arginine transporter, encoded by an expression vector. Also described herein is a genetically modified T-cell modified to express an amino acid transporter, for example, an arginine transporter encoded by an expression vector. Also described herein is a genetically modified T-cell modified to express a CAR encoded by a first expression vector and an amino acid transporter, for example, an arginine transporter encoded by a second expression vector. In embodiments described herein, a CAR encoded by an expression vector can include an antigen-specific targeting region, a transmembrane domain, optionally at least one co-stimulatory domain, and an intracellular signaling domain. A genetically modified T-cell modified to express an amino acid transporter, for example, an arginine transporter, encoded by an expression vector can include a genetically modified T-cell modified to express a recombinant amino acid transporter, for example, a recombinant arginine transporter.

Also described herein is a genetically modified T-cell modified to express a CAR encoded by a virus-derived transgene. Also described herein is a genetically modified T-cell modified to express a CAR and an amino acid transporter, for example, an arginine transporter, encoded by a virus-derived transgene. Also described herein is a genetically modified T-cell modified to express an amino acid transporter, for example, an arginine transporter encoded by a virus-derived transgene. Also described herein is a genetically modified T-cell modified to express a CAR encoded by a first virus-derived transgene and an amino acid transporter, for example, an arginine transporter encoded by a second virus-derived transgene. In embodiments described herein, a CAR encoded by a virus-derived transgene can include an antigen-specific targeting region, a transmembrane domain, optionally at least one co-stimulatory domain, and an intracellular signaling domain. A genetically modified T-cell modified to express an amino acid transporter, for example, an arginine transporter, encoded by a virus-derived transgene can include a genetically modified T-cell modified to express a recombinant amino acid transporter, for example, a recombinant arginine transporter.

A genetically modified T-cell described herein can express a specific arginine transporter. In some embodiments, an arginine transporter comprises a single arginine transporter protein. In some embodiments, an arginine transporter comprises two arginine transporter proteins. For example, a genetically modified T-cell described herein can express an arginine transporter selected from the group consisting of CAT-1, CAT-2, CAT-3, CAT-4, y⁺LAT1, 4F2hc, y⁺LAT2, y⁺LAT1 and 4F2hc, y⁺LAT2 and 4F2hc, b^{0,+}AT, rBAT, b^{0,+}AT and rBAT, and ATB^{0,+}, or a combination thereof.

In some embodiments, an expression vector described herein comprises an isolated nucleic acid sequence encoding an arginine transporter. In some embodiments, an expression vector described herein comprises two or more isolated nucleic acid sequences encoding proteins that together comprise an arginine transporter. In some embodiments, the arginine transporter nucleic acid sequence or sequences is selected from the group consisting of the nucleic acid sequence or sequences of CAT-1, CAT-2, CAT-3, CAT-4, y⁺LAT1, 4F2hc, y⁺LAT2, y⁺LAT1 and 4F2hc, y⁺LAT2 and 4F2hc, b^{0,+}AT, rBAT, b^{0,+}AT and rBAT, and ATB^{0,+}, or a combination thereof.

In some embodiments, a virus-derived transgene described herein comprises an isolated nucleic acid sequence encoding an arginine transporter. In some embodiments, a virus-derived transgene described herein comprises two or more isolated nucleic acid sequences encoding proteins that together comprise an arginine transporter. In some embodiments, the arginine transporter nucleic acid sequence or sequences is selected from the group consisting of the nucleic acid sequence or sequences of selected from the group consisting of CAT-1, CAT-2, CAT-3, CAT-4, y⁺LAT1, 4F2hc, y⁺LAT2, y⁺LAT1 and 4F2hc, y⁺LAT2 and 4F2hc, b^{0,+}AT, rBAT, b^{0,+}AT and rBAT, and ATB^{0,+}, or a combination thereof.

Also described herein are expression vectors that include a nucleic acid sequence encoding an amino acid transporter sequence and genetically modified T-cells comprising a recombinant nucleic acid sequence encoding an amino acid transporter. For example, described herein is an expression vector comprising a nucleic acid sequence selected from the group consisting of SEQ ID NO: 180, 184-188, 204, 205, 210, 214, 215, 220-222, 227-230, 234-236, 242, and 246, or a fragment or a variant thereof. Also described herein is an expression vector comprising the nucleotide sequence of any one of SEQ ID NO:220-222 and the nucleotide sequence of any one of SEQ ID NO:227-230. Also described herein is an expression vector comprising the nucleotide sequence of any one of SEQ ID NO:214 and 215 and the nucleotide sequence of any one of SEQ ID NO:227-230. Also described herein is an expression vector comprising the nucleotide sequence of any one of SEQ ID NO:234-236 and the nucleotide sequence of SEQ ID NO:242.

Also described herein is a genetically modified T-cell comprising a recombinant nucleic acid sequence comprising a sequence selected from the group consisting of: SEQ ID NO: 180, 184-188, 204, 205, 210, 214, 215, 220-222, 227-230, 234-236, 242, and 246, or a fragment or a variant thereof. Also described herein is a genetically modified T-cell comprising a recombinant nucleic acid comprising the nucleotide sequence of any one of SEQ ID NO:220-222 and the nucleotide sequence of any one of SEQ ID NO:227-230. Also described herein is a genetically modified T-cell comprising a recombinant nucleic acid comprising the nucleotide sequence of any one of SEQ ID NO:214 and 215 and the nucleotide sequence of any one of SEQ ID NO:227-230. Also described herein is a genetically modified T-cell comprising a recombinant nucleic acid comprising the nucleotide sequence of any one of SEQ ID NO:234-236 and the nucleotide sequence of SEQ ID NO:242.

In some embodiments, a genetically modified T-cell described herein is genetically modified to comprise a recombinant nucleic acid sequence comprising a sequence selected from the group consisting of: SEQ ID NO: 180, 184-188, 204, 205, 210, 214, 215, 220-222, 227-230, 234-236, 242, and 246, or a fragment or a variant thereof. For example, in some embodiments, a genetically modified T-cell described herein is modified to comprise one or more additional copies of a nucleic acid sequence selected from the group consisting of: SEQ ID NO: 180, 184-188, 204, 205, 210, 214, 215, 220-222, 227-230, 234-236, 242, and 246, or a fragment or a variant thereof. Also described herein is a genetically modified T-cell comprising an expression vector comprising a nucleic acid sequence selected from the group consisting of: SEQ ID NO: 180, 184-188, 204, 205, 210, 214, 215, 220-222, 227-230, 234-236, 242, and 246, or a fragment or a variant thereof. In some embodiments, an expression vector, a genetically modified T-cell, or a genetically modified T-cell comprising an expression vector described herein comprises a combination of nucleic acid sequences selected from the group consisting of: SEQ ID NO: 180, 184-188, 204, 205, 210, 214, 215, 220-222, 227-230, 234-236, 242, and 246, or a fragment or a variant thereof. In some embodiments described herein, an expression vector, a genetically modified T-cell comprising a recombinant nucleic acid sequence, a genetically modified T-cell that is genetically modified to comprise a recombinant nucleic acid sequence, a genetically modified T-cell that is modified to comprise one or more additional copies of a nucleic acid sequence, or a genetically modified T-cell comprising an expression vector comprising a nucleic acid sequence, comprises at least two nucleic acid sequences selected from the group consisting of: SEQ ID NO: 180, 184-188, 204, 205, 210, 214, 215, 220-222, 227-230, 234-236, 242, and 246, or a fragment or a variant thereof. For example, in some embodiments described herein, an expression vector, a genetically modified T-cell comprising a recombinant nucleic acid sequence, a genetically modified T-cell that is genetically modified to comprise a recombinant nucleic acid sequence, a genetically modified T-cell that is modified to comprise one or more additional copies of a nucleic acid sequence, or a genetically modified T-cell comprising an expression vector comprising a nucleic acid sequence, comprises one of the following pairs of nucleotide sequences: the nucleotide sequence of any one of SEQ ID NO:220-222 and the nucleotide sequence of any one of SEQ ID NO:227-230; the nucleotide sequence of any one of SEQ ID NO:214 and 215 and the nucleotide sequence of any one of SEQ ID NO:227-230; or the nucleotide sequence of any one of SEQ ID NO:234-236 and the nucleotide sequence of SEQ ID NO:242.

A genetically modified T-cell described herein can include a recombinant nucleic acid sequence that shares similarity with a nucleic acid sequence of one of SEQ ID NO: 180, 184-188, 204, 205, 210, 214, 215, 220-222, 227-230, 234-236, 242, and 246. For example, in some embodiments, a genetically modified T-cell described herein, comprises a nucleic acid sequence having about 90%, 91%, 92%, 93%, 94%, 95%, 96%, 97%, 98%, 99%, from about 90% to about 95%, from about 95% to about 99%, or from about 90% to about 99% percent identity to one of SEQ ID NO: 180, 184-188, 204, 205, 210, 214, 215, 220-222, 227-230, 234-236, 242, and 246. In some embodiments, a genetically modified T-cell described herein, comprises a nucleic acid sequence having about 90%, 95%, or 99% percent identity to one of SEQ ID NO: 180, 184-188, 204, 205, 210, 214, 215, 220-222, 227-230, 234-236, 242, and 246. In some embodiments, a genetically modified T-cell described herein, comprises an expression vector that comprises a nucleic acid sequence having about 90%, 91%, 92%, 93%, 94%, 95%, 96%, 97%, 98%, 99%, from about 90% to about 95%, from about 95% to about 99%, or from about 90% to about 99% percent identity to one of SEQ ID NO: 180, 184-188, 204, 205, 210, 214, 215, 220-222, 227-230, 234-236, 242, and 246. In some embodiments, a genetically modified T-cell described herein, comprises an expression vector that comprises a nucleic acid sequence having about 90%, 95%, or 99% percent identity to one of SEQ ID NO: 180, 184-188, 204, 205, 210, 214, 215, 220-222, 227-230, 234-236, 242, and 246.

In another aspect, described herein is a pharmaceutically acceptable composition comprising a genetically modified T-cell described herein and a pharmaceutically acceptable excipient.

Also described herein is a priming medium comprising L-arginine for priming a genetically modified T-cell, for example, a genetically modified T-cell described herein. A priming medium described herein can increase intracellular arginine concentration in a genetically modified T-cell, for example, a genetically modified T-cell expressing an arginine transporter. A priming medium described herein can prime genetically modified T-cells for treatment. For example, a priming medium described herein can increase intracellular arginine concentration in genetically modified T-cells prior to administration of the genetically modified T-cells to a patient in need thereof, for example, a patient in need of treatment of a cancer. In some embodiments described herein is a priming medium comprising a genetically modified T-cell described herein and L-arginine.

Also described herein are pharmaceutical compositions comprising CAR-T cells. For example, described herein is a pharmaceutical composition comprising a CAR-T cell which expresses a recombinant arginine transporter and a chimeric antigen receptor protein. In some embodiments, a pharmaceutical composition of the invention comprises a CAR-T cell, wherein the CAR-T cell comprises one or more expression vectors that comprise a nucleic acid sequence encoding an arginine transporter and/or a chimeric antigen receptor protein. In some embodiments, a pharmaceutical composition described herein comprises a CAR-T cell which expresses an arginine transporter. In some embodiments, a pharmaceutical composition of the invention comprises a CAR-T cell, wherein the CAR-T cell comprises one or more recombinant nucleic acid sequences encoding an arginine transporter and/or a chimeric antigen receptor protein. In some embodiments, a pharmaceutical composition described herein comprises a CAR-T cell which expresses an arginine transporter, for example, a recombinant protein arginine transporter. In various embodiments, the arginine transporter is selected from the group consisting of CAT-1, CAT-2, CAT-3, CAT-4, y⁺LAT1, 4F2hc, y⁺LAT2, y⁺LAT1 and 4F2hc, y⁺LAT2 and 4F2hc, b^{0,+}AT, rBAT, b^{0,+}AT and rBAT, and ATB^{0,+}, or a combination thereof. In some embodiments, the one or more nucleic acid sequences encoding an arginine transporter comprises one or more recombinant arginine transporter nucleic acid sequences, for example a recombinant CAT-1 nucleic acid sequence, a recombinant CAT-2 nucleic acid sequence, a recombinant CAT-3 nucleic acid sequence, a recombinant CAT-4 nucleic acid sequence, a recombinant y⁺LAT1 nucleic acid sequence, a recombinant 4F2hc nucleic acid sequence, a recombinant y⁺LAT2 nucleic acid sequence, a recombinant y⁺LAT1 nucleic acid sequence and a recombinant 4F2hc nucleic acid sequence, a recombinant y⁺LAT2 nucleic acid sequence and a recombinant 4F2hc nucleic acid sequence, a recombinant b^{0,+}AT nucleic acid sequence, a recombinant rBAT nucleic acid sequence, a recombinant b^{0,+}AT nucleic acid sequence and a recombinant rBAT nucleic acid sequence, or a recombinant ATB^{0,+} nucleic acid sequence. In some embodiments, the arginine transporter is a recombinant arginine transporter protein, for example, a recombinant CAT-1, a recombinant CAT-2, a recombinant CAT-3, a recombinant CAT-4, a recombinant y⁺LAT1, a recombinant 4F2hc, a recombinant y⁺LAT2, a recombinant y⁺LAT1 and a recombinant 4F2hc, a recombinant y⁺LAT2 and a recombinant 4F2hc, a recombinant b^{0,+}AT, a recombinant rBAT, a recombinant b^{0,+}AT and a recombinant rBAT, or a recombinant ATB^{0,+}.

In another aspect, a pharmaceutical composition described herein is packaged as a kit. For example, in some embodiments, a pharmaceutical composition comprising a CAR-T cell which expresses an arginine transporter and a chimeric antigen receptor protein (for example, a genetically modified CAR-T cell which expresses an arginine transporter and a chimeric antigen receptor protein) is packaged as a kit. In some embodiments, a pharmaceutical composition comprising a T-cell which expresses an arginine transporter (for example, a genetically modified T-cell which expresses an arginine transporter, for example, a recombinant arginine transporter protein) is packaged as a kit. A kit described herein can include instructions for administering the CAR-T cells to a patient in need of treatment. A kit described herein can include instructions for priming CAR-T cells for administration to a patient in need of treatment. In some embodiments, the kit may include at least one of buffers (for example, a buffer comprising levels of L-arginine sufficient for priming T-cells), reagents and detailed instructions for producing, administering, and/or priming CAR-T cells. In some embodiments, a kit described herein can include agents for producing CAR-T cells, including expression vectors, viral constructs, cells, transfection reagents and media, agents for cell selection (for example, antibodies), and/or growth media.

Also described herein are methods of treating cancer using a pharmaceutical composition described herein. For example, described herein is a method of treating a solid tumor cancer in a patient in need thereof, the method comprising administering to the patient an effective amount of a pharmaceutical composition described herein. For example, described herein is a method of treating a solid tumor cancer in a patient in need thereof, the method comprising administering to the patient an effective amount of a pharmaceutical composition comprising a genetically modified T-cell described herein (for example, a CAR-T cell or a T-cell genetically modified to express an amino acid transporter) and a pharmaceutically acceptable excipient.

Also described herein are methods of treating a hematological cancer using a pharmaceutical composition described herein. For example, described herein is a method of treating a hematological cancer in a patient in need thereof, the method comprising administering to the patient an effective amount of a pharmaceutical composition described herein. For example, described herein is a method of treating a hematological cancer in a patient in need thereof, the method comprising administering to the patient an effective amount of a pharmaceutical composition comprising a genetically modified T-cell described herein (for example, a CAR-T cell or a T-cell genetically modified to express an amino acid transporter) and a pharmaceutically acceptable excipient.

Also described herein are methods of modulating intracellular arginine levels (for example, intracellular T-cell arginine levels) to effect a T cell-mediated immune response in a patient in need of treatment. For example, described herein is a method of modulating intracellular arginine levels to effect a T cell-mediated immune response in a patient in need thereof, the method comprising modulating intracellular arginine levels of a genetically modified T-cell. In some embodiments, the method of modulating intracellular arginine levels to effect a T cell-mediated immune response in a patient in need thereof further comprises administering to the patient an effective amount of a pharmaceutical composition described herein (e.g., a pharmaceutical composition comprising a genetically modified T-cell described herein and a pharmaceutically acceptable excipient, wherein the genetically modified T-cell has been subjected to conditions effective to increase intracellular arginine levels). In some embodiments, the method of modulating intracellular arginine levels to effect a T cell-mediated immune response in a patient in need thereof comprises modulating intracellular arginine levels of a genetically modified T-cell and administering to the patient an effective amount of a pharmaceutical composition comprising the genetically modified T-cells and a pharmaceutically acceptable excipient.

In yet another aspect, described herein is a method for treating a condition in a human patient in need thereof, the method comprising: administering to the human patient a therapeutically effective amount of a composition comprising a CAR-T cell (for example, a genetically modified CAR-T cell) which expresses an arginine transporter (for example, a recombinant arginine transporter) and a chimeric antigen receptor protein. In some embodiments, a method for treating a condition in a human patient in need thereof comprises administering to the human patient a therapeutically effective amount of a composition comprising a CAR-T cell described herein, for example, a genetically modified CAR-T cell described herein. For example, in some embodiments, a method for treating a condition in a human patient in need thereof comprises administering to the human patient a therapeutically effective amount of a composition comprising a CAR-T cell wherein the CAR-T cell comprises one or more recombinant nucleic acid sequences encoding an arginine transporter and/or a chimeric antigen receptor protein. In some embodiments described herein, a method for treating a condition in a human patient in need thereof comprises administering to the human patient a therapeutically effective amount of a composition comprising a genetically modified T cell that is genetically modified to express or overexpress an amino acid transporter, for example, an arginine transporter.

Also described herein is a method for modulating a T-cell-mediated immune response to a target cell population expressing a cell surface antigen in a patient in need thereof, the method comprising administering to the patient a therapeutically effective amount of genetically modified T-cells. In some embodiments the T-cells are: a) genetically modified to express a chimeric antigen receptor, wherein the chimeric antigen receptor comprises: at least one antigen-specific targeting region that specifically binds the cell surface antigen present on the target cell population, a transmembrane domain, an intracellular signaling domain; and b) genetically modified to express an arginine transporter (for example, a recombinant arginine transporter). In some embodiments the T-cells are genetically modified to express an arginine transporter (for example, a recombinant arginine transporter). For example, in some embodiments a T-cell for administering comprises one or more recombinant nucleic acid sequences encoding a chimeric antigen receptor, wherein the chimeric antigen receptor comprises: at least one antigen-specific targeting region that specifically binds the cell surface antigen present on the target cell population, a transmembrane domain, an intracellular signaling domain; and an arginine transporter. In some embodiments a T-cell for administering comprises a recombinant chimeric antigen receptor protein, wherein the recombinant chimeric antigen receptor protein comprises: at least one antigen-specific targeting region that specifically binds the cell surface antigen present on the target cell population, a transmembrane domain, an intracellular signaling domain; and a recombinant arginine transporter protein. In some embodiments a T-cell for administering comprises one or more recombinant nucleic acid sequences encoding an arginine transporter. In some embodiments a T-cell for administering comprises a recombinant arginine transporter protein.

In another aspect, the disclosure relates to a method of increasing T cell survival in a low arginine environment, the method comprising: administering a T cell comprising a recombinant arginine transporter to a low arginine environment. In certain embodiments, prior to the administering step, the method comprises transfecting the T cell with a DNA construct comprising a nucleotide sequence encoding the recombinant arginine transporter. In certain embodiments, the T-cell comprises a chimeric antigen receptor and/or comprises a DNA construct comprising a nucleotide sequence encoding a chimeric antigen receptor. In certain embodiments, the T cell is a CAR-T cell. In certain embodiments, prior to the administering step, the method comprises culturing the T cell or the CAR-T cell in a culture medium comprising arginine, for example, until the intracellular arginine level of the T cell or CAR-T cell accumulates to a certain level. In certain embodiments, the low arginine environment is a cell culture medium. In certain embodiments, the low arginine environment is a tumor microenvironment.

In embodiments described herein, a disclosed method can include a step of culturing T-cells in a culture medium comprising arginine before administering (for example, administering to a patient in need of treatment). For example, described herein is a method for treating a condition in a human patient in need thereof, the method comprising: culturing T-cells in a culture medium comprising arginine before administering a therapeutically effective amount of a composition comprising the T-cells to the human patient. Also described herein is a method for modulating a T cell-mediated immune response to a target cell population expressing a cell surface antigen in a patient in need thereof, the method comprising: culturing genetically modified T-cells in a culture medium comprising arginine before administering to the patient a therapeutically effective amount of the T-cells.

In a method described herein, the arginine transporter is selected from the group consisting of CAT-1, CAT-2, CAT-3, CAT-4, y⁺LAT1, 4F2hc, y⁺LAT2, y⁺LAT1 and 4F2hc, y⁺LAT2 and 4F2hc, b^{0,+}AT, rBAT, b^{0,+}AT and rBAT, and ATB^{0,+}, or a combination thereof. For example, described herein is a method for treating a condition in a human patient in need thereof, the method comprising: administering to the human patient a therapeutically effective amount of a composition comprising a CAR-T cell which expresses a chimeric antigen receptor protein and an arginine transporter (for example, a recombinant arginine transporter) selected from the group consisting of CAT-1, CAT-2, CAT-3, CAT-4, y⁺LAT1, 4F2hc, y⁺LAT2, y⁺LAT1 and 4F2hc, y⁺LAT2 and 4F2hc, b^{0,+}AT, rBAT, b^{0,+}AT and rBAT, and ATB^{0,+}, or a combination thereof. Also described herein is a method for modulating a T cell-mediated immune response to a target cell population expressing a cell surface antigen in a patient in need thereof, the method comprising administering to the patient a therapeutically effective amount of T-cells genetically modified to express a chimeric antigen receptor and an arginine transporter (for example, a recombinant arginine transporter) selected from the group consisting of CAT-1, CAT-2, CAT-3, CAT-4, y⁺LAT1, 4F2hc, y⁺LAT2, y⁺LAT1 and 4F2hc, y⁺LAT2 and 4F2hc, b^{0,+}AT, rBAT, b^{0,+}AT and rBAT, and ATB^{0,+}, or a combination thereof. In some embodiments, the arginine transporter is a recombinant arginine transporter protein, for example a recombinant CAT-1, a recombinant CAT-2, a recombinant CAT-3, a recombinant CAT-4, a recombinant y⁺LAT1, a recombinant 4F2hc, a recombinant y⁺LAT2, a recombinant y⁺LAT1 and a recombinant 4F2hc, a recombinant y⁺LAT2 and a recombinant 4F2hc, a recombinant b^{0,+}AT, a recombinant rBAT, a recombinant b^{0,+}AT and a recombinant rBAT, or a recombinant ATB^{0,+}. In certain embodiments, the arginine transporter comprises a nucleic acid sequence selected from the group consisting of: SEQ ID NO: 180, 184-188, 204, 205, 210, 214, 215, 220-222, 227-230, 234-236, 242, and 246, or a fragment or a variant thereof. In certain embodiments, the arginine transporter comprises a nucleic acid expressing a sequence having about 90%, 95%, or 99% percent identity to one of SEQ ID NO: 180, 184-188, 204, 205, 210, 214, 215, 220-222, 227-230, 234-236, 242, and 246.

In some embodiments, the method further comprises administering a second therapeutic agent to the human patient. For example, described herein are methods for treating a condition in a human patient in need thereof, the methods comprising administering a therapeutically effective amount of a composition comprising a CAR-T cell and administering a second therapeutic agent to the human patient. In some embodiments, a method described herein comprises administering the second therapeutic agent before, during, or after the administering of a composition comprising a CAR-T cell. Also described herein are methods for modulating a T cell-mediated immune response to a target cell population expressing a cell surface antigen in a patient in need thereof, the methods comprising administering to the patient a therapeutically effective amount of genetically modified T-cells and administering a second therapeutic agent to the human patient. In some embodiments, a method described herein comprises administering the second therapeutic agent before, during or after the administering of a therapeutically effective amount of T-cells.

In some embodiments, the second therapeutic agent is a checkpoint protein inhibitor, for example, a checkpoint protein inhibitor that inhibits checkpoint protein activity or checkpoint protein signaling, for example, an antibody that inhibits a checkpoint protein or checkpoint protein signaling. For example, in some embodiments, the second therapeutic agent is an anti-PD-1 antibody, an anti-PD-L1 antibody, or an anti-CTLA-4 antibody. In some embodiments, the second therapeutic agent is a DNA damage and repair inhibitor. For example, in some embodiments, the DNA damage and repair inhibitor is an ATM/ATR inhibitor, a PARP inhibitor, a WEE1 inhibitor, a Chk1 inhibitor, a Chk2 inhibitor, or a DNA-dependent protein kinase (DNA-PK) inhibitor.

In embodiments described herein, a composition comprising CAR-T cells is administered to a human patient once every week, once every 2 weeks, once every 3 weeks, or once every 4 weeks. For example, described herein is a method for treating a condition in a human patient in need thereof, the method comprising administering to the human patient a therapeutically effective amount of a composition comprising a CAR-T cell once every week, once every 2 weeks, once every 3 weeks, or once every 4 weeks. Also described herein is a method for modulating a T cell-mediated immune response to a target cell population expressing a cell surface antigen in a patient in need thereof, the method comprising administering to the patient a therapeutically effective amount of genetically modified T-cells once every week, once every 2 weeks, once every 3 weeks, or once every 4 weeks.

In some embodiments, the methods described herein comprise administering a specified number of CAR-T cells based on the weight of the patient or a specified range of CAR-T cells based on the weight of the patient. In some embodiments, a method described herein comprises administering about 10², about 10³, about 10⁴, about 10⁵, about 10⁶, about 10⁷, about 10⁸, about 10⁹, about 10¹⁰, about 10¹¹, about 10¹², about 10¹³, about 10¹⁴, about 10¹⁵, about 10¹⁶, about 10¹⁷, about 10¹⁸, about 10¹⁹, about 10²⁰, about 10²⁵, about 10³⁰, about 10³⁵, about 10⁴⁰, about 10⁴⁵, or about 10⁵⁰ CAR-T cells per kilogram of the patient. In some embodiments, a method described herein comprises administering about 10² to 10⁷, about 10² to 10¹⁰, about 10³ to 10¹⁰, about 10⁴ to 10¹⁰, about 10⁵ to 10¹⁰, about 10⁶ to 10¹⁰, about 10⁷ to 10¹⁰, about 10⁸ to 10¹¹, about 10⁹ to 10¹², about 10¹⁰ to 10¹³, about 10⁷ to 10¹⁵, about 10⁵ to 10¹⁵, about 10¹⁰ to 10²⁰, about 10¹⁰ to 10²⁵, about 10¹⁰ to 10³⁰, about 10⁷ to 10²⁰, about 10⁷ to 10²⁵, about 10¹⁰ to 10⁵⁰, or about 10⁷ to 10⁵⁰ CAR-T cells per kilogram of the patient. For example, in some embodiments, a method described herein comprises administering about 10⁷ to 10¹⁰ CAR-T cells per kilogram of the patient.

Embodiments described herein include a method of making a genetically modified CAR-T cell that expresses an arginine transporter, the method comprising: transfecting a T-cell with a DNA construct comprising a nucleotide sequence for a specific chimeric antigen receptor and for an arginine transporter thereby producing a genetically modified CAR-T cell that expresses both the chimeric antigen receptor and the arginine transporter; and culturing the genetically modified CAR-T cell in a culture medium comprising arginine. Embodiments described herein also include a method of making a genetically modified CAR-T cell that expresses an arginine transporter, the method comprising: transducing a T-cell with a virus that includes a nucleotide construct comprising a nucleotide sequence for a specific chimeric antigen receptor and for an arginine transporter thereby producing a genetically modified CAR-T cell that expresses both the chimeric antigen receptor and the arginine transporter; and culturing the genetically modified CAR-T cell in a culture medium comprising arginine. In some embodiments, the genetically modified CAR-T cell expresses a recombinant arginine transporter nucleotide sequence. In some embodiments, the genetically modified CAR-T cell expresses a recombinant arginine transporter protein. In some embodiments, culturing comprises culturing the genetically modified CAR-T cell in the culture medium until the intracellular arginine level of the CAR-T cell accumulates to a certain level. In some embodiments, the intracellular arginine level of the CAR-T cell is an intracellular arginine level that allows the CAR-T cell to survive in a tumor microenvironment. For example, in some embodiments, culturing comprises culturing the genetically modified CAR-T cell in the culture medium until the intracellular arginine level of the CAR-T cell is about 500 µM, about 600 µM, about 700 µM, about 800 µM, about 900 µM, about 1,000 µM, about 1,100 µM, about 1,200 µM, about 1,300 µM, about 1,400 µM, about 1,500 µM, about 1,600 µM, about 1,700 µM, about 1,800 µM, about 1,900 µM, about 2,000 µM, about 2,500 µM, about 3,000 µM, about 3,500 µM, or about 4,000 µM. In some embodiments, culturing comprises culturing the genetically modified CAR-T cell in the culture medium until the intracellular arginine level of the CAR-T cell is about 500 µM to about 1,000 µM, about 800 µM to about 1,200 µM, about 1,000 µM to about 1,500 µM, about 1,000 µM to about 2,000 µM, about 1,500 µM to about 2,000 µM, about 700 µM to about 900 µM, about 900 µM to about 1,100 µM, about 900 µM to about 1,200 µM, or about 1,300 µM to about 1,500 µM.

Embodiments described herein also include a method of making a genetically modified T cell that expresses an arginine transporter, the method comprising: transfecting a T-cell with a DNA construct comprising a nucleotide sequence for an arginine transporter thereby producing a genetically modified T cell that expresses the arginine transporter; and culturing the genetically modified T cell in a culture medium comprising arginine. Embodiments described herein also include a method of making a genetically modified T cell that expresses an arginine transporter, the method comprising: transducing a T-cell with a virus that includes a nucleotide construct comprising a nucleotide sequence for an arginine transporter thereby producing a genetically modified T cell that expresses the arginine transporter; and culturing the genetically modified T cell in a culture medium comprising arginine. In some embodiments, culturing comprises culturing the genetically modified T cell in the culture medium until the intracellular arginine level of the T cell accumulates to a certain level. In some embodiments, the intracellular arginine level of the T cell is an intracellular arginine level that allows the T cell to survive in a tumor microenvironment or an arginine-depleted environment. For example, in some embodiments, culturing comprises culturing the genetically modified T cell in the culture medium until the intracellular arginine level of the T cell is about 500 µM, about 600 µM, about 700 µM, about 800 µM, about 900 µM, about 1,000 µM, about 1,100 µM, about 1,200 µM, about 1,300 µM, about 1,400 µM, about 1,500 µM, about 1,600 µM, about 1,700 µM, about 1,800 µM, about 1,900 µM, about 2,000 µM, about 2,500 µM, about 3,000 µM, about 3,500 µM, or about 4,000 µM. In some embodiments, culturing comprises culturing the genetically modified T cell in the culture medium until the intracellular arginine level of the T cell is about 500 µM to about 1,000 µM, about 800 µM to about 1,200 µM, about 1,000 µM to about 1,500 µM, about 1,000 µM to about 2,000 µM, about 1,500 µM to about 2,000 µM, about 700 µM to about 900 µM, about 900 µM to about 1,100 µM, about 900 µM to about 1,200 µM, or about 1,300 µM to about 1,500 µM.

### BRIEF DESCRIPTION OF THE FIGURES

FIG. 1 is a map of the pBCTex01G expression vector. FIG. 1 discloses “(G4S)3” as SEQ ID NO: 30.

FIG. 2 is a map of the pBCTex02mini expression vector.

FIG. 3A is a schematic showing transfection and arginine depletion steps of the experiment described in Example 1.

FIG. 3B is a schematic showing cell filtering and counting steps of the experiment described in Example 1.

FIG. 3C is a set of graphs showing the estimated change in percent of cells transfected with an expression construct (Control, CAT, or ASS) after 72 hours in an arginine-rich (left) or arginine-depleted (right) environment. Each data point represents the estimated percent change in cell number of one isolated well of independently transfected cells.

FIG. 4 is a set of graphs showing the estimated change in percent of primary human T cells transfected with control (mNeonGreen) or CAT (arginine transporter) mRNA in control (top) or arginine-depleted (bottom) media. An increase in percentage of cells was seen in both GFP control (~100%) and CAT (~200%) transfected cells after 24 hours in arginine-rich medium. In contrast, in arginine-depleted medium, a net decrease in GFP control cells was seen while a ~15% increase was seen in cells transfected with CAT mRNA.

### DETAILED DESCRIPTION

### Definitions

As used herein, the term “chimeric antigen receptor” (CAR) in general refers to a genetically engineered receptor that is designed to bind to a specific antigen, for example, an antigen presented on the surface of a cancer cell. A CAR can be introduced to immune cells to help them identify and kill cancer cells that express the specific antigen.

As used herein the term “T-lymphocyte” or “T-cell” in general refers to a type of immune cell that is distinguished from other lymphocytes by the presence of a T-cell receptor on the cell surface. Differentiated T-cells play many important roles in controlling and shaping the immune response through several immune related functions such as immune-mediated cell death, recruiting cells when mounting an immune response through cytokines, determining if and how other parts of the immune system respond to a specific perceived threat, influencing regulatory B-cells, and distinguishing foreign cells from themselves among other functions.

As used herein the term “co-stimulatory signaling region” refers to a portion of a CAR comprising the intracellular domain of a costimulatory molecule. Co-stimulatory molecules are cell surface molecules other than antigen receptors or their ligands that are required for an efficient response of lymphocytes to antigen. Examples of co-stimulatory signaling molecules include CD28, ICOS (CD278), 4-1BB (CD137), OX40 (CD134), CD27, CD40, CD40L, TLRs (e.g., TLR2), DAP10, IL-2RB, IL-2RA, and MYD88.

As used herein the term “CAR-T cell therapy” in general refers to a genetically engineered T-cell (CAR-T cell) in which receptor proteins have been genetically incorporated into an existing lymphocyte. Such receptor proteins can give the engineered CAR-T cells the ability to target a specific protein. “CAR-T cell therapy” can also refer to methods of treatment that include administration of a CAR-T cell or a CAR-T cell pharmaceutical composition.

As used herein, the term “host cell” means any cell of an organism that is selected, modified, transformed, grown, used or manipulated for the production of a substance by the cell, for example the expression by the cell of a gene, a DNA or RNA sequence, a protein or an enzyme. Host cells of the present invention include T-cells and NK cells that contain the DNA or RNA sequences encoding the chimeric receptor and express the chimeric receptor on the cell surface. Host cells may be used for enhancing T lymphocyte activity in the treatment of cancer.

As used herein, the terms “express” and “expression” mean allowing or causing the information in a gene or DNA sequence to become manifest, for example producing a protein such as a CAR or an amino acid transporter by activating the cellular functions involved in transcription and translation of a corresponding gene or DNA sequence. As used herein the terms “overexpression” and “overexpressing” generally refer to the enhanced expression of a protein by engineered ectopic expression, which results in artificial induction or enhancement of gene and subsequent protein expression of the target modalities at higher than normal levels. A DNA sequence is expressed in or by a cell to form an “expression product” such as a protein. The expression product itself, e.g., the resulting protein, may also be said to be “expressed” by the cell. An expression product can be characterized as intracellular, extracellular or transmembrane. The term “intracellular” means something that is inside a cell. The term “extracellular” means something that is outside a cell. The term transmembrane means something that has an extracellular domain outside the cell, a portion embedded in the cell membrane and an intracellular domain inside the cell.

As used herein, the term “expression construct coding” or “expression vector engineering” refers to a plasmid designed for gene expression in cells. This vector is used to introduce specific gene(s) into a target cell and can commandeer the cell’s mechanism for protein synthesis to produce a protein encoded by the gene. The vector is typically engineered to contain regulatory sequences that act as enhancer and promoter regions and lead to efficient transcription of the gene(s) carried on the expression vector. An expression vector can produce the protein of interest efficiently through production of modalities such as messenger RNA which can be translated into protein(s).

As used herein, the term “amino acid” in general refers to organic compounds that contain at least one amino group, —NH₂, which may be present in its ionized form, —NH₃⁺, and one carboxyl group, —COOH, which may be present in its ionized form, —COO⁻, where the carboxylic acids are deprotonated at neutral pH, having the basic formula of NH₂CHRCOOH. An amino acid and thus a peptide has an N (amino)-terminal residue region and a C (carboxy)-terminal residue region. Types of amino acids include at least 20 amino acids that are considered “natural” as they comprise the majority of biological proteins in mammals and include amino acids such as lysine, cysteine, tyrosine, threonine, etc. Amino acids may also be grouped based upon their side chains, such as those with a carboxylic acid groups (at neutral pH), including aspartic acid or aspartate (Asp; D) and glutamic acid or glutamate (Glu; E); and basic amino acids (at neutral pH), including lysine (Lys; L), arginine (Arg; N), and histidine (His; H).

As used herein the term “amino acid transporters” (AATs) refers to a membrane transport protein that can transport an amino acid, for example, arginine. More specifically these are membrane transport proteins that mediate transfer of amino acids into and out of cells or cellular organelles. As used herein the term “arginine transporters” refers to membrane transport proteins that are capable of transporting arginine across a cell membrane. “Arginine transporters” may transport other amino acids in addition to arginine. Non-limiting examples of arginine transporters are shown on Table 1. They play diverse functional roles in various biological systems which can modulate metabolic reprogramming, acid-base balance, and anabolic and catabolic reactions among others.

As used herein, the term “tumor microenvironment” (TME) in general refers to the environment within and surrounding a solid tumor including blood vessels, immune cells, fibroblasts, signaling molecules, and extracellular matrix. Tumor progression is profoundly influenced by interactions of cancer cells with this microenvironment and can determine metastasis, growth, and disease progression. The TME can shape therapeutic response and resistance by physically or chemically inhibiting therapeutic factors or contributing to metastasis.

As used herein, the term “metabolic reprogramming of T-cells” refers to their metabolic reprogramming during activation which is relevant for their acquisition of distinct differentiation profiles. During antigen encounter and activation, T-cells have increased bioenergetic and anabolic needs to support their rapid replication and production of soluble factors. To meet these needs, T-cells increase their uptake of glucose and amino acids for their utilization through a variety of processes including, but not limited to, glycolysis, glutaminolysis, catabolism of branched chain amino acids, uptake of fatty acids, lipid synthesis, and fatty acid oxidation. The role of amino acids as key metabolic regulators of T-cell differentiation and functional fate is well documented. Amino acids are able to serve as both a source of fuel during these metabolic demands as well as precursors for synthesis of proteins and nucleic acids.

As used herein, the term “myeloid derived suppressor cells” is used to refer to a heterogenous group of immune cells from the myeloid lineage. These cells are strongly expanded in pathological situations as a result of altered hematopoiesis. These cells possess strong immunosuppressive activities and interact with other immune cell types such as T-cells, dendritic cells, macrophages, and natural killer cells to regulate their functions. These cells are particularly relevant in cancer where their presence and upregulation are associated with poor patient prognosis and therapeutic resistance.

As used in the specification and claims of this application, the term “administering” includes any method which is effective to result in expression of a chimeric antigen receptor and arginine transporter(s) in T lymphocytes of the subject individual. One method for administering the chimeric antigen receptor is therefore by ex vivo transfection or transduction of peripheral blood T cells or hematopoietic progenitor cells (which would eventually be allogeneic) with a nucleic acid construct in accordance with the invention and returning the transfected or transduced cells, preferably after expansion to the subject individual. In embodiments described herein, administering an agent, for example, administering a CAR-T cell or a CAR expression vector, can include contacting a body fluid of a patient containing cells. For example, administering an agent can include contacting a body fluid of a patient containing a cancer cell (for example, a tumor cell) with the agent ex vivo. In embodiments described herein, “administering” an agent, for example, administering a CAR-T cell or a CAR expression vector, can include contacting a body fluid of a patient containing cells, for example, a cancer cell (for example, a tumor cell) with the agent in vivo.

As used herein, the term “administered in combination,” “combined administration,” or “co-administered” means that two or more agents are administered to a subject at the same time or within an interval such that there may be an additive or improved therapeutic effect of each agent on the patient when both are given as part of the same treatment regimen. Two or more agents that are administered in combination can be administered simultaneously or nearly simultaneously. Two or more agents that are administered in combination need not be administered together. In some embodiments, the agents are administered within 90 days (e.g., within 80, 70, 60, 50, 40, 30, 20, 10, 5, 4, 3, 2, or 1 day(s)), within 28 days (e.g., with 14, 7, 6, 5, 4, 3, 2, or 1 day(s)), within 24 hours (e.g., 12, 6, 5, 4, 3, 2, or 1 hour(s)), or within about 60, 30, 15, 10, 5, or 1 minute(s) of one another. In some embodiments, administrations of the agents are spaced sufficiently closely together such that a combinatorial effect is achieved.

The term “cancer” refers to any disease caused by the proliferation of malignant neoplastic cells, such as tumors, neoplasms, carcinomas, sarcomas, leukemias, and lymphomas. A “solid tumor cancer” is a cancer comprising an abnormal mass of tissue, e.g., sarcomas, carcinomas, and lymphomas. A “hematological cancer” or “liquid cancer,” as used interchangeably herein, is a cancer present in a body fluid, e.g., lymphomas and leukemias.

The term “refractory cancer” refers to a form of cancer that is unresponsive or which may be unresponsive to treatment with a currently used anti-cancer agent or current anti-cancer regimen. A refractory cancer may initially demonstrate responsiveness to treatment with an anti-cancer agent and later become unresponsive to treatment. For example, a refractory cancer can include a form of cancer where cancer cells fail to stop proliferating in response to treatment or which initially stop proliferating in response to treatment but re-commence proliferating despite further treatment with an anti-cancer agent. Apparent regression with a high frequency of recurrence is also considered refractory. A refractory cancer may be unresponsive to a specific anti-cancer treatment with first-line, second-line, or even third-line current treatments. A patient suffering from a refractory cancer may be referred to herein as a “refractory cancer patient.” Methods of the invention described herein can be used to treat, prevent, or ameliorate a refractory cancer or to treat a patient suffering from a refractory cancer.

The term an “effective amount” of an agent (e.g., a genetically modified T-cell), as used herein, is that amount sufficient to effect beneficial or desired results, such as clinical results, and, as such, an “effective amount” depends upon the context in which it is being applied.

The term “pharmaceutical composition,” as used herein, represents a composition containing a compound described herein formulated with a pharmaceutically acceptable excipient. In some embodiments, the pharmaceutical composition is manufactured or sold with the approval of a governmental regulatory agency as part of a therapeutic regimen for the treatment of disease in a mammal. Pharmaceutical compositions can be formulated, for example, parenteral, oral, pulmonary, intratracheal, intranasal, transdermal, or intraduodenal administration. In various embodiments, pharmaceutical compositions described herein can be administered by one or several routes, including parenterally, e.g., by subcutaneous or intravenous injection. The term parenteral as used herein includes subcutaneous injections, intrapancreatic administration, and intravenous, intramuscular, intraperitoneal, and intrasternal injection or infusion techniques. In embodiments described herein, pharmaceutical compositions can be administered intravenously to a patient in need of treatment of a cancer.

A “pharmaceutically acceptable excipient,” as used herein, refers any ingredient (for example, a vehicle capable of suspending or dissolving an active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, radioprotectants, sorbents, suspending or dispersing agents, sweeteners, or waters of hydration. Exemplary excipients include, but are not limited to: ascorbic acid, histidine, phosphate buffer, butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “polypeptide” as used herein refers to a string of at least two amino acids attached to one another by a peptide bond. In some embodiments, a polypeptide can include at least 3-5 amino acids, each of which is attached to others by way of at least one peptide bond. Those of ordinary skill in the art will appreciate that polypeptides can include one or more “non-natural” amino acids or other entities that nonetheless are capable of integrating into a polypeptide chain. In some embodiments, a polypeptide may be glycosylated, e.g., a polypeptide may contain one or more covalently linked sugar moieties. In some embodiments, a single “polypeptide” (e.g., an antibody polypeptide) may comprise two or more individual polypeptide chains, which may in some cases be linked to one another, for example by one or more disulfide bonds or other means.

By “patient” or “subject” is meant a human or non-human animal (e.g., a mammal). In some embodiments described herein, a patient is in need of treatment of a cancer. Such a patient may also be referred to as a “cancer patient.”

By “substantial identity” or “substantially identical” is meant a polypeptide or nucleotide sequence that has the same polypeptide or nucleotide sequence, respectively, as a reference sequence, or has a specified percentage of amino acid residues or nucleotides, respectively, that are the same at the corresponding location within a reference sequence when the two sequences are optimally aligned. For example, an amino acid sequence that is “substantially identical” to a reference sequence has at least 50%, 60%, 70%, 75%, 80%, 85%, 90%, 95%, 96%, 97%, 98%, 99%, or 100% identity to the reference amino acid sequence. For polypeptides, the length of comparison sequences will generally be at least 5, 6, 7, 8, 9, 10, 11, 12, 13, 14, 15, 16, 17, 18, 19, 20, 25, 50, 75, 90, 100, 150, 200, 250, 300, or 350 contiguous amino acids (e.g., a full-length sequence). Similarly, a nucleotide sequence that is “substantially identical” to a reference sequence has at least 50%, 60%, 70%, 75%, 80%, 85%, 90%, 95%, 96%, 97%, 98%, 99%, or 100% identity to the reference nucleotide sequence. For nucleotides, the length of comparison sequences will generally be at least 5, 6, 7, 8, 9, 10, 11, 12, 13, 14, 15, 16, 17, 18, 19, 20, 25, 50, 75, 90, 100, 150, 200, 250, 300, 350, 400, 500, 600, 700, 800, 900, 1000, 1500, 2000, 2500, 3000, 3500, 4000, 4500, 5000, 10000, or more than 10,000 contiguous nucleotides (e.g., a full-length sequence). Sequence identity may be measured using sequence analysis software on the default setting (e.g., Sequence Analysis Software Package of the Genetics Computer Group, University of Wisconsin Biotechnology Center, 1710 University Avenue, Madison, WI 53705). Such software may match similar sequences by assigning degrees of homology to various substitutions, deletions, and other modifications.

As used herein, and as well understood in the art, “to treat” a condition or “treatment” of the condition (e.g., the conditions described herein such as cancer) is an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment.

As used herein, the terms “decrease,” “decreased,” “increase,” “increased,” or “reduction,” “reduced,” (e.g., in reference to therapeutic outcomes or effects) have meanings relative to a reference level. In some embodiments, the reference level is a level as determined by the use of said method with a control in an experimental animal model or clinical trial. In some embodiments, the reference level is a level in the same subject before or at the beginning of treatment. In some embodiments, the reference level is the average level in a population not being treated by said method of treatment.

The term “DNA damage and repair inhibitor” (DDRi) refers to an agent which prevents the repair of cellular DNA damage caused by endogenous or exogenous chromosomal insults, and which acts through the inhibition of normally occurring DNA repair mechanisms and associated processes necessary for the maintenance of cellular viability.

The term “checkpoint inhibitor,” also known as “immune checkpoint inhibitor” or “ICI,” refers to an agent which blocks the action of an immune checkpoint protein, e.g., blocks such immune checkpoint proteins from binding to their partner proteins. Cancer cells are known to express immune checkpoint proteins, resulting in failure of T-cells to recognize such cancer cells as targets for destruction. In general, checkpoint inhibitors facilitate destruction of cancer cells by T-cells by blocking interactions between specific immune checkpoint proteins on T-cells and targeted cells, where such interactions would otherwise act as a signal to inhibit targeted cell destruction by T-cells. Checkpoint inhibitors include agents that block the interaction of PD-1 and PD-L1 or which block the interaction of CTLA-4 and B7-1/B7-2. Examples of specific checkpoint inhibitors include the following antibody-based drugs: ipilimumab, nivolumab, pembrolizumab, atezolizumab, avelumab, durvalumab, and cemiplimab.

As used herein, the term “tumor-associated antigen” or “tumor associated antigen” means an antigen that is present on tumor cells at a significantly greater amount than on normal cells.

As used herein, the term “tumor-specific antigen” or “tumor specific antigen” refers to an antigen that is endogenously present only on tumor cells.

As used herein, the term “cancer cell antigen” means an antigen that is present on cells that form part of a cancer (for example, malignant neoplastic cells, such as tumors, neoplasms, carcinomas, sarcomas, leukemias, and lymphomas). Individual cancer cells can express one or more cancer cell antigens. A preferred cancer cell antigen for targeting is one with significant differential expression on the cancer cells relative to healthy cells in a subject.

As used herein, the term “bind” or “binding,” for example, of an antibody, an antigen-binding fragment thereof, or an antigen-specific binding domain of a CAR, means an at least temporary interaction or association with or to a target antigen. For example, “bind” or “binding” can refer to the process of an antigen-binding portion of a CAR coming into temporary or sustained contact with a cancer cell expressing a cancer cell antigen. In some embodiments described herein, a CAR is capable of binding a cancer cell antigen. In such embodiments, binding occurs via interaction between the cancer cell antigen and the antigen-specific binding region of the CAR.

As used herein, a “primary tumor” refers to an original tumor growth at a primary site of origin and is not the product of metastasis.

As used herein, a “secondary tumor” refers to tumor growth that has spread from a primary site of origin to a secondary anatomical site, often through the process of metastasis.

A “solid tumor” is an abnormal mass of tissue, e.g., sarcomas, carcinomas, and lymphomas. A “liquid tumor” as used herein, is a cancer present in a body fluid, e.g., lymphomas and leukemias.

A “cold tumor” as used herein, refers to a tumor characterized by a lack of T-cell infiltration. Cold tumors are also characterized by ineffectiveness of checkpoint inhibitors with respect to treatment efficacy when used as a monotherapy. Examples of cold tumors include, without limitation, glioblastomas, ovarian cancer, prostate cancer, pancreatic cancer, and breast cancer tumors that are characterized by a lack of T cell infiltration.

### Detailed Description

### Chimeric Antigen Receptors

Chimeric antigen receptors (CARs) are genetically engineered cell surface receptor proteins designed to bind specific antigens, for example, antigens presented on the surface of a cancer cell. CARs can be expressed in immune cells, for example, T lymphocyte cells (T cells or T-cells), in order to direct T cells to target cells expressing the CAR-binding antigen and to target antigen-expressing cells for destruction. CARs described herein can bind to, for example, protein, carbohydrate, or glycolipid antigens. For example, CARs described herein can bind to any one of the following antigens: α-Folate receptor, CAIX, CD19, CD20, CD22, CD24, CD30, CD33, CD38, CD44v7/8, carcinoembryonic antigen (CEA), EGFRvIII, EGP-2, EGP-40, EphA2, EphA3, Erb-B2, Erb-B 2,3,4, FBP, Fetal acetylcholine receptor, G_{D2}, G_{D3}, HER2, HMW-MAA, IL-11Rα, IL-13Rα2, KDR, κ-light chain, Lewis Y, L1-cell adhesion molecule, Melanoma-associated antigen (MAGE), Mesothelin, Murine CMV infected cells, MUC1, MUC16, NKG2D, NY-ESO-1/LAGE-1, Oncofetal antigen, PSCA, PSMA, ROR1, mAb IgE, TAG-72, VEGF-R2, Insulin-like Growth Factor 1 Receptor (IGF-1R), Tumor Endothelial Marker 1 (TEM-1), alpha-fetoprotein (AFP), cancer antigen 125 (CA125), cancer antigen 15-3 (CA15-3), carbohydrate antigen 19-9 (CA19-9), human chorionic gonadotropin (hCG or beta-hCG), prostate-specific antigen (PSA), Epithelial tumor antigen (ETA), Immature laminin receptor, HPV E6, HPV E7, BING-4, Calcium-activated chloride channel 2, Cyclin-B1, 9D7, Ep-CAM, Telomerase, Mesothelin, SAP-1, Survivin, livin, BAGE family proteins, CAGE family proteins, GAGE family proteins, MAGE family proteins, SAGE family proteins, XAGE family proteins, PRAME, SSX-2, Melan-A/MART-1, MART-2, Gp100/pmel17, Tyrosinase, TRP-1/-2, P.polypeptide, MC1R, β-catenin, β-catenin-m, β-actin/4/m, myosin/m, HSP70-2/m, GM2, sTn, globo-H, HLA-A2-R17OJ, BRCA1/2, CDK4, CML66, Fibronectin, p53, Ras, TGF-βRII, or Mammaglobin-A. A CAR described herein can bind to a cancer cell antigen, including a tumor associated antigen or a tumor specific antigen.

CARs described herein include at least the following components: an antigen-binding fragment, a transmembrane domain component, and a cytoplasmic activation domain. CARs can also include one or more cytoplasmic co-stimulatory domains. Exemplary CARs are described, for example, in Feins et al. (2018) “An introduction to chimeric antigen receptor (CAR) T-cell immunotherapy for human cancer” Am. J. Hematol. 94:S3-9; Stoiber et al. (2019) “Limitations in the Design of Chimeric Antigen Receptors for Cancer Therapy” Cells, 8(472): 1-26; and Sadelain et al. (2013) “The Basic Principles of Chimeric Antigen Receptor Design” Cancer Discovery, 3(4):388-98.

In embodiments described herein, a CAR can include a hinge or spacer region. CAR antigen-binding fragments are generally connected to the CAR transmembrane domain via a hinge or space region. A hinge region can be an amino acid sequence of or derived from immunoglobulin G (IgG) or a CD8α or CD28 extracellular domain. Exemplary hinge domains are described in, for example, Stoiber et al. (2019) “Limitations in the Design of Chimeric Antigen Receptors for Cancer Therapy” Cells, 8(472): 1-26.

### CAR Antigen-Binding Fragments or Domains

CAR antigen-binding fragments can be single-chain variable fragments (scFv), antigen-binding fragments (Fab), F(ab′)₂s fragments, or ligands, for example, naturally occurring, artificial, or engineered ligands. scFvs are fusion proteins comprised of the variable regions of the heavy (V_{H}) and light chains (V_{L}) of an immunoglobulin, and held together by a peptide linker. Linkers of scFv’s can be, for example, 10, 11, 12, 13, 14, 15, 16, 17, 18, 19, 20, 21, 22, 23, 24, or 25 residues in length, for example, 10-20, 15-20, 15-25, or 10-25 residues in length. scFvs can be expressed as single chain peptides in mammalian or bacterial cells. scFvs can also be cloned in tandem with a linker region to create bivalent and trivalent scFvs. Additionally, two or more V_{H} and V_{L} pairs can be expressed where each V_{H} and V_{L} pair is attached by a short linker and each V_{H} dimerizes with a V_{L} of another linked V_{H} and V_{L} pair to form diabodies (i.e., two scFv’s formed by V_{H}/V_{L} dimerization) or triabodies (i.e., three scFv’s formed by V_{H}/V_{L} dimerization). Diabodies and triabodies can include linkers of short length, for example, about 5 amino acids. scFv linkers can include glycine and serine repeats, for example, the pentapeptide (Gly₄Ser) (SEQ ID NO: 275), (Gly₄Ser)₂ (SEQ ID NO: 276), (Gly₄Ser)₃ (SEQ ID NO: 30), or (Gly₄Ser)₄ (SEQ ID NO: 277). scFv amino acid sequences can be murine antibody sequences, human antibody sequences, or humanized antibody sequences. In some embodiments, a CAR can include two or three antigen-specific targeting regions, for example, two or three scFvs, Fabs, F(ab′)₂s, or ligands (for example, muteins) that bind to distinct cell surface antigens.

Fabs are comprised of a constant domain and a variable domain of each of a heavy and light antibody chain. Fabs can be prepared by direct cleavage of antibodies using enzymes such as papain, pepsin, or IdeS.

Examples of naturally occurring ligands that can be included in CARs include, but are not limited to, CD8, CD4, CD25, and CD16.

CARs include antigen-binding fragments or antigen-binding domains that recognize and bind to specific cell surface antigens. Exemplary CD33 antigen-recognition domain nucleotide sequences include the following:

GAAGTGCAGCTGGTGCAGAGCGGAGCAGAAGTGAAGAAGCCCGGAAGCAG
CGTGAAGGTGTCTTGCAAGGCCAGCGGCTACACCATCACCGACAGCAACA
TCCATTGGGTCCGGCAGGCTCCAGGACAGTCTCTGGAGTGGATCGGCTAC
ATCTACCCCTACAACGGCGGCACCGACTACAACCAGAAGTTCAAGAACCG
GGCCACCCTGACCGTGGATAACCCCACCAACACCGCCTACATGGAGCTGA
GCAGCCTGAGAAGCGAGGACACCGCCTTCTACTATTGCGTGAACGGCAAC
CCTTGGCTGGCCTATTGGGGACAGGGAACACTGGTGACCGTGTCCTCT (
SEQ ID NO:1); and

GACATCCAGCTGACCCAGTCTCCTAGCACCCTGAGCGCTAGCGTGGGAGA
TAGAGTGACCATCACTTGCAGAGCCAGCGAGAGCCTGGACAACTACGGCA
TCCGGTTCCTGACTTGGTTCCAGCAGAAACCCGGCAAGGCCCCTAAACTG
CTGATGTACGCCGCCTCTAACCAGGGAAGCGGAGTGCCTAGCAGATTCAG
CGGCAGCGGAAGCGGAACCGAGTTCACCCTGACCATCAGCTCTCTGCAGC
CAGACGACTTCGCCACCTACTACTGCCAGCAGACCAAGGAGGTGCCTTGG
AGCTTCGGCCAGGGAACCAAGGTGGAAGTGAAGCGGACAGTG (SEQ ID
NO:2).

Exemplary CD33 antigen-binding fragment amino acid sequences include the following: anti-CD33 heavy chain variable domain:

EVQLVQSGAEVKKPGSSVKVSCKASGYTITDSNIHWVRQAPGQSLEWIGY
IYPYNGGTDYNQKFKNRATLTVDNPTNTAYMELSSLRSEDTAFYYCVNGN
PWLAYWGQGTLVTVSS

(SEQ ID NO:3); and anti-CD33 light chain variable domain:

DIQLTQSPSTLSASVGDRVTITCRASESLDNYGIRFLTWFQQKPGKAPKL
LMYAASNQGSGVPSRFSGSGSGTEFTLTISSLQPDDFATYYCQQTKEVPW
SFGQGTKVEVKRTV (SEQ ID NO:4).

### CAR Hinge or Spacer Regions

Exemplary CD8α-derived hinge region nucleotide sequences include:

ACCACGACGCCAGCGCCGCGACCACCAACACCGGCGCCCACCATCGCGTC
GCAGCCCCTGTCCCTGCGCCCAGAGGCGTGCCGGCCAGCGGCGGGGGGCG
CAGTGCACACGAGGGGGCTGGACTTCGCCTGTGAT (SEQ ID NO:5);
and

GCGAAGCCCACCACGACGCCAGCGCCGCGACCACCAACACCGGCGCCCAC
CATCGCGTCGCAGCCCCTGTCCCTGCGCCCAGAGGCGTGCCGGCCAGCGG
CGGGGGGCGCAGTGCACACGAGGGGGCTGGACTTCGCCTGTGAT (SEQ
ID NO: 6).

Exemplary CD8α-derived hinge region amino acid sequences include:

TTTPAPRPPTPAPTIASQPLSLRPEACRPAAGGAVHTRGLDFACD (SEQ
ID NO: 7); and

AKPTTTPAPRPPTPAPTIASQPLSLRPEACRPAAGGAVHTRGLDFACD (
SEQ ID NO: 8).

An exemplary CD28-derived hinge region nucleotide sequence is the sequence of SEQ ID NO:9:

ATTGAAGTTATGTATCCTCCTCCTTACCTAGACAATGAGAAGAGCAATGG
AACCATTATCCATGTGAAAGGGAAACACCTTTGTCCAAGTCCCCTATTTC
CCGGACCTTCTAAGCCC.

An exemplary CD28-derived hinge region amino acid sequence is the sequence of SEQ ID NO:10:

IEVMYPPPYLDNEKSNGTIIHVKGKHLCPSPLFPGPSKP.

An exemplary IgG1-derived hinge region nucleotide sequence is the sequence of SEQ ID NO:11:

GAGCCCAAGAGCTGCGACAAGACCCACACCTGCCCCCCCTGCCCC.

An exemplary IgG1-derived hinge region amino acid sequence is the sequence of SEQ ID NO:12:

EPKSCDKTHTCPPCP.

An exemplary IgG2-derived hinge region nucleotide sequence is the sequence of SEQ ID NO:13:

ATTGAAGTTATGTATCCTCCTCCTTACCTAGACAATGAGAAGAGCAATGG
AACCATTATCCATGTGAAAGGGAAACACCTTTGTCCAAGTCCCCTATTTC
CCGGACCTTCTAAGCCC.

An exemplary IgG2-derived hinge region amino acid sequence is the sequence of SEQ ID NO:14:

ERKCCVECPPCP.

An exemplary IgG3-derived hinge region nucleotide sequence is the sequence of SEQ ID NO:15:

GAGCTCAAAACCCCACTTGGTGACACAACTCACACATGCCCACGGTGCCC
AGAGCCCAAATCTTGTGACACACCTCCCCCGTGCCCACGGTGCCCAGAGC
CCAAATCTTGTGACACACCTCCCCCATGCCCACGGTGCCCAGAGCCCAAA
TCTTGTGACACACCTCCCCCGTGCCCAAGGTGCCCA.

An exemplary IgG3-derived hinge region amino acid sequence is the sequence of SEQ ID NO:16:

ELKTPLGDTTHTCPRCPEPKSCDTPPPCPRCPEPKSCDTPPPCPRCPEPK
SCDTPPPCPRCP.

Exemplary IgG4-derived hinge region nucleotide sequences include:

GAGTCCAAATATGGTCCCCCATGCCCATCATGCCCA (SEQ ID NO: 1
7);

GAGTCCAAATATGGTCCCCCATGCCCATCATGCCCAGCA (SEQ ID NO
: 18);

GGGCAGCCCCGAGAGCCACAGGTGTACACCCTGCCCCCATCCCAGGAGGA
GATGACCAAGAACCAGGTCAGCCTGACCTGCCTGGTCAAAGGCTTCTACC
CCAGCGACATCGCCGTGGAGTGGGAGAGCAATGGGCAGCCGGAGAACAAC
TACAAGACCACGCCTCCCGTGCTGGACTCCGACGGCTCCTTCTTCCTCTA
CAGCAGGCTCACCGTGGACAAGAGCAGGTGGCAGGAGGGGAATGTCTTCT
CATGCTCCGTGATGCATGAGGCTCTGCACAACCACTACACACAGAAGAGC
CTCTCCCTGTCTCTGGGTAAA (SEQ ID NO:19);

GAGAGCAAGTACGGCCCCCCCTGCCCCCCCTGCCCCGGCGGCGGCAGCAG
CGGCGGCGGCAGCGGCGGCCAGCCCAGAGAGCCCCAGGTGTACACCCTGC
CCCCCAGCCAGGAGGAGATGACCAAGAACCAGGTGAGCCTGACCTGCCTG
GTGAAGGGCTTCTACCCCAGCGACATCGCCGTGGAGTGGGAGAGCAACGG
CCAGCCCGAGAACAACTACAAGACCACCCCCCCCGTGCTGGACAGCGACG
GCAGCTTCTTCCTGTACAGCAGACTGACCGTGGACAAGAGCAGATGGCAG
GAGGGCAACGTGTTCAGCTGCAGCGTGATGCACGAGGCCCTGCACAACCA
CTACACCCAGAAGAGCCTGAGCCTGAGCCTGGGCAAG (SEQ ID NO:2
0);

GAGAGCAAGTACGGCCCCCCCTGCCCCAGCTGCCCCGCCCCCGAGTTCGA
GGGCGGCCCCAGCGTGTTCCTGTTCCCCCCCAAGCCCAAGGACACCCTGA
TGATCAGCAGAACCCCCGAGGTGACCTGCGTGGTGGTGGACGTGAGCCAG
GAGGACCCCGAGGTGCAGTTCAACTGGTACGTGGACGGCGTGGAGGTGCA
CCAGGCCAAGACCAAGCCCAGAGAGGAGCAGTTCAACAGCACCTACAGAG
TGGTGAGCGTGCTGACCGTGCTGCACCAGGACTGGCTGAACGGCAAGGAG
TACAAGTGCAAGGTGAGCAACAAGGGCCTGCCCAGCAGCATCGAGAAGAC
CATCAGCAAGGCCAAGGGCCAGCCCAGAGAGCCCCAGGTGTACACCCTGC
CCCCCAGCCAGGAGGAGATGACCAAGAACCAGGTGAGCCTGACCTGCCTG
GTGAAGGGCTTCTACCCCAGCGACATCGCCGTGGAGTGGGAGAGCAACGG
CCAGCCCGAGAACAACTACAAGACCACCCCCCCCGTGCTGGACAGCGACG
GCAGCTTCTTCCTGTACAGCAGACTGACCGTGGACAAGAGCAGATGGCAG
GAGGGCAACGTGTTCAGCTGCAGCGTGATGCACGAGGCCCTGCACAACCA
CTACACCCAGAAGAGCCTGAGCCTGAGCCTGGGCAAG (SEQ ID NO:2
1); and

GAGAGCAAGTACGGCCCCCCCTGCCCCCCCTGCCCCGCCCCCGAGTTCGA
GGGCGGCCCCAGCGTGTTCCTGTTCCCCCCCAAGCCCAAGGACACCCTGA
TGATCAGCAGAACCCCCGAGGTGACCTGCGTGGTGGTGGACGTGAGCCAG
GAGGACCCCGAGGTGCAGTTCAACTGGTACGTGGACGGCGTGGAGGTGCA
CCAGGCCAAGACCAAGCCCAGAGAGGAGCAGTTCAACAGCACCTACAGAG
TGGTGAGCGTGCTGACCGTGCTGCACCAGGACTGGCTGAACGGCAAGGAG
TACAAGTGCAAGGTGAGCAACAAGGGCCTGCCCAGCAGCATCGAGAAGAC
CATCAGCAAGGCCAAGGGCCAGCCCAGAGAGCCCCAGGTGTACACCCTGC
CCCCCAGCCAGGAGGAGATGACCAAGAACCAGGTGAGCCTGACCTGCCTG
GTGAAGGGCTTCTACCCCAGCGACATCGCCGTGGAGTGGGAGAGCAACGG
CCAGCCCGAGAACAACTACAAGACCACCCCCCCCGTGCTGGACAGCGACG
GCAGCTTCTTCCTGTACAGCAGACTGACCGTGGACAAGAGCAGATGGCAG
GAGGGCAACGTGTTCAGCTGCAGCGTGATGCACGAGGCCCTGCACAACCA
CTACACCCAGAAGAGCCTGAGCCTGAGCCTGGGCAAG (SEQ ID NO:2
2).

Exemplary IgG4-derived hinge region amino acid sequences include:

ESKYGPPCPSCP (SEQ ID NO:23);

ESKYGPPCPSCPA (SEQ ID NO:24);

GQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKS
LSLSLGK (SEQ ID NO:25);

ESKYGPPCPPCPGGGSSGGGSGGQPREPQVYTLPPSQEEMTKNQVSLTCL
VKGFYPSDIAVEWESNGQPENNYKTTPPVLDSDGSFFLYSRLTVDKSRWQ
EGNVFSCSVMHEALHNHYTQKSLSLSLGK (SEQ ID NO:26);

ESKYGPPCPSCPAPEFEGGPSVFLFPPKPKDTLMISRTPEVTCVVVDVSQ
EDPEVQFNWYVDGVEVHQAKTKPREEQFNSTYRVVSVLTVLHQDWLNGKE
YKCKVSNKGLPSSIEKTISKAKGQPREPQVYTLPPSQEEMTKNQVSLTCL
VKGFYPSDIAVEWESNGQPENNYKTTPPVLDSDGSFFLYSRLTVDKSRWQ
EGNVFSCSVMHEALHNHYTQKSLSLSLGK (SEQ ID NO:27); and

ESKYGPPCPPCPAPEFEGGPSVFLFPPKPKDTLMISRTPEVTCVVVDVSQ
EDPEVQFNWYVDGVEVHQAKTKPREEQFNSTYRVVSVLTVLHQDWLNGKE
YKCKVSNKGLPSSIEKTISKAKGQPREPQVYTLPPSQEEMTKNQVSLTCL
VKGFYPSDIAVEWESNGQPENNYKTTPPVLDSDGSFFLYSRLTVDKSRWQ
EGNVFSCSVMHEALHNHYTQKSLSLSLGK (SEQ ID NO:28).

In embodiments described herein, a CAR can include a spacer region. An exemplary spacer region nucleotide sequence is:

GGCGGCGGAGGATCTGGCGGAGGTGGAAGCGGAGGCGGTGGAAGC (SEQ
ID NO:29).

An exemplary spacer region amino acid sequence is:

GGGGSGGGGSGGGGS (SEQ ID NO:30).

### CAR Transmembrane Domains

Transmembrane domains of CARs described herein link the antigen-binding domain and the intracellular signaling domain. A CAR transmembrane domain can be, for example, an amino acid sequence from or derived from CD4, CD8α, CD28, CD3ζ, or inducible T cell costimulator (ICOS). Transmembrane domains can contribute to CAR dimerization with the T-cell receptor (TCR) complex as well as CAR surface expression. Exemplary CAR transmembrane domains are described in, for example, Stoiber et al. (2019) “Limitations in the Design of Chimeric Antigen Receptors for Cancer Therapy” Cells, 8(472):1-26.

“CD4” (also known as T-cell surface glycoprotein CD4 and CD4mut) as used herein refers to the gene identified by Entrez Gene ID No. 920, allelic variants thereof, orthologs thereof, protein products thereof, and mRNA transcripts encoded by the gene, including the nucleotide sequence of NCBI Reference Sequence: NM_000616.5, NM_001195014.3, NM_001195015.3, NM_001195016.3, and NM_001195017.3. CD4 protein products include proteins encoded by CD4, for example, proteins comprising the amino acid sequence of NCBI Reference Sequence: NP_000607.1, NP_001181943.1, NP_001181944.1, NP_001181945.1, or NP_001181946.1. The transmembrane region of CD4 includes, for example, amino acids 397-418 of the amino acid sequence of NCBI Reference Sequence NP_000607.1, encoded by the following nucleotide sequence:

ATGGCCCTGATTGTGCTGGGGGGCGTCGCCGGCCTCCTGCTTTTCATTGG
GCTAGGCATCTTCTTC (SEQ ID NO:42).

“CD8α” (also known as CD8a molecule, T-cell surface glycoprotein CD8, p32, Leu2, and CD8a) as used herein refers to the gene identified by Entrez Gene ID No. 925, allelic variants thereof, orthologs thereof, protein products thereof, and mRNA transcripts encoded by the gene, including the nucleotide sequences of NCBI Reference Sequence: NM_001145873.1, NM_001768.6, and NM_171827.3. CD8 protein products include proteins encoded by CD8, for example, a protein comprising the amino acid sequence of NCBI Reference Sequence: NP_001139345.1, NP_001759.3, or NP_741969.1. The transmembrane region of CD8α includes, for example, amino acids 183-203, 183-205, or 183-206 of the amino acid sequence of NCBI Reference Sequence NP_001139345.1, respectively:

IYIWAPLAGTCGVLLLSLVIT (SEQ ID NO:49);

IYIWAPLAGTCGVLLLSLVITLY (SEQ ID NO:50); and

IYIWAPLAGTCGVLLLSLVITLYC (SEQ ID NO:51).

The transmembrane region of CD8α is encoded by the following nucleotide sequences:

ATCTACATCTGGGCGCCCTTGGCCGGGACTTGTGGGGTCCTTCTCCTGTC
ACTGGTTATCACC (SEQ ID NO:52);

ATCTACATCTGGGCGCCCTTGGCCGGGACTTGTGGGGTCCTTCTCCTGTC
ACTGGTTATCACCCTTTAC (SEQ ID NO:53); and

ATCTACATCTGGGCGCCCTTGGCCGGGACTTGTGGGGTCCTTCTCCTGTC
ACTGGTTATCACCCTTTACTGC (SEQ ID NO:54).

“CD28” (also known as T-cell-specific surface glycoprotein CD28, CD28 molecule, Tp44) as used herein refers to the gene identified by Entrez Gene ID No. 940, allelic variants thereof, orthologs thereof, protein products thereof, and mRNA transcripts encoded by the gene, including the nucleotide sequences of NCBI Reference Sequence: NM_001243077.2, NM_001243078.1, and NM_006139.4. CD28 protein products include proteins encoded by CD28, for example, a protein comprising the amino acid sequence of NCBI Reference Sequence: NP_001230006.1, NP_001230007.1, or NP_006130.1 or the mature CD28 protein comprising amino acids 19-220 of the amino acid sequence of NCBI Reference Sequence NP_006130.1. The transmembrane region of CD28 includes, for example, amino acids 153-179 of the amino acid sequence of NCBI Reference Sequence NP_006130.1:

FWVLVVVGGVLACYSLLVTVAFIIFWV (SEQ ID NO:62).

The transmembrane region of CD28 is encoded by the following nucleotide sequence:

TTTTGGGTGCTGGTGGTGGTTGGTGGAGTCCTGGCTTGCTATAGCTTGCT
AGTAACAGTGGCCTTTATTATTTTCTGGGTG (SEQ ID NO:63).

“CD3ζ” (also known as CD247, CD247 molecule, T-cell surface glycoprotein CD3 zeta chain, T3Z, CD3H, CD3Q, CD3Z, TCRZ, IMD25, and CD3-ZETA) as used herein refers to the gene identified by Entrez Gene ID No. 919, allelic variants thereof, orthologs thereof, protein products thereof, and mRNA transcripts encoded by the gene, including the nucleotide sequences of NCBI Reference Sequence: NM_000734.4 and NM_198053.2. CD3ζ protein products include proteins encoded by CD3ζ, for example, a protein comprising the amino acid sequence of NCBI Reference Sequence: NP_000725.1 or NP_932170.1. The transmembrane region of CD3ζ includes, for example, amino acids 31-51 of the amino acid sequence of NCBI Reference Sequence NP_000725.1: LCYLLDGILFIYGVILTALFL (SEQ ID NO:68).

The transmembrane region of CD3ζ is encoded by the following nucleotide sequence:

CTCTGCTACCTGCTGGATGGAATCCTCTTCATCTATGGTGTCATTCTCAC
TGCCTTGTTCCTG (SEQ ID NO:69).

“ICOS” (also known as inducible T cell costimulatory, inducible T-cell costimulator precursor, AILIM, CD278, and CVID1) as used herein refers to the gene identified by Entrez Gene ID No. 29851, allelic variants thereof, orthologs thereof, protein products thereof, and mRNA transcripts encoded by the gene, including the nucleotide sequence of NCBI Reference Sequence NM_012092.4. ICOS protein products include proteins encoded by ICOS, for example, a protein comprising the amino acid sequence of NCBI Reference Sequence NP_036224.1 (SEQ ID NO:71) or the mature ICOS protein comprising amino acids 21-199 of the amino acid sequence of NCBI Reference Sequence NP_036224.1 (SEQ ID NO:72). The transmembrane region of ICOS includes amino acids 141-161 of the amino acid sequence of NCBI Reference Sequence NP_036224.1:

FWLPIGCAAFVVVCILGCILI (SEQ ID NO:73).

In some embodiments described herein, a CAR can include the entirety of or a portion of a transmembrane domain described herein, for example, a CD4, CD8α, CD28, CD3ζ, or ICOS transmembrane domain described herein. For example, in some embodiments described herein, a CAR comprises a transmembrane domain of about 15, about 16, about 17, about 18, about 19, about 20, about 21, about 22, about 23, about 24, or about 25 amino acids, or from about 15 to about 20, from about 15 to about 25, from about 15 to about 22, from about 18 to about 20, from about 18 to about 22, or from about 18 to about 25 amino acids. For example, in some embodiments described herein, a CAR comprises a transmembrane domain of about 15, about 16, about 17, about 18, about 19, about 20, about 21, about 22, about 23, about 24, or about 25 amino acids, or from about 15 to about 20, from about 15 to about 25, from about 15 to about 22, from about 18 to about 20, from about 18 to about 22, or from about 18 to about 25 amino acids of a CD4, CD8α, CD28, CD3ζ, or ICOS transmembrane domain described herein.

In some embodiments described herein, a CAR includes a transmembrane domain with an amino acid sequence that is about 90%, about 91%, about 92%, about 93%, about 94%, about 95%, about 96%, about 97%, about 98%, about 99%, about 90% to about 95%, about 95% to about 100%, or about 90% to about 100% identical to a CD4, CD8α, CD28, CD3ζ, or ICOS transmembrane domain described herein.

### CAR Intracellular Signaling and Co-Stimulatory Domains

The intracellular portion of CARs described herein can include an intracellular signaling domain and, optionally, one or more co-stimulatory domains. Exemplary intracellular signaling domains include, for example, an amino acid sequence of or derived from an Fc Receptor γ chain subunit (FcRγ) or CD3ζ signaling domain. Exemplary co-stimulatory domains include, for example, an amino acid sequence from or derived from a 4-1BB (C137; TNFRS9), CD27, CD28, CD40, CD40L, TLR2, DAP10, OX40 (CD134), IL-2RB, IL-2RA, MYD88, or ICOS (CD278) intracellular domain. For example, a CAR described herein can include, but is not limited to, combinations of the following signaling and co-stimulatory domains: 4-1BB/CD3ζ, CD27/CD3ζ, CD28/CD3ζ, DAP10/CD3ζ, OX40/CD3ζ, ICOS/CD3ζ, 4-1BB/FcRγ, CD27/FcRγ, CD28/FcRγ, DAP10/FcRγ, OX40/FcRγ, ICOS/FcRγ, 4-1BB/CD28/CD3ζ, 4-1BB/CD28/FcRγ, OX40/CD28/CD3ζ, OX40/CD28/FcRγ, ICOS/4-1BB/CD3ζ, and ICOS/4-1BB/FcRγ.

A CD3ζ signaling domain described herein can include, for example, a protein comprising amino acids 52-163 of the amino acid sequence of NCBI Reference Sequence NP_000725.1:

RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR (SEQ ID NO:74).

A nucleotide sequence encoding a CD3ζ signaling domain is:

AGAGTGAAGTTCAGCAGGAGCGCAGACGCCCCCGCGTACCAGCAGGGCCA
GAACCAGCTCTATAACGAGCTCAATCTAGGACGAAGAGAGGAGTACGATG
TTTTGGACAAGAGACGTGGCCGGGACCCTGAGATGGGGGGAAAGCCGAGA
AGGAAGAACCCTCAGGAAGGCCTGTACAATGAACTGCAGAAAGATAAGAT
GGCGGAGGCCTACAGTGAGATTGGGATGAAAGGCGAGCGCCGGAGGGGCA
AGGGGCACGATGGCCTTTACCAGGGTCTCAGTACAGCCACCAAGGACACC
TACGACGCCCTTCACATGCAGGCCCTGCCCCCTCGC (SEQ ID NO:75
).

An example of a FcRγ protein is the Fc fragment of IgE receptor Ig (also known as FCER1G, FCRG, and high affinity immunoglobulin epsilon receptor subunit gamma), identified by Entrez Gene ID No. 2207. FcRγ nucleotide sequences described herein can include, for example, allelic variants, orthologs, and mRNA transcripts encoded by Entrez Gene ID No. 2207, including the nucleotide sequence of NCBI Reference Sequence NM_004106.2. FcRy protein products include proteins encoded by the nucleotide sequence of NCBI Reference Sequence NM_004106.2, for example, a protein comprising the amino acid sequence of NCBI Reference Sequence NP_004097.1 or the mature FcRγ protein comprising amino acids 19-86 of the amino acid sequence of NCBI Reference Sequence NP_004097.1. A signaling domain of FcRγ includes, for example, amino acids 45-86 of the amino acid sequence of NCBI Reference Sequence NP_001552.2:

CSPCPPNSFSSAGGQRTCDICRQCKGVFRTRKECSSTSNAEC (SEQ ID
NO:79).

FcRγ subunits and CD3ζ contain multiple YXXL immunoreceptor tyrosine-based activation motif (“ITAM”) sequences. Without being bound by theory, it is believed that tyrosine phosphorylation of ITAMs, for example, following cell-surface antigen binding by an antigen-binding portion of a CAR, promotes in T cell activation. An example of a CD3ζ amino acid sequence that includes ITAM sequences is SEQ ID NO:74. An example of a CD3ζ nucleotide sequence that encodes ITAM sequences is SEQ ID NO:75.

An example of a CD3ζ amino acid sequence that includes ITAM sequences is:

RVKFSRSADAPAYKQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR (SEQ ID NO:80).

In some embodiments described herein, a CAR can include the entirety of or a portion of a signaling domain described herein, for example, a CD3ζ or a FcRγ signaling domain described herein. For example, in some embodiments described herein, a CAR comprises a signaling domain of about 20, about 30, about 40, about 50, about 60, about 70, about 80, about 90, about 100, about 110, about 120, or about 130 amino acids, or from about 20 to about 40, from about 30 to about 50, from about 40 to about 50, from about 40 to about 60, from about 100 to about 120, from about 110 to about 120, or from about 110 to about 130 amino acids. For example, in some embodiments described herein, a CAR comprises about 20, about 30, about 40, about 50, about 60, about 70, about 80, about 90, about 100, about 110, about 120, or about 130 amino acids, or from about 20 to about 40, from about 30 to about 50, from about 40 to about 50, from about 40 to about 60, from about 100 to about 120, from about 110 to about 120, or from about 110 to about 130 amino acids comprising a CD3ζ or a FcRγ signaling domain described herein, or a portion thereof.

In some embodiments described herein, a CAR includes a transmembrane domain with an amino acid sequence that is about 90%, about 91%, about 92%, about 93%, about 94%, about 95%, about 96%, about 97%, about 98%, about 99%, about 90% to about 95%, about 95% to about 100%, or about 90% to about 100% identical to a CD3ζ or a FcRγ signaling domain described herein.

“4-1BB” (also known as TNFRSF9, TNF receptor superfamily member 9, CD137, ILA, CDw137, tumor necrosis factor receptor superfamily member 9, and TNFRS9), as used herein refers to the gene identified by Entrez Gene ID No. 3604, allelic variants thereof, orthologs thereof, protein products thereof, and mRNA transcripts encoded by the gene, including the nucleotide sequence of NCBI Reference Sequence NM_001561.6. 4-1BB protein products include proteins encoded by 4-1BB, for example, a protein comprising the amino acid sequence of NCBI Reference Sequence NP_001552.2 or the mature 4-1BB protein comprising amino acids 24-255 of the amino acid sequence of NCBI Reference Sequence NP_001552.2.

The transmembrane region of 4-1BB includes, for example, the following amino acid sequence:

IISFFLALTSTALLFLLFFLTLRFSVV (SEQ ID NO:84).

The transmembrane region of 4-1BB is encoded by the following nucleotide sequence:

ATCATCTCCTTCTTTCTTGCGCTGACGTCGACTGCGTTGCTCTTCCTGCT
GTTCTTCCTCACGCTCCGTTTCTCTGTTGTT (SEQ ID NO:85).

A co-stimulatory domain of 4-1BB includes, for example, amino acids 214-255 of the amino acid sequence of NCBI Reference Sequence NP_001552.2:

KRGRKKLLYIFKQPFMRPVQTTQEEDGCSCRFPEEEEGGCEL (SEQ ID
NO:86).

The co-stimulatory domain of 4-1BB is encoded by the following nucleotide sequence:

AAACGGGGCAGAAAGAAACTCCTGTATATATTCAAACAACCATTTATGAG
ACCAGTACAAACTACTCAAGAGGAAGATGGCTGTAGCTGCCGATTTCCAG
AAGAAGAAGAAGGAGGATGTGAACTG (SEQ ID NO:87).

“CD27” (also known as CD27 molecule, T14, S152, Tp55, TNFRSF7, S152, LPFS2, and CD27 antigen), as used herein refers to the gene identified by Entrez Gene ID No. 939, allelic variants thereof, orthologs thereof, protein products thereof, and mRNA transcripts encoded by the gene, including the nucleotide sequence of NCBI Reference Sequence NM_001242.4. CD27 protein products include proteins encoded by CD27, for example, a protein comprising the amino acid sequence of NCBI Reference Sequence NP_001233.1 or the mature CD27 protein comprising amino acids 21-260 of the amino acid sequence of NCBI Reference Sequence NP_001233.1. A co-stimulatory domain of CD27 includes, for example, amino acids 213-260 of the amino acid sequence of NCBI Reference Sequence NP_001233.1:

QRRKYRSNKGESPVEPAEPCRYSCPREEEGSTIPIQEDYRKPEPACSP (
SEQ ID NO:91).

A co-stimulatory domain of CD28 includes, for example, amino acids 180-220 of the amino acid sequence of NCBI Reference Sequence NP_006130.1:

RSKRSRLLHSDYMNMTPRRPGPTRKHYQPYAPPRDFAAYRS (SEQ ID
NO:92).

CD28 co-stimulatory domains described herein also include:

RSKRSRGGHSDYMNMTPRRPGPTRKHYQPYAPPRDFAAYRS (SEQ ID
NO:93).

Nucleotide sequences encoding a CD28 co-stimulatory domain include:

AGGAGTAAGAGGAGCAGGCTCCTGCACAGTGACTACATGAACATGACTCC
CCGCCGCCCCGGGCCCACCCGCAAGCATTACCAGCCCTATGCCCCACCAC
GCGACTTCGCAGCCTATCGCTCC (SEQ ID NO:94); and

AGGAGTAAGAGGAGCAGGGGCGGCCACAGTGACTACATGAACATGACTCC
CCGCCGCCCCGGGCCCACCCGCAAGCATTACCAGCCCTATGCCCCACCAC
GCGACTTCGCAGCCTATCGCTCC (SEQ ID NO:95).

“CD40” (also known as CD40 molecule, p50, Bp50, CDW40, TNFRSF5, and tumor necrosis factor receptor superfamily member 5), as used herein refers to the gene identified by Entrez Gene ID No. 958, allelic variants thereof, orthologs thereof, protein products thereof, and mRNA transcripts encoded by the gene, including the nucleotide sequence of NCBI Reference Sequence: NM_001250.6, NM_001302753.2, NM_001322421.2, NM_001322422.2, NM_001362758.2, or NM_152854.4. CD40 protein products include proteins encoded by CD40, for example, a protein comprising the amino acid sequence of NCBI Reference Sequence: NP_001241.1, NP_001289682.1, NP_001309350.1, NP_001309351.1, NP_001349687.1, NP_690593.1, or, for example, the mature CD40 protein comprising amino acids 21-277 of the amino acid sequence of NCBI Reference Sequence NP_001241.1. A co-stimulatory domain of CD40 includes, for example, amino acids 216-277 of the amino acid sequence of NCBI Reference Sequence NP_001241.1:

KKVAKKPTNKAPHPKQEPQEINFPDDLPGSNTAAPVQETLHGCQPVTQED
GKESRISVQERQ (SEQ ID NO:109).

“CD40L” (also known as CD40 ligand, CD40LG, IGM, IMD3, TRAP, gp39, CD154, HIGM1, T-BAM, TNFSF5, and hCD40L), as used herein refers to the gene identified by Entrez Gene ID No. 959, allelic variants thereof, orthologs thereof, protein products thereof, and mRNA transcripts encoded by the gene, including the nucleotide sequence of NCBI Reference Sequence NM_000074.3. CD40L protein products include proteins encoded by CD40L, for example, a protein comprising the amino acid sequence of NCBI Reference Sequence NP_000065.1. A co-stimulatory domain of CD40L includes, for example, amino acids 1-22 of the amino acid sequence of NCBI Reference Sequence NP_000065.1:

MIETYNQTSPRSAATGLPISMK (SEQ ID NO: 112).

“TLR2” (also known as toll like receptor 2, TIL4, and CD282), as used herein refers to the gene identified by Entrez Gene ID No. 7097, allelic variants thereof, orthologs thereof, protein products thereof, and mRNA transcripts encoded by the gene, including the nucleotide sequence of NCBI Reference Sequence: NM_001318787.2, NM_001318789.2, NM_001318790.2, NM_001318791.2, NM_001318793.2, NM_001318795.2, NM_001318796.2, and NM_003264.5. TLR2 protein products include proteins encoded by TLR2, for example, a protein comprising the amino acid sequence of NCBI Reference Sequence: NP_001305716.1, NP_001305718.1, NP_001305719.1, NP_001305720.1, NP_001305722.1, NP_001305724.1, NP_001305725.1, NP_003255.2, or the mature TLR2 protein comprising amino acids 21-784 of the amino acid sequence of NCBI Reference Sequence NP_001305716.1. A co-stimulatory domain of TLR2 includes, for example, amino acids 610-784 of NCBI Reference Sequence NP_001305716.1:

HRFHGLWYMKMMWAWLQAKRKPRKAPSRNICYDAFVSYSERDAYWVENLM
VQELENFNPPFKLCLHKRDFIPGKWIIDNIIDSIEKSHKTVFVLSENFVK
EWCKYELDFSHFRLFDENNDAAILILLEPIEKKAIPQRFCKLRKIMNTKT
YLEWPMDEAQREGFWVNLRAAIKS (SEQ ID NO:130); or

amino acids 640-784 of the amino acid sequence of NCBI Reference Sequence NP_001305716.1:

CYDAFVSYSERDAYWVENLMVQELENFNPPFKLCLHKRDFIPGKWIIDNI
IDSIEKSHKTVFVLSENFVKSEWCKYELDFSHFRLFDENNDAAILILLEP
IEKKAIPQRFCKLRKIMNTKTYLEWPMDEAQREGFWVNLRAAIKS (SEQ
ID NO:131).

“DAP10” (also known as HCST, hematopoietic cell signal transducer, KAP10, and PIK3AP), as used herein refers to the gene identified by Entrez Gene ID No. 10870, allelic variants thereof, orthologs thereof, protein products thereof, and mRNA transcripts encoded by the gene, including the nucleotide sequence of NCBI Reference Sequence: NM_001007469.2 or NM_014266.4. DAP10 protein products include proteins encoded by DAP10, for example, a protein comprising the amino acid sequence of NCBI Reference Sequence: NP_001007470.1 or NP_055081.1, or the mature DAP10 protein comprising amino acids 20-92 of the amino acid sequence of NCBI Reference Sequence NP_001007470.1. A co-stimulatory domain of DAP10 includes, for example, amino acids 70-92 of the amino acid sequence of NCBI Reference Sequence NP_001007470.1: CARPRRSPAQDGKVYINMPGRG (SEQ ID NO:137).

“OX40” (also known as TNFRSF4, TNF receptor superfamily member 4, CD134, ACT35, IMD16, tumor necrosis factor receptor superfamily member 4, and TXGP1L), as used herein refers to the gene identified by Entrez Gene ID No. 7293, allelic variants thereof, orthologs thereof, protein products thereof, and mRNA transcripts encoded by the gene, including the nucleotide sequence of NCBI Reference Sequence NM_003327.4. OX40 protein products include proteins encoded by OX40, for example, a protein comprising the amino acid sequence of NCBI Reference Sequence NP_003318.1 or the mature OX40 protein comprising amino acids 29-277 of the amino acid sequence of NCBI Reference Sequence NP_003318.1. A co-stimulatory domain of OX40 includes, for example, amino acids 236-277 of the amino acid sequence of NCBI Reference Sequence NP_003318.1:

ALYLLRRDQRLPPDAHKPPGGGSFRTPIQEEQADAHSTLAKI (SEQ ID
NO:141).

A co-stimulatory domain of OX40 is encoded by the following nucleotide sequence:

GCCCTGTACCTGCTCCGGAGGGACCAGAGGCTGCCCCCCGATGCCCACAA
GCCCCCTGGGGGAGGCAGTTTCCGGACCCCCATCCAAGAGGAGCAGGCCG
ACGCCCACTCCACCCTGGCCAAGATC (SEQ ID NO:142).

A co-stimulatory domain of ICOS includes, for example, amino acids 162-199 or 165-199 of the amino acid sequence of NCBI Reference Sequence NP_036224.1, for example:

TKKKYSSSYHDPNGEYMFMRAVNTAKKSRLTDVTL (SEQ ID NO:143
).

A co-stimulatory domain of ICOS is encoded by the following nucleotide sequence:

ACAAAAAAGAAGTATTCATCCAGTGTGCACGACCCTAACGGTGAATACAT
GTTCATGAGAGCAGTGAACACAGCCAAAAAATCTAGACTCACAGATGTGA
CCCTA (SEQ ID NO:144).

“IL-2Rβ” (also known as IL2RB, interleukin 2 receptor subunit beta, CD122, IMD63, IL15RB, and P70-75), as used herein refers to the gene identified by Entrez Gene ID No. 3560, allelic variants thereof, orthologs thereof, protein products thereof, and mRNA transcripts encoded by the gene, including the nucleotide sequence of NCBI Reference Sequence: NM_000878.5, NM_001346222.1, or NM_001346223.2. IL-2Rβ protein products include proteins encoded by IL-2Rβ, for example, a protein comprising the amino acid sequence of NCBI Reference Sequence: NP_000869.1, NP_001333151.1, or NP_001333152.1, or the mature IL-2Rβ protein comprising amino acids 27-551 of the amino acid sequence of NCBI Reference Sequence NP_000869.1. A co-stimulatory domain of IL-2Rβ includes, for example, amino acids 266-551 of the amino acid sequence of NCBI Reference Sequence NP_000869.1:

NCRNTGPWLKKVLKCNTPDPSKFFSQLSSEHGGDVQKWLSSPFPSSSFSP
GGLAPEISPLEVLERDKVTQLLLQQDKVPEPASLSSNHSLTSCFTNQGYF
FFHLPDALEIEACQVYFTYDPYSEEDPDEGVAGAPTGSSPQPLQPLSGED
DAYCTFPSRDDLLLFSPSLLGGPSPPSTAPGGSGAGEERMPPSLQERVPR
DWDPQPLGPPTPGVPDLVDFQPPPELVLREAGEEVPDAGPREGVSFPWSR
PPGQGEFRALNARLPLNTDAYLSLQELQGQDPTHLV (SEQ ID NO:15
2).

“IL2RA” (also known as IL2RA, interleukin 2 receptor subunit alpha, p55, CD25, IL2R, IMD41, TCGFR, and IDDM10), as used herein refers to the gene identified by Entrez Gene ID No. 3559, allelic variants thereof, orthologs thereof, protein products thereof, and mRNA transcripts encoded by the gene, including the nucleotide sequence of NCBI Reference Sequence: NM_000417.3, NM_001308242.2, or NM_001308243.2. IL2RA protein products include proteins encoded by IL2RA, for example, a protein comprising the amino acid sequence of NCBI Reference Sequence: NP_000408.1, NP_001295171.1, or NP_001295172.1, or the mature IL2RA protein comprising amino acids 22-272 of the amino acid sequence of NCBI Reference Sequence NP_000408.1. A co-stimulatory domain of IL2RA includes, for example, amino acids 260-272 of the amino acid sequence of NCBI Reference Sequence NP_000408.1.

“MYD88” (also known as MYD88 innate immune signal transduction adaptor, myeloid differentiation primary response protein MyD88, and MYD88D), as used herein refers to the gene identified by Entrez Gene ID No. 4615, allelic variants thereof, orthologs thereof, protein products thereof, and mRNA transcripts encoded by the gene, including the nucleotide sequence of NCBI Reference Sequence: NM_001172566.2, NM_001172567.2, NM_001172568.2, NM_001172569.3, NM_001365876.1, NM_001365877.1, NM_001374787.1, NM_001374788.1, or NM_002468.5. MYD88 protein products include proteins encoded by MYD88, for example, a protein comprising the amino acid sequence of NCBI Reference Sequence: NP_001166037.2, NP_001166038.2, NP_001166039.2, NP_001166040.2, NP_001352805.1, NP_001352806.1, NP_001361716.1, NP_001361717.1, and NP_002459.3. A co-stimulatory domain of MYD88 includes, for example, amino acids 160-304 of the amino acid sequence of NCBI Reference Sequence NP_001166038.2:

RFDAFICYCPSDIQFVQEMIRQLEQTNYRLKLCVSDRDVLPGTCVWSIAS
ELIEKRLARRPRGGCRRMVVVVSDDYLQSKECDFQTKFALSLSPGAHQKR
LIPIKYKAMKKEFPSILRFITVCDYTNPCTKSWFWTRLAKALSLP (SEQ
ID NO:179).

In some embodiments described herein, a CAR can include the entirety of or a portion of a co-stimulatory domain described herein, for example, a 4-1BB, CD27, CD28, CD40, CD40L, TLR2, DAP10, OX40, IL-2RB, IL-2RA, MYD88, or ICOS co-stimulatory domain described herein. For example, in some embodiments described herein, a CAR comprises a co-stimulatory domain of about 10, about 12, about 15, about 20, about 22, about 25, about 30, about 35, about 37, about 40, about 41, about 45, about 47, about 50, about 60, about 61, about 70, about 80, about 90, about 100, about 110, about 120, about 130, about 134, about 140, about 144, about 150, about 160, about 170, about 174, about 180, about 190, about 200, about 225, about 250, about 275, about 285, about 290, or about 300 amino acids in length. For example, in some embodiments described herein, a CAR comprises a co-stimulatory domain of about 10, about 12, about 15, about 20, about 22, about 25, about 30, about 35, about 37, about 40, about 41, about 45, about 47, about 50, about 60, about 61, about 70, about 80, about 90, about 100, about 110, about 120, about 130, about 134, about 140, about 144, about 150, about 160, about 170, about 174, about 180, about 190, about 200, about 225, about 250, about 275, about 285, about 290, or about 300 amino acids in length comprising a 4-1BB, CD27, CD28, CD40, CD40L, TLR2, DAP10, OX40, IL-2RB, IL-2RA, MYD88, or ICOS co-stimulatory domain described herein, or a portion thereof.

In some embodiments described herein, a CAR includes a co-stimulatory domain with an amino acid sequence that is about 90%, about 91%, about 92%, about 93%, about 94%, about 95%, about 96%, about 97%, about 98%, about 99%, about 90% to about 95%, about 95% to about 100%, or about 90% to about 100% identical to a 4-1BB, CD27, CD28, CD40, CD40L, TLR2, DAP10, OX40, IL-2RB, IL-2RA, MYD88, or ICOS co-stimulatory domain described herein.

Orthologs of genes, nucleotide sequences (e.g., mRNA sequences), and proteins described herein include, for example, mammalian orthologs, including, but not limited to mouse (i.e., Mus musculus) orthologs.

### CAR-T Cells

CAR-expressing T cells (CAR-T cells) are T lymphocyte cells (T-cells or T cells) which are isolated and genetically engineered to express one or more CARs. CAR-T cells can be T cells isolated from a patient, for example, a patient in need of treatment of a cancer, that are genetically engineered to express one or more CARs. Cells can be collected from patients using any suitable method, for example, leukapheresis or apheresis, followed by elutriation to remove myeloid and other contaminating cells and enrichment of T cells. Once isolated, T cells can be expanded and genetically engineered by any suitable means, for example, viral transduction (for example, lentiviral or gamma-retroviral transduction), or transfection or electroporation with a suitable expression vector. Genetically modified T cells can then be expanded in culture ex vivo before being administered to a patient.

Without being bound by theory, it is believed that expression of CARs allows targeting by CAR-T cells of a target cell population, for example, cancer cells that express a specific cell surface antigen to which an antigen-specific targeting region of a CAR (for example, an scFv, a Fab fragment, a F(ab′)₂ fragment, or a ligand) specifically binds. Binding by the CAR to said specific cell surface antigen in complex with a major histocompatibility complex molecule is believed to result in activation of a signaling cascade through an intracellular signaling domain (for example, an FcRγ or CD3ζ signaling domain) and, if present, one or more co-stimulatory domains (for example, a 4-1BB, CD27, CD28, CD40, CD40L, TLR2, DAP10, OX40, IL-2RB, IL-2RA, MYD88, and/or ICOS co-stimulatory domain) of the CAR. Thus, introduction of a CAR into a T cell is believed to allow the T cell to target and kill a target cell population expressing a cell surface antigen recognized by the CAR through the same effector functions (for example, FcRγ, CD3ζ, or co-stimulatory protein signaling) used by wild type T cells to eliminate infected or transformed cells. For example, introduction of a CAR described herein into a T cell is effective to allow the T cell to target and kill a target cancer cell population expressing a cell surface antigen recognized by the CAR (for example, a cancer cell antigen, a tumor associated antigen, or a tumor specific antigen) through FcRγ, CD3ζ, and/or co-stimulatory protein signaling.

In some embodiments described herein, CAR-T cells of the invention can be produced by introduction of one or more viral vectors to an isolated T cell or an isolated T cell population. In some embodiments, a viral vector delivers a transgene encoding a CAR nucleotide sequence to a T-cell. In some embodiments, a CAR nucleotide sequence includes nucleotide sequences encoding an antigen-specific targeting region, a transmembrane domain, and an intracellular signaling domain. In some embodiments, the CAR nucleotide sequence also includes nucleotide sequences encoding one or more co-stimulatory domains, a hinge domain, a spacer domain, and/or an amino acid transporter domain, for example, an arginine transporter domain.

Provided herein are CAR-T cells expressing an arginine transporter and a chimeric antigen receptor protein (referred to herein, alternatively, as “arg+CAR-T cells”) that can be used for treatment in solid tumor cancers and hematological cancers.

In addition to expression of CARs, CAR-T cells can also be genetically modified to co-express one or more separate co-stimulatory proteins, including cytokines, that enhance CAR function and persistence. For example, CAR-T cells can be programmed to co-express CD28, CD80, 4-1BB, 4-1BBL, CD86, OX40L, IL-12, IL-15, IL-18, and/or CD70 proteins. Additionally, in some embodiments, a CAR-T cell can be genetically modified to express 2 or more CARs targeting different cell surface antigens. In some embodiments, a CAR-T cell can be genetically modified to express a single CAR targeting a single cell surface antigen.

CAR-T cells of the invention include first, second, third, fourth, and fifth-generation CARs. CAR-T technology is described, for example, in Petersen and Krenciute, (2019) “Next Generation CAR T Cells for the Immunotherapy of High-Grade Glioma” Frontiers in Oncology, 9:1-9.

First generation CARs include fusions of an antigen-binding protein domain (e.g., CD8, CD4, CD25, CD16, or an antibody-derived scFv), a hinge/spacer domain, a transmembrane domain, and a signaling domain such as a CD3ζ or FcRγ intracellular signaling domain.

Second generation CARs include an antigen-binding protein domain, a hinge/spacer domain, a transmembrane domain, and a CD3ζ or FcRγ signaling domain, and further include an intracellular co-stimulatory domain (for example, a 4-1BB, CD27, CD28, CD40, CD40L, TLR2, DAP10, OX40, IL-2RB, IL-2RA, MYD88, or ICOS intracellular co-stimulatory domain).

Third-generation CARs include all components found in second generation CARs, but include multiple co-stimulatory domains (for example, more than a single 4-1BB, CD27, CD28, CD40, CD40L, TLR2, DAP10, OX40, IL-2RB, IL-2RA, MYD88, and/or ICOS intracellular co-stimulatory domain).

Fourth and fifth generation CARs (also known as armored CARs or TRUCKs) are further genetically engineered to express a CAR and to express a transgene encoding one or more signaling proteins, for example, cytokines or cytokine receptor proteins. For example, in some embodiments, a CAR-T cell is genetically engineered to overexpress IL-12, IL-15, IL-18, IL-7R, CD28, CD80, 4-1BB, 4-1BBL, CD86, OX40L, or CD70. In some embodiments, overexpression of a signaling protein such as a cytokine or cytokine receptor protein is effective to provide the CAR-T with enhanced persistence, proliferation, or anti-tumor activity.

Additionally, CAR-T cells can be genetically engineered using, for example, CRISPR/Cas9 gene editing tools, to delete genes that inhibit cell-intrinsic checkpoints, for example, PD-1 or CTLA4. Thus, in some embodiments, CAR-T cells described herein are genetically engineered to delete or decrease PD-1 or CTLA4 gene expression. CAR-T cells can also be genetically engineered to delete diacylglycerol kinase (DGK). Thus, in some embodiments, CAR-T cells described herein are genetically engineered to delete or decrease DGK gene expression, for example, DGKα and/or DGKζ isoform expression.

In some embodiments, CAR-T cells can be genetically engineered using, for example, CRISPR/Cas9 gene editing tools, to allow targeted CAR transgene insertion into the genome of a T cell. In some embodiments, CAR transgene insertion is mediated by an adeno-associated virus (AAV) vector, for example, an AAV6 vector, encoding a CAR nucleotide sequence. For example, in some embodiments, a CAR-T cell is genetically engineered to insert the CAR transgene into the endogenous TCR gene sequence, for example, the TCR alpha chain locus. In some embodiments, CAR-T cells can be genetically engineered using, for example, CRISPR/Cas9 gene editing tools, to replace a native T cell gene sequence with a mutant gene sequence. For example, in some embodiments, a CAR-T cell described herein is genetically engineered to replace a PD-1 or CXCR4 gene sequence with, respectively, a mutant PD-1 or CXCR4 gene sequence.

In some embodiments, a CAR-T cell described herein comprises an episome encoding a CAR. In some embodiments, a CAR-T cell described herein comprises an integrated transgene encoding a CAR.

Also described herein are CAR-T cells that are genetically engineered to express an amino acid transporter protein, for example, an arginine transporter protein. In some embodiments described herein, a CAR-T cell is genetically engineered to express an arginine transporter protein selected from the group consisting of CAT-1, CAT-2, CAT-3, CAT-4, y⁺LAT1, 4F2hc, y⁺LAT2, y⁺LAT1 and 4F2hc, y⁺LAT2 and 4F2hc, b^{0,+}AT, rBAT, b^{0,+}AT and rBAT, and ATB^{0,+}, or a combination thereof. For example, in some embodiments, a CAR-T cell described herein is genetically engineered to include a nucleotide sequence encoding an amino acid transporter, for example, a nucleotide sequence encoding an arginine transporter. In some embodiments, a CAR-T cell described herein comprises an episome encoding an amino acid transporter, for example, an arginine transporter. In some embodiments, a CAR-T cell described herein comprises a transgene encoding an amino acid transporter, for example, an arginine transporter.

Also described herein are CAR-T cells that are genetically engineered to express a CAR and an amino acid transporter protein, for example, an arginine transporter protein. For example, in some embodiments, a CAR-T cell described herein is genetically engineered to include a nucleotide sequence encoding a CAR and an amino acid transporter, for example, a nucleotide sequence encoding an arginine transporter. In some embodiments, a CAR-T cell described herein comprises an episome encoding a CAR and an amino acid transporter, for example, an arginine transporter. In some embodiments, a CAR-T cell described herein comprises a transgene encoding a CAR and an amino acid transporter, for example, an arginine transporter. In some embodiments, a CAR-T cell described herein comprises an episome encoding a CAR and an episome encoding an amino acid transporter, for example, an arginine transporter. In some embodiments, a CAR-T cell described herein comprises a transgene encoding a CAR and a transgene encoding an amino acid transporter, for example, an arginine transporter.

### Amino Acid Transporter Proteins

Described herein are CAR-T cells that are genetically modified to express one or more amino acid transporter proteins (AATs). Amino acid transporters are membrane transport proteins that play vital roles by regulating energy metabolism, protein synthesis, gene expression, redox balance signal transduction pathways, and growth at the cellular and whole organism levels through the transport of amino acids. Amino acids do not readily diffuse across lipid membranes, so membrane spanning transporter proteins are required to move amino acids in and out of a cell and between membrane bound intracellular compartments. Amino acid transport may be coupled to movements of ions including Na⁺, H⁺, K⁺, and/or Cl⁻ as well as movement of other amino acids by antiport. Dysregulation of AATs leads to metabolic reprogramming which changes intracellular amino acid levels contributing to pathogenesis. Dysregulation of AATs are implicated in a variety of pathological conditions such as, but not limited to, autophagy and tumor cell proliferation via metabolic reprogramming and inheritable human metabolic disorders such as cystinuria. Due to these metabolic abilities AATs may provide a potential target in anticancer drugs.

Amino acid transporter proteins are encoded by genes that belong to a number of families, including: the Solute Carrier (SLC) proteins; the Amino Acid-Polyamine-Organocation (APC) Superfamily; the Amino Acid/Auxin Permease (AAAP) Family; the Dicarboxylate/Amino Acid:Cation (Na+ or H+) Symporter (DAACS) Family; the Branched Chain Amino Acid:Cation Symporter (LIVCS) Family; the Hydroxy/Aromatic Amino Acid Permease (HAAAP) Family; the Branched Chain Amino Acid Exporter (LIV-E) Family; the 6TMS Neutral Amino Acid Transporter (NAAT) Family; the Basic Amino Acid Antiporter (ArcD) Family; and the Putative Amino Acid Permease (PAAP) Family. The SLC proteins comprise the largest group of amino acid transporter proteins and include over 400 proteins distributed between 65 families.

Amino acid transporter proteins can be classified as: sodium-dependent neutral amino acid transporters, sodium-independent neutral amino acid transporters, sodium-dependent anionic amino acid transporters-system X⁻_{AG}, sodium-independent anionic amino acid transporters system x_{C}⁻, sodium-dependent cationic amino acid transporters, and sodium-independent cationic amino acid transporters. Amino acid transporter proteins control transport of amino acids across the cell membrane, including transport of arginine, glutamine, and leucine, as well as signaling compounds such as gamma-aminobutyric acid (GABA). Examples of amino acid transporter proteins are described, for example, in Ren et al., (2017) “Amino-acid transporters in T-cell activation and differentiation,” Cell Death and Disease, 8, e2655.

### Arginine Transporter Proteins

Also described herein are CAR-T cells that are genetically modified to express one or more arginine transporter proteins. Arginine transporter proteins are encoded by genes that belong to the solute carrier gene (SLC) families. Most SLCs encode proteins that localize to the cell membrane, though some members localize to the mitochondria or other intracellular organelles. SLC family protein products can transport, for example, charged organic molecules, uncharged organic molecules, inorganic ions, and/or ammonia across the cell membrane. SLC families that specifically encode transporter proteins capable of transporting arginine across the cell membrane include the SLC3, SLC6, and SLC7 families.

In mammals, cellular arginine availability is largely regulated by members of the SLC7 family, although there are 6 major families of AATs in the solute carrier gene superfamily. The protein products of these transporter genes are characterized by having multiple transmembrane domains organized around a central pore region. Their efficiency and capacity in the plasma membrane significantly determines arginine availability in the cell.

The SLC7 family is divided into two subgroups: the cationic amino acid transporters (CATs), and the L-type amino acid transporters (LATs). CATs function as monomers in the plasma membrane while LATs are obligate heterodimers which form a disulphide-linked dimer with a single transmembrane spanning glycoprotein (SLC3) which traffics the transporter to the plasma membrane and aid in protein stability. CAT and LAT families show various differences in their interaction with the SLC3 family, substrate specificity, and transport mechanism. CATs are specific for cationic amino acids, including arginine. Originally designated as system y⁺, CATs mediate Na⁺-independent uptake of cationic amino acids with high affinity. In mammals CATs operate as exchangers or facilitators. Arginine metabolism is significantly regulated through the expression of these y⁺ system of cationic amino acid transporters.

Arginine transporter proteins include: CAT-1, CAT-2, CAT-3, CAT-4, y⁺LAT2, 4F2hc, y⁺LAT1, b^{0,+}AT, rBAT, and ATB^{0,+}. In some embodiments described herein, an arginine transporter is comprised of a single SLC family protein, for example: CAT-1, CAT-2, CAT-3, CAT-4, or ATB^{0,+}. In some embodiments, an arginine transporter is comprised of a combination of SLC family proteins, for example: y⁺LAT2 and 4F2hc, y⁺LAT1 and 4F2hc, or b^{0,+}AT and rBAT.

Arginine transporter proteins can be sodium- and chloride-dependent or sodium independent amino acid transporter proteins. Examples of sodium-independent amino acid transporter proteins include members of the y⁺ (for example, CAT-1, CAT-2, CAT-3), y⁺L (for example, 4F2hc in combination with y⁺LAT1 or y⁺LAT2), and b^{0,+} transport systems. Examples of sodium-dependent amino acid transporter proteins include members of the B^{0,+} transport system. Arginine transporter systems comprised of a single protein include members of the y⁺ and B^{0,+} transporter systems. By contrast, the y⁺L and b^{0,+} arginine transporter systems are comprised of a glycoprotein (for example, 4F2hc) and a protein.

“SLC7A1” (also known as solute carrier family 7 member 1, ERR, ATRC1, CAT-1, HCAT1, and REC1L) as used herein refers to the gene identified by Entrez Gene ID No. 6541, allelic variants thereof, orthologs thereof, and mRNA transcripts encoded by the gene, including the nucleotide sequence of NCBI Reference Sequence NM_003045.5(SEQ ID NO: 180).

Cationic amino acid transporter 1 (CAT-1) proteins described herein include protein sequences encoded by SLC7A1, the amino acid sequence of NCBI Reference Sequence NP_003036.1 and the amino acid sequence of NCBI Consensus Coding Sequence (CCDS) ID NO. CCDS9333.1:

MGCKVLLNIGQQMLRRKVVDCSREETRLSRCLNTFDLVALGVGSTLGAGV
YVLAGAVARENAGPAIVISFLIAALASVLAGLCYGEFGARVPKTGSAYLY
SYVTVGELWAFITGWNLILSYIIGTSSVARAWSATFDELIGRPIGEFSRT
HMTLNAPGVLAENPDIFAVIIILILTGLLTLGVKESAMVNKIFTCINVLV
LGFIMVSGFVKGSVKNWQLTEEDFGNTSGRLCLNNDTKEGKPGVGGFMPF
GFSGVLSGAATCFYAFVGFDCIATTGEEVKNPQKAIPVGIVASLLICFIA
YFGVSAALTLMMPYFCLDNNSPLPDAFKHVGWEGAKYAVAVGSLCALSAS
LLGSMFPMPRVIYAMAEDGLLFKFLANVNDRTKTPIIATLASGAVAAVMA
FLFDLKDLVDLMSIGTLLAYSLVAACVLVLRYQPEQPNLVYQMASTSDEL
DPADQNELASTNDSQLGFLPEAEMFSLKTILSPKNMEPSKISGLIVNIST
SLIAVLIITFCIVTVLGREALTKGALWAVFLLAGSALLCAVVTGVIWRQP
ESKTKLSFKVPFLPVLPILSIFVNVYLMMQLDQGTWVRFAVWMLIGFIIY
FGYGLWHSEEASLDADQARTPDGNLDQCK (SEQ ID NO: 182).

An exemplary CAT-1 nucleotide sequence is the nucleotide sequence of NCBI CCDS ID NO. CCDS9333.1:

ATGGGGTGCAAAGTCCTGCTCAACATTGGGCAGCAGATGCTGCGGCGGAA
GGTGGTGGACTGTAGCCGGGAGGAGACGCGGCTGTCTCGCTGCCTGAACA
CTTTTGATCTGGTGGCCCTCGGGGTGGGCAGCACACTGGGTGCTGGTGTC
TACGTCCTGGCTGGAGCTGTGGCCCGTGAGAATGCAGGCCCTGCCATTGT
CATCTCCTTCCTGATCGCTGCGCTGGCCTCAGTGCTGGCTGGCCTGTGCT
ATGGCGAGTTTGGTGCTCGGGTCCCCAAGACGGGCTCAGCTTACCTCTAC
AGCTATGTCACCGTTGGAGAGCTCTGGGCCTTCATCACCGGCTGGAACTT
AATCCTCTCCTACATCATCGGTACTTCAAGCGTAGCGAGGGCCTGGAGCG
CCACCTTCGACGAGCTGATAGGCAGACCCATCGGGGAGTTCTCACGGACA
CACATGACTCTGAACGCCCCCGGCGTGCTGGCTGAAAACCCCGACATATT
CGCAGTGATCATAATTCTCATCTTGACAGGACTTTTAACTCTTGGTGTGA
AAGAGTCGGCCATGGTCAACAAAATATTCACTTGTATTAACGTCCTGGTC
CTGGGCTTCATAATGGTGTCAGGATTTGTGAAAGGATCGGTTAAAAACTG
GCAGCTCACGGAGGAGGATTTTGGGAACACATCAGGCCGTCTCTGTTTGA
ACAATGACACAAAAGAAGGGAAGCCCGGTGTTGGTGGATTCATGCCCTTC
GGGTTCTCTGGTGTCCTGTCGGGGGCAGCGACTTGCTTCTATGCCTTCGT
GGGCTTTGACTGCATCGCCACCACAGGTGAAGAGGTGAAGAACCCACAGA
AGGCCATCCCCGTGGGGATCGTGGCGTCCCTCTTGATCTGCTTCATCGCC
TACTTTGGGGTGTCGGCTGCCCTCACGCTCATGATGCCCTACTTCTGCCT
GGACAATAACAGCCCCCTGCCCGACGCCTTTAAGCACGTGGGCTGGGAAG
GTGCCAAGTACGCAGTGGCCGTGGGCTCCCTCTGCGCTCTTTCCGCCAGT
CTTCTAGGTTCCATGTTTCCCATGCCTCGGGTTATCTATGCCATGGCTGA
GGATGGACTGCTATTTAAATTCTTAGCCAACGTCAATGATAGGACCAAAA
CACCAATAATCGCCACATTAGCCTCGGGTGCCGTTGCTGCTGTGATGGCC
TTCCTCTTTGACCTGAAGGACTTGGTGGACCTCATGTCCATTGGCACTCT
CCTGGCTTACTCGTTGGTGGCTGCCTGTGTGTTGGTCTTACGGTACCAGC
CAGAGCAGCCTAACCTGGTATACCAGATGGCCAGTACTTCCGACGAGTTA
GATCCAGCAGACCAAAATGAATTGGCAAGCACCAATGATTCCCAGCTGGG
CTTTTTACCAGAGGCAGAGATGTTCTCTTTGAAAACCATACTCTCACCCA
AAAACATGGAGCCTTCCAAAATCTCTGGGCTAATTGTGAACATTTCAACC
AGCCTCATAGCTGTTCTCATCATCACCTTCTGCATTGTGACCGTGCTTGG
AAGGGAGGCTCTCACCAAAGGGGCGCTGTGGGCAGTCTTTCTGCTCGCAG
GGTCTGCCCTCCTCTGTGCCGTGGTCACGGGCGTCATCTGGAGGCAGCCC
GAGAGCAAGACCAAGCTCTCATTTAAGGTTCCCTTCCTGCCAGTGCTCCC
CATCCTGAGCATCTTCGTGAACGTCTATCTCATGATGCAGCTGGACCAGG
GCACCTGGGTCCGGTTTGCTGTGTGGATGCTGATAGGCTTCATCATCTAC
TTTGGCTATGGCCTGTGGCACAGCGAGGAGGCGTCCCTGGATGCCGACCA
AGCAAGGACTCCTGACGGCAACTTGGACCAGTGCAAGTGA (SEQ ID N
O: 183).

“SLC7A2” (also known as solute carrier family 7 member 2, CAT2, ATRC2, and HCAT2) as used herein refers to the gene identified by Entrez Gene ID No. 6542, allelic variants thereof, orthologs thereof, and mRNA transcripts encoded by the gene, including the nucleotide sequence of NCBI Reference Sequence: NM_001008539.4 (SEQ ID NO:184), NM_001164771.2 (SEQ ID NO:185), NM_001370337.1 (SEQ ID NO:186), NM_001370338.1 (SEQ ID NO:187), or NM_003046.6 (SEQ ID NO:188).

Cationic amino acid transporter 2 (CAT-2) proteins described herein include protein sequences encoded by SLC7A2 and the amino acid sequence of NCBI Reference Sequence: NP_001008539.3, NP_001158243.1, NP_001357266.1, NP_001357267.1, or NP_003037.4. CAT-2 can be expressed as multiple isoforms, including CAT-2A (identified as the amino acid sequence of NCBI CCDS ID NO. CCDS6002.2:

MKIETSGYNSDKLICRGFIGTPAPPVCDSKFLLSPSSDVRMIPCRAALTF
ARCLIRRKIVTLDSLEDTKLCRCLSTMDLIALGVGSTLGAGVYVLAGEVA
KADSGPSIVVSFLIAALASVMAGLCYAEFGARVPKTGSAYLYTYVTVGEL
WAFITGWNLILSYVIGTSSVARAWSGTFDELLSKQIGQFLRTYFRMNYTG
LAEYPDFFAVCLILLLAGLLSFGVKESAWVNKVFTAVNILVLLFVMVAGF
VKGNVANWKISEEFLKNISASAREPPSENGTSIYGAGGFMPYGFTGTLAG
AATCFYAFVGFDCIATTGEEVRNPQKAIPIGIVTSLLVCFMAYFGVSAAL
TLMMPYYLLDEKSPLPVAFEYVGWGPAKYVVAAGSLCALSTSLLGSMFPL
PRILFAMARDGLLFRFLARVSKRQSPVAATLTAGVISALMAFLFDLKALV
DMMSIGTLMAYSLVAACVLIL,RYQPGLSYDQPKCSPEKDGLGSSPRVTS
KSESQVTMLQRQGFSMRTLFCPSLLPTQQSASLVSFLVGFLAFLVLGLSV
LTTYGVHAITRLEAWSLALLALFLVLFVAIVLTIWRQPQNQQKVAFMVPF
LPFLPAFSILVNIYLMVQLSADTWVRFSIWMAIGFLIYFSYGIRHSLEGH
LRDENNEEDAYPDNVHAAAEEKSAIQANDHHPRNLSSPFIFHEKTSEF (
SEQ ID NO:194)); and

CAT-2B (identified as the amino acid sequence of NCBI CCDS ID NO. CCDS34852.1:

MIPCRAALTFARCLIRRKIVTLDSLEDTKLCRCLSTMDLIALGVGSTLGA
GVYVLAGEVAKADSGPSIVVSFLIAALASVMAGLCYAEFGARVPKTGSAY
LYTYVTVGELWAFITGWNLILSYVIGTSSVARAWSGTFDELLSKQIGQFL
RTYFRMNYTGLAEYPDFFAVCLILLLAGLLSFGVKESAWVNKVFTAVNIL
VLLFVMVAGFVKGNVANWKISEEFLKNISASAREPPSENGTSIYGAGGFM
PYGFTGTLAGAATCFYAFVGFDCIATTGEEVRNPQKAIPIGIVTSLLVCF
MAYFGVSAALTLMMPYYLLDEKSPLPVAFEYVGWGPAKYVVAAGSLCALS
TSLLGSIFPMPRVIYAMAEDGLLFKCLAQINSKTKTPIIATLSSGAVAAL
MAFLFDLKALVDMMSIGTLMAYSLVAACVLILRYQPGLSYDQPKCSPEKD
GLGSSPRVTSKSESQVTMLQRQGFSMRTLFCPSLLPTQQSASLVSFLVGF
LAFLVLGLSVLTTYGVHAITRLEAWSLALLALFLVLFVAIVLTIWRQPQN
QQKVAFMVPFLPFLPAFSILVNIYLMVQLSADTWVRFSIWMAIGFLIYFS
YGIRHSLEGHLRDENNEEDAYPDNVHAAAEEKSAIQANDHHPRNLSSPFI
FHEKTSEF (SEQ ID NO:195)).

The CAT-2A nucleotide sequence is the nucleotide sequence of CCDS ID NO. CCDS6002.2:

ATGAAGATAGAAACAAGTGGTTATAACTCAGACAAACTAATTTGTCGAGG
GTTTATTGGAACACCTGCCCCACCGGTTTGCGACAGCAAGTTTCTCCTGT
CGCCTTCGTCAGACGTCAGAATGATTCCTTGCAGAGCCGCGCTGACCTTT
GCCCGATGTCTGATCCGGAGAAAAATCGTGACCCTGGACAGTCTAGAAGA
CACCAAATTATGCCGCTGCTTATCCACCATGGACCTCATTGCCCTGGGCG
TTGGAAGCACCCTTGGGGCCGGGGTTTATGTCCTCGCTGGGGAGGTGGCC
AAGGCAGACTCGGGCCCCAGCATCGTGGTGTCCTTCCTCATTGCTGCCCT
GGCTTCAGTGATGGCTGGCCTCTGCTATGCCGAATTTGGGGCCCGTGTTC
CCAAGACGGGGTCTGCATATTTGTACACCTACGTGACTGTCGGAGAGCTG
TGGGCCTTCATCACTGGCTGGAATCTCATTTTATCGTATGTGATAGGTAC
ATCAAGTGTTGCAAGAGCCTGGAGTGGCACCTTTGATGAACTTCTTAGCA
AACAGATTGGTCAGTTTTTGAGGACATACTTCAGAATGAATTACACTGGT
CTTGCAGAATATCCCGATTTTTTTGCTGTGTGCCTTATATTACTTCTAGC
AGGTCTTTTGTCTTTTGGAGTAAAAGAGTCTGCTTGGGTGAATAAAGTCT
TCACAGCTGTTAATATTCTCGTCCTTCTGTTTGTGATGGTTGCTGGGTTT
GTGAAAGGAAATGTGGCAAACTGGAAGATTAGTGAAGAGTTTCTCAAAAA
TATATCAGCAAGTGCCAGAGAGCCACCTTCTGAAAACGGAACAAGTATCT
ATGGGGCTGGTGGCTTTATGCCTTATGGCTTTACGGGAACGTTGGCTGGT
GCTGCAACTTGCTTTTATGCCTTTGTGGGATTTGACTGCATTGCAACAAC
TGGTGAAGAAGTTCGGAATCCCCAGAAAGCTATTCCCATTGGAATTGTGA
CGTCTTTGCTTGTTTGCTTTATGGCCTATTTTGGGGTCTCTGCAGCTTTA
ACACTTATGATGCCGTACTACCTCCTCGATGAAAAAAGCCCCCTTCCTGT
AGCGTTTGAATATGTGGGATGGGGTCCTGCCAAATATGTCGTCGCAGCTG
GTTCTCTCTGCGCCTTGTCAACAAGTCTTCTGGGCTCTATGTTTCCTTTA
CCCCGAATTCTGTTTGCCATGGCCCGGGATGGCTTACTGTTTAGATTTCT
TGCCAGAGTGAGTAAGAGGCAGTCACCAGTTGCTGCCACGTTGACTGCAG
GGGTCATTTCTGCTTTGATGGCCTTTCTGTTTGACCTGAAGGCGCTTGTG
GACATGATGTCCATTGGCACACTCATGGCCTACTCTCTGGTGGCAGCCTG
TGTTCTCATCCTCAGGTACCAGCCTGGCTTATCTTACGACCAGCCCAAAT
GTTCTCCTGAGAAAGATGGTCTGGGATCGTCTCCCAGGGTAACCTCGAAG
AGTGAGTCCCAGGTCACCATGCTGCAGAGACAGGGCTTCAGCATGCGGAC
CCTCTTCTGCCCCTCCCTTCTGCCAACACAGCAGTCAGCTTCTCTCGTGA
GCTTTCTGGTAGGATTCCTAGCTTTCCTCGTGTTGGGCCTGAGTGTCTTG
ACCACTTACGGAGTTCATGCCATCACCAGGCTGGAGGCCTGGAGCCTCGC
TCTCCTCGCGCTGTTTCTTGTTCTCTTCGTTGCCATCGTTCTCACCATCT
GGAGGCAGCCCCAGAATCAGCAAAAAGTAGCCTTCATGGTTCCATTCTTA
CCATTTTTGCCAGCGTTCAGCATCTTGGTGAACATTTACTTGATGGTCCA
GTTAAGTGCAGACACTTGGGTCAGATTCAGCATTTGGATGGCAATTGGCT
TCCTGATTTACTTTTCTTATGGCATTAGACACAGCCTGGAGGGTCATCTG
AGAGATGAAAACAATGAAGAAGATGCTTATCCAGACAACGTTCATGCAGC
AGCAGAAGAAAAATCTGCCATTCAAGCAAATGACCATCACCCAAGAAATC
TCAGTTCACCTTTCATATTCCATGAAAAGACAAGTGAATTCTAA (SEQ
ID NO:196).

The CAT-2B nucleotide sequence is the nucleotide sequence of CCDS ID NO. CCDS34852.1:

ATGATTCCTTGCAGAGCCGCGCTGACCTTTGCCCGATGTCTGATCCGGAG
AAAAATCGTGACCCTGGACAGTCTAGAAGACACCAAATTATGCCGCTGCT
TATCCACCATGGACCTCATTGCCCTGGGCGTTGGAAGCACCCTTGGGGCC
GGGGTTTATGTCCTCGCTGGGGAGGTGGCCAAGGCAGACTCGGGCCCCAG
CATCGTGGTGTCCTTCCTCATTGCTGCCCTGGCTTCAGTGATGGCTGGCC
TCTGCTATGCCGAATTTGGGGCCCGTGTTCCCAAGACGGGGTCTGCATAT
TTGTACACCTACGTGACTGTCGGAGAGCTGTGGGCCTTCATCACTGGCTG
GAATCTCATTTTATCGTATGTGATAGGTACATCAAGTGTTGCAAGAGCCT
GGAGTGGCACCTTTGATGAACTTCTTAGCAAACAGATTGGTCAGTTTTTG
AGGACATACTTCAGAATGAATTACACTGGTCTTGCAGAATATCCCGATTT
TTTTGCTGTGTGCCTTATATTACTTCTAGCAGGTCTTTTGTCTTTTGGAG
TAAAAGAGTCTGCTTGGGTGAATAAAGTCTTCACAGCTGTTAATATTCTC
GTCCTTCTGTTTGTGATGGTTGCTGGGTTTGTGAAAGGAAATGTGGCAAA
CTGGAAGATTAGTGAAGAGTTTCTCAAAAATATATCAGCAAGTGCCAGAG
AGCCACCTTCTGAAAACGGAACAAGTATCTATGGGGCTGGTGGCTTTATG
CCTTATGGCTTTACGGGAACGTTGGCTGGTGCTGCAACTTGCTTTTATGC
CTTTGTGGGATTTGACTGCATTGCAACAACTGGTGAAGAAGTTCGGAATC
CCCAGAAAGCTATTCCCATTGGAATTGTGACGTCTTTGCTTGTTTGCTTT
ATGGCCTATTTTGGGGTCTCTGCAGCTTTAACACTTATGATGCCGTACTA
CCTCCTCGATGAAAAAAGCCCCCTTCCTGTAGCGTTTGAATATGTGGGAT
GGGGTCCTGCCAAATATGTCGTCGCAGCTGGTTCTCTCTGCGCCTTGTCA
ACAAGTCTTCTTGGATCCATTTTCCCAATGCCTCGTGTAATCTATGCTAT
GGCGGAGGATGGGTTGCTTTTCAAATGTCTAGCTCAAATCAATTCCAAAA
CGAAGACACCAATAATTGCTACTTTATCATCGGGTGCAGTGGCAGCTTTG
ATGGCCTTTCTGTTTGACCTGAAGGCGCTTGTGGACATGATGTCCATTGG
CACACTCATGGCCTACTCTCTGGTGGCAGCCTGTGTTCTCATCCTCAGGT
ACCAGCCTGGCTTATCTTACGACCAGCCCAAATGTTCTCCTGAGAAAGAT
GGTCTGGGATCGTCTCCCAGGGTAACCTCGAAGAGTGAGTCCCAGGTCAC
CATGCTGCAGAGACAGGGCTTCAGCATGCGGACCCTCTTCTGCCCCTCCC
TTCTGCCAACACAGCAGTCAGCTTCTCTCGTGAGCTTTCTGGTAGGATTC
CTAGCTTTCCTCGTGTTGGGCCTGAGTGTCTTGACCACTTACGGAGTTCA
TGCCATCACCAGGCTGGAGGCCTGGAGCCTCGCTCTCCTCGCGCTGTTTC
TTGTTCTCTTCGTTGCCATCGTTCTCACCATCTGGAGGCAGCCCCAGAAT
CAGCAAAAAGTAGCCTTCATGGTTCCATTCTTACCATTTTTGCCAGCGTT
CAGCATCTTGGTGAACATTTACTTGATGGTCCAGTTAAGTGCAGACACTT
GGGTCAGATTCAGCATTTGGATGGCAATTGGCTTCCTGATTTACTTTTCT
TATGGCATTAGACACAGCCTGGAGGGTCATCTGAGAGATGAAAACAATGA
AGAAGATGCTTATCCAGACAACGTTCATGCAGCAGCAGAAGAAAAATCTG
CCATTCAAGCAAATGACCATCACCCAAGAAATCTCAGTTCACCTTTCATA
TTCCATGAAAAGACAAGTGAATTCTAA (SEQ ID NO:197).

Also described herein are CAT-2A proteins that include one or more naturally occurring or engineered amino acid mutations. For example, described herein are CAT-2A proteins that include substitution and/or insertion mutations. CAT-2A amino acid sequences can include, for example, the amino acid mutations R369E, N381i, or R369E and N381i. CAT-2A amino acid sequences that include R369E, N381i, and R369E/N381i mutations include the following:

MIPCRAALTFARCLIRRKIVTLDSLEDTKLCRCLSTMDLIALGVGSTLGA
GVYVLAGEVAKADSGPSIVVSFLIAALASVMAGLCYAEFGARVPKTGSAY
LYTYVTVGELWAFITGWNLILSYVIGTSSVARAWSGTFDELLSKQIGQFL
RTYFRMNYTGLAEYPDFFAVCLILLLAGLLSFGVKESAWVNKVFTAVNIL
VLLFVMVAGFVKGNVANWKISEEFLKNISASAREPPSENGTSIYGAGGFM
PYGFTGTLAGAATCFYAFVGFDCIATTGEEVRNPQKAIPIGIVTSLLVCF
MAYFGVSAALTLMMPYYLLDEKSPLPVAFEYVGWGPAKYVVAAGSLCALS
TSLLGSMFPLPRILFAMAEDGLLFRFLARVSKRQSPVAATLTAGVISALM
AFLFDLKALVDMMSIGTLMAYSLVAACVLILRYQPGLSYDQPKCSPEKDG
LGSSPRVTSKSESQVTMLQRQGFSMRTLFCPSLLPTQQSASLVSFLVGFL
AFLVLGLSVLTTYGVHAITRLEAWSLALLALFLVLFVAIVLTIWRQPQNQ
QKVAFMVPFLPFLPAFSILVNIYLMVQLSADTWVRFSIWMAIGFLIYFSY
GIRHSLEGHLRDENNEEDAYPDNVHAAAEEKSAIQANDHHPRNLSSPFIF
HEKTSEF (SEQ ID NO: 198);

MIPCRAALTFARCLIRRKIVTLDSLEDTKLCRCLSTMDLIALGVGSTLGA
GVYVLAGEVAKADSGPSIVVSFLIAALASVMAGLCYAEFGARVPKTGSAY
LYTYVTVGELWAFITGWNLILSYVIGTSSVARAWSGTFDELLSKQIGQFL
RTYFRMNYTGLAEYPDFFAVCLILLLAGLLSFGVKESAWVNKVFTAVNIL
VLLFVMVAGFVKGNVANWKISEEFLKNISASAREPPSENGTSIYGAGGFM
PYGFTGTLAGAATCFYAFVGFDCIATTGEEVRNPQKAIPIGIVTSLLVCF
MAYFGVSAALTLMMPYYLLDEKSPLPVAFEYVGWGPAKYVVAAGSLCALS
TSLLGSMFPLPRILFAMARDGLLFRFLARVNSKRQSPVAATLTAGVISAL
MAFLFDLKALVDMMSIGTLMAYSLVAACVLILRYQPGLSYDQPKCSPEKD
GLGSSPRVTSKSESQVTMLQRQGFSMRTLFCPSLLPTQQSASLVSFLVGF
LAFLVLGLSVLTTYGVHAITRLEAWSLALLALFLVLFVAIVLTIWRQPQN
QQKVAFMVPFLPFLPAFSILVNIYLMVQLSADTWVRFSIWMAIGFLIYFS
YGIRHSLEGHLRDENNEEDAYPDNVHAAAEEKSAIQANDHHPRNLSSPFI
FHEKTSEF (SEQ ID NO:199); and

MIPCRAALTFARCLIRRKIVTLDSLEDTKLCRCLSTMDLIALGVGSTLGA
GVYVLAGEVAKADSGPSIVVSFLIAALASVMAGLCYAEFGARVPKTGSAY
LYTYVTVGELWAFITGWNLILSYVIGTSSVARAWSGTFDELLSKQIGQFL
RTYFRMNYTGLAEYPDFFAVCLILLLAGLLSFGVKESAWVNKVFTAVNIL
VLLFVMVAGFVKGNVANWKISEEFLKNISASAREPPSENGTSIYGAGGFM
PYGFTGTLAGAATCFYAFVGFDCIATTGEEVRNPQKAIPIGIVTSLLVCF
MAYFGVSAALTLMMPYYLLDEKSPLPVAFEYVGWGPAKYVVAAGSLCALS
TSLLGSMFPLPRILFAMAEDGLLFRFLARVNSKRQSPVAATLTAGVISAL
MAFLFDLKALVDMMSIGTLMAYSLVAACVLILRYQPGLSYDQPKCSPEKD
GLGSSPRVTSKSESQVTMLQRQGFSMRTLFCPSLLPTQQSASLVSFLVGF
LAFLVLGLSVLTTYGVHAITRLEAWSLALLALFLVLFVAIVLTIWRQPQN
QQKVAFMVPFLPFLPAFSILVNIYLMVQLSADTWVRFSIWMAIGFLIYFS
YGIRHSLEGHLRDENNEEDAYPDNVHAAAEEKSAIQANDHHPRNLSSPFI
FHEKTSEF (SEQ ID NO:200).

Nucleic acid sequences encoding R369E, N381i, and R369E/N381i mutations include the following:

ATGATTCCCTGCAGAGCCGCTCTGACCTTCGCCAGATGCCTGATCAGACG
GAAGATCGTGACCCTGGACAGCCTGGAAGATACCAAGCTGTGCCGGTGCC
TGAGCACCATGGATCTGATTGCCCTCGGCGTGGGCTCTACACTTGGAGCT
GGTGTTTATGTGCTGGCTGGCGAGGTGGCCAAGGCCGATTCTGGACCTTC
TATCGTGGTGTCCTTCCTGATCGCCGCTCTGGCCTCTGTTATGGCCGGAC
TGTGTTACGCCGAGTTCGGAGCCAGAGTGCCTAAGACAGGCAGCGCCTAC
CTGTACACCTACGTGACAGTGGGAGAGCTGTGGGCCTTTATCACCGGCTG
GAACCTGATCCTGAGCTACGTGATCGGCACCTCCTCTGTGGCTAGAGCTT
GGAGCGGCACCTTTGACGAGCTGCTGTCTAAGCAGATCGGCCAGTTCCTG
CGGACCTACTTCCGGATGAATTACACCGGCCTGGCCGAGTATCCCGACTT
CTTCGCCGTGTGTCTGATCCTGCTGCTTGCCGGACTGCTGAGCTTCGGCG
TGAAAGAGTCTGCCTGGGTCAACAAGGTGTTCACCGCCGTGAATATCCTG
GTGCTGCTGTTCGTGATGGTGGCCGGCTTCGTGAAGGGCAACGTGGCCAA
TTGGAAGATCAGCGAAGAGTTCCTGAAGAACATCAGCGCCAGCGCCAGAG
AGCCTCCTTCTGAAAACGGCACCAGCATCTATGGCGCAGGCGGCTTTATG
CCCTACGGCTTTACTGGAACACTGGCAGGCGCCGCTACCTGCTTCTATGC
CTTCGTGGGCTTCGACTGTATCGCCACCACTGGGGAAGAAGTGCGGAACC
CTCAGAAGGCTATCCCCATCGGCATCGTGACAAGCCTGCTCGTGTGCTTC
ATGGCCTACTTCGGAGTGTCCGCCGCACTGACCCTGATGATGCCTTACTA
CCTGCTGGACGAGAAGTCCCCTCTGCCTGTGGCCTTTGAGTATGTTGGCT
GGGGCCCTGCCAAATACGTGGTGGCTGCTGGATCTCTGTGCGCCCTGTCT
ACATCTCTGCTGGGCAGCATGTTCCCTCTGCCAAGAATCCTGTTCGCCAT
GGCCGAGGATGGCCTGCTGTTCAGATTCCTGGCCAGAGTGAGCAAGCGGC
AGTCTCCTGTGGCCGCTACACTTACAGCTGGCGTGATCTCTGCCCTGATG
GCTTTCCTGTTCGACCTGAAGGCCCTGGTGGACATGATGAGCATCGGCAC
ACTGATGGCCTACAGCCTGGTGGCAGCCTGCGTGCTGATTCTGAGATACC
AGCCAGGCCTGTCCTACGACCAGCCTAAGTGTTCCCCTGAGAAGGACGGC
CTGGGCAGCTCTCCTAGAGTGACAAGCAAGAGCGAGAGCCAAGTGACCAT
GCTGCAGAGACAGGGCTTCAGCATGCGGACCCTGTTCTGCCCTTCTCTGC
TGCCTACACAGCAGTCTGCTAGCCTGGTGTCTTTCCTCGTGGGATTTCTG
GCCTTTCTGGTGCTGGGCCTGAGCGTGCTGACAACATATGGGGTGCACGC
CATCACCAGACTGGAAGCTTGGAGTCTGGCTCTGCTGGCCCTGTTCCTGG
TTCTGTTTGTGGCCATCGTGCTGACCATTTGGCGGCAGCCCCAGAACCAG
CAGAAAGTGGCTTTCATGGTGCCCTTTCTGCCTTTCCTGCCAGCCTTCAG
CATCCTGGTCAACATCTACCTGATGGTGCAGCTGAGCGCCGACACCTGGG
TCCGATTTTCCATCTGGATGGCTATCGGCTTCCTCATCTACTTCAGCTAC
GGCATCCGGCACTCCCTGGAAGGCCATCTGAGAGATGAGAACAACGAAGA
GGACGCTTACCCCGACAACGTGCACGCCGCTGCCGAAGAGAAATCTGCCA
TCCAGGCCAACGACCACCATCCAAGAAACCTGAGCAGCCCCTTCATCTTC
CACGAGAAAACCAGCGAGTTT (SEQ ID NO:201);

ATGATTCCCTGCAGAGCCGCTCTGACCTTCGCCAGATGCCTGATCAGACG
GAAGATCGTGACCCTGGACAGCCTGGAAGATACCAAGCTGTGCCGGTGCC
TGAGCACCATGGATCTGATTGCCCTCGGCGTGGGCTCTACACTTGGAGCT
GGTGTTTATGTGCTGGCTGGCGAGGTGGCCAAGGCCGATTCTGGACCTTC
TATCGTGGTGTCCTTCCTGATCGCCGCTCTGGCCTCTGTTATGGCCGGAC
TGTGTTACGCCGAGTTCGGAGCCAGAGTGCCTAAGACAGGCAGCGCCTAC
CTGTACACCTACGTGACAGTGGGAGAGCTGTGGGCCTTTATCACCGGCTG
GAACCTGATCCTGAGCTACGTGATCGGCACCTCCTCTGTGGCTAGAGCTT
GGAGCGGCACCTTTGACGAGCTGCTGTCTAAGCAGATCGGCCAGTTCCTG
CGGACCTACTTCCGGATGAATTACACCGGCCTGGCCGAGTATCCCGACTT
CTTCGCCGTGTGTCTGATCCTGCTGCTTGCCGGACTGCTGAGCTTCGGCG
TGAAAGAGTCTGCCTGGGTCAACAAGGTGTTCACCGCCGTGAATATCCTG
GTGCTGCTGTTCGTGATGGTGGCCGGCTTCGTGAAGGGCAACGTGGCCAA
TTGGAAGATCAGCGAAGAGTTCCTGAAGAACATCAGCGCCAGCGCCAGAG
AGCCTCCTTCTGAAAACGGCACCAGCATCTATGGCGCAGGCGGCTTTATG
CCCTACGGCTTTACTGGAACACTGGCAGGCGCCGCTACCTGCTTCTATGC
CTTCGTGGGCTTCGACTGTATCGCCACCACTGGGGAAGAAGTGCGGAACC
CTCAGAAGGCTATCCCCATCGGCATCGTGACAAGCCTGCTCGTGTGCTTC
ATGGCCTACTTCGGAGTGTCCGCCGCACTGACCCTGATGATGCCTTACTA
CCTGCTGGACGAGAAGTCCCCTCTGCCTGTGGCCTTTGAGTATGTTGGCT
GGGGCCCTGCCAAATACGTGGTGGCTGCTGGATCTCTGTGCGCCCTGTCT
ACATCTCTGCTGGGCAGCATGTTCCCTCTGCCAAGAATCCTGTTCGCCAT
GGCCCGGGATGGCCTGCTGTTCAGATTCCTGGCCAGAGTGAACAGCAAGC
GGCAGTCTCCTGTGGCCGCTACACTTACAGCTGGCGTGATCTCTGCCCTG
ATGGCTTTCCTGTTCGACCTGAAGGCCCTGGTGGACATGATGAGCATCGG
CACACTGATGGCCTACAGCCTGGTGGCAGCCTGCGTGCTGATTCTGAGAT
ACCAGCCAGGCCTGTCCTACGACCAGCCTAAGTGTTCCCCTGAGAAGGAC
GGCCTGGGCAGCTCTCCTAGAGTGACAAGCAAGAGCGAGAGCCAAGTGAC
CATGCTGCAGAGACAGGGCTTCAGCATGCGGACCCTGTTCTGCCCTTCTC
TGCTGCCTACACAGCAGTCTGCTAGCCTGGTGTCTTTCCTCGTGGGATTT
CTGGCCTTTCTGGTGCTGGGCCTGAGCGTGCTGACAACATATGGGGTGCA
CGCCATCACCAGACTGGAAGCTTGGAGTCTGGCTCTGCTGGCCCTGTTCC
TGGTTCTGTTTGTGGCCATCGTGCTGACCATTTGGCGGCAGCCCCAGAAC
CAGCAGAAAGTGGCTTTCATGGTGCCCTTTCTGCCTTTCCTGCCAGCCTT
CAGCATCCTGGTCAACATCTACCTGATGGTGCAGCTGAGCGCCGACACCT
GGGTCCGATTTTCCATCTGGATGGCTATCGGCTTCCTCATCTACTTCAGC
TACGGCATCCGGCACTCCCTGGAAGGCCATCTGAGAGATGAGAACAACGA
AGAGGACGCTTACCCCGACAACGTGCACGCCGCTGCCGAAGAGAAATCTG
CCATCCAGGCCAACGACCACCATCCAAGAAACCTGAGCAGCCCCTTCATC
TTCCACGAGAAAACCAGCGAGTTT (SEQ ID NO:202); and

ATGATTCCCTGCAGAGCCGCTCTGACCTTCGCCAGATGCCTGATCAGACG
GAAGATCGTGACCCTGGACAGCCTGGAAGATACCAAGCTGTGCCGGTGCC
TGAGCACCATGGATCTGATTGCCCTCGGCGTGGGCTCTACACTTGGAGCT
GGTGTTTATGTGCTGGCTGGCGAGGTGGCCAAGGCCGATTCTGGACCTTC
TATCGTGGTGTCCTTCCTGATCGCCGCTCTGGCCTCTGTTATGGCCGGAC
TGTGTTACGCCGAGTTCGGAGCCAGAGTGCCTAAGACAGGCAGCGCCTAC
CTGTACACCTACGTGACAGTGGGAGAGCTGTGGGCCTTTATCACCGGCTG
GAACCTGATCCTGAGCTACGTGATCGGCACCTCCTCTGTGGCTAGAGCTT
GGAGCGGCACCTTTGACGAGCTGCTGTCTAAGCAGATCGGCCAGTTCCTG
CGGACCTACTTCCGGATGAATTACACCGGCCTGGCCGAGTATCCCGACTT
CTTCGCCGTGTGTCTGATCCTGCTGCTTGCCGGACTGCTGAGCTTCGGCG
TGAAAGAGTCTGCCTGGGTCAACAAGGTGTTCACCGCCGTGAATATCCTG
GTGCTGCTGTTCGTGATGGTGGCCGGCTTCGTGAAGGGCAACGTGGCCAA
TTGGAAGATCAGCGAAGAGTTCCTGAAGAACATCAGCGCCAGCGCCAGAG
AGCCTCCTTCTGAAAACGGCACCAGCATCTATGGCGCAGGCGGCTTTATG
CCCTACGGCTTTACTGGAACACTGGCAGGCGCCGCTACCTGCTTCTATGC
CTTCGTGGGCTTCGACTGTATCGCCACCACTGGGGAAGAAGTGCGGAACC
CTCAGAAGGCTATCCCCATCGGCATCGTGACAAGCCTGCTCGTGTGCTTC
ATGGCCTACTTCGGAGTGTCCGCCGCACTGACCCTGATGATGCCTTACTA
CCTGCTGGACGAGAAGTCCCCTCTGCCTGTGGCCTTTGAGTATGTTGGCT
GGGGCCCTGCCAAATACGTGGTGGCTGCTGGATCTCTGTGCGCCCTGTCT
ACATCTCTGCTGGGCAGCATGTTCCCTCTGCCAAGAATCCTGTTCGCCAT
GGCCGAGGATGGCCTGCTGTTCAGATTCCTGGCCAGAGTGAACAGCAAGC
GGCAGTCTCCTGTGGCCGCTACACTTACAGCTGGCGTGATCTCTGCCCTG
ATGGCTTTCCTGTTCGACCTGAAGGCCCTGGTGGACATGATGAGCATCGG
CACACTGATGGCCTACAGCCTGGTGGCAGCCTGCGTGCTGATTCTGAGAT
ACCAGCCAGGCCTGTCCTACGACCAGCCTAAGTGTTCCCCTGAGAAGGAC
GGCCTGGGCAGCTCTCCTAGAGTGACAAGCAAGAGCGAGAGCCAAGTGAC
CATGCTGCAGAGACAGGGCTTCAGCATGCGGACCCTGTTCTGCCCTTCTC
TGCTGCCTACACAGCAGTCTGCTAGCCTGGTGTCTTTCCTCGTGGGATTT
CTGGCCTTTCTGGTGCTGGGCCTGAGCGTGCTGACAACATATGGGGTGCA
CGCCATCACCAGACTGGAAGCTTGGAGTCTGGCTCTGCTGGCCCTGTTCC
TGGTTCTGTTTGTGGCCATCGTGCTGACCATTTGGCGGCAGCCCCAGAAC
CAGCAGAAAGTGGCTTTCATGGTGCCCTTTCTGCCTTTCCTGCCAGCCTT
CAGCATCCTGGTCAACATCTACCTGATGGTGCAGCTGAGCGCCGACACCT
GGGTCCGATTTTCCATCTGGATGGCTATCGGCTTCCTCATCTACTTCAGC
TACGGCATCCGGCACTCCCTGGAAGGCCATCTGAGAGATGAGAACAACGA
AGAGGACGCTTACCCCGACAACGTGCACGCCGCTGCCGAAGAGAAATCTG
CCATCCAGGCCAACGACCACCATCCAAGAAACCTGAGCAGCCCCTTCATC
TTCCACGAGAAAACCAGCGAGTTT (SEQ ID NO:203).

“SLC7A3” (also known as solute carrier family 7 member 3, CAT3, ATRC3, and CAT-3) as used herein refers to gene identified by Entrez Gene ID No. 84889, allelic variants thereof, orthologs thereof, and mRNA transcripts encoded by the gene, including the nucleotide sequence of NCBI Reference Sequence: NM_001048164.3 (SEQ ID NO:204) or NM_032803.6 (SEQ ID NO:205).

Cationic amino acid transporter 3 (CAT-3) proteins described herein include protein sequences encoded by SLC7A3, the amino acid sequence of NCBI Reference Sequences NP_001041629.1 and NP_116192.4, and the amino acid sequence of NCBI CCDS ID NO. CCDS 14404.1:

MPWQAFRRFGQKLVRRRTLESGMAETRLARCLSTLDLVALGVGSTLGAGV
YVLAGEVAKDKAGPSIVICFLVAALSSVLAGLCYAEFGARVPRSGSAYLY
SYVTVGELWAFTTGWNLILSYVIGTASVARAWSSAFDNLIGNHISKTLQG
SIALHVPHVLAEYPDFFALGLVLLLTGLLALGASESALVTKVFTGVNLLV
LGFVMISGFVKGDVHNWKLTEEDYELAMAELNDTYSLGPLGSGGFVPFGF
EGILRGAATCFYAFVGFDCIATTGEEAQNPQRSIPMGIVISLSVCFLAYF
AVSSALTLMMPYYQLQPESPLPEAFLYIGWAPARYVVAVGSLCALSTSLL
GSMFPMPRVIYAMAEDGLLFRVLARIHTGTRTPIIATVVSGIIAAFMAFL
FKLTDLVDLMSIGTLLAYSLVSICVLILRYQPDQETKTGEEVELQEEAIT
TESEKLTLWGLFFPLNSIPTPLSGQIVYVCSSLLAVLLTALCLVLAQWSV
PLLSGDLLWTAVVVLLLLLIIGIIVVIWRQPQSSTPLHFKVPALPLLPLM
SIFVNIYLMMQMTAGTWARFGVWMLIGFAIYFGYGIQHSLEEIKSNQPSR
KSRAKTVDLDPGTLYVHSV (SEQ ID NO:208).

CAT-3 nucleotide sequences include the nucleotide sequence of NCBI CCDS ID NO. CCDS 14404.1:

ATGCCGTGGCAAGCATTTCGCAGATTTGGTCAAAAGCTGGTACGCAGACG
TACACTGGAGTCAGGCATGGCTGAGACTCGCCTTGCCAGATGCCTAAGCA
CCCTGGATTTAGTGGCCCTGGGTGTGGGCAGCACATTGGGTGCAGGCGTG
TATGTCCTAGCTGGCGAGGTGGCCAAAGATAAAGCAGGGCCATCCATTGT
GATCTGCTTTTTGGTGGCTGCCCTGTCTTCTGTGTTGGCTGGGCTGTGCT
ATGCGGAGTTTGGTGCCCGGGTTCCCCGTTCTGGTTCGGCATATCTCTAC
AGCTATGTCACTGTGGGTGAACTCTGGGCCTTCACCACTGGCTGGAACCT
CATCCTCTCCTATGTCATTGGTACAGCCAGTGTGGCCCGGGCCTGGAGCT
CTGCTTTTGACAACCTGATTGGGAACCACATCTCTAAGACTCTGCAGGGG
TCCATTGCACTGCACGTGCCCCATGTCCTTGCAGAATATCCAGATTTCTT
TGCTTTGGGCCTCGTGTTGCTGCTCACTGGATTGTTGGCTCTCGGGGCTA
GTGAGTCGGCCCTGGTTACCAAAGTGTTCACAGGCGTGAACCTTTTGGTT
CTTGGGTTCGTCATGATCTCTGGCTTCGTTAAGGGGGACGTGCACAACTG
GAAGCTCACAGAAGAGGACTACGAATTGGCCATGGCTGAACTCAATGACA
CCTATAGCTTGGGTCCTCTGGGCTCTGGAGGATTTGTGCCTTTCGGCTTC
GAGGGAATTCTCCGTGGAGCAGCGACCTGTTTCTATGCATTTGTTGGTTT
CGACTGTATTGCTACCACTGGAGAAGAAGCCCAGAATCCCCAGCGTTCCA
TCCCGATGGGCATTGTGATCTCACTGTCTGTCTGCTTTTTGGCGTATTTT
GCTGTCTCTTCTGCACTCACCCTGATGATGCCTTACTACCAGCTTCAGCC
TGAGAGCCCTTTGCCTGAGGCATTTCTCTACATTGGATGGGCTCCTGCCC
GCTATGTTGTGGCTGTTGGCTCCCTCTGTGCTCTTTCTACCAGCCTCCTG
GGCTCCATGTTCCCCATGCCTCGGGTGATCTACGCGATGGCAGAGGATGG
CCTCCTGTTCCGTGTACTTGCTCGGATCCACACCGGCACACGCACCCCAA
TCATAGCCACCGTGGTCTCTGGCATTATTGCAGCATTCATGGCATTCCTC
TTCAAACTCACTGATCTTGTGGACCTCATGTCAATTGGGACCCTGCTTGC
TTACTCCCTGGTGTCGATTTGTGTTCTCATCCTCAGGTATCAACCTGATC
AGGAGACAAAGACTGGGGAAGAAGTGGAGTTGCAGGAGGAGGCAATAACT
ACTGAATCAGAGAAGTTGACCCTATGGGGACTATTTTTCCCACTCAACTC
CATCCCCACTCCACTCTCTGGCCAAATTGTCTATGTTTGTTCCTCATTGC
TTGCTGTCCTGCTGACTGCTCTTTGCCTGGTGCTGGCCCAGTGGTCAGTT
CCATTGCTTTCTGGAGACCTGCTGTGGACTGCAGTGGTTGTGCTGCTCCT
GCTGCTCATTATTGGGATCATTGTGGTCATCTGGAGACAGCCACAGAGTT
CCACTCCCCTTCACTTTAAGGTGCCTGCTTTGCCTCTCCTCCCACTAATG
AGCATCTTTGTGAATATTTACCTTATGATGCAGATGACAGCTGGTACCTG
GGCCCGATTTGGGGTCTGGATGCTGATTGGCTTTGCTATCTACTTCGGCT
ATGGGATCCAGCACAGCCTGGAAGAGATTAAGAGTAACCAACCCTCACGC
AAGTCTAGAGCCAAAACTGTAGACCTTGATCCCGGCACTCTCTATGTCCA
CTCAGTTTGA (SEQ ID NO:209).

“SLC7A4” (also known as solute carrier family 7 member 4, VH, CAT4, CAT-4, and HCAT3) as used herein refers to gene identified by Entrez Gene ID No. 6545, allelic variants thereof, orthologs thereof, and mRNA transcripts encoded by the gene, including the nucleotide sequence of NCBI Reference Sequence NM_004173.3 (SEQ ID NO:210).

Cationic amino acid transporter 4 (CAT-4) proteins described herein include protein sequences encoded by SLC7A4, the amino acid sequence of NCBI Reference Sequence NP_004164.2, and the amino acid sequence of NCBI CCDS ID NO. CCDS33608.1:

MARGLPTIASLARLCQKLNRLKPLEDSTMETSLRRCLSTLDLTLLGVGGM
VGSGLYVLTGAVAKEVAGPAVLLSFGVAAVASLLAALCYAEFGARVPRTG
SAYLFTYVSMGELWAFLIGWNVLLEYIIGGAAVARAWSGYLDSMFSHSIR
NFTETHVGSWQVPLLGHYPDFLAAGIILLASAFVSCGARVSSWLNHTFSA
ISLLVILFIVILGFILAQPHNWSADEGGFAPFGFSGVMAGTASCFYAFVG
FDVIAASSEEAQNPRRSVPLAIAISLAIAAGAYILVSTVLTLMVPWHSLD
PDSALADAFYQRGYRWAGFIVAAGSICAMNTVLLSLLFSLPRIVYAMAAD
GLFFQVFAHVHPRTQVPVAGTLAFGLLTAFLALLLDLESLVQFLSLGTLL
AYTFVATSIIVLRFQKSSPPSSPGPASPGPLTKQQSSFSDHLQLVGTVHA
SVPEPGELKPALRPYLGFLDGYSPGAVVTWALGVMLASAITIGCVLVFGN
STLHLPHWGYILLLLLTSVMFLLSLLVLGAHQQQYREDLFQIPMVPLIPA
LSIVLNICLMLKLSYLTWVRFSIWLLMGLAVYFGYGIRHSKENQRELPGL
NSTHYVVFPRGSLEETVQAMQPPSQAPAQDPGHME (SEQ ID NO:212
).

CAT-4 nucleotide sequences include the nucleotide sequence of NCBI CCDS ID NO. CCDS33608.1:

ATGGCCCGGGGGCTGCCCACCATTGCTAGCCTGGCACGCTTATGCCAGAA
GCTGAACCGCCTGAAGCCGCTGGAGGACTCCACCATGGAGACGTCACTGC
GGCGCTGCCTGTCCACGCTGGACCTGACTCTTCTGGGCGTGGGTGGCATG
GTGGGCTCGGGTCTCTACGTGCTCACAGGTGCCGTGGCCAAGGAGGTGGC
TGGCCCTGCTGTGCTCTTGTCCTTCGGTGTGGCCGCTGTGGCCTCCCTGC
TGGCAGCCCTATGCTATGCAGAATTTGGGGCACGTGTGCCACGCACGGGC
TCTGCCTACCTGTTCACCTACGTATCCATGGGCGAGCTGTGGGCCTTCCT
CATCGGCTGGAATGTTCTCCTCGAATACATCATCGGTGGCGCCGCCGTGG
CCCGTGCCTGGAGTGGCTACCTGGACTCTATGTTCAGCCACAGCATCCGC
AACTTCACTGAGACCCACGTGGGTTCTTGGCAGGTGCCCCTCCTGGGCCA
CTACCCGGACTTCCTGGCTGCTGGCATCATCCTCCTGGCCTCTGCCTTTG
TCTCCTGTGGAGCCCGCGTGTCCTCCTGGCTCAATCACACCTTCTCGGCC
ATCAGCCTGCTTGTCATTCTCTTCATTGTCATCCTGGGCTTCATCCTGGC
CCAGCCTCACAACTGGAGCGCTGACGAAGGCGGCTTTGCACCCTTCGGCT
TCTCCGGCGTCATGGCCGGCACTGCCTCCTGCTTCTATGCTTTCGTGGGC
TTCGACGTCATTGCCGCCTCCAGTGAGGAGGCCCAGAACCCACGGCGGTC
TGTGCCTCTGGCCATCGCCATCTCGCTTGCCATTGCAGCTGGTGCCTACA
TCCTTGTCTCCACCGTGCTAACCCTCATGGTGCCCTGGCACAGCCTGGAC
CCCGACTCAGCGCTTGCAGATGCCTTCTACCAGCGGGGCTACAGGTGGGC
TGGCTTCATCGTGGCAGCTGGCTCCATCTGCGCCATGAACACCGTCCTGC
TCAGCCTCCTCTTCTCCCTGCCACGCATTGTCTATGCCATGGCCGCCGAT
GGGCTCTTCTTCCAGGTGTTTGCCCATGTGCACCCCCGGACACAGGTGCC
TGTGGCGGGCACCCTGGCGTTCGGGCTCCTCACGGCCTTCCTGGCACTGC
TGCTGGACCTGGAGTCGCTGGTTCAGTTCCTGTCCCTTGGCACACTCCTG
GCCTACACATTCGTGGCCACCAGTATCATTGTGCTGCGCTTCCAGAAGTC
TTCCCCGCCCAGCTCCCCAGGCCCAGCCAGCCCTGGCCCCCTGACCAAGC
AGCAGAGCTCCTTCTCAGACCACCTACAGCTGGTGGGCACTGTACACGCC
TCCGTCCCTGAGCCAGGGGAGCTGAAGCCAGCCCTGAGGCCCTACCTGGG
CTTCTTGGATGGGTACAGCCCTGGAGCAGTGGTGACTTGGGCGCTTGGCG
TTATGTTGGCCTCAGCCATCACCATAGGCTGCGTGCTTGTCTTTGGGAAC
TCGACCCTGCACCTCCCACACTGGGGTTACATCCTGCTGCTCCTGCTCAC
CAGTGTCATGTTTCTGCTCAGCCTCCTTGTCCTGGGGGCTCACCAGCAAC
AGTATCGGGAAGACTTATTTCAGATCCCCATGGTTCCCCTGATTCCAGCC
CTGAGCATCGTCCTCAACATCTGCCTCATGCTGAAACTTAGCTATCTGAC
CTGGGTGCGCTTCTCCATCTGGCTGCTGATGGGACTTGCAGTGTATTTCG
GCTATGGCATCCGGCATAGCAAGGAGAACCAGCGGGAGCTGCCAGGGCTG
AACTCCACACACTACGTGGTATTCCCCAGGGGCAGCCTGGAGGAGACAGT
GCAGGCTATGCAGCCCCCCAGCCAGGCACCAGCACAGGACCCTGGCCATA
TGGAGTAG (SEQ ID NO:213).

“SLC7A6” (also known as solute carrier family 7 member 6, LAT3, LAT-2, and y⁺LAT-2) as used herein refers to gene identified by Entrez Gene ID No. 9057, allelic variants thereof, orthologs thereof, mRNA transcripts encoded by the gene, including the nucleotide sequence of NCBI Reference Sequence: NM_001076785.3 (SEQ ID NO:214) or NM_003983.6 (SEQ ID NO:215).

y⁺L amino acid transporter 2 (y⁺LAT2) proteins include protein sequences encoded by SLC7A6, the amino acid sequence of NCBI Reference Sequence NP_001070253.1 and NP_003974.3, and the amino acid sequence of NCBI CCDS ID NO. CCDS32470.1:

MEAREPGRPTPTYHLVPNTSQSQVEEDVSSPPQRSSETMQLKKEISLLNG
VSLVVGNMIGSGIFVSPKGVLVHTASYGMSLIVWAIGGLFSVVGALCYAE
LGTTITKSGASYAYILEAFGGFIAFIRLWVSLLVVEPTGQAIIAITFANY
IIQPSFPSCDPPYLACRLLAAACICLLTFVNCAYVKWGTRVQDTFTYAKV
VALIAIIVMGLVKLCQGHSEHFQDAFEGSSWDMGNLSLALYSALFSYSGW
DTLNFVTEEIKNPERNLPLAIGISMPIVTLIYILTNVAYYTVLNISDVLS
SDAVAVTFADQTFGMFSWTIPIAVALSCFGGLNASIFASSRLFFVGSREG
HLPDLLSMIHIERFTPIPALLFNCTMALIYLIVEDVFQLINYFSFSYWFF
VGLSVVGQLYLRWKEPKRPRPLKLSVFFPIVFCICSVFLVIVPLFTDTIN
SLIGIGIALSGVPFYFMGVYLPESRRPLFIRNVLAAITRGTQQLCFCVLT
ELDVAEEKKDERKTD (SEQ ID NO:218).

y⁺LAT2 nucleotide sequences include the nucleotide sequence of NCBI CCDS ID NO. CCDS32470.1:

ATGGAAGCCAGGGAGCCTGGGAGGCCCACACCCACCTACCATCTTGTCCC
TAACACCAGCCAGTCCCAGGTGGAAGAAGATGTCAGCTCGCCACCTCAAA
GGTCCTCCGAAACTATGCAGCTGAAGAAGGAGATCTCCCTGCTGAATGGG
GTCAGCCTGGTGGTGGGCAACATGATCGGCTCAGGGATCTTTGTCTCACC
CAAGGGTGTGCTGGTACACACTGCCTCCTATGGGATGTCACTGATTGTGT
GGGCCATTGGTGGGCTCTTCTCTGTTGTGGGTGCCCTTTGTTATGCAGAG
CTGGGGACCACCATCACCAAGTCGGGAGCCAGCTACGCTTATATTCTAGA
GGCCTTTGGGGGCTTCATTGCCTTCATCCGCCTGTGGGTCTCACTGCTAG
TTGTTGAGCCCACCGGTCAGGCCATCATCGCCATCACCTTTGCCAACTAC
ATCATCCAGCCGTCCTTCCCCAGCTGTGATCCCCCATACCTGGCCTGCCG
TCTCCTGGCTGCTGCTTGCATATGTCTGCTGACATTTGTGAACTGTGCCT
ATGTCAAGTGGGGCACACGTGTGCAGGACACGTTCACTTACGCCAAGGTC
GTAGCGCTCATTGCCATCATTGTCATGGGCCTTGTTAAACTGTGCCAGGG
ACACTCTGAGCACTTTCAGGACGCCTTTGAGGGTTCCTCCTGGGACATGG
GAAACCTCTCTCTTGCCCTCTACTCTGCCCTCTTCTCTTACTCAGGTTGG
GACACCCTTAATTTTGTAACAGAAGAAATCAAAAACCCAGAAAGAAATTT
GCCCTTGGCCATTGGGATTTCTATGCCAATTGTGACGCTCATCTACATCC
TGACCAATGTGGCCTATTACACAGTGCTGAACATTTCAGATGTCCTTAGC
AGTGATGCTGTGGCTGTGACATTTGCTGACCAGACGTTTGGCATGTTCAG
CTGGACCATCCCCATTGCTGTTGCCCTGTCCTGCTTTGGGGGCCTCAATG
CATCCATCTTTGCTTCATCAAGGTTGTTCTTCGTGGGCTCCCGGGAGGGC
CACCTACCGGACCTTCTGTCCATGATCCACATTGAGCGTTTTACACCTAT
CCCTGCTTTACTGTTCAATTGCACCATGGCACTCATCTACCTCATCGTGG
AGGATGTTTTCCAGCTTATCAACTACTTCAGCTTCAGCTACTGGTTCTTC
GTGGGCCTGTCTGTTGTTGGACAGCTCTACCTCCGCTGGAAGGAGCCCAA
GCGGCCCCGGCCTCTCAAGCTGAGCGTGTTTTTCCCCATCGTGTTCTGCA
TATGCTCCGTGTTTCTGGTGATAGTGCCCCTCTTCACTGACACCATTAAT
TCCCTCATTGGCATCGGGATTGCCCTTTCTGGAGTCCCTTTCTACTTCAT
GGGTGTTTACCTGCCAGAGTCCCGGAGGCCATTGTTTATTCGGAATGTCC
TGGCTGCTATCACCAGAGGCACCCAGCAGCTTTGCTTTTGTGTCCTGACT
GAGCTTGATGTAGCCGAAGAAAAAAAGGATGAGAGGAAAACTGACTAG (
SEQ ID NO:219).

“SLC7A7” (also known as solute carrier family 7 member 7, LPI, LAT3, MOP-2, Y+LAT1, and y⁺LAT-1) as used herein refers to gene identified by Entrez Gene ID No. 9056, allelic variants thereof, orthologs thereof, mRNA transcripts encoded by the gene, including the nucleotide sequence of NCBI Reference Sequence: NM_001126105.3 (SEQ ID NO:220), NM_003982.4 (SEQ ID NO:221), and NM_001126106.4 (SEQ ID NO:222).

y⁺L amino acid transporter 1 (y⁺LAT1) proteins described herein include the protein encoded by SLC7A7, the amino acid sequence of NCBI Reference Sequence NP_001119578.1 and NP_003973.3, and the amino acid sequence of NCBI CCDS ID NO. CCDS9574.1:

MVDSTEYEVASQPEVETSPLGDGASPGPEQVKLKKEISLLNGVCLIVGNM
IGSGIFVSPKGVLIYSASFGLSLVIWAVGGLFSVFGALCYAELGTTIKKS
GASYAYILEAFGGFLAFIRLWTSLLIIEPTSQAIIAITFANYMVQPLFPS
CFAPYAASRLLAAACICLLTFINCAYVKWGTLVQDIFTYAKVLALIAVIV
AGIVRLGQGASTHFENSFEGSSFAVGDIALALYSALFSYSGWDTLNYVTE
EIKNPERNLPLSIGISMPIVTIIYILTNVAYYTVLDMRDILASDAVAVTF
ADQIFGIFNWIIPLSVALSCFGGLNASIVAASRLFFVGSREGHLPDAICM
IHVERFTPVPSLLFNGIMALIYLCVEDIFQLINYYSFSYWFFVGLSIVGQ
LYLRWKEPDRPRPLKLSVFFPIVFCLCTIFLVAVPLYSDTINSLIGIAIA
LSGLPFYFLIIRVPEHKRPLYLRRIVGSATRYLQVLCMSVAAEMDLEDGG
EMPKQRDPKSN (SEQ ID NO:225).

y⁺LAT1 nucleotide sequences include the nucleotide sequence of NCBI CCDS ID NO. CCDS9574.1:

ATGGTTGACAGCACTGAGTATGAAGTGGCCTCCCAGCCTGAGGTGGAAAC
CTCCCCTTTGGGTGATGGGGCCAGCCCAGGGCCGGAGCAGGTGAAGCTGA
AGAAGGAGATCTCACTGCTTAACGGCGTGTGCCTGATTGTGGGGAACATG
ATCGGCTCGGGCATCTTTGTTTCCCCCAAGGGTGTGCTCATATACAGTGC
CTCCTTTGGTCTCTCTCTGGTCATCTGGGCTGTCGGGGGCCTCTTCTCCG
TCTTTGGGGCCCTTTGTTATGCGGAACTGGGCACCACCATTAAGAAATCT
GGGGCCAGCTATGCCTATATCCTGGAGGCCTTTGGAGGATTCCTTGCTTT
CATCAGACTCTGGACCTCCCTGCTCATCATTGAGCCCACCAGCCAGGCCA
TCATTGCCATCACCTTTGCCAACTACATGGTACAGCCTCTCTTCCCGAGC
TGCTTCGCCCCTTATGCTGCCAGCCGCCTGCTGGCTGCTGCCTGCATTTG
TCTCTTAACCTTCATTAACTGTGCCTATGTCAAATGGGGAACCCTGGTAC
AAGATATTTTCACCTATGCTAAAGTATTGGCACTGATCGCGGTCATCGTT
GCAGGCATTGTTAGACTTGGCCAGGGAGCCTCTACTCATTTTGAGAATTC
CTTTGAGGGTTCATCATTTGCAGTGGGTGACATTGCCCTGGCACTGTACT
CAGCTCTGTTCTCCTACTCAGGCTGGGACACCCTCAACTATGTCACTGAA
GAGATCAAGAATCCTGAGAGGAACCTGCCCCTCTCCATTGGCATCTCCAT
GCCCATTGTCACCATCATCTATATCTTGACCAATGTGGCCTATTATACTG
TGCTAGACATGAGAGACATCTTGGCCAGTGATGCTGTTGCTGTGACTTTT
GCAGATCAGATATTTGGAATATTTAACTGGATAATTCCACTGTCAGTTGC
ATTATCCTGTTTTGGTGGCCTCAATGCCTCCATTGTGGCTGCTTCTAGGC
TTTTCTTTGTGGGCTCAAGAGAAGGCCATCTCCCTGATGCCATCTGCATG
ATCCATGTTGAGCGGTTCACACCAGTGCCTTCTCTGCTCTTCAATGGTAT
CATGGCATTGATCTACTTGTGCGTGGAAGACATCTTCCAGCTCATTAACT
ACTACAGCTTCAGCTACTGGTTCTTTGTGGGGCTTTCTATTGTGGGTCAG
CTTTATCTGCGCTGGAAGGAGCCTGATCGACCTCGTCCCCTCAAGCTCAG
CGTTTTCTTCCCGATTGTCTTCTGCCTCTGCACCATCTTCCTGGTGGCTG
TTCCACTTTACAGTGATACTATCAACTCCCTCATCGGCATTGCCATTGCC
CTCTCAGGCCTGCCCTTTTACTTCCTCATCATCAGAGTGCCAGAACATAA
GCGACCGCTTTACCTCCGAAGGATCGTGGGGTCTGCCACAAGGTACCTCC
AGGTCCTGTGTATGTCAGTTGCTGCAGAAATGGATTTGGAAGATGGAGGA
GAGATGCCCAAGCAACGGGATCCCAAATCTAACTAA (SEQ ID NO:22
6).

“SLC3A2” (also known as solute carrier family 3 member 2, 4F2, CD98, MDU1, 4F2HC, 4T2HC, NACAE, and CD98HC) as used herein refers to gene identified by Entrez Gene ID No. 6520, allelic variants thereof, orthologs thereof, mRNA transcripts encoded by the gene, including the nucleotide sequence of NCBI Reference Sequence: NM_001012662.3 (SEQ ID NO:227), NM_001012664.3 (SEQ ID NO:228), NM_001013251.3 (SEQ ID NO:229), or NM_002394.6 (SEQ ID NO:230).

4F2 cell-surface antigen heavy chain (4F2hc) proteins described herein include the protein encoded by SLC3A2 and the amino acid sequences of NCBI Reference Sequence NP_002385.3 and NCBI CCDS ID NO. CCDS8039.2:

MELQPPEASIAVVSIPRQLPGSHSEAGVQGLSAGDDSELGSHCVAQTGLE
LLASGDPLPSASQNAEMIETGSDCVTQAGLQLLASSDPPALASKNAEVTG
TMSQDTEVDMKEVELNELEPEKQPMNAASGAAMSLAGAEKNGLVKIKVAE
DEAEAAAAAKFTGLSKEELLKVAGSPGWVRTRWALLLLFWLGWLGMLAGA
VVIIVRAPRCRELPAQKWWHTGALYRIGDLQAFQGHGAGNLAGLKGRLDY
LSSLKVKGLVLGPIHKNQKDDVAQTDLLQIDPNFGSKEDFDSLLQSAKKK
SIRVILDLTPNYRGENSWFSTQVDTVATKVKDALEFWLQAGVDGFQVRDI
ENLKDASSFLAEWQNITKGFSEDRLLIAGTNSSDLQQILSLLESNKDLLL
TSSYLSDSGSTGEHTKSLVTQYLNATGNRWCSWSLSQARLLTSFLPAQLL
RLYQLMLFTLPGTPVFSYGDEIGLDAAALPGQPMEAPVMLWDESSFPDIP
GAVSANMTVKGQSEDPGSLLSLFRRLSDQRSKERSLLHGDFHAFSAGPGL
FSYIRHWDQNERFLVVLNFGDVGLSAGLQASDLPASASLPAKADLLLSTQ
PGREEGSPLELERLKLEPHEGLLLRFPYAA (SEQ ID NO:232).

4F2hc nucleotide sequences include the nucleotide sequence of NCBI CCDS ID NO. CCDS8039.2:

ATGGAGCTACAGCCTCCTGAAGCCTCGATCGCCGTCGTGTCGATTCCGCG
CCAGTTGCCTGGCTCACATTCGGAGGCTGGTGTCCAGGGTCTCAGCGCGG
GGGACGACTCAGAGTTGGGGTCTCACTGTGTTGCCCAGACTGGTCTCGAA
CTCTTGGCCTCAGGTGATCCTCTTCCCTCAGCTTCCCAGAATGCCGAGAT
GATAGAGACGGGGTCTGACTGTGTTACCCAGGCTGGTCTTCAACTCTTGG
CCTCAAGTGATCCTCCTGCCTTAGCTTCCAAGAATGCTGAGGTTACAGGC
ACCATGAGCCAGGACACCGAGGTGGATATGAAGGAGGTGGAGCTGAATGA
GTTAGAGCCCGAGAAGCAGCCGATGAACGCGGCGTCTGGGGCGGCCATGT
CCCTGGCGGGAGCCGAGAAGAATGGTCTGGTGAAGATCAAGGTGGCGGAA
GACGAGGCGGAGGCGGCAGCCGCGGCTAAGTTCACGGGCCTGTCCAAGGA
GGAGCTGCTGAAGGTGGCAGGCAGCCCCGGCTGGGTACGCACCCGCTGGG
CACTGCTGCTGCTCTTCTGGCTCGGCTGGCTCGGCATGCTTGCTGGTGCC
GTGGTCATAATCGTGCGAGCGCCGCGTTGTCGCGAGCTACCGGCGCAGAA
GTGGTGGCACACGGGCGCCCTCTACCGCATCGGCGACCTTCAGGCCTTCC
AGGGCCACGGCGCGGGCAACCTGGCGGGTCTGAAGGGGCGTCTCGATTAC
CTGAGCTCTCTGAAGGTGAAGGGCCTTGTGCTGGGTCCAATTCACAAGAA
CCAGAAGGATGATGTCGCTCAGACTGACTTGCTGCAGATCGACCCCAATT
TTGGCTCCAAGGAAGATTTTGACAGTCTCTTGCAATCGGCTAAAAAAAAG
AGCATCCGTGTCATTCTGGACCTTACTCCCAACTACCGGGGTGAGAACTC
GTGGTTCTCCACTCAGGTTGACACTGTGGCCACCAAGGTGAAGGATGCTC
TGGAGTTTTGGCTGCAAGCTGGCGTGGATGGGTTCCAGGTTCGGGACATA
GAGAATCTGAAGGATGCATCCTCATTCTTGGCTGAGTGGCAAAATATCAC
CAAGGGCTTCAGTGAAGACAGGCTCTTGATTGCGGGGACTAACTCCTCCG
ACCTTCAGCAGATCCTGAGCCTACTCGAATCCAACAAAGACTTGCTGTTG
ACTAGCTCATACCTGTCTGATTCTGGTTCTACTGGGGAGCATACAAAATC
CCTAGTCACACAGTATTTGAATGCCACTGGCAATCGCTGGTGCAGCTGGA
GTTTGTCTCAGGCAAGGCTCCTGACTTCCTTCTTGCCGGCTCAACTTCTC
CGACTCTACCAGCTGATGCTCTTCACCCTGCCAGGGACCCCTGTTTTCAG
CTACGGGGATGAGATTGGCCTGGATGCAGCTGCCCTTCCTGGACAGCCTA
TGGAGGCTCCAGTCATGCTGTGGGATGAGTCCAGCTTCCCTGACATCCCA
GGGGCTGTAAGTGCCAACATGACTGTGAAGGGCCAGAGTGAAGACCCTGG
CTCCCTCCTTTCCTTGTTCCGGCGGCTGAGTGACCAGCGGAGTAAGGAGC
GCTCCCTACTGCATGGGGACTTCCACGCGTTCTCCGCTGGGCCTGGACTC
TTCTCCTATATCCGCCACTGGGACCAGAATGAGCGTTTTCTGGTAGTGCT
TAACTTTGGGGATGTGGGCCTCTCGGCTGGACTGCAGGCCTCCGACCTGC
CTGCCAGCGCCAGCCTGCCAGCCAAGGCTGACCTCCTGCTCAGCACCCAG
CCAGGCCGTGAGGAGGGCTCCCCTCTTGAGCTGGAACGCCTGAAACTGGA
GCCTCACGAAGGGCTGCTGCTCCGCTTCCCCTACGCGGCCTGA (SEQ I
D NO:233).

“SLC7A9” (also known as solute carrier family 7 member 9, BAT1, and CSNU3) as used herein refers to gene identified by Entrez Gene ID No. 11136, allelic variants thereof, orthologs thereof, mRNA transcripts encoded by the gene, including the nucleotide sequence of NCBI Reference Sequence NM_001126335.2 (SEQ ID NO:234), NM_001243036.2 (SEQ ID NO:235), NM_014270.5 (SEQ ID NO:236).

Sodium-dependent neutral amino acid transporter BAT1 (b^{0,+}AT) proteins described herein include the protein encoded by SLC7A9 and the amino acid sequences of NCBI Reference Sequence NP_001119807.1, NP_001229965.1, NP_055085.1, and NCBI CCDS ID NO. CCDS12425.1:

MGDTGLRKRREDEKSIQSQEPKTTSLQKELGLISGISIIVGTIIGSGIFV
SPKSVLSNTEAVGPCLIIWAACGVLATLGALCFAELGTMITKSGGEYPYL
MEAYGPIPAYLFSWASLIVIKPTSFAIICLSFSEYVCAPFYVGCKPPQIV
VKCLAAAAILFISTVNSLSVRLGSYVQNIFTAAKLVIVAIIIISGLVLLA
QGNTKNFDNSFEGAQLSVGAISLAFYNGLWAYDGWNQLNYITEELRNPYR
NLPLAIIIGIPLVTACYILMNVSYFTVMTATELLQSQAVAVTFGDRVLYP
ASWIVPLFVAFSTIGAANGTCFTAGRLIYVAGREGHMLKVLSYISVRRLT
PAPAIIFYGIIATIYIIPGDINSLVNYFSFAAWLFYGLTILGLIVMRFTR
KELERPIKVPVVIPVLMTLISVFLVLAPIISKPTWEYLYCVLFILSGLLF
YFLFVHYKFGWAQKISKPITMHLQMLMEVVPPEEDPE (SEQ ID NO:2
40).

b^{0,+}AT nucleotide sequences include the nucleotide sequence of NCBI CCDS ID NO. CCDS12425.1:

ATGGGGGATACTGGCCTGAGAAAGCGGAGAGAGGATGAGAAGTCGATCCA
GAGCCAAGAGCCTAAGACCACCAGTCTCCAAAAGGAGCTGGGCCTCATCA
GTGGCATCTCCATCATCGTGGGCACCATCATTGGCTCTGGGATCTTCGTT
TCCCCCAAGTCTGTGCTCAGCAACACGGAAGCTGTGGGGCCCTGCCTCAT
CATATGGGCGGCTTGCGGGGTCCTCGCGACGCTGGGTGCCCTGTGCTTTG
CGGAGCTTGGCACAATGATCACCAAGTCAGGGGGAGAGTATCCCTACCTG
ATGGAGGCCTACGGGCCCATCCCCGCCTACCTCTTCTCCTGGGCCAGCCT
GATCGTCATTAAGCCCACGTCCTTCGCCATCATCTGCCTCAGCTTCTCCG
AGTATGTGGTGCGCCCTTCTATGTGGGCTGCAAGCCTCCTCAAATCGTTG
TGAAATGCCTGGCCGCCGCCGCCATCTTGTTCATCTCGACAGTGAACTCA
CTGAGCGTGCGGCTGGGAAGCTACGTCCAGAACATCTTCACCGCGGCCAA
GCTGGTGATCGTGGCCATCATCATCATCAGCGGGCTGGTGCTCCTGGCCC
AAGGAAACACAAAGAATTTTGATAATTCTTTCGAGGGCGCCCAGCTGTCT
GTGGGAGCCATCAGCCTGGCGTTTTACAATGGACTCTGGGCCTATGATGG
ATGGAATCAACTCAATTACATCACAGAAGAACTTAGAAACCCTTACAGAA
ACCTGCCTTTGGCCATTATCATCGGGATCCCCCTGGTGACGGCGTGCTAC
ATCCTCATGAACGTGTCCTACTTCACCGTGATGACTGCCACCGAACTCCT
GCAGTCCCAGGCGGTGGCTGTGACATTTGGTGACCGTGTTCTCTATCCTG
CTTCTTGGATCGTTCCACTTTTTGTGGCATTTTCAACCATCGGTGCTGCT
AACGGGACCTGCTTCACAGCGGGCAGACTCATTTACGTGGCGGGCCGGGA
GGGTCACATGCTCAAAGTGCTTTCTTACATCAGCGTCAGGCGCCTCACTC
CAGCCCCCGCCATCATCTTTTATGGTATCATAGCAACGATTTATATCATC
CCTGGTGACATAAACTCGTTAGTCAATTATTTCAGCTTTGCCGCATGGCT
GTTTTATGGCCTGACGATTCTAGGACTCATCGTGATGAGATTTACAAGGA
AAGAGCTGGAAAGGCCTATCAAGGTGCCCGTAGTCATTCCCGTCTTGATG
ACACTCATCTCTGTGTTTTTGGTTCTGGCTCCAATCATCAGCAAGCCCAC
CTGGGAGTACCTCTACTGTGTGCTGTTTATATTAAGCGGCCTTTTATTTT
ACTTCCTGTTTGTCCACTACAAGTTTGGATGGGCTCAGAAAATCTCAAAG
CCGATTACCATGCACCTTCAGATGCTAATGGAAGTGGTCCCACCGGAGGA
AGACCCTGAGTAA (SEQ ID NO:241).

“SLC3A1” (also known as solute carrier family 3 member 1, D2H, ATR1, NBAT, RBAT, and CSNU1) as used herein refers to gene identified by Entrez Gene ID No. 6519, allelic variants thereof, orthologs thereof, mRNA transcripts encoded by the gene, including the nucleotide sequence of NCBI Reference Sequence NM_000341.4 (SEQ ID NO:242).

Neutral and basic amino acid transport protein rBAT (rBAT) proteins described herein include the protein encoded by SLC3A1 and the amino acid sequences of NCBI Reference Sequence NP_000332.2 and NCBI CCDS ID NO. CCDS1819.1:

MAEDKSKRDSIEMSMKGCQTNNGFVHNEDILEQTPDPGSSTDNLKHSTRG
ILGSQEPDFKGVQPYAGMPKEVLFQFSGQARYRIPREILFWLTVASVLVL
IAATIAIIALSPKCLDWWQEGPMYQIYPRSFKDSNKDGNGDLKGIQDKLD
YITALNIKTVWITSFYKSSLKDFRYGVEDFREVDPIFGTMEDFENLVAAI
HDKGLKLIIDFIPNHTSDKHIWFQLSRTRTGKYTDYYIWHDCTHENGKTI
PPNNWLSVYGNSSWHFDEVRNQCYFHQFMKEQPDLNFRNPDVQEEIKEIL
RFWLTKGVDGFSLDAVKFLLEAKHLRDEIQVNKTQIPDTVTQYSELYHDF
TTTQVGMHDIVRSFRQTMDQYSTEPGRYRFMGTEAYAESIDRTVMYYGLP
FIQEADFPFNNYLSMLDTVSGNSVYEVITSWMENMPEGKWPNWMIGGPDS
SRLTSRLGNQYVNVMNMLLFTLPGTPITYYGEEIGMGNIVAANLNESYDI
NTLRSKSPMQWDNSSNAGFSEASNTWLPTNSDYHTVNVDVQKTQPRSALK
LYQDLSLLHANELLLNRGWFCHLRNDSHYVVYTRELDGIDRIFIVVLNFG
ESTLLNLHNMISGLPAKMRIRLSTNSADKGSKVDTSGIFLDKGEGLIFEH
NTKNLLHRQTAFRDRCFVSNRACYSSVLNILYTSC (SEQ ID NO:244
).

rBAT nucleotide sequences include the nucleotide sequence of NCBI CCDS ID NO. CCDS1819.1:

ATGGCTGAAGATAAAAGCAAGAGAGACTCCATCGAGATGAGTATGAAGGG
ATGCCAGACAAACAACGGGTTTGTCCATAATGAAGACATTCTGGAGCAGA
CCCCGGATCCAGGAAGCTCAACAGACAACCTGAAGCACAGCACCAGGGGC
ATCCTTGGCTCCCAGGAGCCCGACTTCAAGGGCGTCCAGCCCTATGCGGG
GATGCCCAAGGAGGTGCTGTTCCAGTTCTCTGGCCAGGCCCGCTACCGCA
TACCTCGGGAGATCCTCTTCTGGCTCACAGTGGCTTCTGTGCTGGTGCTC
ATCGCGGCCACCATAGCCATCATTGCCCTCTCTCCAAAGTGCCTAGACTG
GTGGCAGGAGGGGCCCATGTACCAGATCTACCCAAGGTCTTTCAAGGACA
GTAACAAGGATGGGAACGGAGATCTGAAAGGTATTCAAGATAAACTGGAC
TACATCACAGCTTTAAATATAAAAACTGTTTGGATTACTTCATTTTATAA
ATCGTCCCTTAAAGATTTCAGATATGGTGTTGAAGATTTCCGGGAAGTTG
ATCCCATTTTTGGAACGATGGAAGATTTTGAGAATCTGGTTGCAGCCATA
CATGATAAAGGTTTAAAATTAATCATCGATTTCATACCAAACCACACGAG
TGATAAACATATTTGGTTTCAATTGAGTCGGACACGGACAGGAAAATATA
CTGATTATTATATCTGGCATGACTGTACCCATGAAAATGGCAAAACCATT
CCACCCAACAACTGGTTAAGTGTGTATGGAAACTCCAGTTGGCACTTTGA
CGAAGTGCGAAACCAATGTTATTTTCATCAGTTTATGAAAGAGCAACCTG
ATTTAAATTTCCGCAATCCTGATGTTCAAGAAGAAATAAAAGAAATTTTA
CGGTTCTGGCTCACAAAGGGTGTTGATGGTTTTAGTTTGGATGCTGTTAA
ATTCCTCCTAGAAGCAAAGCACCTGAGAGATGAGATCCAAGTAAATAAGA
CCCAAATCCCGGACACGGTCACACAATACTCGGAGCTGTACCATGACTTC
ACCACCACGCAGGTGGGAATGCACGACATTGTCCGCAGCTTCCGGCAGAC
CATGGACCAATACAGCACGGAGCCCGGCAGATACAGGTTCATGGGGACTG
AAGCCTATGCAGAGAGTATTGACAGGACCGTGATGTACTATGGATTGCCA
TTTATCCAAGAAGCTGATTTTCCCTTCAACAATTACCTCAGCATGCTAGA
CACTGTTTCTGGGAACAGCGTGTATGAGGTTATCACATCCTGGATGGAAA
ACATGCCAGAAGGAAAATGGCCTAACTGGATGATTGGTGGACCAGACAGT
TCACGGCTGACTTCGCGTTTGGGGAATCAGTATGTCAACGTGATGAACAT
GCTTCTTTTCACACTCCCTGGAACTCCTATAACTTACTATGGAGAAGAAA
TTGGAATGGGAAATATTGTAGCCGCAAATCTCAATGAAAGCTATGATATT
AATACCCTTCGCTCAAAGTCACCAATGCAGTGGGACAATAGTTCAAATGC
TGGTTTTTCTGAAGCTAGTAACACCTGGTTACCTACCAATTCAGATTACC
ACACTGTGAATGTTGATGTCCAAAAGACTCAGCCCAGATCGGCTTTGAAG
TTATATCAAGATTTAAGTCTACTTCATGCCAATGAGCTACTCCTCAACAG
GGGCTGGTTTTGCCATTTGAGGAATGACAGCCACTATGTTGTGTACACAA
GAGAGCTGGATGGCATCGACAGAATCTTTATCGTGGTTCTGAATTTTGGA
GAATCAACACTGTTAAATCTACATAATATGATTTCGGGCCTTCCCGCTAA
AATGAGAATAAGGTTAAGTACCAATTCTGCCGACAAAGGCAGTAAAGTTG
ATACAAGTGGCATTTTTCTGGACAAGGGAGAGGGACTCATCTTTGAACAC
AACACGAAGAATCTCCTTCATCGCCAAACAGCTTTCAGAGATAGATGCTT
TGTTTCCAATCGAGCATGCTATTCCAGTGTACTGAACATACTGTATACCT
CGTGTTAG (SEQ ID NO:245).

“SLC6A14” (also known as solute carrier family 6 member 14 and BMIQ11) as used herein refers to gene identified by Entrez Gene ID No. 11254, allelic variants thereof, orthologs thereof, mRNA transcripts encoded by the gene, including the nucleotide sequence of NCBI Reference Sequence NM_007231.5 (SEQ ID NO:246).

Sodium- and chloride-dependent neutral and basic amino acid transporter B^{0,+} (ATB^{0,+}) proteins described herein include the protein encoded by SLC6A14 and the amino acid sequence of NCBI Reference Sequence NP_009162.1 and NCBI CCDS ID NO. CCDS14570.1:

MDKLKCPSFFKCREKEKVSASSENFHVGENDENQDRGNWSKKSDYLLSMI
GYAVGLGNVWRFPYLTYSNGGGAFLIPYAIMLALAGLPLFFLECSLGQFA
SLGPVSVWRILPLFQGVGITMVLISIFVTIYYNVIIAYSLYYMFASFQSE
LPWKNCSSWSDKNCSRSPIVTHCNVSTVNKGIQEIIQMNKSWVDINNFTC
INGSEIYQPGQLPSEQYWNKVALQRSSGMNETGVIVWYLALCLLLAWLIV
GAALFKGIKSSGKVVYFTALFPYVVLLILLVRGATLEGASKGISYYIGAQ
SNFTKLKEAEVWKDAATQIFYSLSVAWGGLVALSSYNKFKNNCFSDAIVV
CLTNCLTSVFAGFAIFSILGHMAHISGKEVSQVVKSGFDLAFIAYPEALA
QLPGGPFWSILFFFMLLTLGLDSQFASIETITTTIQDLFPKVMKKMRVPI
TLGCCLVLFLLGLVCVTQAGIYWVHLIDHFCAGWGILIAAILELVGIIWI
YGGNRFIEDTEMMIGAKRWIFWLWWRACWFVITPILLIAIFIWSLVQFHR
PNYGAIPYPDWGVALGWCMIVFCIIWIPIMAIIKIIQAKGNIFQRLISCC
RPASNWGPYLEQHRGERYKDMVDPKKEADHEIPTVSGSRKPE (SEQ ID
NO:248).

ATB^{0,+} nucleotide sequences include the nucleotide sequence of NCBI CCDS ID NO. CCDS14570.1:

ATGGACAAGTTGAAATGCCCGAGTTTCTTCAAGTGCAGGGAGAAGGAGAA
AGTGTCGGCTTCATCAGAGAATTTCCATGTTGGTGAAAATGATGAGAATC
AGGACCGTGGTAACTGGTCCAAAAAATCGGATTATCTTCTATCTATGATT
GGATACGCAGTGGGATTAGGAAATGTGTGGAGATTTCCATATCTGACCTA
CAGCAATGGTGGAGGCGCCTTCTTGATACCTTATGCAATTATGTTAGCAT
TGGCTGGTTTACCTTTGTTCTTTCTGGAGTGTTCACTGGGACAATTTGCT
AGCTTAGGTCCAGTTTCAGTTTGGAGGATTCTTCCATTGTTTCAAGGTGT
GGGAATTACAATGGTCCTGATCTCCATTTTTGTGACAATCTATTACAATG
TCATAATTGCCTATAGTCTTTACTACATGTTTGCTTCTTTTCAAAGTGAA
CTACCATGGAAAAATTGTTCTTCGTGGTCAGATAAAAACTGTAGCAGATC
ACCAATAGTAACTCACTGTAATGTGAGTACAGTGAATAAAGGAATACAAG
AGATCATCCAAATGAATAAAAGCTGGGTAGACATCAACAATTTTACCTGC
ATCAACGGCAGTGAAATTTATCAGCCAGGGCAGCTTCCCAGTGAACAATA
TTGGAATAAAGTGGCGCTCCAACGGTCAAGTGGAATGAATGAGACTGGAG
TAATTGTTTGGTATTTAGCACTTTGTCTTCTTCTGGCTTGGCTCATAGTT
GGAGCAGCACTATTTAAAGGAATCAAATCGTCTGGCAAGGTGGTATATTT
TACAGCTCTTTTCCCCTATGTGGTCCTACTCATCCTGTTAGTACGAGGTG
CAACTCTGGAGGGTGCTTCAAAAGGCATTTCATACTATATTGGAGCCCAG
TCAAATTTTACAAAACTTAAGGAAGCTGAGGTATGGAAAGATGCTGCCAC
TCAGATATTTTACTCCCTTTCAGTGGCTTGGGGTGGCTTAGTTGCTCTAT
CATCTTACAATAAGTTCAAAAACAACTGCTTCTCTGATGCCATTGTGGTT
TGTTTGACAAACTGTCTCACTAGCGTGTTTGCTGGATTTGCTATTTTTTC
TATATTGGGACACATGGCCCATATATCTGGAAAGGAAGTTTCTCAAGTTG
TAAAATCAGGTTTTGATTTGGCATTCATTGCCTATCCAGAGGCTCTAGCC
CAACTCCCAGGTGGTCCATTTTGGTCCATATTATTTTTTTTCATGCTTTT
AACTTTGGGTCTCGATTCTCAGTTTGCTTCGATTGAAACGATCACAACAA
CAATTCAAGATTTATTTCCCAAAGTGATGAAGAAAATGAGGGTTCCCATA
ACTTTGGGCTGCTGCTTGGTTTTGTTTCTCCTTGGTCTCGTCTGTGTGAC
TCAGGCTGGAATTTACTGGGTTCATCTGATTGACCACTTCTGTGCTGGAT
GGGGCATTTTAATTGCAGCTATACTGGAGCTAGTTGGAATCATCTGGATT
TATGGAGGGAACAGATTCATTGAGGATACAGAAATGATGATTGGAGCAAA
GAGGTGGATATTCTGGCTATGGTGGAGAGCTTGCTGGTTTGTAATTACGC
CTATCCTTTTGATTGCAATATTTATCTGGTCATTGGTGCAATTTCATAGA
CCTAATTATGGCGCAATTCCATACCCTGACTGGGGAGTTGCTTTAGGCTG
GTGTATGATTGTTTTCTGCATTATTTGGATTCCAATTATGGCTATCATAA
AAATAATTCAGGCTAAAGGAAACATCTTTCAACGCCTTATAAGTTGCTGC
AGACCAGCTTCTAACTGGGGTCCATACCTGGAACAACATCGTGGGGAAAG
ATATAAAGACATGGTAGATCCTAAAAAAGAGGCTGACCATGAAATACCTA
CTGTTAGTGGCAGCAGAAAACCGGAATGA (SEQ ID NO:249).

Activated T-cells dramatically increase arginine import through the upregulation of cationic amino acid transporters. Upregulation of CATs facilitates T-cell proliferation. Arginine deficiency is fundamental in inflammation- and cancer-associated immunosuppression, and causes profound impairment of T-cell function. In response to arginine deprivation, T-cells induce autophagy to increase access to arginine intracellularly. This cytoprotective mechanism preserves T cell viability but cannot sustain cell proliferation.

Myeloid-derived suppressor cells may directly promote immune dysfunction by depriving T-cells of essential metabolites such as arginine or interfering with T-cell viability, migration, or activation. MDSCs can also indirectly suppress T-cells by inducing other immune regulatory cells such as T-regulatory cells and tumor-associated macrophages, increasing competition for resources. Arginine availability modulates much of these activities. Polymorphonuclear MDSCs, a major source of arginase 1 in tumor-bearing hosts, reduce extracellular arginine by secreting arginase 1 and enhancing arginine uptake through cationic amino acid transporters. Reconstitution of adaptive immune functions in the context of arginine-mediated tumor immune escape is a potential therapeutic strategy to boost the immunological anti-tumor response.

Described herein are methods of rescuing T-cell proliferation and activity in an environment of limited arginine availability that occurs when myeloid cells and cancer cells out-compete T-cells for arginine (for example, in the TME). In some embodiments, described herein are CAR-T cells that overexpress a specific amino acid transporter or combination of amino acid transporters. In some embodiments, described herein are CAR-T cells that overexpress an arginine transporter. In some embodiments, described herein are CAR-T cells that express or overexpress an amino acid transporter that can transport arginine from the extracellular space into the cytosol of the CAR-T cell. In some embodiments, the amino acid transporter is a human amino acid transporter to reduce immunogenicity but may be modified from other species. For example, in some embodiments, the amino acid transporter is a humanized amino acid transporter. Table 1 describes human amino acid transporters capable of bidirectional transport of cationic amino acids such as arginine.

TABLE 1

| Protein | Gene(s) | mRNA Sequence Accession No (SEQ ID NO) | Apparent K_{M} (mmol/L) | Na⁺-dependent | Trans-stimulation |
|---|---|---|---|---|---|
| CAT-1 | SLC7A1 | NM_003045.5 (SEQ ID NO:180) | 0.1-0.16 | No | Yes |
| CAT-2 | SLC7A2 | NM_001008539.4 (SEQ ID NO:184) | 3.4-3.9 | No | No |
| | | NM_001164771.2 (SEQ ID NO:185) | | | |
| | | NM_001370337.1 (SEQ ID NO:186) | | | |
| | | NM_001370338.1 (SEQ ID NO:187) | | | |
| | | NM_003046.6 (SEQ ID NO:188) | | | |
| CAT-3 | SLC7A3 | NM_001048164.3 (SEQ IDNO:204) | 0.2-0.5 | No | Moderate |
| | | NM_032803.6 (SEQ ID NO:205) | | | |
| CAT-4 | SLC7A4 | NM_004173.3 (SEQ ID NO:210) | NA | NA | NA |
| y⁺LAT1 & 4F2hc | SLC7A7 & SLC3A2 | NM_001126105.3 (SEQ ID NO:220) | 0.34 | No | Yes |
| | | NM_003982.4 (SEQ ID NO:221) | | | |
| | | NM_001126106.4 (SEQ ID NO:222) | | | |
| | | & | | | |
| | | NM_001012662.3 (SEQ ID NO:227) | | | |
| | | NM_001012664.3 (SEQ ID NO:228) | | | |
| | | NM_001013251.3 (SEQ ID NO:229) | | | |
| | | NM_002394.6 (SEQ ID NO:230) | | | |
| y⁺LAT2 & 4F2hc | SLC7A6 & SLC3A2 | NM_001076785.3 (SEQ ID NO:214) | 0.12-0.14 | No | Yes |
| | | NM_003983.6 (SEQ ID NO:215) | | | |
| | | & | | | |
| | | NM_001012662.3 (SEQ ID NO:227) | | | |
| | | NM_001012664.3 (SEQ ID NO:228) | | | |
| | | NM_001013251.3 (SEQ ID NO:229) | | | |
| | | NM_002394.6 (SEQ ID NO:230) | | | |
| b^{0,+}AT & rBAT | SLC7A9 & SLC3A1 | NM_001126335.2 (SEQ ID NO:234) | 0.08-0.2 | No | Yes |
| | | NM_001243036.2 (SEQ ID NO:235) | | | |
| | | NM_014270.5 (SEQ ID NO:236) | | | |
| | | & | | | |
| | | NM_000341.4 (SEQ ID NO:242) | | | |
| ATB⁰⁺ | SLC6A14 | NM_007231.5 (SEQ ID NO:246) | 0.1-0.15 | Yes | No |

Members of the CAT family transport essentially cationic amino acids by facilitated diffusion with differential trans-stimulation by intracellular substrates. In some cells they may regulate the rate of NO synthesis by controlling the uptake of L-arginine as the substrate for nitric oxide synthase. At normal physiological concentrations, the biochemical system y⁺ carrier, principally represented by the cationic amino acid transporter type 1 (CAT-1), is the predominant cellular transport system through the plasma membrane. CAT-1 is encoded by the SLC7A1 gene and is widely distributed in a number of systems and crucial for a variety of cellular functions. CAT-1 is an Na⁺-independent transporter and has the highest affinity (lowest K_{M}) for arginine, which allows efficient transport even when arginine concentration is low. The strong trans-stimulation of CAT-1 indicates it works better in exchange than in uniport mode.

The disclosure also contemplates artificial variants of the CAT-2A isoform, such as CAT-2A^{R369E}, CAT-2A^{N381i}, and CAT-2A^{R369E/N381i}. While the apparent K_{M} values for cationic amino acids and the sensitivity to trans-stimulation of CAT-1, CAT-2B, and CAT-3 are characteristic of system y⁺, CAT-2A exhibits a 10-fold lower substrate affinity and is largely independent of substrate at the trans-side of the membrane. This variant is artificially created by transplanting two amino acids from an intracellular domain of CAT-1 to the homologous domain in CAT-2A. Specifically, the Arg residue at position 369 is replaced by a Glu residue (R369E) while an Asn residue is inserted into position 381. The resultant variant has a K_{M} comparable to CAT-1 while bearing no trans-stimulation.

CAR-T cells display target specificity comparable to a monoclonal antibody and the display effector functions of a cytotoxic T-cell, making CAR-T treatment appealing for a variety of diseases. These characteristics allow for antigen recognition independent of the major histocompatibility complex and can be designed to specifically target the conserved and essential epitopes of the antigen.

The TME in solid tumors is a hostile environment where barrages of immunosuppressive signals and a shortage of essential nutrients result in T-cell exhaustion. In particular, arginine is rapidly consumed by active cancer cells and degraded by various arginases secreted from infiltrated myeloid derived suppressor cells. Moreover, T-cells are incapable of regenerating arginine from other amino acids and rely on exogenous arginine supply. The inventors have discovered that augmenting CAR-T cells with arginine transporter(s) can allow these cells to better compete for arginine in these hostile microenvironments.

The overexpression of arginine transporters can also be exploited, for example, to prime the augmented CAR-T cells before being reinfused into the patient. CAR-T cells expressing the arginine transporters can be cultured ex vivo in arginine-rich conditions until they acquire sufficient arginine to sustain expression and subsequent anti-tumor activity within the TME. Intracellular arginine enrichment via in vitro priming of the T cells can facilitate the survival, life-span, activity and therapeutic efficacy of CAR-T cells.

Exemplary arginine transporters include, CAT-1, CAT-2, CAT-3, and ATB^{0,+}, which may not require any subunit. Also contemplated are arginine transporters y⁺LAT1+4F2hc, y⁺LAT2+4F2hc, or b^{0,+}AT+rBAT. For example, CAT-1, CAT-2 and CAT-3 do not co-transport Na⁺ or Cl⁻, and may have minimal impact on membrane potential when overexpressed. CAT-1 has high affinity (i.e., lowest Kₘ) for arginine, which may allow efficient transport even when arginine concentration is low. The activity of CAT-2 may be unaffected by trans-stimulation.

An arg+CAR-T cell can express an arginine transporter comprising one or more mutations. Suitable amino acid modifications for improving the expression of an arginine transporter can be conservative or non-conservative mutations. A mutation can be made such that the encoded transporter is modified to a polar, non-polar, basic or acidic amino acid transporter. An engineered CAR-T cell can be generated from the subject’s whole blood where T-cells are separated from the whole blood product and re-engineered in a lab by inserting genes through a vector into the cells to make chimeric antigen receptors on their surface which specifically target antigens of interest. These modified T cells are multiplied and put back into the subject’s blood stream where they continue to multiply. Without being bound by theory, it is believed that once administered to the subject, the CAR-T cells are attracted to targets on the surface of the cancer cells. Without being bound by theory, it is believed that the CAR-T cells identify cells expressing the target antigen and kill them. CAR-T cells can remain in the body after the acute attack and prevent the target cells from returning.

### Methods of CAR-T Cell Production

CAR-T cells described herein can be produced from immune cells, for example, CD4+ and CD8+ T cells, harvested from a subject, for example, a patient in need of treatment. Appropriate T-cell populations can be harvested and isolated from whole blood using apheresis/leukapheresis in combination with cell separation methods, for example, counterflow centrifugal elutriation. Methods of isolating T-cell populations are known in the art and can be performed using suitable equipment, for example, a Haemonetics Cell Saver (Haemonetics, Boston, MA) and/or a CliniMACS Prodigy (Miltenyi Biotec, Germany). Isolated T-cells can be expanded and stimulated using methods known in the art, including, for example, culturing with feeder cells and/or in a bioreactor and in the presence of, for example, anti-CD3 antibodies, anti-CD28 antibodies, magnetic bead-conjugated anti-CD3 antibodies, magnetic bead-conjugated anti-CD28 antibodies, growth factors (for example, IL-2), and artificial antigen presenting cells. Suitable bioreactor systems include CliniMACS Prodigy (Miltenyi Biotec, Germany), the WAVE Bioreactor (GE Healthcare Life Sciences, Pittsburgh, PA), and the G-Rex (Wilson Wolf Manufacturing, Saint Paul, MN). For example, isolated T-cells can be expanded in TexMACS Medium (Miltenyi Biotec, Germany) supplemented with 200 IU/mL IL-2 and TransAct beads (Miltenyi Biotec, Germany) at 37° C. with 5% CO₂. Methods of isolating and expanding T-cell populations are described in, for example, Levine et al., (2017) “Global Manufacturing of CAR T Cell Therapy” Mol Ther Methods Clin Dev. 4:92-101.

Methods of producing CAR-T cells described herein can also include a step of transfecting an expanded T-cell population with one or more expression vectors encoding a CAR, an amino acid transporter, or a CAR and an amino acid transporter. Suitable methods of transfection are known in the art and include, for example, calcium phosphate transfection, lipofection, polymer transfection, Fugene product-based transfection (Promega Corporation, Madison, WI), and electroporation, for example, using a CliniMACS Electroporator (Miltenyi Biotec, Germany). In some embodiments described herein, methods of producing CAR-T cells can include a step of transfecting an expanded T-cell population with one or more transposon-containing plasmids, for example, a plasmid encoding a Sleeping Beauty transposon and a CAR, an amino acid transporter, or a CAR and an amino acid transporter.

In some embodiments described herein, methods of producing CAR-T cells can include a step of using a virus (for example, a lentivirus, a retrovirus, an adenovirus, or an adeno-associated virus) to transduce an expanded T-cell population with one or more expression vectors encoding a CAR, an amino acid transporter, or a CAR and an amino acid transporter.

T-cells transfected or transduced with an appropriate nucleotide construct can be further nourished in suitable medium (for example, TexMACS Medium (Miltenyi Biotec, Germany) supplemented with 1 mM L-arginine (Sigma-Aldrich, USA)) and assayed for viability.

T-cell purity and the ratio of helper T-cells to killer T-cells can be determined using flow cytometry and fluorescent assisted cell sorting (FACS) methods that employ suitable antibodies (for example, anti-CD19, CD14, CD45, CD3, CD4, and CD8 antibodies). Expressions of CARs and arginine transporter proteins can be determined using custom antibodies which are specific to the antigen-recognizing domain of the CAR or which are specific to the arginine transporter.

CAR-T intracellular arginine content can be determined using an L-Arginine ELISA kit (ALPCO, USA).

Methods described herein can be include a step of harvesting CAR-T cells for downstream application based on the number of cells obtained. For example, in some embodiments, an amount of CAR-T cells for harvesting includes an amount equivalent to about 1×10³, about 1×10⁴, about 1×10⁵, about 1×10⁶, about 1×10⁷, about 1×10⁸, about 1×10⁹, about 1x10^{10,} about 2×10¹⁰, about 3×10¹⁰, about 4×10¹⁰, about 5×10¹⁰, about 6×10¹⁰, about 7×10¹⁰, about 8×10^{10,} about 9×10^{10,} about 1×10¹¹, about 1×10¹², about 1×10¹³, about 1×10¹⁴, about 1×10¹⁵, about 1×10³ to about 3×10¹⁰, about 1×10⁵ to about 3×10¹⁰, about 1×10³ to about 1×10⁵, about 1×10⁵ to about 1×10¹⁵, about 1×10⁵ to about 1×10^{10,} about 1×10⁷ to about 1×10¹², about 1×10⁵ to about 1×10⁷, about 1×10¹⁰ to about 9×10¹⁰, or about 1×10⁹ to about 1×10¹¹ cells per kg body weight of a subject. In some embodiments, an amount of CAR-T cells for harvesting includes about 1×10⁵, about 1×10^{6,} about 1×10^{7,} about 1×10⁸ about 1×10⁹ about 1×10¹⁰ about 1×10¹¹ about 1×10¹², about 1×10⁵ to about 1×10¹², about 1×10⁵ to about 1×10^{10,} about 1×10⁵ to about 1×10⁷, about 1×10⁷ to about 1×10¹⁰, about 1×10⁷ to about 1×10¹², about 1×10⁹ to about 1×10^{10,} about 1×10⁶ to about 1×10⁸, about 1×10⁷ to about 1×10⁹, or about 1×10⁹ to about 1×10¹¹ cells.

Methods described herein can be include a step of harvesting CAR-T cells for downstream application based on the arginine content of cells obtained. For example, in some embodiments, CAR-T cells for harvesting include cells with an intracellular arginine content of about 10 µM, about 20 µM, about 30 µM, about 40 µM, about 50 µM, about 60 µM, about 70 µM, about 80 µM, about 90 µM, about 100 µM, about 200 µM, about 300 µM, about 400 µM, about 500 µM, about 600 µM, about 700 µM, about 800 µM, about 900 µM, about 1000 µM, about 1500 µM, about 2000 µM, about 2500 µM, about 3000 µM, about 3500 µM, about 4000 µM, about 100 µM to about 4000 µM, about 100 µM to about 1000 µM, about 100 µM to about 2000 µM, about 1000 µM to about 2000 µM, about 1000 µM to about 3000 µM, about 1000 µM to about 4000 µM, about 500 µM to about 1000 µM, about 3000 µM to about 4000 µM, about 2000 µM to about 4000 µM, or about 500 µM to about 2000 µM arginine per cell.

CAR-T cells described herein are genetically modified to express specific CARs and/or amino acid transporter proteins, for example, arginine transporter proteins. In some embodiments, an expression cassette coding for an arginine transporter is introduced (for example, by genetic engineering) into a T cell either before, after, or simultaneously with an expression cassette coding for a CAR. Nucleotide sequences coding for an amino acid transporter can be placed alongside those coding for the CAR on the same vector (e.g., one vector for both CAR and transporter). This introduces both CAR and the transporter into the same cell simultaneously such that every resultant arg+CAR-T cell is augmented by the transporter. In some embodiments, a nucleotide construct coding for an amino acid transporter is placed on a separate vector from those coding for the CAR (e.g., individual vectors for both CAR and transporter).

CAR-T cells described herein can be produced by transfection, electroporation, or transformation of T cells with one or more specific expression vectors that encode nucleic acid sequences for a CAR and/or an amino acid transporter protein, for example, an arginine transporter protein. CAR-T cells described herein can also be produced by transduction of T cells with one or more viruses carrying a specific expression vector that encodes a nucleic acid sequence for a CAR and/or an amino acid transporter protein, for example, an arginine transporter protein. Isolated T-cells can be transduced with one or more retroviral vectors, for example, an integrating γ-retrovirus vector or a lentiviral vector. γ-retrovirus vectors and lentiviral vectors integrate randomly into T cell genomes. Isolated T-cells can also be transformed with one or more integrating artificial transposons or via transfection with non-integrating RNA molecules. In some embodiments, isolated T cells can be electroporated with CRISPR/Cas-9 expression constructs and transfected with one or more adenovirus or AAV vectors encoding specific CARs and/or amino acid transporter proteins, for example, arginine transporter proteins.

For example, described herein is a method of producing a genetically modified T-cell (for example, a CAR-T cell) that includes transfecting a T-cell with an expression vector comprising a nucleic acid sequence encoding a CAR and a nucleic acid sequence encoding an amino acid transporter, for example, an arginine transporter. Also described herein is a method of producing a genetically modified T-cell (for example, a CAR-T cell) that includes transfecting a T-cell with a first expression vector comprising a nucleic acid sequence encoding a CAR and a second expression vector comprising a nucleic acid sequence encoding an amino acid transporter, for example, an arginine transporter. In some embodiments, transfecting can be performed by chemical methods of transfection (for example, calcium phosphate transfection, lipofection, polymer transfection (e.g., DEAE-dextran or polyethylenimine (PEI) transfection), or transfection reagents developed by Fugene (Promega Corporation, Madison, WI; e.g., FuGENE HD or FuGENE 6 transfection reagents)), non-chemical methods of transfection (e.g., electroporation, cell squeezing, sonoporation, optical transfection, protoplast fusion, impalefection, or hydrodynamic delivery), particle-based transfection (gene gun transfection, magnet-assisted transfection), nucleofection, or heat shock transfection. In some embodiments, the method includes transfecting a T-cell with a first expression vector and a second expression vector simultaneously or sequentially.

Also described herein is a method of producing a genetically modified T-cell (for example, a CAR-T cell) that includes transducing the T-cell with a virus (for example, an adenovirus, an AAV, a lentivirus, or a retrovirus) carrying a nucleic acid sequence encoding a CAR and a nucleic acid sequence encoding an amino acid transporter, for example, an arginine transporter. Also described herein is a method of producing a genetically modified T-cell (for example, a CAR-T cell) that includes transducing a T-cell with a first virus (for example, an adenovirus, an adeno-associated virus, a lentivirus, or a retrovirus) carrying a nucleic acid sequence encoding a CAR and transducing the T-cell with a second virus (for example, an adenovirus, an adeno-associated virus, a lentivirus, or a retrovirus) carrying a nucleic acid sequence encoding an amino acid transporter, for example, an arginine transporter. In some embodiments, the method includes transducing a T-cell with a first virus and a second virus simultaneously or sequentially.

In some embodiments, where a method of producing a genetically modified T-cell includes transfecting a T-cell with an expression vector or transducing the cell with a virus, the method can also include a step of selecting for transfectants, for example, by antibiotic resistance.

In some embodiments, where a method of producing a genetically modified T-cell includes transfecting the T-cell with a first expression vector and a second expression vector sequentially, the method can also include a step of selecting for transfectants, for example, by antibiotic resistance or expression of a selection marker suitable for FACS, such as a fluorescent protein. For example such methods can include a step of selecting for transfectants of the first expression vector. Such methods can further include a step of selecting for transfectants of the second expression vector. Such methods can further include a step of selecting for transfectants of the first and the second expression vector. In some embodiments, selection for transfectants of the first expression vector is performed prior to transfecting with the second expression vector.

In some embodiments, where a method of producing a genetically modified T-cell includes transducing a T-cell with a first virus and a second virus sequentially, the method can also include a step of selecting for a transduced cell, for example, by antibiotic resistance or by FACS. For example such methods can include a step of selecting for a cell transduced with the first virus. Such methods can further include a step of selecting for a cell transduced with a second virus. Such methods can further include a step of selecting for cells transduced with the first and the second virus. In some embodiments, selection for cells transduced with the first virus is performed prior to transduction with the second virus.

In some embodiments, a method of producing a genetically modified T-cell includes transducing the T-cell with a virus and transfecting the T-cell with an expression vector, where the transducing and transfecting can be performed in either order (for example, transducing followed by transfecting, or transfecting followed by transducing). For example, in some embodiments, a method of producing a genetically modified T-cell includes transducing the T-cell with a virus carrying a nucleic acid sequence encoding a CAR and transfecting the T-cell with an expression vector comprising a nucleic acid sequence encoding an amino acid transporter, for example, an arginine transporter. In some embodiments, a method of producing a genetically modified T-cell includes transducing the T-cell with a virus carrying a nucleic acid sequence encoding an amino acid transporter, for example, an arginine transporter, and transfecting the T-cell with an expression vector comprising a nucleic acid sequence encoding a CAR.

### CAR and Amino Acid Transporter Expression Vectors and Transgenes

CAR-T nucleotide constructs described herein (e.g., nucleotide expression vectors and virus nucleotide constructs) can include standard components such as, but not limited to, promoters, Kozak sequences, gene expression cassettes, self-cleavage sites, markers for selection (e.g., fluorescent protein expression cassettes or antibiotic resistance cassettes), inverted tandem repeat sequences, and transcription termination and polyA signal sequences.

Exemplary promoter sequences include the following:

EF1α:GGCTCCGGTGCCCGTCAGTGGGCAGAGCGCACATCGCCCACAGTC
CCCGAGAAGTTGGGGGGAGGGGTCGGCAATTGAACCGGTGCCTAGAGAAG
GTGGCGCGGGGTAAACTGGGAAAGTGATGTCGTGTACTGGCTCCGCCTTT
TTCCCGAGGGTGGGGGAGAACCGTATATAAGTGCAGTAGTCGCCGTGAAC
GTTCTTTTTCGCAACGGGTTTGCCGCCAGAACACAGGTAAGTGCCGTGTG
TGGTTCCCGCGGGCCTGGCCTCTTTACGGGTTATGGCCCTTGCGTGCCTT
GAATTACTTCCACCTGGCTGCAGTACGTGATTCTTGATCCCGAGCTTCGG
GTTGGAAGTGGGTGGGAGAGTTCGAGGCCTTGCGCTTAAGGAGCCCCTTC
GCCTCGTGCTTGAGTTGAGGCCTGGCCTGGGCGCTGGGGCCGCCGCGTGC
GAATCTGGTGGCACCTTCGCGCCTGTCTCGCTGCTTTCGATAAGTCTCTA
GCCATTTAAAATTTTTGATGACCTGCTGCGACGCTTTTTTTCTGGCAAGA
TAGTCTTGTAAATGCGGGCCAAGATCTGCACACTGGTATTTCGGTTTTTG
GGGCCGCGGGCGGCGACGGGGCCCGTGCGTCCCAGCGCACATGTTCGGCG
AGGCGGGGCCTGCGAGCGCGGCCACCGAGAATCGGACGGGGGTAGTCTCA
AGCTGGCCGGCCTGCTCTGGTGCCTGGCCTCGCGCCGCCGTGTATCGCCC
CGCCCTGGGCGGCAAGGCTGGCCCGGTCGGCACCAGTTGCGTGAGCGGAA
AGATGGCCGCTTCCCGGCCCTGCTGCAGGGAGCTCAAAATGGAGGACGCG
GCGCTCGGGAGAGCGGGCGGGTGAGTCACCCACACAAAGGAAAAGGGCCT
TTCCGTCCTCAGCCGTCGCTTCATGTGACTCCACGGAGTACCGGGCGCCG
TCCAGGCACCTCGATTAGTTCTCGAGCTTTTGGAGTACGTCGTCTTTAGG
TTGGGGGGAGGGGTTTTATGCGATGGAGTTTCCCCACACTGAGTGGGTGG
AGACTGAAGTTAGGCCAGCTTGGCACTTGATGTAATTCTCCTTGGAATTT
GCCCTTTTTGAGTTTGGATCTTGGTTCATTCTCAAGCCTCAGACAGTGGT
TCAAAGTTTTTTTCTTCCATTTCAGGTGTCGTGA (SEQ ID NO:250)
;

PGK:GGGTAGGGGAGGCGCTTTTCCCAAGGCAGTCTGGAGCATGCGCTTT
AGCAGCCCCGCTGGGCACTTGGCGCTACACAAGTGGCCTCTGGCCTCGCA
CACATTCCACATCCACCGGTAGGCGCCAACCGGCTCCGTTCTTTGGTGGC
CCCTTCGCGCCACCTTCTACTCCTCCCCTAGTCAGGAAGTTCCCCCCCGC
CCCGCAGCTCGCGTCGTGCAGGACGTGACAAATGGAAGTAGCACGTCTCA
CTAGTCTCGTGCAGATGGACAGCACCGCTGAGCAATGGAAGCGGGTAGGC
CTTTGGGGCAGCGGCCAATAGCAGCTTTGCTCCTTCGCTTTCTGGGCTCA
GAGGCTGGGAAGGGGTGGGTCCGGGGGCGGGCTCAGGGGCGGGCTCAGGG
GCGGGGCGGGCGCCCGAAGGTCCTCCGGAGGCCCGGCATTCTGCACGCTT
CAAAAGCGCACGTCTGCCGCGCTGTTCTCCTCTTCCTCATCTCCGGGCCT
TTCG (SEQ ID NO:251);

CMV:CGTTACATAACTTACGGTAAATGGCCCGCCTGGCTGACCGCCCAAC
GACCCCCGCCCATTGACGTCAATAATGACGTATGTTCCCATAGTAACGCC
AATAGGGACTTTCCATTGACGTCAATGGGTGGAGTATTTACGGTAAACTG
CCCACTTGGCAGTACATCAAGTGTATCATATGCCAAGTACGCCCCCTATT
GACGTCAATGACGGTAAATGGCCCGCCTGGCATTATGCCCAGTACATGAC
CTTATGGGACTTTCCTACTTGGCAGTACATCTACGTATTAGTCATCGCTA
TTACCATGGTGATGCGGTTTTGGCAGTACATCAATGGGCGTGGATAGCGG
TTTGACTCACGGGGATTTCCAAGTCTCCACCCCATTGACGTCAATGGGAG
TTTGTTTTGGCACCAAAATCAACGGGACTTTCCAAAATGTCGTAACAACT
CCGCCCCATTGACGCAAATGGGCGGTAGGCGTGTACGGTGGGAGGTCTAT
ATAAGCAGAGCT (SEQ ID NO:252); and

CAG:GCGTTACATAACTTACGGTAAATGGCCCGCCTGGCTGACCGCCCAA
CGACCCCCGCCCATTGACGTCAATAATGACGTATGTTCCCATAGTAACGC
CAATAGGGACTTTCCATTGACGTCAATGGGTGGAGTATTTACGGTAAACT
GCCCACTTGGCAGTACATCAAGTGTATCATATGCCAAGTACGCCCCCTAT
TGACGTCAATGACGGTAAATGGCCCGCCTGGCATTATGCCCAGTACATGA
CCTTATGGGACTTTCCTACTTGGCAGTACATCTACGTATTAGTCATCGCT
ATTACCATGGTCGAGGTGAGCCCCACGTTCTGCTTCACTCTCCCCATCTC
CCCCCCCTCCCCACCCCCAATTTTGTATTTATTTATTTTTTAATTATTTT
GTGCAGCGATGGGGGCGGGGGGGGGGGGGGGGCGCGCGCCAGGCGGGGCG
GGGCGGGGCGAGGGGCGGGGCGGGGCGAGGCGGAGAGGTGCGGCGGCAGC
CAATCAGAGCGGCGCGCTCCGAAAGTTTCCTTTTATGGCGAGGCGGCGGC
GGCGGCGGCCCTATAAAAAGCGAAGCGCGCGGCGGGCG (SEQ ID NO:
253).

Exemplary transcription termination and polyA signal sequences include the following:

bGH pA:CTGTGCCTTCTAGTTGCCAGCCATCTGTTGTTTGCCCCTCCCC
CGTGCCTTCCTTGACCCTGGAAGGTGCCACTCCCACTGTCCTTTCCTAAT
AAAATGAGGAAATTGCATCGCATTGTCTGAGTAGGTGTCATTCTATTCTG
GGGGGTGGGGTGGGGCAGGACAGCAAGGGGGAGGATTGGGAAGACAATAG
CAGGCATGCTGGGGATGCGGTGGGCTCTATGG (SEQ ID NO:254);

rbHBB pA: AATAAAAGATCTTTATTTTCATTAGATCTGTGTGTTGGTT
TTTTGTGTG (SEQ ID NO:255);

SV40 pA:CTAGAGCTCGCTGATCAGCCTCGACTGTGCCTTCTAGTTGCC
AGCCATCTGTTGTTTGCCCCTCCCCCGTGCCTTCCTTGACCCTGGAAGGT
GCCACTCCCACTGTCCTTTCCTAATAAAATGAGGAAATTGCATCGCATTG
TCTGAGTAGGTGTCATTCTATTCTGGGGGGTGGGGTGGGGCAGGACAGCA
AGGGGGAGGATTGGGAAGACAATAGCAGGCATGCTGGGGATATGCA (SE
Q ID NO:256); and

hGH pA:GACGGGTGGCATCCCTGTGACCCCTCCCCAGTGCCTCTCCTGG
CCCTGGAAGTTGCCACTCCAGTGCCCACCAGCCTTGTCCTAATAAAATTA
AGTTGCATCATTTTGTCTGACTAGGTGTCCTTCTATAATATTATGGGGTG
GAGGGGGGTGGTATGGAGCAAGGGGCAAGTTGGGAAGACAACCTGTAGGG
CCTGCGGGGTCTATTGGGAACCAAGCTGGAGTGCAGTGGCACAATCTTGG
CTCACTGCAATCTCCGCCTCCTGGGTTCAAGCGATTCTCCTGCCTCAGCC
TCCCGAGTTGTTGGGATTCCAGGCATGCATGACCAGGCTCAGCTAATTTT
TGTTTTTTTGGTAGAGACGGGGTTTCACCATATTGGCCAGGCTGGTCTCC
AACTCCTAATCTCAGGTGATCTACCCACCTTGGCCTCCCAAATTGCTGGG
ATTACAGGCGTGAACCACTGCTCCCTTCCCTGTCCTTT (SEQ ID NO:
257).

Exemplary inverted tandem repeat (TIR) sequences include the following pT4 left inverted repeat (LIR) and right inverted repeat (RIR) sequences:

pT4 LIR:TACAGTTGAAGTCGGAAGTTTACATACACTTAAGTTGGAGTC
ATTAAAACTCGTTTTTCAACTACTCCACAAATTTCTTGTTAACAAACAAT
AGTTTTGGCAAGTCAGTTAGGACATCTACTTTGTGCATGACACAAGTCAT
TTTTCCAACAATTGTTTACAGACAGATTATTTCACTTATAATTCACTGTA
TCACAATTCCAGTGGGTCAGAAGTGTACATACACGCGCTTGACTGTGCCT
TT (SEQ ID NO:258);

and

pT4 RIR:TTAAACAATTTAAAGGCAATGCTACCAAATACTAAGCGCGTG
TATGTACACTTCTGACCCACTGGGAATGTGATGAAAGAAATAAAAGCTGA
AATGAATCATTCTCTCTACTATTATTCTGATATTTCACATTCTTAAAATA
AAGTGGTGATCCTAACTGACCTTAAGACAGGGAATCTTTACTCGGATTAA
ATGTCAGGAATTGTGAAAAAGTGAGTTTAAATGTATTTGGCTAAGGTGTA
TGTAAACTTCCGACTTCAACTGTA (SEQ ID NO:259).

Exemplary self-cleavage site nucleotide sequences include the following:

P2A:GCCACCAATTTCAGCCTGCTGAAACAGGCTGGCGACGTGGAAGAGA
ACCCTGGACCT (SEQ ID NO:260);

T2A:GGCAGCGGCGAGGGCAGAGGCAGCCTGCTGACCTGCGGCGACGTGG
AGGAGAACCCCGGCCCC (SEQ ID NO:261);

E2A:GGCAGCGGCCAGTGCACCAACTACGCCCTGCTGAAGCTGGCCGGCG
ACGTGGAGAGCAAC (SEQ ID NO:262); and

F2A:GGCAGCGGCGTGAAGCAGACCCTGAACTTCGACCTGCTGAAGCTGG
CCGGCGACGTGGAGAGCAACCCCGGCCCC (SEQ ID NO:263).

Exemplary selection marker nucleotide sequences include the following fluorescent protein and antibiotic resistance protein encoding sequences: mEGFP (fluorescent protein coding sequence):

GTGTCCAAGGGCGAAGAACTGTTTACCGGCGTGGTGCCCATCCTGGTGGA
ACTGGATGGGGATGTGAACGGCCACAAGTTCAGCGTTAGCGGAGAAGGCG
AAGGCGACGCCACATACGGAAAGCTGACACTGAAGTTCATCTGCACCACC
GGCAAGCTGCCTGTGCCATGGCCAACACTGGTCACCACACTGACATACGG
CGTGCAGTGCTTCAGCAGATACCCCGACCATATGAAGCAGCATGACTTCT
TCAAGAGCGCCATGCCTGAGGGCTACGTGCAAGAGCGGACCATCTTCTTT
AAGGACGACGGCAACTACAAGACCAGGGCCGAAGTGAAGTTCGAGGGCGA
CACCCTCGTGAACCGGATCGAGCTGAAGGGCATCGACTTCAAAGAGGACG
GCAACATCCTGGGCCACAAGCTCGAGTACAACTACAACAGCCACAACGTG
TACATCATGGCCGACAAGCAGAAAAACGGCATCAAAGTGAACTTCAAGAT
CCGGCACAACATCGAGGACGGCTCAGTGCAGCTGGCCGACCACTATCAGC
AGAACACACCCATCGGAGATGGCCCCGTTCTGCTGCCCGATAACCACTAC
CTGAGCACACAGAGCAAGCTGAGCAAGGACCCCAACGAGAAGCGGGACCA
CATGGTCCTGCTGGAATTTGTGACAGCCGCCGGAATCACCCTCGGCATGG
ACGAGCTTTACAAA (SEQ ID NO:264);

mEmerald (fluorescent protein coding sequence):

GTGAGCAAGGGCGAGGAGCTGTTCACCGGCGTGGTGCCCATCCTGGTGGA
GCTGGACGGCGACGTGAACGGCCACAAGTTCAGCGTGAGCGGCGAGGGCG
AGGGCGACGCCACCTACGGCAAGCTGACCCTGAAGTTCATCTGCACCACC
GGCAAGCTGCCCGTGCCCTGGCCCACCCTGGTGACCACCCTGACCTACGG
CGTGCAGTGCTTCGCCAGATACCCCGACCACATGAAGCAGCACGACTTCT
TCAAGAGCGCCATGCCCGAGGGCTACGTGCAGGAGAGAACCATCTTCTTC
AAGGACGACGGCAACTACAAGACCAGAGCCGAGGTGAAGTTCGAGGGCGA
CACCCTGGTGAACAGAATCGAGCTGAAGGGCATCGACTTCAAGGAGGACG
GCAACATCCTGGGCCACAAGCTGGAGTACAACTACAACAGCCACAAGGTG
TACATCACCGCCGACAAGCAGAAGAACGGCATCAAGGTGAACTTCAAGAC
CAGACACAACATCGAGGACGGCAGCGTGCAGCTGGCCGACCACTACCAGC
AGAACACCCCCATCGGCGACGGCCCCGTGCTGCTGCCCGACAACCACTAC
CTGAGCACCCAGAGCAAGCTGAGCAAGGACCCCAACGAGAAGAGAGACCA
CATGGTGCTGCTGGAGTTCGTGACCGCCGCCGGCATCACCCTGGGCATGG
ACGAGCTGTACAAG (SEQ ID NO:265);

mCherry2 (fluorescent protein coding sequence):

GTGTCTAAGGGCGAAGAGGACAACATGGCCATCATCAAAGAATTCATGCG
GTTCAAGGTGCACATGGAAGGCAGCGTGAACGGCCACGAGTTCGAGATTG
AAGGCGAAGGCGAGGGCAGACCTTACGAGGGAACACAGACCGCCAAGCTG
AAAGTCACCAAAGGCGGCCCTCTGCCTTTTGCCTGGGACATTCTGAGCCC
TCAGTTTATGTACGGCTCCAAGGCCTACGTGAAGCACCCCGCCGATATTC
CCGACTATCTGAAGCTGAGCTTCCCCGAGGGCTTCAACTGGGAGCGCGTG
ATGAATTTCGAGGACGGCGGCGTGGTCACCGTGACTCAAGATAGCTCTCT
GCAGGACGGCGAGTTCATCTACAAAGTGAAGCTGCGGGGCACAAACTTCC
CCAGCGACGGACCTGTGATGCAGTGCAGAACAATGGGCTGGGAAGCCAGC
ACCGAGAGAATGTACCCAGAAGATGGCGCCCTGAAGGGCGAGATTAAGCA
GCGGCTGAAACTCAAGGATGGCGGCCACTACGACGCCGAAGTGAAAACCA
CCTACAAGGCCAAGAAACCCGTGCAGCTGCCTGGCGCCTACAACGTGGAC
ATCAAGCTGGATATCCTGAGCCACAATGAGGACTACACCATCGTCGAGCA
GTACGAGAGAGCCGAGGGGAGACATTCTACCGGCGGAATGGACGAGCTGT
ACAAA (SEQ ID NO:266);

mScarlet-i (fluorescent protein coding sequence):

GTGTCTAAGGGCGAAGCCGTGATCAAAGAATTCATGCGGTTCAAGGTGCA
CATGGAAGGCAGCATGAACGGCCACGAGTTCGAGATCGAAGGCGAAGGCG
AGGGCAGACCTTATGAGGGAACACAGACCGCCAAGCTGAAAGTGACCAAA
GGCGGCCCTCTGCCTTTCAGCTGGGACATTCTGAGCCCTCAGTTTATGTA
CGGCAGCCGGGCCTTCATCAAGCACCCTGCCGATATTCCCGACTACTACA
AGCAGAGCTTCCCCGAGGGCTTCAAGTGGGAGAGAGTGATGAACTTCGAG
GACGGCGGAGCCGTGACCGTGACACAGGATACAAGCCTGGAAGATGGCAC
CCTGATCTACAAAGTGAAGCTGCGGGGCACCAACTTTCCACCTGATGGCC
CCGTGATGCAGAAAAAGACCATGGGCTGGGAAGCCAGCACCGAGAGACTG
TATCCTGAGGATGGCGTGCTGAAGGGCGACATCAAGATGGCCCTGAGACT
GAAGGATGGCGGCAGATACCTGGCCGACTTCAAGACCACCTACAAGGCCA
AGAAACCCGTGCAGATGCCTGGCGCCTACAACGTGGACAGAAAGCTGGAC
ATCACCAGCCACAACGAGGACTACACCGTGGTGGAACAGTACGAGCGGAG
CGAAGGCAGACACTCTACAGGCGGAATGGACGAGCTGTACAAA (SEQ I
D NO:267);

Puromycin N-acetyltransferase (puromycin resistance coding sequence):

ACAGAGTACAAACCTACAGTGCGCCTGGCCACCAGGGACGATGTTCCTAG
AGCCGTCAGAACTCTGGCCGCTGCCTTCGCCGATTATCCAGCCACAAGAC
ACACCGTGGATCCCGACAGACACATCGAGAGAGTGACCGAGCTGCAAGAG
CTGTTTCTGACCAGAGTCGGCCTGGACATCGGCAAAGTGTGGGTTGCAGA
TGATGGCGCCGCTGTGGCTGTGTGGACAACACCTGAATCTGTGGAAGCCG
GCGCAGTGTTTGCCGAGATCGGACCTAGAATGGCCGAGCTGAGCGGATCT
AGACTGGCTGCTCAACAGCAGATGGAAGGCCTGCTGGCTCCCCACAGACC
AAAAGAGCCTGCTTGGTTTCTGGCCACCGTGGGCGTTAGCCCTGACCACC
AAGGCAAAGGACTGGGATCTGCTGTGGTGCTGCCTGGCGTTGAAGCCGCT
GAAAGAGCTGGCGTTCCAGCCTTCCTGGAAACAAGCGCCCCTCGGAACCT
GCCTTTCTACGAGAGACTGGGCTTTACCGTGACCGCCGATGTGGAAGTGC
CAGAGGGACCAAGAACCTGGTGCATGACCAGAAAGCCTGGCGCC (SEQ
ID NO:268);

Aminoglycoside 3′-phosphotransferase II (G418 resistance coding sequence):

ATTGAACAAGATGGATTGCACGCAGGTTCTCCGGCCGCTTGGGTGGAGAG
GCTATTCGGCTATGACTGGGCACAACAGACAATCGGCTGCTCTGATGCCG
CCGTGTTCCGGCTGTCAGCGCAGGGGCGCCCGGTTCTTTTTGTCAAGACC
GACCTGTCCGGTGCCCTGAATGAACTGCAGGACGAGGCAGCGCGGCTATC
GTGGCTGGCCACGACGGGCGTTCCTTGCGCAGCTGTGCTCGACGTTGTCA
CTGAAGCGGGAAGGGACTGGCTGCTATTGGGCGAAGTGCCGGGGCAGGAT
CTCCTGTCATCTCACCTTGCTCCTGCCGAGAAAGTATCCATCATGGCTGA
TGCAATGCGGCGGCTGCATACGCTTGATCCGGCTACCTGCCCATTCGACC
ACCAAGCGAAACATCGCATCGAGCGAGCACGTACTCGGATGGAAGCCGGT
CTTGTCGATCAGGATGATCTGGACGAAGAGCATCAGGGGCTCGCGCCAGC
CGAACTGTTCGCCAGGCTCAAGGCGCGCATGCCCGACGGCGAGGATCTCG
TCGTGACCCATGGCGATGCCTGCTTGCCGAATATCATGGTGGAAAATGGC
CGCTTTTCTGGATTCATCGACTGTGGCCGGCTGGGTGTGGCGGACCGCTA
TCAGGACATAGCGTTGGCTACCCGTGATATTGCTGAAGAGCTTGGCGGCG
AATGGGCTGACCGCTTCCTCGTGCTTTACGGTATCGCCGCTCCCGATTCG
CAGCGCATCGCCTTCTATCGCCTTCTTGACGAGTTCTTC (SEQ ID NO
:269); and

Hygromycin B phosphotransferase (hygromycin resistance coding sequence):

CCTGAACTCACCGCGACGTCTGTCGAGAAGTTTCTGATCGAAAAGTTCGA
CAGCGTCTCCGACCTGATGCAGCTCTCGGAGGGCGAAGAATCTCGTGCTT
TCAGCTTCGATGTAGGAGGGCGTGGATATGTCCTGCGGGTAAATAGCTGC
GCCGATGGTTTCTACAAAGATCGTTATGTTTATCGGCACTTTGCATCGGC
CGCGCTCCCGATTCCGGAAGTGCTTGACATTGGGGAATTCAGCGAGAGCC
TGACCTATTGCATCTCCCGCCGTGCACAGGGTGTCACGTTGCAAGACCTG
CCTGAAACCGAACTGCCCGCTGTTCTGCAGCCGGTCGCGGAGGCCATGGA
TGCGATCGCTGCGGCCGATCTTAGCCAGACGAGCGGGTTCGGCCCATTCG
GACCGCAAGGAATCGGTCAATACACTACATGGCGTGATTTCATATGCGCG
ATTGCTGATCCCCATGTGTATCACTGGCAAACTGTGATGGACGACACCGT
CAGTGCGTCCGTCGCGCAGGCTCTCGATGAGCTGATGCTTTGGGCCGAGG
ACTGCCCCGAAGTCCGGCACCTCGTGCACGCGGATTTCGGCTCCAACAAT
GTCCTGACGGACAATGGCCGCATAACAGCGGTCATTGACTGGAGCGAGGC
GATGTTCGGGGATTCCCAATACGAGGTCGCCAACATCTTCTTCTGGAGGC
CGTGGTTGGCTTGTATGGAGCAGCAGACGCGCTACTTCGAGCGGAGGCAT
CCGGAGCTTGCAGGATCGCCGCGGCTCCGGGCGTATATGCTCCGCATTGG
TCTTGACCAACTCTATCAGAGCTTGGTTGACGGCAATTTCGATGATGCAG
CTTGGGCGCAGGGTCGATGCGACGCAATCGTCCGATCCGGAGCCGGGACT
GTCGGGCGTACACAAATCGCCCGCAGAAGCGCGGCCGTCTGGACCGATGG
CTGTGTAGAAGTACTCGCCGATAGTGGAAACCGACGCCCCAGCACTCGTC
CGAGGGCAAAGGAA (SEQ ID NO:270).

For example, CAR-T expression vectors described herein can include the following nucleotide components: a promoter sequence (for example, an EF1α, cumate, CAG, CMV, UbC, or PGK promoter sequence), an antigen-specific targeting sequence, a transmembrane domain sequence (for example a CD4, CD8α, CD28, CD3ζ, or ICOS nucleotide sequence), a transmembrane domain sequence (for example, a CD4, CD8α, CD28, CD3ζ, or ICOS transmembrane domain nucleotide sequence), and an intracellular signaling domain sequence (for example, an FcRγ or CD3ζ intracellular signaling domain sequence). CAR-T expression vectors described herein can further include one or more of the following components: one or more co-stimulatory domain sequences (for example, a 4-1BB, CD27, CD28, CD40, CD40L, TLR2, DAP10, OX40, IL-2RB, IL-2RA, MYD88, or ICOS co-stimulatory domain sequence), an arginine transporter sequence (for example, an SLC7A1, SLC7A2, SLC7A3, SLC7A4, SLC7A6, SLC7A7, SLC3A2, SLC3A1, SLC7A9, or SLC6A14 nucleotide sequence), and a hinge or spacer domain sequence(s).

In some embodiments, a CAR-T expression vector described herein includes a promoter sequence (for example, an EF1α, cumate, CMV, CAG, UbC, or PGK promoter sequence) and an arginine transporter sequence (for example, an SLC7A1, SLC7A2, SLC7A3, SLC7A4, SLC7A6, SLC7A7, SLC3A2, SLC3A1, SLC7A9, or SLC6A14 nucleotide sequence).

In some embodiments, a CAR-T expression vector described herein can also include one or more of the following: an antibiotic selection cassette (for example, an ampicillin, geneticin, zeocin, hygromycin, blasticidin, puromycin, or kanamycin resistance cassette), and an origin of replication sequence (for example, pUC, pMB1, pBR322, ColE1, R6K, p15A, pSC101, pMSCV, or F1 sequence). Lentiviral and γ-retroviral vectors described herein can include one or more of the following: a 5′ long-terminal repeat (LTR) sequence (including one or more of U3, R, and U5 sequences), a 3′ LTR sequence (including one or more of U3, R, and U5 sequences), a psi (Ψ) sequence, a trans-activating response (TAR) element sequence, a central polypurine tract (cPPT) sequence, a woodchuck hepatitis virus post-transcriptional regulatory element (WPRE) sequence, and a Rev response element (RRE) sequence. Adenovirus and AAV vectors described herein can also include inverted terminal repeat (ITR) sequences.

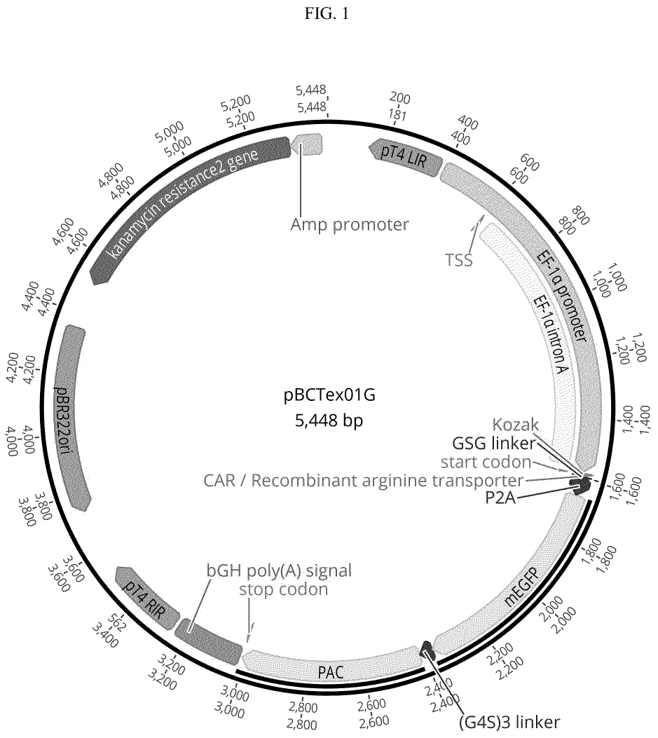

In some embodiments, a CAR-T expression vector described herein can include the following ordered components: a promoter sequence, a Kozak sequence, a start codon, one or more nucleotide sequences encoding a protein of interest (for example, a CAR nucleotide sequence and/or an amino acid transporter nucleotide sequence, for example an arginine transporter nucleotide sequence), a 2A self-cleavage site, one or more selection marker nucleotide sequences (for example, an antibiotic resistance nucleotide sequence and/or a fluorescent protein nucleotide sequence), a stop codon, and a termination and polyA signal nucleotide sequence. An exemplary CAR-T expression vector is pBCTex01G, shown in FIG. 1. The nucleotide sequence of pBCTex01G is the following:

GCCACCTGACGTCTAAGAAACCATTATTATCATGACATTAACCTATAAAA
ATAGGCGTATCACGAGGCCCTTTCGTTGTAAAACGACGGCCAGTCGAACC
ACGCAATGCGTCTCGATCCGCAGTGTCTTGCGTCTCTTACAGTTGAAGTC
GGAAGTTTACATACACTTAAGTTGGAGTCATTAAAACTCGTTTTTCAACT
ACTCCACAAATTTCTTGTTAACAAACAATAGTTTTGGCAAGTCAGTTAGG
ACATCTACTTTGTGCATGACACAAGTCATTTTTCCAACAATTGTTTACAG
ACAGATTATTTCACTTATAATTCACTGTATCACAATTCCAGTGGGTCAGA
AGTGTACATACACGCGCTTGACTGTGCCTTTGCTCTTCAATGGGAGGGCT
CCGGTGCCCGTCAGTGGGCAGAGCGCACATCGCCCACAGTCCCCGAGAAG
TTGGGGGGAGGGGTCGGCAATTGAACCGGTGCCTAGAGAAGGTGGCGCGG
GGTAAACTGGGAAAGTGATGTCGTGTACTGGCTCCGCCTTTTTCCCGAGG
GTGGGGGAGAACCGTATATAAGTGCAGTAGTCGCCGTGAACGTTCTTTTT
CGCAACGGGTTTGCCGCCAGAACACAGGTAAGTGCCGTGTGTGGTTCCCG
CGGGCCTGGCCTCTTTACGGGTTATGGCCCTTGCGTGCCTTGAATTACTT
CCACCTGGCTGCAGTACGTGATTCTTGATCCCGAGCTTCGGGTTGGAAGT
GGGTGGGAGAGTTCGAGGCCTTGCGCTTAAGGAGCCCCTTCGCCTCGTGC
TTGAGTTGAGGCCTGGCCTGGGCGCTGGGGCCGCCGCGTGCGAATCTGGT
GGCACCTTCGCGCCTGTCTCGCTGCTTTCGATAAGTCTCTAGCCATTTAA
AATTTTTGATGACCTGCTGCGACGCTTTTTTTCTGGCAAGATAGTCTTGT
AAATGCGGGCCAAGATCTGCACACTGGTATTTCGGTTTTTGGGGCCGCGG
GCGGCGACGGGGCCCGTGCGTCCCAGCGCACATGTTCGGCGAGGCGGGGC
CTGCGAGCGCGGCCACCGAGAATCGGACGGGGGTAGTCTCAAGCTGGCCG
GCCTGCTCTGGTGCCTGGCCTCGCGCCGCCGTGTATCGCCCCGCCCTGGG
CGGCAAGGCTGGCCCGGTCGGCACCAGTTGCGTGAGCGGAAAGATGGCCG
CTTCCCGGCCCTGCTGCAGGGAGCTCAAAATGGAGGACGCGGCGCTCGGG
AGAGCGGGCGGGTGAGTCACCCACACAAAGGAAAAGGGCCTTTCCGTCCT
CAGCCGTCGCTTCATGTGACTCCACGGAGTACCGGGCGCCGTCCAGGCAC
CTCGATTAGTTCTCGAGCTTTTGGAGTACGTCGTCTTTAGGTTGGGGGGA
GGGGTTTTATGCGATGGAGTTTCCCCACACTGAGTGGGTGGAGACTGAAG
TTAGGCCAGCTTGGCACTTGATGTAATTCTCCTTGGAATTTGCCCTTTTT
GAGTTTGGATCTTGGTTCATTCTCAAGCCTCAGACAGTGGTTCAAAGTTT
TTTTCTTCCATTTCAGGTGTCGTGATACTGCCGCCACCATGGGCTCCGGC
GCCACCAACTTTAGCCTGCTGAAACAGGCAGGCGACGTGGAAGAGAACCC
TGGACCTGTGTCCAAGGGCGAAGAACTGTTTACCGGCGTGGTGCCCATCC
TGGTGGAACTGGATGGGGATGTGAACGGCCACAAGTTCAGCGTTAGCGGA
GAAGGCGAAGGCGACGCCACATACGGAAAGCTGACACTGAAGTTCATCTG
CACCACCGGCAAGCTGCCTGTGCCATGGCCAACACTGGTCACCACACTGA
CATACGGCGTGCAGTGCTTCAGCAGATACCCCGACCATATGAAGCAGCAT
GACTTCTTCAAGAGCGCCATGCCTGAGGGCTACGTGCAAGAGCGGACCAT
CTTCTTTAAGGACGACGGCAACTACAAGACCAGGGCCGAAGTGAAGTTCG
AGGGCGACACCCTCGTGAACCGGATCGAGCTGAAGGGCATCGACTTCAAA
GAGGACGGCAACATCCTGGGCCACAAGCTCGAGTACAACTACAACAGCCA
CAACGTGTACATCATGGCCGACAAGCAGAAAAACGGCATCAAAGTGAACT
TCAAGATCCGGCACAACATCGAGGACGGCTCAGTGCAGCTGGCCGACCAC
TATCAGCAGAACACACCCATCGGAGATGGCCCCGTTCTGCTGCCCGATAA
CCACTACCTGAGCACACAGAGCAAGCTGAGCAAGGACCCCAACGAGAAGC
GGGACCACATGGTCCTGCTGGAATTTGTGACAGCCGCCGGAATCACCCTC
GGCATGGACGAGCTTTACAAAGGCGGCGGAGGATCTGGCGGAGGTGGAAG
CGGAGGCGGTGGAAGCACAGAGTACAAACCTACAGTGCGCCTGGCCACCA
GGGACGATGTTCCTAGAGCCGTCAGAACTCTGGCCGCTGCCTTCGCCGAT
TATCCAGCCACAAGACACACCGTGGATCCCGACAGACACATCGAGAGAGT
GACCGAGCTGCAAGAGCTGTTTCTGACCAGAGTCGGCCTGGACATCGGCA
AAGTGTGGGTTGCAGATGATGGCGCCGCTGTGGCTGTGTGGACAACACCT
GAATCTGTGGAAGCCGGCGCAGTGTTTGCCGAGATCGGACCTAGAATGGC
CGAGCTGAGCGGATCTAGACTGGCTGCTCAACAGCAGATGGAAGGCCTGC
TGGCTCCCCACAGACCAAAAGAGCCTGCTTGGTTTCTGGCCACCGTGGGC
GTTAGCCCTGACCACCAAGGCAAAGGACTGGGATCTGCTGTGGTGCTGCC
TGGCGTTGAAGCCGCTGAAAGAGCTGGCGTTCCAGCCTTCCTGGAAACAA
GCGCCCCTCGGAACCTGCCTTTCTACGAGAGACTGGGCTTTACCGTGACC
GCCGATGTGGAAGTGCCAGAGGGACCAAGAACCTGGTGCATGACCAGAAA
GCCTGGCGCCTGAGCTTCTGTGCCTTCTAGTTGCCAGCCATCTGTTGTTT
GCCCCTCCCCCGTGCCTTCCTTGACCCTGGAAGGTGCCACTCCCACTGTC
CTTTCCTAATAAAATGAGGAAATTGCATCGCATTGTCTGAGTAGGTGTCA
TTCTATTCTGGGGGGTGGGGTGGGGCAGGACAGCAAGGGGGAGGATTGGG
AAGACAATAGCAGGCATGCTGGGGATGCGGTGGGCTCTATGGCGCTGCAT
GAAGAGCTTAAACAATTTAAAGGCAATGCTACCAAATACTAAGCGCGTGT
ATGTACACTTCTGACCCACTGGGAATGTGATGAAAGAAATAAAAGCTGAA
ATGAATCATTCTCTCTACTATTATTCTGATATTTCACATTCTTAAAATAA
AGTGGTGATCCTAACTGACCTTAAGACAGGGAATCTTTACTCGGATTAAA
TGTCAGGAATTGTGAAAAAGTGAGTTTAAATGTATTTGGCTAAGGTGTAT
GTAAACTTCCGACTTCAACTGTAAGAGACGGAGTCACTGCCAACCGAGAC
GGTCATAGCTGTTTCCTGTGTGCCGCTTCCTCGCTCACTGACTCGCTGCG
CTCGGTCGTTCGGCTGCGGCGAGCGGTATCAGCTCACTCAAAGGCGGTAA
TACGGTTACCCACAGAATCAGGGGATAACGCAGGAAAGAACATGTGAGCA
AAAGGCCAGCAAAAGGCCAGGAACCGTAAAAAGGCCGCGTTGCTGGCGTT
TTTCCATAGGCTCCGCCCCCCTGACGAGCATCACAAAAATCGACGCTCAA
GTCAGAGGTGGCGAAACCCGACAGGACTATAAAGATACCAGGCGTTTCCC
CCTGGAAGCTCCCTCGTGCGCTCTCCTGTTCCGACCCTGCCGCTTACCGG
ATACCTGTCCGCCTTTCTCCCTTCGGGAAGCGTGGCGCTTTCTCATAGCT
CACGCTGTAGGTATCTCAGTTCGGTGTAGGTCGTTCGCTCCAAGCTGGGC
TGTGTGCACGAACCCCCCGTTCAGCCCGACCGCTGCGCCTTATCCGGTAA
CTATCGTCTTGAGTCCAACCCGGTAAGACACGACTTATCGCCACTGGCAG
CAGCCACTGGTAACAGGATTAGCAGAGCGAGGTATGTAGGCGGTGCTACA
GAGTTCTTGAAGTGGTGGCCTAACTACGGCTACACTAGAAGGACAGTATT
TGGTATCTGCGCTCTGCTGAAGCCAGTTACCTTCGGAAAAAGAGTTGGTA
GCTCTTGATCCGGCAAACAAACCACCGCTGGTAGCGGTGGTTTTTTTGTT
TGCAAGCAGCAGATTACGCGCAGAAAAAAAGGATCTCAAGAAGATCCTTT
GATCTTTTCTACGGGGTCTGACGCTCAGTGGAACGAAAACTCACGTTAAG
GGATTTTGGTCATGAGATTATCAAAAAGGATCTTCACCTAGATCCTTTTA
AATTAAAAATGAAGTTTTAAATCAATCTAAAGTATATATGAGTAAACTTG
GTCTGACAGTTAGAAAAACTCATCGAGCATCAAATGAAACTGCAATTTAT
TCATATCAGGATTATCAATACCATATTTTTGAAAAAGCCGTTTCTGTAAT
GAAGGAGAAAACTCACCGAGGCAGTTCCATAGGATGGCAAGATCCTGGTA
TCGGTCTGCGATTCCGACTCGTCCAACATCAATACAACCTATTAATTTCC
CCTCGTCAAAAATAAGGTTATCAAGTGAGAAATCACCATGAGTGACGACT
GAATCCGGTGAGAATGGCAAAAGTTTATGCATTTCTTTCCAGACTTGTTC
AACAGGCCAGCCATTACGCTCGTCATCAAAATCACTCGCATCAACCAAAC
CGTTATTCATTCGTGATTGCGCCTGAGCGAGTCGAAATACGCGATCGCTG
TTAAAAGGACAATTACAAACAGGAATCGAATGCAACCGGCGCAGGAACAC
GGCCAGCGCATCAACAATATTTTCACCTGAATCAGGATATTCTTCTAATA
CCTGGAATGCTGTTTTCCCGGGGATCGCTGTGGTGAGTAACCATGCATCA
TCAGGAGTACGGATAAAATGCTTGATGGTCGGAAGAGGCATAAATTCCGT
CAGCCAGTTTAGTCTGACCATCTCATCTGTAACATCATTGGCAACGCTAC
CTTTGCCATGTTTCAGAAACAACTCTGGCGCATCGGGCTTCCCATACAAT
CGATAGATTGTCGCACCTGATTGCCCGACATTATCGCGAGCCCATTTATA
CCCATATAAATCAGCATCCATGTTGGAATTTAATCGCGGCCTAGAGCAAG
ACGTTTCCCGTTGAATATGGCTCATACTCTTCCTTTTTCAATATTATTGA
AGCATTTATCAGGGTTATTGTCTCATGAGCGGATACATATTTGAATGTAT
TTAGAAAAATAAACAAATAGGGGTTCCGCGCACATTTCCCCGAAAAGT (
SEQ ID NO:271).

In some embodiments, a CAR-T expression vector described herein can include the following ordered components: a promoter sequence, a Kozak sequence, a start codon, one or more nucleotide sequences encoding a protein of interest (for example, a CAR nucleotide sequence and/or an amino acid transporter nucleotide sequence, for example an arginine transporter nucleotide sequence), a stop codon, and a termination and polyA signal nucleotide sequence.

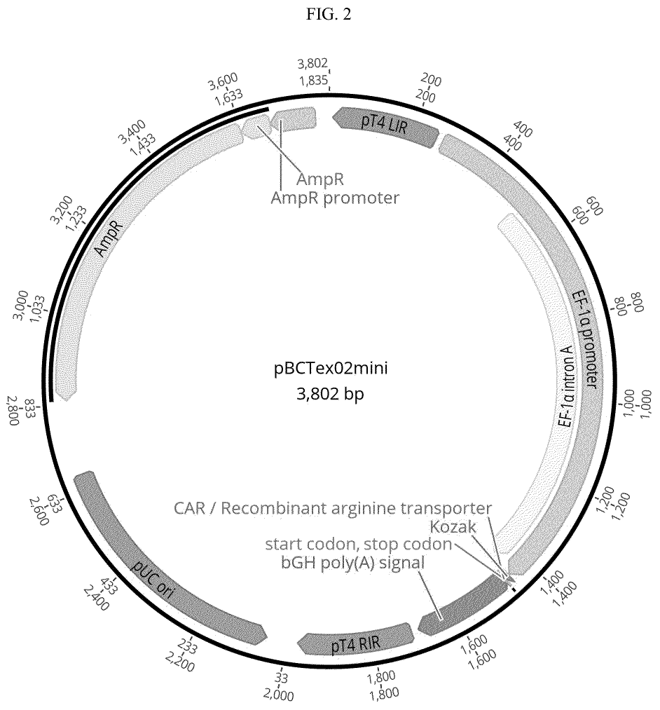

In some embodiments, a CAR-T expression vector described herein can include the following ordered components: a left inverted repeat sequence, a promoter sequence (for example, an EF-1α promoter sequence), a Kozak sequence, a start codon, one or more nucleotide sequences encoding a protein of interest (for example, a CAR nucleotide sequence and/or an amino acid transporter nucleotide sequence, for example an arginine transporter nucleotide sequence), a stop codon, a termination and polyA signal nucleotide sequence (for example, a bGH polyA signal sequence), and a right inverted terminal repeat sequence. An exemplary CAR-T expression vector is pBCTex02mini, shown in FIG. 2. The nucleotide sequence of pBCTex02mini is the following:

CCAATGATTACAGTTGAAGTCGGAAGTTTACATACACTTAAGTTGGAGTC
ATTAAAACTCGTTTTTCAACTACTCCACAAATTTCTTGTTAACAAACAAT
AGTTTTGGCAAGTCAGTTAGGACATCTACTTTGTGCATGACACAAGTCAT
TTTTCCAACAATTGTTTACAGACAGATTATTTCACTTATAATTCACTGTA
TCACAATTCCAGTGGGTCAGAAGTGTACATACACGCGCTTGACTGTGCCT
TTGCTCTTCAATGGGAGGGCTCCGGTGCCCGTCAGTGGGCAGAGCGCACA
TCGCCCACAGTCCCCGAGAAGTTGGGGGGAGGGGTCGGCAATTGAACCGG
TGCCTAGAGAAGGTGGCGCGGGGTAAACTGGGAAAGTGATGTCGTGTACT
GGCTCCGCCTTTTTCCCGAGGGTGGGGGAGAACCGTATATAAGTGCAGTA
GTCGCCGTGAACGTTCTTTTTCGCAACGGGTTTGCCGCCAGAACACAGGT
AAGTGCCGTGTGTGGTTCCCGCGGGCCTGGCCTCTTTACGGGTTATGGCC
CTTGCGTGCCTTGAATTACTTCCACCTGGCTGCAGTACGTGATTCTTGAT
CCCGAGCTTCGGGTTGGAAGTGGGTGGGAGAGTTCGAGGCCTTGCGCTTA
AGGAGCCCCTTCGCCTCGTGCTTGAGTTGAGGCCTGGCCTGGGCGCTGGG
GCCGCCGCGTGCGAATCTGGTGGCACCTTCGCGCCTGTCTCGCTGCTTTC
GATAAGTCTCTAGCCATTTAAAATTTTTGATGACCTGCTGCGACGCTTTT
TTTCTGGCAAGATAGTCTTGTAAATGCGGGCCAAGATCTGCACACTGGTA
TTTCGGTTTTTGGGGCCGCGGGCGGCGACGGGGCCCGTGCGTCCCAGCGC
ACATGTTCGGCGAGGCGGGGCCTGCGAGCGCGGCCACCGAGAATCGGACG
GGGGTAGTCTCAAGCTGGCCGGCCTGCTCTGGTGCCTGGCCTCGCGCCGC
CGTGTATCGCCCCGCCCTGGGCGGCAAGGCTGGCCCGGTCGGCACCAGTT
GCGTGAGCGGAAAGATGGCCGCTTCCCGGCCCTGCTGCAGGGAGCTCAAA
ATGGAGGACGCGGCGCTCGGGAGAGCGGGCGGGTGAGTCACCCACACAAA
GGAAAAGGGCCTTTCCGTCCTCAGCCGTCGCTTCATGTGACTCCACGGAG
TACCGGGCGCCGTCCAGGCACCTCGATTAGTTCTCGAGCTTTTGGAGTAC
GTCGTCTTTAGGTTGGGGGGAGGGGTTTTATGCGATGGAGTTTCCCCACA
CTGAGTGGGTGGAGACTGAAGTTAGGCCAGCTTGGCACTTGATGTAATTC
TCCTTGGAATTTGCCCTTTTTGAGTTTGGATCTTGGTTCATTCTCAAGCC
TCAGACAGTGGTTCAAAGTTTTTTTCTTCCATTTCAGGTGTCGTGATACT
GCCGCCACCATGTAAGCTTCTGTGCCTTCTAGTTGCCAGCCATCTGTTGT
TTGCCCCTCCCCCGTGCCTTCCTTGACCCTGGAAGGTGCCACTCCCACTG
TCCTTTCCTAATAAAATGAGGAAATTGCATCGCATTGTCTGAGTAGGTGT
CATTCTATTCTGGGGGGTGGGGTGGGGCAGGACAGCAAGGGGGAGGATTG
GGAAGACAATAGCAGGCATGCTGGGGATGCGGTGGGCTCTATGGCGCTGC
ATGAAGAGCTTAAACAATTTAAAGGCAATGCTACCAAATACTAAGCGCGT
GTATGTACACTTCTGACCCACTGGGAATGTGATGAAAGAAATAAAAGCTG
AAATGAATCATTCTCTCTACTATTATTCTGATATTTCACATTCTTAAAAT
AAAGTGGTGATCCTAACTGACCTTAAGACAGGGAATCTTTACTCGGATTA
AATGTCAGGAATTGTGAAAAAGTGAGTTTAAATGTATTTGGCTAAGGTGT
ATGTAAACTTCCGACTTCAACTGTAATCGGAAAGAACATGTGAGCAAAAG
GCCAGCAAAAGGCCAGGAACCGTAAAAAGGCCGCGTTGCTGGCGTTTTTC
CATAGGCTCCGCCCCCCTGACGAGCATCACAAAAATCGACGCTCAAGTCA
GAGGTGGCGAAACCCGACAGGACTATAAAGATACCAGGCGTTTCCCCCTG
GAAGCTCCCTCGTGCGCTCTCCTGTTCCGACCCTGCCGCTTACCGGATAC
CTGTCCGCCTTTCTCCCTTCGGGAAGCGTGGCGCTTTCTCATAGCTCACG
CTGTAGGTATCTCAGTTCGGTGTAGGTCGTTCGCTCCAAGCTGGGCTGTG
TGCACGAACCCCCCGTTCAGCCCGACCGCTGCGCCTTATCCGGTAACTAT
CGTCTTGAGTCCAACCCGGTAAGACACGACTTATCGCCACTGGCAGCAGC
CACTGGTAACAGGATTAGCAGAGCGAGGTATGTAGGCGGTGCTACAGAGT
TCTTGAAGTGGTGGCCTAACTACGGCTACACTAGAAGAACAGTATTTGGT
ATCTGCGCTCTGCTGAAGCCAGTTACCTTCGGAAAAAGAGTTGGTAGCTC
TTGATCCGGCAAACAAACCACCGCTGGTAGCGGTGGTTTTTTTGTTTGCA
AGCAGCAGATTACGCGCAGAAAAAAAGGATCTCAAGAAGATCCTTTGATC
TTTTCTACGGGGTCTGACGCTCAGTGGAACGAAAACTCACGTTAAGGGAT
TTTGGTCATGAGATTATCAAAAAGGATCTTCACCTAGATCCTTTTAAATT
AAAAATGAAGTTTTAAATCAATCTAAAGTATATATGAGTAAACTTGGTCT
GACAGTTACCAATGCTTAATCAGTGAGGCACCTATCTCAGCGATCTGTCT
ATTTCGTTCATCCATAGTTGCCTGACTCCCCGTCGTGTAGATAACTACGA
TACGGGAGGGCTTACCATCTGGCCCCAGTGCTGCAATGATACCGCGAGAC
CCACGCTCACCGGCTCCAGATTTATCAGCAATAAACCAGCCAGCCGGAAG
GGCCGAGCGCAGAAGTGGTCCTGCAACTTTATCCGCCTCCATCCAGTCTA
TTAATTGTTGCCGGGAAGCTAGAGTAAGTAGTTCGCCAGTTAATAGTTTG
CGCAACGTTGTTGCCATTGCTACAGGCATCGTGGTGTCACGCTCGTCGTT
TGGTATGGCTTCATTCAGCTCCGGTTCCCAACGATCAAGGCGAGTTACAT
GATCCCCCATGTTGTGCAAAAAAGCGGTTAGCTCCTTCGGTCCTCCGATC
GTTGTCAGAAGTAAGTTGGCCGCAGTGTTATCACTCATGGTTATGGCAGC
ACTGCATAATTCTCTTACTGTCATGCCATCCGTAAGATGCTTTTCTGTGA
CTGGTGAGTACTCAACCAAGTCATTCTGAGAATAGTGTATGCGGCGACCG
AGTTGCTCTTGCCCGGCGTCAATACGGGATAATACCGCGCCACATAGCAG
AACTTTAAAAGTGCTCATCATTGGAAAACGTTCTTCGGGGCGAAAACTCT
CAAGGATCTTACCGCTGTTGAGATCCAGTTCGATGTAACCCACTCGTGCA
CCCAACTGATCTTCAGCATCTTTTACTTTCACCAGCGTTTCTGGGTGAGC
AAAAACAGGAAGGCAAAATGCCGCAAAAAAGGGAATAAGGGCGACACGGA
AATGTTGAATACTCATACTCTTCCTTTTTCAATATTATTGAAGCATTTAT
CAGGGTTATTGTCTCATGAGCGGATACATATTTGAATGTATTTAGAAAAA
TAAACAAATAGGGGTTCCGCGCACATTTCCCCGAAAAGTGCCACCTGACG
TC (SEQ ID NO:272).

CAR-T integrated lentiviral-derived transgenes described herein can include the following nucleotide components: a 5′ long-terminal repeat (LTR) sequence (including one or more of U3, R, and U5 sequences), a promoter sequence (for example, an EF1α, cumate, CAG, CMV, UbC, or PGK promoter sequence), an antigen-specific targeting sequence, a transmembrane domain sequence (for example a CD4, CD8α, CD28, CD3ζ, or ICOS transmembrane domain nucleotide sequence), an intracellular signaling domain sequence (for example, an FcRγ or CD3ζ intracellular signaling domain sequence), and a 3′ LTR sequence (including one or more of U3, R, and U5 sequences). CAR-T integrated transgenes described herein can further include one or more of the following components: a psi (Ψ) sequence, an RRE sequence, one or more co-stimulatory domain sequences (for example, a 4-1BB, CD27, CD28, CD40, CD40L, TLR2, DAP10, OX40, IL-2RB, IL-2RA, MYD88, or ICOS co-stimulatory domain sequence), an arginine transporter sequence (for example, an SLC7A1, SLC7A2, SLC7A3, SLC7A4, SLC7A6, SLC7A7, SLC3A2, SLC3A1, SLC7A9, or SLC6A14 nucleotide sequence), or a hinge or spacer domain sequence.

In some embodiments, a CAR-T integrated transgene described herein includes the following nucleotide components: a 5′ long-terminal repeat (LTR) sequence (including one or more of U3, R, and U5 sequences), a promoter sequence (for example, an EF1α, cumate, CMV, CAG, UbC, or PGK promoter sequence), an arginine transporter sequence (for example, an SLC7A1, SLC7A2, SLC7A3, SLC7A4, SLC7A6, SLC7A7, SLC3A2, SLC3A1, SLC7A9, or SLC6A14 nucleotide sequence), and a 3′ LTR sequence (including one or more of U3, R, and U5 sequences). CAR-T integrated transgenes described herein can further include one or more of a psi (Ψ) sequence and an RRE sequence.

In some embodiments, an expression cassette described herein can include a eukaryotic promoter that functions in T-cells (e.g., an EF-1α, PGK, CAG, or CMV promoter), a coding sequence of an amino acid transporter with or without a preceding Kozak sequence, and a eukaryotic transcription terminator and polyA signal (e.g., SV40, hGH, bGH, rbHBB, and rbGlob). The expression cassette can be embedded in a transposon (e.g., Sleeping Beauty, piggyBac, Tol2) to enable genomic integration without the use of lentivirus or retrovirus.

Antibiotic resistance genes (e.g., puromycin N-acetyltransferase), protein tags (e.g., 6xHis (SEQ ID NO: 278), FLAG), and/or reporters such as, but not limited to, a fluorescent protein can also be included in expression vectors described herein, either in tandem with an amino acid transporter (for example, in the form of a fusion protein) or as a separate entity (for example, separated by an IRES or a 2A cleavage sequence from the amino acid transporter coding sequence) to facilitate downstream selection.

In some embodiments, an amino acid transporter expression vector described herein can have the following ordered components: IR/DR(SB) – P_{EF1α}::Kozak – transporter – P2A – PAC-(G₄S)₃-mEGFP – BGHpolyA – DR/IR(SB) (″(G₄S)₃ disclosed as SEQ ID NO: 30).

### Tumor Microenvironment

Cancer cells create a tumor microenvironment (TME) that is permissive for tumor growth and proliferation in part by depleting essential nutrients from their environs. The metabolic state of the TME is regulated by the metabolic activity of the cancer cells which alter the availability of nutrients in the microenvironment such as glucose, lipids, and amino acids. For example, the TME is characterized by low levels of the amino acid arginine. Arginine depletion is caused in part by uptake of arginine from the TME by tumor cells. Arginine depletion is also mediated by activation of arginase and inducible nitric oxide synthase (iNOS) in tumor cells, local macrophages, granulocytes, and myeloid derived suppressor cells.

Notably, naturally occurring T cells are unable to synthesize arginine. Therefore, T cells depend on a sustainable supply of exogenous arginine. However, T cell activation, survival, and persistence is compromised by the relatively low levels of arginine in the TME. In particular, conditions of the TME, including low arginine levels, impair T-cell receptor signaling, glycolytic metabolism, amino acid uptake, and metabolism resulting in impaired anti-tumor effector functions of tumor-specific T-effector cells. Furthermore, Treg cells, which rely mainly on fatty acid oxidation as opposed to amino acid uptake, can survive under TME conditions and exert immunosuppressive effects on tumor-specific T-effector cells. Thus, the conditions of the TME suppress T-effector cell differentiation and promote immunosuppression. Currently available CAR-T cells are susceptible to the same adversities in the TME as their native T-cell counterparts resulting poor efficacy for CAR-T treatments in solid tumors. The present invention provides CAR-T cells capable of competing with cancer cells and MDSCs for arginine, increasing their survival, persistence and anti-tumor activity in solid tumors compared to CAR-T cells known in the art.

In particular, the present invention provides CAR-T cells that have an enhanced ability to transport amino acids, particularly arginine, from the extracellular space into the cytosol. For example, CAR-T cells described herein are genetically engineered to express an amino acid transporter capable of transporting an amino acid, for example, arginine, into the CAR-T cell. CAR-T cells described herein that are genetically engineered to express an amino acid transporter are characterized by higher T cell activation, persistence, proliferation, and/or anti-tumor efficacy compared to T cells and CAR-T cells that are not genetically engineered to express an amino acid transporter. Additionally, CAR-T cells described herein that are genetically engineered to express an amino acid transporter are characterized by a higher rate of survival and persistence in a TME compared to T cells and CAR-T cells that are not genetically engineered to express an amino acid transporter.

### CAR-T Cell Priming

In one aspect, the invention includes a method of modulating intracellular arginine levels in a CAR-T cell (for example, a CAR-T cell described herein) to effect a T cell-mediated immune response in a patient in need thereof. For example, in some embodiments, the invention includes exposing a CAR-T cell which expresses an arginine transporter and a CAR to a medium that includes arginine, wherein exposing the CAR-T cell to the medium is effective to increase the intracellular arginine concentration of the CAR-T cell. Exposing a CAR-T cell which expresses an arginine transporter and a CAR to a medium that includes arginine, for example, culturing in vitro such a CAR-T cell in an arginine-rich medium, can result in an increased CAR-T intracellular arginine concentration relative to CAR-T cells not exposed to the medium. Such intracellular arginine-enriched CAR-T cells can compete with cancer cells and MDSCs for extracellular arginine, for example, in the extracellular space of the TME. Thus, in some embodiments, the invention includes exposing a CAR-T cell which expresses an arginine transporter and a CAR to a medium that includes arginine, wherein exposing the CAR-T cell to the medium is effective to increase CAR-T survival, life-span, and functional activity. For example, in some embodiments, exposing the CAR-T cell to the medium is effective to increase CAR-T anti-tumor activity (for example, exposing the CAR-T cell to the medium is effective to increase CAR-T anti-tumor activity in the TME of a solid tumor). Thus, also described herein is are methods of administering intracellular arginine-enriched CAR-T cells, wherein the method is effective to treat hematological malignancies as well as solid tumors.

In some embodiments, a medium that is effective to increase the intracellular arginine concentration of a CAR-T cell contains a physiological level of L-arginine including, but not limited to, 0.2 g/L or 100 µmol/L, or a supraphysiological level of L-arginine such as, but not limited to, 100 µmol/L, 200 µmol/L, 300 µmol/L, 400 µmol/L, 500 µmol/L, 600 µmol/L, 700 µmol/L, 800 µmol/L, 900 µmol/L, 1000 µmol/L, or more than 1000 µmol/L. The medium can be RPMI-1640 with or without supplement. The medium can be supplemented with serums and/or nutrients such as but not limited to fetal bovine serum, human AB serum, or human platelet lysate. The engineered T-cells may be cultured and primed in L-arginine-rich media until intracellular arginine accumulates to a sufficient level such as but not limited to 20 µmol, 30 µmol, 40 µmol, 50 µmol, 60 µmol, 70 µmol, 80 µmol, 90 µmol, 100 µmol, 200 µmol, 2000 µmol, or more than 2000 µmol. In some embodiments, CAR-T cells can be cultured and primed in L-arginine-rich media until intracellular arginine accumulates to about 10 µM, about 20 µM, about 30 µM, about 40 µM, about 50 µM, about 60 µM, about 70 µM, about 80 µM, about 90 µM, about 100 µM, about 200 µM, about 300 µM, about 400 µM, about 500 µM, about 600 µM, about 700 µM, about 800 µM, about 900 µM, about 1000 µM, about 1500 µM, about 2000 µM, about 2500 µM, about 3000 µM, about 3500 µM, about 4000 µM, about 100 µM to about 4000 µM, about 100 µM to about 1000 µM, about 100 µM to about 2000 µM, about 1000 µM to about 2000 µM, about 1000 µM to about 3000 µM, about 1000 µM to about 4000 µM, about 500 µM to about 1000 µM, about 3000 µM to about 4000 µM, about 2000 µM to about 4000 µM, or about 500 µM to about 2000 µM arginine per cell.

### Kits

Also described herein are kits that include a pharmaceutical composition described herein. For example, in some embodiments, a pharmaceutical composition comprising a CAR-T cell which expresses an arginine transporter and a CAR is packaged as a kit. A kit described herein can include instructions for administering the CAR-T cells to a patient in need of treatment. A kit described herein can include instructions for priming CAR-T cells for administration to a patient in need of treatment. A kit described herein can include instructions for producing CAR-T cells that express an arginine transporter and a CAR. In some embodiments, the kit may include at least one of buffers (for example, a buffer comprising levels of L-arginine sufficient for priming T-cells), reagents and detailed instructions for producing, expanding, administering, and/or priming CAR-T cells.

Kits described herein for producing CAR-T cells can include an expression vector encoding a CAR, an expression vector encoding an arginine transporter, an expression vector encoding a CAR and an arginine transporter, and/or an expression vector encoding a transposase for stable integration of a CAR and/or an arginine transporter. The kit may include polycistronic expression vectors capable of expressing a CAR and an arginine transporter.

Kits described herein for producing CAR-T cells can include reagents, including culture medium, cells, transfection reagents, buffers, and nucleotide constructs for producing a virus that includes a nucleotide construct encoding a CAR, an arginine transporter, or a CAR and an arginine transporter. The kit may include polycistronic expression vectors capable of expressing a CAR and an arginine transporter.

Kits described herein can include reagents for assaying CAR and/or arginine transporter protein expression in a CAR-T cell. For example, kits described herein can include an antibody (for example, a polyclonal antibody) specific to an arginine transporter. Kits described herein can include an antibody (for example, a polyclonal antibody) specific to the CAR antigen-recognition domain.

### Methods of Treating Cancer

The methods of this disclosure include methods of treating, preventing, arresting, reversing, or ameliorating a disease. In some embodiments of the methods described herein, the disease is a cancer. In some embodiments, a method of treating, preventing, arresting, reversing, or ameliorating is achieved by administering a therapeutically effective dose of a CAR-T cell described herein, for example, an arg+CAR-T cell described herein. For example, described herein is a method of treating a solid tumor cancer in a patient in need thereof, comprising administering to the patient an effective amount of a CAR-T cell described herein or a pharmaceutical composition that includes a CAR-T cell described herein. Also described herein is a method of treating a hematological cancer in a patient in need thereof, comprising administering to the patient an effective amount of a CAR-T cell described herein or a pharmaceutical composition that includes a CAR-T cell described herein. Also described herein is a method for treating a condition in a human patient in need thereof, comprising: administering to the human patient a therapeutically effective amount of a composition comprising a CAR-T cell which expresses an arginine transporter and a chimeric antigen receptor protein or a pharmaceutical composition that includes a CAR-T cell which expresses an arginine transporter and a chimeric antigen receptor protein.

The activity of a plurality of cells in the immune system can be modulated by arginine, for example: macrophages, B-cells, T-cells, natural killer cells, neutrophils, and dendritic cells. Modulation of intracellular arginine can effect T-cell-mediated immune responsiveness. Thus, described herein is a method of modulating intracellular arginine levels to effect a T cell-mediated immune response in a patient in need thereof, comprising administering to the patient an effective amount of a CAR-T cell described herein or a pharmaceutical composition that includes a CAR-T cell described herein.

Described herein are methods of treating, preventing, arresting, reversing, or ameliorating a disease in a subject or a patient in need thereof. In embodiments described herein, patients and subjects can be humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. A subject or patient can be of any age. Subjects and patients can be, for example, elderly adults, adults, adolescents, pre-adolescents, children, toddlers, or infants.

Examples of diseases or conditions that can be treated with engineered CAR-T-cells overexpressing arginine transporters, including engineered CAR-T cells overexpressing the arginine transporters of Table 1, include hematological malignancies, solid tumor malignancies, metastatic cancer, benign tumors, cold tumors, primary tumors, and secondary tumors.

In some embodiments, disclosed herein is method of treating a cancer with engineered CAR-T-cells described herein, for example, CAR-T cells overexpressing an arginine transporter, including engineered CAR-T cells overexpressing an arginine transporter of Table 1. Methods of treating a cancer described herein include methods of treating, for example, any of the following: acute lymphoblastic leukemia, acute myeloid leukemia, adrenocortical carcinoma, AIDS-related cancers, AIDS-related lymphoma, anal cancer, appendix cancer, astrocytomas, neuroblastoma, basal cell carcinoma, bile duct cancer, bladder cancer, bone cancers, brain tumors, such as cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas, Burkitt lymphoma, carcinoma of unknown primary origin, central nervous system lymphoma, cerebellar astrocytoma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing’s sarcoma, germ cell tumors, gallbladder cancer, gastric cancer, gastrointestinal carcinoid tumor, gastrointestinal stromal tumor, gliomas, hairy cell leukemia, head and neck cancer, heart cancer, hepatocellular (liver) cancer, Hodgkin lymphoma, Hypopharyngeal cancer, intraocular melanoma, islet cell carcinoma, Kaposi sarcoma, kidney cancer, laryngeal cancer, lip and oral cavity cancer, liposarcoma, liver cancer, lung cancers, such as non-small cell and small cell lung cancer, lymphomas, leukemias, macroglobulinemia, malignant fibrous histiocytoma of bone/osteosarcoma, medulloblastoma, melanomas, mesothelioma, metastatic squamous neck cancer with occult primary, mouth cancer, multiple endocrine neoplasia syndrome, myelodysplastic syndromes, myeloid leukemia, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer, ovarian germ cell tumor, pancreatic cancer, pancreatic cancer islet cell, paranasal sinus and nasal cavity cancer, parathyroid cancer, penile cancer, pharyngeal cancer, pheochromocytoma, pineal astrocytoma, pineal germinoma, pituitary adenoma, pleuropulmonary blastoma, plasma cell neoplasia, primary central nervous system lymphoma, prostate cancer, rectal cancer, renal cell carcinoma, renal pelvis and ureter transitional cell cancer, retinoblastoma, rhabdomyosarcoma, salivary gland cancer, sarcomas, skin cancers, skin carcinoma merkel cell, small intestine cancer, soft tissue sarcoma, squamous cell carcinoma, stomach cancer, T-cell lymphoma, throat cancer, thymoma, thymic carcinoma, thyroid cancer, trophoblastic tumor (gestational), cancers of unknown primary site, urethral cancer, uterine sarcoma, vaginal cancer, vulvar cancer, Waldenstrom macroglobulinemia, and Wilms tumor.

In some embodiments, in methods of treating a cancer that include a step of administering a CAR-T cell, the antigen-specific target region of the CAR can recognize and bind a cell surface antigen. In some embodiments, the CAR can be used in a method of treating a cancer for which a specific monoclonal antibody exists or is capable of being generated. In particular, cancers such as neuroblastoma, small cell lung cancer, melanoma, ovarian cancer, renal cell carcinoma, colon cancer, Hodgkin’s lymphoma, and childhood acute lymphoblastic leukemia have antigens recognized by CARs described herein.

Methods of treating described herein can include treating a subject (e.g. a patient with a disease and/or a lab animal with a condition) with genetically engineered CAR-T-cells overexpressing an amino acid transporter, including engineered CAR-T cells overexpressing an arginine transporter. The disease may be a hematological malignancy. The disease may be a solid tumor malignancy. The subject may be a human. Treatment may be provided to the subject before clinical onset of disease. Treatment may be provided to the subject after clinical onset of disease.

Treatment may be provided to the subject about 1 day, 1 week, 2 weeks, 3 weeks, 4 weeks, 5 weeks, 6 weeks, 7 weeks, 8 weeks, 1 month, 2 months, 3 months, 4 months, 5 months, 6 months, 7 months, 8 months, 9 months, 10 months, 11 months, 12 months, 1 year, 2 years, 3 years, 4 years, 5 years, or more after clinical onset of the disease. Treatment may be provided to the subject for about 1 hour, 2 hours, 3 hours, 4 hours, 5 hours, 6 hours, 12 hours, 15 hours, 18 hours, 1 day, 2 days, 3 days, 4 days, 5 days, 6 days, 1 week, 2 weeks, 3 weeks, 4 weeks, 5 weeks, 6 weeks, 7 weeks, 8 weeks, 1 month, 2 months, 3 months, 4 months, 5 months, 6 months, 7 months, 8 months, 9 months, 10 months, 11 months, 12 months, 1 year, 2 years, 3 years, 4 years, 5 years, or more after clinical onset of disease. Treatment may be provided to the subject for more than 1 hour, 2 hours, 3 hours, 4 hours, 5 hours, 6 hours, 12 hours, 15 hours, 18 hours, 1 day, 2 days, 3 days, 4 days, 5 days, 6 days, 1 week, 2 weeks, 3 weeks, 4 weeks, 5 weeks, 6 weeks, 7 weeks, 8 weeks, 1 month, 2 months, 3 months, 4 months, 5 months, 6 months, 7 months, 8 months, 9 months, 10 months, 11 months, 12 months, 1 year, 2 years, 3 years, 4 years, 5 years, or more after clinical onset of disease. Treatment may be provided to the subject for less than 1 hour, 2 hours, 3 hours, 4 hours, 5 hours, 6 hours, 12 hours, 15 hours, 18 hours, 1 day, 2 days, 3 days, 4 days, 5 days, 6 days, 1 week, 2 weeks, 3 weeks, 4 weeks, 5 weeks, 6 weeks, 7 weeks, 8 weeks, 1 month, 2 months, 3 months, 4 months, 5 months, 6 months, 7 months, 8 months, 9 months, 10 months, 11 months, 12 months, 1 year, 2 years, 3 years, 4 years, 5 years, or more after clinical onset of disease. Treatment may also include treating a human in a clinical trial. A treatment can comprise administering to a subject a pharmaceutical composition, such as one or more of the pharmaceutical compositions described throughout the disclosure. A treatment can comprise modulating the levels of endogenous arginine in vivo.

Methods of re-introducing cellular components are known in the art and include procedures such as those exemplified in U.S. Pat. Nos. 4,844,893 and 4,690,915. The amount of activated T cells used can vary between in vitro and in vivo uses, as well as with the amount and type of the target cells. The amount administered will also vary depending on the condition of the patient and should be determined by considering all appropriate factors by the practitioner.

### Combinations of Immune Checkpoint Therapies with Engineered CAR-T Cells

Also disclosed herein are combination therapies, and methods of using the same, comprising administering engineered CAR-T-cells (or pharmaceutical compositions thereof) for example, CAR-T cells overexpressing an amino acid transporter, for example, an arginine transporter disclosed herein in combination with a second therapeutic. For example, described herein is a method of treating cancer comprising administering: a genetically modified T-cell modified to express a CAR and an amino acid transporter, for example an arginine transporter; and an immunotherapy that targets an immune checkpoint (for example, an immune checkpoint inhibitor). For example, described herein is a method of treating cancer comprising administering: a genetically modified T-cell modified to express a CAR and an amino acid transporter, for example an arginine transporter; and an agent that blocks the interaction of PD-1 and PD-L1 or which blocks the interaction of CTLA-4 and B7-1/B7-2. For example, described herein is a method of treating cancer comprising administering: a genetically modified T-cell modified to express a CAR and an amino acid transporter, for example an arginine transporter; and an anti-PD-1, anti-PD-L1, or an anti-CTLA-4 antibody. Also described herein is a method of treating cancer comprising administering: a genetically modified T-cell modified to express a CAR and an amino acid transporter, for example an arginine transporter; and a compound selected from the group consisting of ipilimumab, nivolumab, pembrolizumab, atezolizumab, avelumab, durvalumab, and cemiplimab. Combination therapies of the disclosure can be co-administered to a subject to improve the outcome of a cancer treatment. In some embodiments, a CAR-T-cell described herein and the immune checkpoint inhibitor are administered simultaneously or sequentially to the patient in need of treatment.

Most immunological checkpoint molecules are members of the immunoglobulin superfamily, and are often inhibitory receptors that prevent uncontrolled immune reactions. The adaptive immune response is controlled by such checkpoint molecules, which are important for maintaining self-tolerance and minimizing collateral tissue damage that can occur during an immune response. In some embodiments, a combination therapy that targets immune checkpoints and promotes amino acid uptake, in particular arginine uptake, by CAR-T cells, can yield better outcomes for subjects afflicted with solid malignancies and hematological malignancies.

Immune checkpoints are co-stimulatory and inhibitory elements intrinsic to the immune system. Immune checkpoints aid in maintaining self-tolerance and modulating the duration and amplitude of physiological immune responses to prevent injury to tissues when the immune system responds to pathogenic infection. An immune response can also be initiated when a T-cell recognizes antigens that are characteristic of a tumor cell. The equilibrium between the costimulatory and inhibitory signals used to control the immune response from T-cells can be modulated by immune checkpoint proteins. After T-cells mature and activate in the thymus, T-cells can travel to sites of inflammation and injury to perform repair functions. T-cell function can occur either via direct action or through the recruitment of cytokines and membrane ligands involved in the immune system. The steps involved in T-cell maturation, activation, proliferation, and function can be regulated through co-stimulatory and inhibitory signals, namely through immune checkpoint proteins. Tumors can dysregulate checkpoint protein function as an immune-resistance mechanism. Thus, the development of modulators of checkpoint proteins can have therapeutic value. Non-limiting examples of immune checkpoint molecules include CTLA4 and PD-1. These checkpoint molecules can operate upstream of IL-2 in a pathway. Checkpoint inhibitors include agents that block the interaction of PD-1 and PD-L1 or which block the interaction of CTLA-4 and B7-1/B7-2. Examples of specific checkpoint inhibitors include the following antibody-based drugs: ipilimumab, nivolumab, pembrolizumab, atezolizumab, avelumab, durvalumab, and cemiplimab.

In some instances the disclosure provides a method for treating a condition in a human subject, comprising: (a) administering to the human subject a therapeutically effective amount of a composition comprising CAR-T cell which ectopically expresses arginine transporter(s) and a chimeric antigen receptor protein; and (b) administering a second therapeutic agent to the human subject, wherein the second therapeutic agent is an anti-PD-1, anti-PD-L1, or an anti-CTLA-4 antibody. The administering of the second therapeutic agent can be performed before, during or after the administration of the composition comprising the CAR-T cell composition.

PD1 is an inhibitory receptor belonging to the CD28/CTLA-4 family and is expressed on the surface of activated T-cells, B-cells, monocytes, DCs, and Natural Killer (NK) cells. In contrast to CTLA-4, the major role of PD-1 is limitation of activity of T-cells in peripheral tissues at the time of an inflammatory response to infection and to limit autoimmunity. Chronic antigen exposure can lead to persistently-high levels of PD-1 expression, which can induce a state of exhaustion or anergy of antigen-specific T-cells, which can be at least partially reversed by PD-1 blockade. In some embodiments, an engineered CAR-T cell of the disclosure and an anti-PD-1 or anti-PD-L1 antibody are co-administered to subjects afflicted with a condition.

CTLA-4 (cytotoxic T-lymphocyte antigen 4) is also known as CD152 (Cluster of differentiation 152). CTLA-4 shares sequence homology and ligands (CD80B7-1 and CD86/B7-2) with the costimulatory molecule CD28, but differs by delivering inhibitory signals to T cells expressing CTLA-4 as a receptor. CTLA-4 has a much higher overall affinity for both ligands and can out-compete CD28 for binding when ligand densities are limiting. CTLA-4 is expressed on the surface of CD8+ effector T-cells, and plays a functional role in the initial activation stages of both naive and memory T cells. CTLA-4 counteracts the activity of CD28 via increased affinity for CD80 and CD86 during the early stages of T-cell activation. The major functions of CTLA-4 include downmodulation of helper T-cells and enhancement of regulatory T-cell immunosuppressive activity.

CTLA-4 can also downregulate immune system functions via inhibition of IL-2 production and IL-2 receptor expression. CTLA-4 can inhibit CD28-dependent upregulation of IL-2, and the inhibition of IL-2 production can lead to cell cycle arrest. The decrease in IL-2 and subsequent cell cycle arrest can account for the reduced T-cell proliferation observed in the presence of CTLA-4.

### Other Combination Therapies

As noted above, also disclosed herein are combination therapies, and methods of using the same, comprising administering engineered CAR-T-cells for example, CAR-T cells overexpressing an amino acid transporter, for example, an arginine transporter disclosed herein, or a pharmaceutical composition thereof, in combination with a second therapeutic. In some embodiments, a CAR-T-cell described herein, or a pharmaceutical composition thereof, and a second therapeutic are administered simultaneously or sequentially to a patient in need of treatment. In some embodiments, the method comprises administering the second therapeutic agent before, during or after the administering of a therapeutically effective amount of T-cells or a composition comprising a therapeutically effective amount of the CAR-T cells.

For example, described herein is a method of treating cancer comprising administering: a genetically modified T-cell modified to express a CAR and an amino acid transporter, for example an arginine transporter; or a pharmaceutical composition thereof, and a DNA damage response inhibitor (DDRi). In some embodiments, the DDRi is selected from the group consisting of an ATM inhibitor, a PARP inhibitor, an ATR inhibitor, a WEE1 inhibitor, a Chk1 inhibitor, a Chk2 inhibitor, and a DNA-protein kinase inhibitor. In some embodiments, the DDRi is a PARP inhibitor (PARPi) selected from the group consisting of: niraparib, olaparib, pamiparib, rucaparib (camsylate), talazoparib, veliparib, and an analog thereof. In some embodiments, the DDRi is an ATM/ATR inhibitor. In some embodiments the ATM/ATR inhibitor is selected from the group consisting of: AZ20, AZD0156, AZD1390, AZD6738, BAY-1895344, EPT-46464, M3541, M4344, M6620 (formerly known as VE-922 or VX-970), NU6027, VE-821, and an analog thereof. In some embodiments, the PARPi is adavosertib, AZD2811, or an analog thereof. In some embodiments, the DDRi is a WEE1 inhibitor, a Chk1 inhibitor, or a Chk2 inhibitor. In some embodiments, the DDRi is a DNA-dependent protein kinase (DNA-PK) inhibitor selected from the group consisting of: AZD7648, KU-0060648, NU7026, NU7441 (KU-57788), PI-103, PIK-75 HCI, PP121, SF2523, and an analog thereof.

In some embodiments, the method comprises administering a genetically modified T-cell modified to express a CAR and an amino acid transporter, for example an arginine transporter, or a pharmaceutical composition thereof; and: a radiotherapy, a chemotherapy, an immunotherapy, a hormone therapy, an angiogenesis inhibitor, a stem cell transplant therapy, a bone marrow transplant therapy, or a targeted therapy.

Examples of radiotherapy include external beam radiation therapy, internal beam radiation therapy, brachytherapy, and systemic radiation therapy.

Examples of chemotherapy agents include alkylating agents (for example, altretamine, bendamustine, busulfan, carboplatin, carmustine, chlorambucil, cisplatin, cyclophosphamide, dacarbazine, ifosfamide, lomustine, mechlorethamine, melphalan, oxaliplatin, temozolomide, thiotepa, and trabectedin), nitrosureas (for example, carmustine, lomustine, and streptozocin), antimetabolites (for example, azacitidine, 5-fluorouracil (5-fu), 6-mercaptopurine (6-mp), capecitabine (xeloda), cladribine, clofarabine, cytarabine (ara-c), decitabine, floxuridine, fludarabine, gemcitabine (gemzar), hydroxyurea, methotrexate, nelarabine, pemetrexed (alimta), pentostatin, pralatrexate, thioguanine, and trifluridien/tipiracil), anthracyclines (for example, daunorubicin, doxorubicin (adriamycin), doxorubicin liposomal, epirubicin, idarubicin, and valrubicin), non-anthracycline anti-tumor antibiotics (for example, bleomycin, dactinomycin, mitomycin-c, and mitoxantrone), topoisomerase inhibitors (for example, irinotecan, irinotecan liposomal, topotecan etoposide (vp-16), mitoxantrone, teniposide), mitotic inhibitors such as taxanes and vinca alkaloids (for example, capazitaxel, docetaxel, nab-paclitaxel, paclitaxel, vinca alkaloids include:, vinblastine, vincristine, vincristine liposomal, vinorelbine) corticosteroids (for example, prednisone, methylprednisolone, and dexamethasone), all-trans-retinoic acid, arsenic trioxide, asparaginase, eribulin, hydroxyurea, ixabepilone, mitotane, omacetaxine, pegasparaginase, procarbazine, romidepsin, and vorinostat.

Examples of immunotherapy agents include immune checkpoint inhibitors, cancer treatment vaccines (for example, human papillomavirus vaccine, hepatitis B vaccine, Sipuleucel-T (Provenge) and Talimogene laherparepvec (T-VEC)), monoclonal antibodies (for example, alemtuzumab, bevacizumab, cetuximab, gemtuzumab ozogamicin, ipilimumab, ofatumumab, panitumumab, pembrolizumab, ranibizumab, rituximab, and trastuzumab), and immune system modulators (for example, interleukins (e.g., IL-2, IL-7, IL-21, and IL-12), cytokines (e.g., interferons (IFN-α, IFN-β, and IFN-γ)and G-CSF), chemokines (e.g., CCL3, CCL26, and CXCL7), immunomodulatory imide drugs (e.g., thalidomide and its analogues (lenalidomide, pomalidomide, and apremilast)), imiquimod, Bacillus Calmette-Guerin (BCG), cytosine phosphate-guanosine, oligodeoxynucleotides, and glucans).

Examples of hormone therapy include abiraterone (Zytiga®), anastrozole (Arimidex®), exemestane (Aromasin®), fulvestrant (Faslodex®), letrozole (Femara®), leuprolide (Eligard®, Lupron Depot®), toremifene (Fareston®), fluoxymesterone (Halotestin®), megestrol acetate (Megace®), bicalutamide (Cased®), nilutamide (Nilandron®), flutamide (Eulexin®), goserelin (Zoladex®), degarelix (Firmagon®), and tamoxifen (Nolvadex®).

Examples of angiogenesis inhibitors include: axitinib (Inlyta®), bevacizumab (Avastin®), cabozantinib (Cometriq®), everolimus (Afinitor®), lenalidomide (Revlimid®), lenvatinib mesylate (Lenvima®), pazopanib (Votrient®), ramucirumab (Cyramza®), regorafenib (Stivarga®), sorafenib (Nexavar®), sunitinib (Sutent®), thalidomide (Synovir, Thalomid®), vandetanib (Caprelsa®), and ziv-aflibercept (Zaltrap®).

Examples of targeted therapy include: EGFR inhibitors (for example, cetuximab (Erbitux®) and panitumumab (Vectibix®)), HER2 inhibitors (for example, trastuzumab (Herceptin®), pertuzumab (Perjeta®), and ado-trastuzumab emtansine (Kadcyla®)), kinase inhibitors (for example, axitinib (Inlyta®), bosutinib (Bosulif®), cabozantinib (Cometriq®), crizotinib (Xalkori®), dabrafenib (Tafinlarâ), dasatinib (Sprycel®), erlotinib (Tarceva®), ibrutinib (Imbruvica®), imatinib (Gleevec®), lapatinib (Tykerb®), nilotinib (Tasigna®), pazopanib (Votrient®), ponatinib (Iclusig®), regorafenib (Stivarga®), sorafenib (Nexavar®), sunitinib (Sutent®), trametinib (Mekinist®), vandetanib (Caprelsa®), and vemurafenib (Zelboraf®)), mTOR inhibitors (for example, sirolimus (Rapamune®), everolimus (Afinitor®), and temsirolimus (Toricel®)), hedgehod pathway inhibitors (for example, vismodegib (Erivedge®)), immune system target inhibitors (for example, alemtuzumab (Campath®), brentuximab vedotin (Adcetris®), ipilimumab (Yervoy®), ibritumomab tiuxetan (Zevalin®), obinutuzumab (Gazyva™), ofatumumab (Azerra®), and rituximab (Rituxan®)), VEGF receptor inhibitors (for example, bevacizumab (Avastin®) and ziv-aflibercept (Zaltrap®)), estrogen target inhibitors (for example, anastrozole (Arimidex®), exemestane (Aromasin), fulvestrant (Faslodex®), letrozole (Femara®), raloxifene (Evista®), tamoxifen citrate, and toremifene citrate (Fareston®)), androgen target inhibitors, (for example, abiraterone acetate (Zytiga®), bicalutamide (Casodex®), enzalutamide (Xtandi®), flutamide, and nilutamide (Nilandron®)), proteasome target inhibitors (for example, bortezomib (Velcade®) and carfilzomib (Kyprolis™)), histone deacetylase target inhibitors (for example, romidepsin (Istodax®) and vorinostat (Zolinza®)), folate target inhibitors (for example, pralatrexate (Folotyn®)), and retinoic acid receptor target inhibitors (for example, isotretinoin, tretinoin, acitretin (Soriatane®), and bexarotene (Targretin®)).

In some embodiments, a method described herein comprises administering a genetically modified T-cell modified to express a CAR and an amino acid transporter, for example an arginine transporter, or a pharmaceutical composition thereof; and performing surgery on a patient. In some embodiments, the method comprises administering a genetically modified T-cell modified to express a CAR and an amino acid transporter, for example an arginine transporter, or a pharmaceutical composition thereof, wherein the administering is to a patient that has undergone an anti-cancer surgery, will undergo an anti-cancer surgery, or is a candidate for an anti-cancer surgery. Anti-cancer surgeries include, for example, cryosurgery, laser surgery, hyperthermia, photodynamic therapy, open surgery, minimally invasive surgery.

### Pharmaceutical Compositions

A pharmaceutical composition of the invention can be a combination of any arginine transporter overexpressing CAR-T cell described herein with other chemical components, such as carriers, stabilizers, diluents, dispersing agents, suspending agents, thickening agents, and/or excipients. The pharmaceutical composition facilitates administration of the engineered CAR-T cells described herein to an organism. Pharmaceutical compositions can be administered in therapeutically-effective amounts as pharmaceutical compositions by various forms and routes including, for example, intravenous, subcutaneous, intramuscular, rectal, aerosol, parenteral, ophthalmic, pulmonary, transdermal, vaginal, optic, nasal, and topical administration. A pharmaceutical composition can be administered in a local or systemic manner, for example, via infusion of the CAR-T cells directly into an organ.

In some embodiments, a CAR-T pharmaceutical composition described herein is administered intravenously, for example, by an intravenous drip. In some embodiments, a dose of a CAR-T pharmaceutical composition is administered over the course of about 20 to about 30 minutes. In some embodiments, a dose of a CAR-T pharmaceutical composition is administered over the course of about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 1 hour, about 2 hours, about 3 hours, about 4 hours, about 5 hours, from about 10 to about 20 minutes, from about 10 to about 30 minutes, from about 10 to about 60 minutes, from about 30 to about 60 minutes, from about 40 to about 60 minutes, from about 20 to about 30 minutes, from about 20 to about 40 minutes, from about 1 hour to about 2 hours, from about 1 hour to about 3 hours, from about 1 hour to about 4 hours, from about 1 hour to about 5 hours, from about 1 hour to about 6 hours, from about 2 hours to about 3 hours, from about 2 hours to about 4 hours, or from about 3 hours to about 6 hours.

In some embodiments a dose of a CAR-T pharmaceutical composition is administered to a subject every day for 1, 2, 3, 4, 5, 6, or 7 days. In some embodiments a dose of a CAR-T pharmaceutical composition is administered to a subject every week for 1 week, 2 weeks, 3 weeks, 4 weeks, 5 weeks, 6 weeks, 7 weeks, 8 weeks, 9 weeks, 10 weeks, 11 weeks, 12 weeks, about 1 month, about 2 months, about 3 months, about 4 months, about 5 months, about 6 months, about 7 months, about 8 months, about 9 months, about 10 months, about 11 months, about 12 months, about 1 to about 2 weeks, about 1 to about 3 weeks, about 2 to about 3 weeks, about 1 to about 4 weeks, about 2 to about 4 weeks, about 3 to about 4 weeks, about 1 to about 12 weeks, about 4 to about 12 weeks, about 6 to about 12 weeks, about 8 to about 12 weeks, about 10 to about 12 weeks, about 6 to about 24 weeks, about 8 to about 24 weeks, about 10 to about 24 weeks, about 12 to about 24 weeks, about 6 to about 18 weeks, about 8 to about 18 weeks, about 10 to about 18 weeks, about 12 to about 18 weeks, about 14 to about 18 weeks, or about 16 to about 18 weeks. In some embodiments a dose of a CAR-T pharmaceutical composition is administered to a subject every 2 weeks for 4 weeks, 5 weeks, 6 weeks, 7 weeks, 8 weeks, 9 weeks, 10 weeks, 11 weeks, 12 weeks, about 1 month, about 2 months, about 3 months, about 4 months, about 5 months, about 6 months, about 7 months, about 8 months, about 9 months, about 10 months, about 11 months, about 12 months, about 1 to about 2 weeks, about 4 to about 12 weeks, about 6 to about 12 weeks, about 8 to about 12 weeks, about 10 to about 12 weeks, about 6 to about 24 weeks, about 8 to about 24 weeks, about 10 to about 24 weeks, about 12 to about 24 weeks, about 6 to about 18 weeks, about 8 to about 18 weeks, about 10 to about 18 weeks, about 12 to about 18 weeks, about 14 to about 18 weeks, or about 16 to about 18 weeks.

In practicing the methods of treatment or use provided herein, therapeutically-effective amounts of arginine transporter overexpressing CAR-T cells described herein are administered in pharmaceutical compositions to a subject suffering from a condition that affects the immune system. In some embodiments, the subject is a mammal such as a human. A therapeutically-effective amount can vary widely depending on the severity of the disease, the age and relative health of the subject, the potency of the compounds used, and other factors.

Pharmaceutical compositions described herein can include live genetically engineered cells, for example, CAR-T cells overexpressing an arginine transporter. CAR-T pharmaceutical compositions described herein can be administered to a subject at a discrete dose. For example, CAR-T cell pharmaceutical compositions described herein can be administered at a dosage of 10⁴ to 10¹¹ cells/kg body weight, 10⁵ to 10¹¹ cells/kg body weight, 10⁶ to 10¹¹ cells/kg body weight, 10⁷ to 10¹¹ cells/kg body weight, 10⁸ to 10¹¹ cells/kg body weight, 10⁹ to 10¹¹ cells/kg body weight, 10¹⁰ to 10¹¹ cells/kg body weight, 10⁴ to 10¹⁰ cells/kg body weight, 10⁵ to 10¹⁰ cells/kg body weight, 10⁶ to 10¹⁰ cells/kg body weight, 10⁷ to 10¹⁰ cells/kg body weight, 10⁸ to 10¹⁰ cells/kg body weight, 10⁹ to 10¹⁰ cells/kg body weight, 10⁴ to 10⁹ cells/kg body weight, 10⁵ to 10⁹ cells/kg body weight, 10⁶ to 10⁹ cells/kg body weight, 10⁷ to 10⁹ cells/kg body weight, 10⁸ to 10⁹ cells/kg body weight, 10⁴ to 10⁸ cells/kg body weight, 10⁵ to 10⁸ cells/kg body weight, 10⁶ to 10⁸ cells/kg body weight, 10⁷ to 10⁸ cells/kg body weight, 10⁴ to 10⁷ cells/kg body weight, 10⁵ to 10⁷ cells/kg body weight, 10⁶ to 10⁷ cells/kg body weight, 10⁴ to 10⁶ cells/kg body weight, 10⁵ to 10⁶ cells/kg body weight, or from 10⁴ to 10⁵ cells/kg body weight of a subject.

In some embodiments, a dose of a CAR-T cell pharmaceutical composition includes about 1×10³, about 1×10⁴, about 1×10⁵, about 1×10⁶, about 1×10⁷, about 1×10⁸, about 1×10⁹, about 1×10¹⁰, about 2×10¹⁰, about 3×10¹⁰, about 4×10¹⁰, about 5×10¹⁰, about 6×10¹⁰, about 7×10¹⁰, about 8×10¹⁰, about 9×10¹⁰, about 1×10¹¹, about 1×10¹², about 1×10¹³, about 1×10¹⁴, about 1×10¹⁵, about 1×10³ to about 3×10¹⁰, about 1×10⁵ to about 3×10¹⁰, about 1×10³ to about 1×10⁵, about 1×10⁵ to about 1×10¹⁵, about 1×10⁵ to about 1×10¹⁰, about 1×10⁷ to about 1×10¹², about 1×10⁵ to about 1×10⁷, about 1×10¹⁰ to about 9×10¹⁰, or about 1×10⁹ to about 1×10¹¹ cells per kg of body weight of a subject.

In some embodiments, a dose of a CAR-T cell pharmaceutical composition includes about 1×10⁵, about 1×10⁶, about 1×10⁷, about 1×10⁸, about 1×10⁹, about 1×10¹⁰, about 1×10¹¹, about 1×10¹², about 1×10¹³, about 1×10¹⁴, about 1×10¹⁵, about 1×10⁵ to about 1×10¹², about 1×10⁵ to about 1×10¹⁰, about 1×10⁵ to about 1×10⁷, about 1×10⁷ to about 1×10¹⁰, about 1×10⁷ to about 1×10¹², about 1×10⁹ to about 1×10¹⁰, about 1×10⁶ to about 1×10⁸, about 1×10⁷to about 1×10⁹, about 1×10⁵ to about 1×10¹⁴, about 1×10¹⁰ to about 1×10¹⁵, or about 1×10⁹ to about 1×10¹¹ cells.

In some embodiments, a patient is administered increasing doses of a CAR-T cell pharmaceutical composition. For example, in some embodiments, a method of treating includes administering an initial dose of a CAR-T pharmaceutical composition that includes a specified number of cells per kg of body weight of a subject, and administering a subsequent dose of the CAR-T pharmaceutical composition that includes more CAR-T cells per kg of body weight of the subject as compared to the initial dose. For example, in some embodiments, a method of treating includes administering an initial dose of a CAR-T pharmaceutical composition that includes about 1×10⁵ cells per kg of body weight of a subject, and administering one or more subsequent doses of the CAR-T pharmaceutical composition that include about 1×10⁶, about 1×10⁷, about 1×10⁸, about 1×10⁹, about 1×10^{10,} about 2×10¹⁰, about 3×10¹⁰, about 4×10¹⁰, about 5×10¹⁰, about 6×10¹⁰, about 7×10¹⁰, about 8×10¹⁰, about 9×10¹⁰, about 1×10¹¹, about 1×10¹⁰, about 1×10¹³, about 1×10¹⁴, about 1×10¹⁵, about 1×10⁶ to about 3×10¹⁰, about 1×10⁶ to about 3×10¹⁰, about 1×10⁶ to about 1×10⁷, about 1×10⁶ to about 1×10¹⁵, about 1×10⁶ to about 1×10¹⁰, about 1×10⁷ to about 1×10¹², about 1×10⁶ to about 1×10⁸, about 1×10^{10,} to about 9×10¹⁰, or about 1×10⁹ to about 1×10¹¹ cells per kg of body weight of a subject.

In some embodiments, an initial dose of a CAR-T cell pharmaceutical composition includes about 1×10³, about 1×10⁴, about 1×10⁵, about 1×10⁶, about 1×10⁷, about 1×10⁸, about 1×10⁹, about 1×10^{10,} about 2×10^{10,} about 3×10¹⁰, about 4×10¹⁰, about 5×10¹⁰, about 6×10¹⁰, about 7×10¹⁰ about 8×10¹⁰ about 9×10¹⁰ about 1×10¹¹, about 1×10¹², about 1×10¹³, about 1×10¹⁴, about 1×10¹⁵, about 1×10³ to about 3×10¹⁰, about 1×10⁵ to about 3×10¹⁰, about 1×10³ to about 1×10⁵, about 1×10⁵ to about 1×10¹⁵, about 1×10⁵ to about 1×10¹⁰, about 1×10⁷ to about 1×10¹², about 1×10⁵ to about 1×10⁷, about 1×10¹⁰ to about 9×10¹⁰, or about 1×10⁹ to about 1×10¹¹ cells per kg of body weight of a subject.

In some embodiments, a subsequent dose of a CAR-T cell pharmaceutical composition includes about 1×10³, about 1×10⁴, about 1×10⁵, about 1×10⁶, about 1×10⁷, about 1×10⁸, about 1×10⁹, about 1×10^{10,} about 2×10¹⁰, about 3×10¹⁰, about 4×10¹⁰, about 5×10¹⁰, about 6×10¹⁰, about 7×10¹⁰ about 8×10¹⁰ about 9×10¹⁰ about 1×10¹¹, about 1×10¹², about 1×10¹³, about 1×10¹⁴, about 1×10¹⁵, about 1×10³ to about 3×10¹⁰, about 1×10⁵ to about 3×10¹⁰, about 1×10³ to about 1×10⁵, about 1×10⁵ to about 1×10¹⁵, about 1×10⁵ to about 1×10¹⁰, about 1×10⁷ to about 1×10¹², about 1×10⁵ to about 1×10⁷, about 1×10¹⁰, to about 9×10¹⁰, or about 1×10⁹ to about 1×10¹¹ cells per kg of body weight of a subject.

Pharmaceutical compositions comprising the CAR-T cells described herein may also be administered multiple times at these dosages. The cells can be administered by using infusion techniques that are commonly known in immunotherapy (see, e.g., Rosenberg et al, New Eng. J. of Med. 319: 1676, 1988). Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington’s Pharmaceutical Sciences, Mack Publishing Co., Easton, Pennsylvania 1975; Liberman, H.A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

### Methods of Administration

Pharmaceutical compositions containing arginine transporter overexpressing CAR-T cells or functional fragments of arginine transporter overexpressing CAR-T cells, described herein can be administered for prophylactic and/or therapeutic treatments. In therapeutic applications, the compositions can be administered to a subject already suffering from a disease or condition, in an amount sufficient to cure or at least partially arrest the symptoms of the disease or condition, or to cure, heal, improve, or ameliorate the condition. Arginine transporter-overexpressing CAR-T cells can also be administered to lessen a likelihood of developing, contracting, or worsening a condition. Amounts effective for this use can vary based on the severity and course of the disease or condition, previous therapy, the subject’s health status, weight, and response to the drugs, and the judgment of the treating physician.

Arginine transporter-overexpressing CAR-T cells, described herein can be administered before, during, or after the occurrence of a disease or condition, and the timing of administering the composition containing arginine transporter overexpressing CAR-T cells can vary. For example, arginine transporter overexpressing CAR-T cells can be used as a prophylactic and can be administered continuously to subjects with a propensity to conditions or diseases in order to lessen a likelihood of the occurrence of the disease or condition. The arginine transporter overexpressing CAR-T cells can be administered to a subject during or as soon as possible after the onset of the symptoms. The administration of arginine transporter overexpressing CAR-T cells can be initiated immediately within the onset of symptoms, within the first 3 hours of the onset of the symptoms, within the first 6 hours of the onset of the symptoms, within the first 24 hours of the onset of the symptoms, within 48 hours of the onset of the symptoms, or within any period of time from the onset of symptoms. The initial administration can be via any route practical, such as by any route described herein using any formulation described herein. Arginine transporter-overexpressing CAR-T cells can be administered as soon as is practicable after the onset of an immune disease or condition is detected or suspected, and for a length of time necessary for the treatment of the immune disease, such as, for example, from about 24 hours to about 48 hours, from about 48 hours to about 1 week, from about 1 week to about 2 weeks, from about 2 weeks to about 1 month, from about 1 month to about 3 months. In some embodiments, arginine transporter overexpressing CAR-T cells can be administered for at least 24 hours, at least 48 hours, at least 72 hours, at least 96 hours, at least 1 week, at least 2 weeks, at least 3 weeks, at least 4 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at least 1 year, at least 2 years at least 3 years, at least 4 years, or at least 5 years. The length of treatment can vary for each subject.

Homology of reference nucleotide sequences to recombinant nucleotide sequences of CAR-T cells described herein can be expressed as a percent of sequence homology. In some embodiments the homology of the reference sequence is about 60% of bases to about 100% of bases of the recombinant sequence. In some embodiments the homology of the reference sequence is about 60% of bases to about 70% of bases, about 60% of bases to about 80% of bases, about 60% of bases to about 90% of bases, about 60% of bases to about 100% bases, about 70% of bases to about 80% of bases, about 70% of bases to about 90% of bases, about 70% of bases to about 100% bases, about 80% of bases to about 90% of bases, about 80% of bases to about 100% of bases, or about 90% of bases to about 100% of bases of the recombinant sequence. In some embodiments the homology of the reference sequence is about 60% of bases, about 70% of bases, about 80% of bases, about 90% of bases, or about 100% of bases of the recombinant sequence. In some embodiments the homology of the sequence is at least about 60% of bases, about 70% of bases, about 80% of bases, or about 90% of bases of the recombinant sequence. In some embodiments, the homology of the reference sequence is at most about 70% of bases, about 80% of bases, about 90% of bases, or about 100% of bases of the recombinant sequence.

### EXAMPLES

The disclosure is further illustrated by the following examples. The examples are provided for illustrative purposes only, and are not to be construed as limiting the scope or content of the disclosure in any way.

### Example 1. Effect of Arginine Transporter and Arginine Synthesis Protein Expression on T-Cell Survival

T-cell survival was analyzed under low environmental arginine conditions in order to assess the effect of exogenous arginine transporter proteins and arginine synthesis proteins. Jurkat E6-1 cells, a human T lymphocyte cell line isolated from peripheral blood of an acute T cell leukemia patient, were transfected with using Lipofectamine LTX (ThermoFisher Scientific, Waltham, MA). Expression constructs were generated by cloning the coding sequence of Cationic Amino Acid Transporter-2 (CAT-2, abbreviated as “CAT”) and Argininosuccinate Synthetase-1 (ASS-1, abbreviated as “ASS”) into pBCTex01G fluorescent expression vector immediately in front of- and in frame with the P2A self-cleavage sequence and the fluorescent protein. Cells were transfected with unmodified pBCTex01G (“Control”) or the expression constructs encoding CAT or ASS. The CAT-2 nucleotide sequence used includes mutations encoding an R369E substitution mutation and an N381i insertion mutation, and corresponds to SEQ ID NO:203. The ASS-1 nucleotide sequence used is the following:

ATGAGCAGCAAGGGATCTGTGGTGCTGGCCTACTCTGGCGGCCTGGATAC
CTCTTGTATCCTCGTGTGGCTGAAAGAACAGGGCTACGACGTGATCGCCT
ACCTGGCCAACATCGGCCAGAAAGAGGACTTCGAGGAAGCCCGGAAGAAG
GCCCTGAAGCTGGGAGCCAAGAAGGTGTTCATCGAGGACGTGTCCCGCGA
GTTCGTGGAAGAGTTTATCTGGCCCGCCATCCAGTCTAGCGCCCTGTACG
AGGATAGATACCTGCTGGGCACCAGCCTGGCCAGACCTTGTATCGCCAGA
AAGCAGGTCGAGATCGCCCAGAGAGAAGGCGCCAAATACGTGTCCCATGG
CGCCACAGGCAAGGGCAACGATCAAGTTCGCTTCGAGCTGAGCTGCTACT
CTCTGGCCCCTCAGATCAAAGTGATCGCCCCTTGGAGAATGCCCGAGTTC
TACAACAGATTCAAGGGCCGCAACGACCTGATGGAATACGCCAAGCAGCA
CGGCATCCCCATTCCAGTGACACCCAAGAATCCTTGGAGCATGGACGAGA
ACCTGATGCACATCAGCTACGAGGCCGGCATCCTGGAAAACCCTAAGAAT
CAGGCCCCTCCTGGCCTGTACACCAAGACACAGGATCCAGCCAAGGCTCC
CAACACACCCGACATTCTGGAAATCGAGTTCAAGAAAGGCGTGCCCGTGA
AAGTGACCAACGTGAAGGATGGCACCACACACCAGACAAGCCTGGAACTG
TTCATGTACCTGAACGAGGTGGCCGGCAAGCACGGCGTGGGAAGAATCGA
CATCGTGGAAAATCGGTTCATCGGCATGAAGTCCCGGGGCATCTATGAGA
CACCAGCCGGCACCATTCTGTATCACGCCCACCTGGACATTGAGGCCTTC
ACCATGGACCGGGAAGTGCGGAAGATCAAGCAAGGCCTGGGCCTGAAGTT
TGCCGAACTGGTGTATACCGGCTTCTGGCACTCTCCTGAGTGCGAGTTTG
TGCGGCACTGCATTGCCAAGAGCCAAGAGCGCGTGGAAGGCAAGGTGCAG
GTTTCCGTGCTGAAAGGCCAGGTGTACATTCTGGGCAGAGAGAGCCCTCT
GAGCCTGTATAACGAGGAACTCGTGTCCATGAACGTGCAGGGCGATTACG
AGCCTACCGATGCCACCGGCTTCATCAACATCAACAGCCTGAGACTGAAA
GAGTACCACCGCCTGCAGTCCAAAGTGACCGCCAAA (SEQ ID NO:27
3).

Jurkat clone E6-1 cells (ATCC, USA) were cultured in RPMI-1640 medium containing 2 mM L-alanyl-L-glutamine (Transgen Biotech, China), 10% filtered, non-heat-inactivated fetal bovine serum (TransSerum EQ Fetal Bovine Serum; Transgen Biotech, China), 100 U/mL penicillin, and 100 µg/mL of streptomycin (Thermo Fisher Scientific, USA), at 37° C., 5% CO₂. After reaching about 80% confluence, cells were resuspended in fresh complete medium at a density of 2×10⁵ cells/mL. Cells were seeded into a 24-well culture plate (500 µL/well, 1×10⁵ cells/well).

4 wells of cells were transfected with each construct (Control, CAT, or ASS). 5 µg of each purified plasmid was diluted in 1 mL of OptiMEM reduced serum medium (Thermo Fisher Scientific, Waltham, MA) with 1 µL of PLUS reagent. The mixture was incubated at room temperature for 15 minutes, after which 2.75 µL of LTX reagent was added to initiate complex formation. The complexes were allowed to form at room temperature for 25 minutes. 100 µL of vector-liposome complex was added to each well. Plates were rocked for 2 minutes after adding the transfection complex. Cells were then incubated at 37° C., 5% CO₂ for 24 hours. The cells were gauged for viability (> 90%) using trypan blue staining (Thermo Fisher Scientific, Waltham, MA) and for transfection efficiency by analyzing fluorescent protein expression under a microscope (Zeiss Axio Observer, Germany).

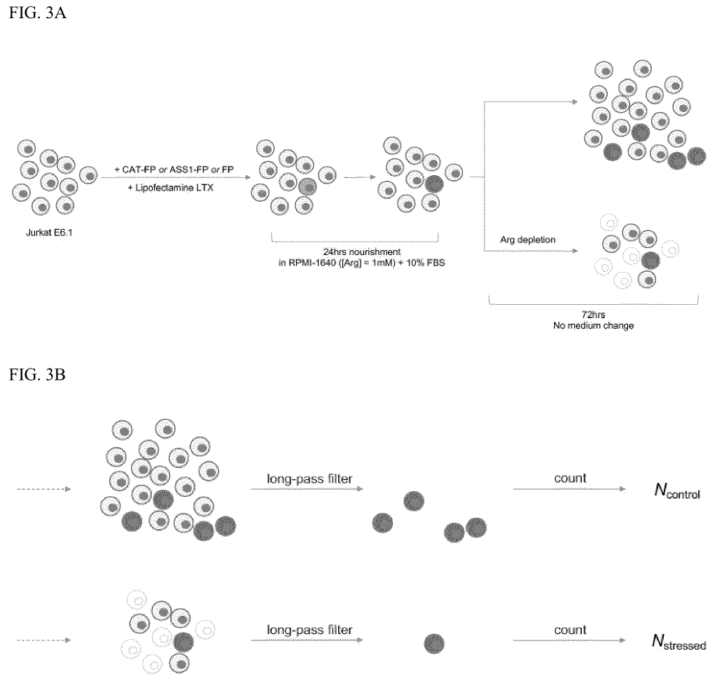

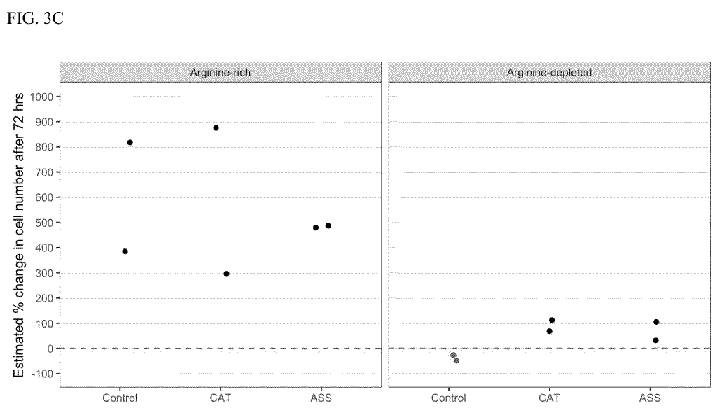

Transfection efficiency of Jurkat E6-1 cells was between 7% and 14% (mean transfection efficiency of 10%). Following transfection, cells were cultured for 72 hours at 37° C., 5% CO₂ in the same medium supplemented or not supplemented with 400 ng/mL BCT-100 to achieve arginine depletion in the medium (FIG. 3A). The total number of surviving transfected cells was counted in each sample. Cell counting was performed for each well using Countess II FL (Thermo Fisher Scientific, Waltham, MA) with default gating parameters to count only cells showing fluorescence (FIG. 3B).

The percent change in cell number after 72 hours of culturing in arginine-rich and arginine-depleted media was calculated for cells transfected with Control, CAT, or ASS constructs (FIG. 3C). The percent change in cell number was calculated as the number of transfected cells after 72 hours in culture minus the initial number of transfected cells, divided by the initial number of transfected cells, all multiplied by 100 (100 × ((# of transfected cells after 72 hours - # of transfected cells)/(# of transfected cells))). The initial number of transfected cells was estimated from the initial number of viable cells multiplied by the estimated transfection efficiency (# of initial viable cells * transfection efficiency). Each data point plotted in FIG. 3C denotes the estimated percent change in cell number of one isolated well of independently transfected cells. Transfection of cells with control, CAT, or ASS constructs all resulted in increased cell number after 72 hours in arginine-rich medium (FIG. 3C, left). By contrast, while transfection of cells with the control construct resulted in an overall decrease in cell number after 72 hours in arginine-depleted medium, transfection of cells with CAT or ASS expression constructs both resulted in an overall increase in cell number after 72 hours in arginine-depleted medium (FIG. 3C, right).

These results demonstrate that expression of proteins that either facilitate cellular arginine uptake or intracellular arginine synthesis increased survival and proliferation of T-cells under conditions of low extracellular arginine concentration.

### Example 2. Effect of Arginine Transporter on Primary Human T-Cell Survival

Primary human T-cell survival was analyzed under low environmental arginine conditions in order to assess the effect of exogenous arginine transporter proteins. Frozen primary human CD4+ T cells were acquired from StemExpress (California, USA), thawed and resuspended at 1×10⁶ cells/mL in RPMI-1640 supplemented with GlutaMAX and HEPES (Thermo Fisher Scientific). The cells were stimulated with 25 µL/mL ImmunoCult Human CD3/CD28 T Cell Activator (STEMCELL Technologies, Canada) and 10 ng/mL rIL-2 (Solarbio, China) for 3 days at 37° C., 5% CO₂. Activated T cells were harvested, resuspended in Opti-MEM I Reduced Serum Media (Thermo Fisher Scientific) at a density of 1×10⁷ cells/mL. One hundred microliters of the cell suspension were transferred to Fisherbrand Electroporation Cuvettes Plus (Fisher Scientific, Pennsylvania, USA) and electroporated with in vitro transcribed mRNA coding for either mNeonGreen (SEQ ID NO:274) as control or CAT (SEQ ID NO:203) in ECM 830 Square Wave Electroporation System (BTX, USA). The mNeonGreen nucleotide sequence used is the following:

ATGGTGTCCAAGGGTGAAGAGGACAACATGGCTTCCTTGCCTGCCACCCA
TGAACTCCATATCTTCGGGTCTATTAACGGAGTCGACTTTGATATGGTGG
GGCAGGGTACGGGCAACCCTAACGACGGCTACGAAGAGCTGAACCTGAAG
TCCACTAAGGGCGACCTCCAGTTTTCTCCTTGGATTCTGGTGCCACACAT
CGGTTATGGTTTTCATCAGTACCTTCCATACCCGGACGGCATGTCCCCGT
TCCAGGCGGCTATGGTCGACGGATCTGGCTACCAGGTGCACCGCACTATG
CAGTTTGAAGACGGCGCATCTCTGACCGTGAACTACCGTTACACTTATGA
GGGCTCCCATATCAAGGGTGAGGCGCAAGTCAAGGGCACCGGTTTCCCGG
CGGATGGACCAGTGATGACCAACAGTCTTACCGCAGCCGACTGGTGTCGC
AGCAAAAAGACATATCCCAACGACAAGACCATTATCAGCACCTTTAAATG
GTCTTACACGACCGGGAACGGTAAACGCTATAGGAGCACAGCCCGCACTA
CCTATACCTTTGCAAAACCTATGGCCGCGAACTATCTGAAAAACCAGCCG
ATGTACGTCTTCCGGAAGACCGAGCTGAAGCACAGTAAGACAGAGCTGAA
CTTCAAAGAGTGGCAAAAAGCTTTTACGGACGTGATGGGCATGGATGAAT
TGTACAAG (SEQ ID NO:274)

Electroporated cells were transferred to one well in 6-well plate containing 900 µL RPMI-1640 supplemented with GlutaMAX, HEPES and rIL-2 and were cultured overnight. The cells were gauged for viability (50-60%) using trypan blue staining (Thermo Fisher Scientific) and for transfection efficiency by analyzing fluorescent protein expression under a microscope (Zeiss Axio Observer). Transfection efficiency was over 80%. Five hundred microliters of the culture were aliquoted to an adjacent empty well and supplemented with 400 ng/ml BCT-100 to achieve arginine depletion. The plate was cultured at 37° C., 5% CO₂ overnight. Cell viability was determined again as above.

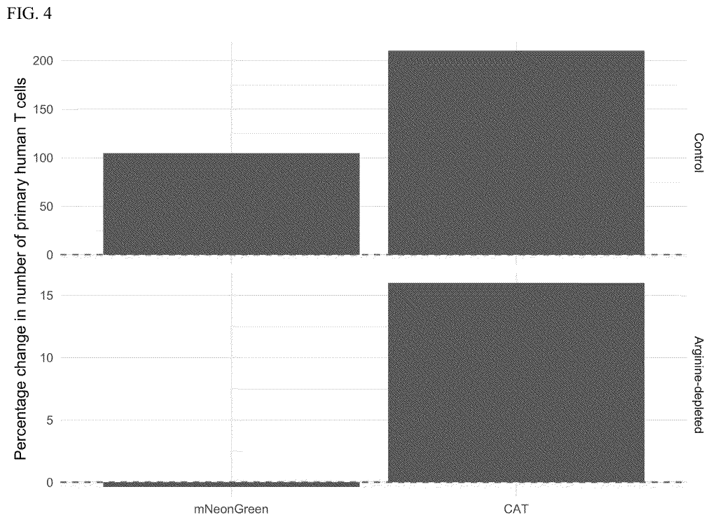

The percent change in cell number after 24 hours of culturing in arginine-rich and arginine-depleted media was calculated for cells transfected with control or CAT mRNA (FIG. 4). The percent change in cell number was calculated as the number of cells after 24 hours in culture minus the initial number of cells, divided by the initial number of cells, all multiplied by 100.

Transfection of primary human T cells with control or CAT mRNA all resulted in increased cell number after 24 hours in arginine-rich medium (FIG. 4, top). In contrast, while transfection of cells with the GFP control mRNA resulted in a net decrease in cell number after 24 hours in arginine-depleted medium, transfection of cells with CAT mRNA resulted in an overall increase in cell number after 24 hours in arginine-depleted medium (FIG. 4, bottom).

These results demonstrate that expression of arginine transporter proteins that facilitate cellular arginine uptake increased survival and proliferation of primary human T-cells under conditions of low extracellular arginine concentration.

### Example 3. Production of CAR-T Cells

This example contemplates a method for producing CAR-T cells described herein.

CD4+ and CD8+ T-cells are isolated from whole blood using a CliniMACS Prodigy with Tubing Set TS520 and CD4/CD8 Microbeads (Miltenyi Biotec, Germany). Approximately 1×10⁸ isolated cells are cultured to expansion in 70 mL TexMACS Medium supplemented with 200 IU/mL IL-2 and TransAct beads (Miltenyi Biotec, Germany) at 37° C. with 5% CO₂ for 3 days.

Expanded cells are transfected with an expression vector encoding a CAR, an arginine transporter, or a CAR and an arginine transporter using a CliniMACS Electroporator (Miltenyi Biotec, Germany). Expanded cells can also be co-transfected with a first expression vector encoding a CAR and a second expression vector encoding an arginine transporter. Once transfected, cells are cultured in TexMACS Medium (Miltenyi Biotec, Germany) supplemented with 1mM L-arginine (Sigma-Aldrich, USA). Cells are sampled daily to gauge cell number and viability using the Live/Dead Cell Double Staining Kit (Sigma-Aldrich, USA). Fresh medium is added daily to maintain a cell density of 2×10⁵ to 1×10⁶ cells per mL. Half of the medium is replaced every other day.

T-cell purity and the ratio of helper T-cells to killer T-cells are determined using a BD FACSAria III flow cytometer and labelled anti-CD19, CD14, CD45, CD3, CD4, and CD8 antibodies (BD Biosciences, USA). CAR and arginine transporter protein expression are determined using custom antibodies specific to, respectively, the antigen-recognizing domain of the CAR and the arginine transporter (GenScript, USA).

Intracellular arginine content is determined by collecting an aliquot of about 1×10⁵ CAR-T cells. Cells are pelleted and washed twice in 10 mL of PBS, and then lysed in 100 µL RIPA buffer. Arginine level of the cell lysate is determined using the L-Arginine ELISA kit (ALPCO, USA). Total arginine levels are normalized to the number of cells lysed.

Cells are harvested for downstream application once about 1×10⁵ to about 3×10¹⁰ cells per kg of subject body weight are obtained, where the cells have an intracellular arginine content of from about 100 µM to about 4000 µM per cell.

### Example 4. Ex Vivo Nourishment and Priming of CAR-T Cells Overexpressing Arginine Transporters

This example contemplates a method for priming for treatment genetically modified CAR-T cells expressing an arginine transporter protein.

CAR-T cells genetically modified to express an arginine transporter and a CAR are cultured in a culture medium containing or supplemented with L-arginine. This medium contains between 0.2 g/L and 1000 µmol/L L-arginine. The engineered T-cells are cultured in the L-arginine medium until intracellular arginine levels are between 100 µmol and 4000 µmol.

Unless defined otherwise, all technical and scientific terms used herein have the same meaning as commonly understood by one of ordinary skill in the art to which this invention belongs.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

The articles “a” and “an” are used in this disclosure to refer to one or more than one (i.e., to at least one) of the grammatical object of the article, unless the context is inappropriate. By way of example, “an element” means one element or more than one element.

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present disclosure also includes the specific quantitative value itself, unless specifically stated otherwise.

Where a molecular weight is provided and not an absolute value, for example, of a polymer, then the molecular weight should be understood to be an average molecule weight, unless otherwise stated or understood from the context.

It should be understood that the order of steps or order for performing certain actions is immaterial so long as the present invention remain operable. Moreover, two or more steps or actions may be conducted simultaneously.

At various places in the present specification, substituents are disclosed in groups or in ranges. It is specifically intended that the description include each and every individual subcombination of the members of such groups and ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

As a general matter, compositions specifying a percentage are by weight unless otherwise specified. Further, if a variable is not accompanied by a definition, then the previous definition of the variable controls.

### Incorporation by Reference

All scientific articles, publications, and patent documents mentioned herein are hereby incorporated by reference in their entirety for all purposes as if each individual publication or patent was specifically and individually incorporated by reference. In case of conflict, the present application, including any definitions herein, will control.

### Equivalents

While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention.

| | |
|---|---|
| SEQ ID NO: | Sequence |
| 180 | GCACTGCTGATGAAACCTGGCGCCGGAACCCGCCAGCCCTCGGCGCCCATTCAGTCCGCG CAGGCAGGTGTGAGCAGCGGGTCAACTACCTGGCAGGCGCGCACGCGGCCGCGGGCTCCC GCTAACCGCAGCCTCCACTCCTCTCCCCGCGCGCCGCGCCCCCGCCCCGCCCCGCCCCGCC CGGTCTCGCCGGCCGAGCGTCCGTTGGTCCTTGAGCGCGTCCGACAGTCTGTCTGTTCGCG ATCCTGCCGGAGCCCCGCCGCCGCCGGCTTGGATTCTGAAACCTTCCTTGTATCCCTCCTG AGACATCTTTGCTGCAAGATCGAGGCTGTCCTCTGGTGAGAAGGTGGTGAGGCTTCCCGTC ATATTCCAGCTCTGAACAGCAACATGGGGTGCAAAGTCCTGCTCAACATTGGGCAGCAGA TGCTGCGGCGGAAGGTGGTGGACTGTAGCCGGGAGGAGACGCGGCTGTCTCGCTGCCTGA ACACTTTTGATCTGGTGGCCCTCGGGGTGGGCAGCACACTGGGTGCTGGTGTCTACGTCCT GGCTGGAGCTGTGGCCCGTGAGAATGCAGGCCCTGCCATTGTCATCTCCTTCCTGATCGCT GCGCTGGCCTCAGTGCTGGCTGGCCTGTGCTATGGCGAGTTTGGTGCTCGGGTCCCCAAGA CGGGCTCAGCTTACCTCTACAGCTATGTCACCGTTGGAGAGCTCTGGGCCTTCATCACCGG CTGGAACTTAATCCTCTCCTACATCATCGGTACTTCAAGCGTAGCGAGGGCCTGGAGCGCC ACCTTCGACGAGCTGATAGGCAGACCCATCGGGGAGTTCTCACGGACACACATGACTCTG AACGCCCCCGGCGTGCTGGCTGAAAACCCCGACATATTCGCAGTGATCATAATTCTCATCT TGACAGGACTTTTAACTCTTGGTGTGAAAGAGTCGGCCATGGTCAACAAAATATTCACTTG TATTAACGTCCTGGTCCTGGGCTTCATAATGGTGTCAGGATTTGTGAAAGGATCGGTTAAA AACTGGCAGCTCACGGAGGAGGATTTTGGGAACACATCAGGCCGTCTCTGTTTGAACAAT GACACAAAAGAAGGGAAGCCCGGTGTTGGTGGATTCATGCCCTTCGGGTTCTCTGGTGTCC TGTCGGGGGCAGCGACTTGCTTCTATGCCTTCGTGGGCTTTGACTGCATCGCCACCACAGG TGAAGAGGTGAAGAACCCACAGAAGGCCATCCCCGTGGGGATCGTGGCGTCCCTCTTGAT CTGCTTCATCGCCTACTTTGGGGTGTCGGCTGCCCTCACGCTCATGATGCCCTACTTCTGCC TGGACAATAACAGCCCCCTGCCCGACGCCTTTAAGCACGTGGGCTGGGAAGGTGCCAAGT ACGCAGTGGCCGTGGGCTCCCTCTGCGCTCTTTCCGCCAGTCTTCTAGGTTCCATGTTTCCC ATGCCTCGGGTTATCTATGCCATGGCTGAGGATGGACTGCTATTTAAATTCTTAGCCAACG TCAATGATAGGACCAAAACACCAATAATCGCCACATTAGCCTCGGGTGCCGTTGCTGCTGT GATGGCCTTCCTCTTTGACCTGAAGGACTTGGTGGACCTCATGTCCATTGGCACTCTCCTGG CTTACTCGTTGGTGGCTGCCTGTGTGTTGGTCTTACGGTACCAGCCAGAGCAGCCTAACCT GGTATACCAGATGGCCAGTACTTCCGACGAGTTAGATCCAGCAGACCAAAATGAATTGGC AAGCACCAATGATTCCCAGCTGGGCTTTTTACCAGAGGCAGAGATGTTCTCTTTGAAAACC ATACTCTCACCCAAAAACATGGAGCCTTCCAAAATCTCTGGGCTAATTGTGAACATTTCAA CCAGCCTCATAGCTGTTCTCATCATCACCTTCTGCATTGTGACCGTGCTTGGAAGGGAGGC TCTCACCAAAGGGGCGCTGTGGGCAGTCTTTCTGCTCGCAGGGTCTGCCCTCCTCTGTGCC GTGGTCACGGGCGTCATCTGGAGGCAGCCCGAGAGCAAGACCAAGCTCTCATTTAAGGTT CCCTTCCTGCCAGTGCTCCCCATCCTGAGCATCTTCGTGAACGTCTATCTCATGATGCAGCT GGACCAGGGCACCTGGGTCCGGTTTGCTGTGTGGATGCTGATAGGCTTCATCATCTACTTT GGCTATGGCCTGTGGCACAGCGAGGAGGCGTCCCTGGATGCCGACCAAGCAAGGACTCCT GACGGCAACTTGGACCAGTGCAAGTGACGCACAGCCCCGCCCCCCGGAGGTGGCAGCAGC CCCGAGGGACGCCCCCAGAGGACCGGGAGGCACCCCACCCTCCCCACCAGTGCAACAGAA ACCACCTGCGTCCACACCCTCACTGCAGCCAAAGGTGCAATTACTTGACCTGCAGCCCCAG CCCACCCTCGGCTCTGCAGCCGGTTCTCCGGGCCCTGGTCACCTCCAGACAGCTGCCTGGC CGGGGCCACTAGGCTGCGGCTGGCCACTGTGTCTCCTCACTTCTCTGAACAAAGCAGTTCC TCCCCTACCAGCTCAGCCCCGAGCTGCCGCAGCCTCAGGCAGAACGGAGGTCACCTTCTCT CCTTATCTTGGGAACCAGGCCTTCCTCCCGGGGACTGTTCTGGGATTGAAATTGTGCATAC TCCAAACTTTCGCAGCCATCTTCCCGCTCAGCCCCAGACACCCAGCAATCAAGCCAGATGA GTACCACAAAACAGTGTGTCCCCAGCAGCTCCCCACCCCAGAGCCAAATGACAGTAGTGC ACTTAAAAAGGAAAATCAGGCCTGTTGTCCTTCTCCGGTTGCATTCAGATGGGTCATTAGG GCCGGACCCTGCCTGCCCCTTGGCTTCTCAGGGCTTTGCTCTGACACCATGACAGCTGCCC GGGGCTGAGGGCAGCTGGCTCCACTCAAATGAGGAAGAAGGGATCACTCCCATTAGGGCC |
| | TGCTTTGCTTATGCATGTGTGTGCACATGCATGTAAACCAGGGACCTTCAGCTCACGGCCT CCAGGCCTGGGCCAGTTCTTGCTGCTCCTGCCGTCTCCCCCGACTGGCTGTGTCCTGAGTA ACTGGAACATGAGACAGTATCTGCAGGACTGGCCCCATGGTGGCCGAGTCAGAAGTCTGT TTCCTGTGAGTCGCCACCGTTCACTCAGTCTTGCCCTCCCATGCTTTGGAGCCAGTCTGGTG GCTCCTGTAAGGTTCTCAAGGCTGGTGGCAGCTCAGTCTGGGGTCAGGACATGTCGGGGTC ATGCGTTTCTGGCCCTGACATAAGCTGTCTGGCCTCTCTGTGACATGATGAAATTGAAATC AATCCACAGTCCATGAAATTGTGACACTCCACCAGATTAAGTTAGGGCATAACATTAACTT GGAAATGGCCATGTCATCACCCCTGCGGCTGTCCTATAGCTGAGATGCGTGGGTCGCAGG GGAGGTGATTTCTAGGCATATTGCTGTCCCTTTTGTGTATCTGTCATCCGGATGCTTCGGAC CCCACGCCTCTGCAAGTGGGAGAGACCCGAGCATCCTCCCCACCCCCATAGCTCCAGTGCA CGCCACCCCCGTCTTGCCTGGGTCGGGGCCTGCGGCCAGCACCATTTCACACACACTCCTT GTAGATGGGAGCCAGAGGAAACCTGAACGTGGGTGGAGCGTTCCACTGAGTCTACTTCAG GAGACAGAAGGCCCATGCTGATGGGGGAGGAGGAGGGATGTGGGCATTTTGGACACCAG GGGAAATGGAAATGCTGCTTTCAAAACTTAGTTTCCTTTCCATTTCTTCCTAGTCTGGCCTT TGACACAAATCTGGTAGAAAGAAGCCTGATAAATTGAGGGCACTTGTACCCTCCCTGTGCC CCCAGAAGGTTCTTGGAGAGAAGTGCAAGAATTTGTGAACACGGCGGTGGAGGGCGGGTG GATGGCCATGGGCTGAGCCTCCGTATCAGGCCTGCTCACCTTGCTGGGAGCTTTATTCTGA TCTCATTTTGAATGTTCCAGAGGGAGCATCATAAGAGCCCAGAGCTCCGATTTCCAAAGAG TGATATTGACATTTATGGAGATTGGTGTTGTAACATATTTTGATAAATACTAACTTATTTTG TTGGGGTTTTGGTTGTCTCTTGTCTTAGGACCTGGTAGTTATTTGCTTGATTTTTTTTTCCGT TATTTTCTACATAGGCAAAGAGAATTCGAGGGATAGACAGTCTCCAAGAAAAGTGAAGTG GTGGGAGAGAATTGCTTTTTTCTTTTTTTTCTTTTCTCTAGTTTTTCTTTCTGGCTGAGATTT CCGTGCAAGACAGCACCCAATAGACTATTTAGAGTTGACATTTGACATTTTAATGGGCGCC ATGGCTCATTTTGTAGATTGAGAAGGTGCGTCTCCCCTGCTCCAAGTCTCATCATGACAGC GTGCTGACAGCTGGGAGTCTGTGGCCTTCCTCACGCAGAGGCCTTAAAGCTGGACACAGA AGCACGCCTAGGCTGGGCAGGGATGGGACCCATGCCCCCTCCTTAGAGGACGGGCTTCCT GGTTAGGAAAGGACACGTGGGGGTGCCTTGCATAATAGTTCACTGGTCACCGTGCTTTTAT GAGTAGTGTTTTTGTGCACTTGCCAGGGGTTTTCTCTCTGTGTGAGAGGGGAGTGATTTAA GCAATGGTGTCTGGAGTAAGCCTTACAATTTTAATAGACTTTTTCTTATCATATCCCTCATT TCTTTCCCTGAAATAAAAATACACACAAGCAAAAAAAAAATGATAGTTTCACATCTCTTAG TTCCCTTGCCCAAACAAGAATATTCTTAGTTCCACTGGCCAGGATTTTCCTACATAGTCAG AACTTACACATTACTAGAGGCACACCCACCAAGGAGTATTGTGTCTACTTTTATCTGTGCA CCAGCCACAAATACCCACATTGGAAAGACCCATTTGTGATGGGTAAACATCCCTTCCTGTC TCCCACAACCCCTGTGACTGCCCTGCATGTGTTCATGACCTCCGAAGGCCCAAATTCATGA AGCAGCAAACCCAGCAGATCTCCACCCCCCTGCCTCAGGACCTCTGCTGAAGAGGGGGAT GAAGTGGGTCTCCAGGGAGGCAGTGGGGGCCTTGTTGGCAGCTGGCTCGGGAGCCGGCTT ACAGGAGGGCAGCTCTGCAGTTGGGAGGGGCACCGTCCGGAGGAGACCAGGCCTCTACAC ACCCCCCACTCTACTTATCATCCCTGCTCACACACCCTTGTCCAAGGCTTTATGCATCGGAT TTATTTTTCCAAATCAAGAGGACAGTGATAGATGCATTTTCCCCAGGCTGTCTCAGAAAGG TCGCTAAATGTATACTGTTGTCAGAATTGCTGAGATCTCCCCCCACTTTTGGTTTTTGCAGC AGTAAAAACTCTTTCCACTGTGACTTATTTTCTCTCTCAGGCAGCCAGCCACCTGGTCCCTT GTGCTGACTCTAGCACAGTGGCCAGGATCCAATACGAGTCCAGGGGTGACCGCAGGATGG TGGGGGCAGCGGGCTTCTCCACCTACCCCAGCCACCAAGGCCCTGACGCACTGCCTCCTGC ACCTTCAGCACATCCCTGTGCACAGCTGGAAGGGTGCATGGCCCGCTCACCTTTGTTCAGA TGGGTGGAAACGCTGATGATACCAGCTCCTCCCTGCCGTGCCCCTGCCACGGAGCAGGCAT TGTGAACTGGCTGGTGTTTGCAGTCCCACGTGGCATGGCCTCCAGCCCAACCCACAGTGGA GACTGGAGACAGGGCAATGAGTCTGGTGGGGGGCACGTGGACATGCCCCATAGGGGCCCC ACCCAGACTTAACAGGCAAGGTCCTGGGCATTGCGCGACGCAGGACTCAATGCTAAAGCA AGCCTGCCTGGCTCTGTGCCAGGGCCCCTCTTCTGATTCACACATCCCATTTTTACACAGAC CCTTCCTTCTTAATAAAGGCTGACAGTTCTGTTGGCAGCCAAGAACCCACACCATGAAGAC AGGGAGTGAGGGGCCTTTGTGCCCAACTCCAGCACAGCTGCGTTCTGGGGTGTGTGAGAG GCATGTTCGTGTCTGTGCGCTGGTGGTCTCGTGAGACAGTTCCGAGGACGGGGAAATTGCA GGGTGGTGGGGGCGTGAGGCTTATATGTGGAACTGATGCAGAGTTCGCCTGCAGACGGAT |
| | CTGGATATACACTATGTATAATTGTTACGTGTAATTTAAAATATATCTGTTTGCCATCGTCA TGAGAAGATTATATGTAAGGCTCTGAAGGGAGAGGGAGATGTACATTCTGCCAGGCTCCT GGGGACCTTATCCGAGTCATGAAATTGATTACTGTTGATCCAGTGGTGCAAGAAGCTACAC TCCATGTGTCATCACGCTTATGACTCCTAATGTATTTTTAAGGCAAAAAATGTCAGCCGAC TCCATCTTCACCCCTCGATTCCTCGAGTCCAGCCTTTCTGTGCCAGTGCTTCACTGAGCCAC AACGCTCTCGCCATCGGGACCCGGCTGGGCCTGGAGTCTCGGGGCACAGTTGCCATGGAG CCCTCCTGGGTCATTCTACAAATGTGCTGAGTGCCAGCTGAAAACCCCACAGGAGATGGA GTACCTTGGCCAAGCTTAAAGAGAAGATTTTCTCAGGGTATTTATTAGTGTGTCCAGCAGG GTCAGGAAGCAGGATGGAAAGATGCACTCAGACTGTTAATTTATTAACAAGGCAAATGAT TTTGTGTTTCTTGATGACAGACTATTAAGTTTGGGACTTATTTTCCCATTTGAGAAGTTATA ATATATATTTAAGATGATAAGTTTCCTGCTTAAGTTGTGCCTTTCAGCTTCAATGAGTTTAA GGAGCACTAAGGGTAATGATACCAATGAGGGTTGGTTTATTATCAAACCTGAATAGCTGT GGTTTCTCCAGTAAATATTTTCTTCTACTGAACATGGAGCCATTATTAAGAGTTGTGTGTTT TTTATTATGTACATTTGTATATTTTTTTGCTTGTTTGATGTTCTATTTTTCTAATAGTTTTCTT TTAGTTTCTTAAAGTTGTGATACTAGATTTAGATTCTGATGCTAACTGCAAATCAGGTTGGT CTCTGCTGGGTCTCTCCTGCTTTTATTTTACTTTAAGGACAAGTGTAGTTGTCGTCCACCAC CTTTCAAAAAATGTGAAACTGCCCTGCCTCCCCTTTTTGCTGACAACACTGTGTACATTGAC CACTTCCTACCATACTTTATGTTGTAAAATCAAACTCTTTTGTGGTACATTATCTCATGCTT CTGCAAATTCGAATAAATTCTATGGCTTCCA |
| 184 | CTCCTTCTGCAGCGCGGCCGGCGGGCGCTCCTCTTCGCGGGACCAGCGAGGCGGGCCG CTGCTCCAGCGTCCCCCAGCCGCGGGCCCCCGACGCGCTGCAGCCGGCAGCCCACCGCCG CCTTCTTGGCGCGACCCCAACCCAGCCCCAGTCGCCTTCGTCAGACGTCAGAATGATTCCT TGCAGAGCCGCGCTGACCTTTGCCCGATGTCTGATCCGGAGAAAAATCGTGACCCTGGAC AGTCTAGAAGACACCAAATTATGCCGCTGCTTATCCACCATGGACCTCATTGCCCTGGGCG TTGGAAGCACCCTTGGGGCCGGGGTTTATGTCCTCGCTGGGGAGGTGGCCAAGGCAGACT CGGGCCCCAGCATCGTGGTGTCCTTCCTCATTGCTGCCCTGGCTTCAGTGATGGCTGGCCTC TGCTATGCCGAATTTGGGGCCCGTGTTCCCAAGACGGGGTCTGCATATTTGTACACCTACG TGACTGTCGGAGAGCTGTGGGCCTTCATCACTGGCTGGAATCTCATTTTATCGTATGTGAT AGGTACATCAAGTGTTGCAAGAGCCTGGAGTGGCACCTTTGATGAACTTCTTAGCAAACA GATTGGTCAGTTTTTGAGGACATACTTCAGAATGAATTACACTGGTCTTGCAGAATATCCC GATTTTTTTGCTGTGTGCCTTATATTACTTCTAGCAGGTCTTTTGTCTTTTGGAGTAAAAGA GTCTGCTTGGGTGAATAAAGTCTTCACAGCTGTTAATATTCTCGTCCTTCTGTTTGTGATGG TTGCTGGGTTTGTGAAAGGAAATGTGGCAAACTGGAAGATTAGTGAAGAGTTTCTCAAAA ATATATCAGCAAGTGCCAGAGAGCCACCTTCTGAAAACGGAACAAGTATCTATGGGGCTG GTGGCTTTATGCCTTATGGCTTTACGGGAACGTTGGCTGGTGCTGCAACTTGCTTTTATGCC TTTGTGGGATTTGACTGCATTGCAACAACTGGTGAAGAAGTTCGGAATCCCCAGAAAGCTA TTCCCATTGGAATTGTGACGTCTTTGCTTGTTTGCTTTATGGCCTATTTTGGGGTCTCTGCA GCTTTAACACTTATGATGCCGTACTACCTCCTCGATGAAAAAAGCCCCCTTCCTGTAGCGT TTGAATATGTGGGATGGGGTCCTGCCAAATATGTCGTCGCAGCTGGTTCTCTCTGCGCCTT GTCAACAAGTCTTCTTGGATCCATTTTCCCAATGCCTCGTGTAATCTATGCTATGGCGGAG GATGGGTTGCTTTTCAAATGTCTAGCTCAAATCAATTCCAAAACGAAGACACCAATAATTG CTACTTTATCATCGGGTGCAGTGGCAGCTTTGATGGCCTTTCTGTTTGACCTGAAGGCGCTT GTGGACATGATGTCCATTGGCACACTCATGGCCTACTCTCTGGTGGCAGCCTGTGTTCTCA TCCTCAGGTACCAGCCTGGCTTATCTTACGACCAGCCCAAATGTTCTCCTGAGAAAGATGG TCTGGGATCGTCTCCCAGGGTAACCTCGAAGAGTGAGTCCCAGGTCACCATGCTGCAGAG ACAGGGCTTCAGCATGCGGACCCTCTTCTGCCCCTCCCTTCTGCCAACACAGCAGTCAGCT TCTCTCGTGAGCTTTCTGGTAGGATTCCTAGCTTTCCTCGTGTTGGGCCTGAGTGTCTTGAC CACTTACGGAGTTCATGCCATCACCAGGCTGGAGGCCTGGAGCCTCGCTCTCCTCGCGCTG TTTCTTGTTCTCTTCGTTGCCATCGTTCTCACCATCTGGAGGCAGCCCCAGAATCAGCAAAA AGTAGCCTTCATGGTTCCATTCTTACCATTTTTGCCAGCGTTCAGCATCTTGGTGAACATTT ACTTGATGGTCCAGTTAAGTGCAGACACTTGGGTCAGATTCAGCATTTGGATGGCAATTGG CTTCCTGATTTACTTTTCTTATGGCATTAGACACAGCCTGGAGGGTCATCTGAGAGATGAA |
| | AACAATGAAGAAGATGCTTATCCAGACAACGTTCATGCAGCAGCAGAAGAAAAATCTGCC ATTCAAGCAAATGACCATCACCCAAGAAATCTCAGTTCACCTTTCATATTCCATGAAAAGA CAAGTGAATTCTAACACTTGCAGGAGCAGAGCTGGTCATCGTCTTAGCATACATATCCTAC ACTGAGTAAACCGTAACGGGATGTCATCAGCATGCTGGGTTGTCATGGGTTTGCTGCATAC ATAGTTCACCCTAATTTATACTTACTCATCTGGACAGCATCTCCTCAGATGGTGAATTATGT GCACGGGGAAACCTCCTGAGTGGAAGTTTCATTCATCAGTGATGAATAGCCCCCAAACAG TGGGAGTGTGTATGTATGTGTGTATGTATGTATCTATGTATATGCTTGGGAACATGAGTGT TACAAGTTAGCTGGTGTTTTACTATTATTGTGTTACATTTTTCCAGTGTCGTCATTAATCGG TGGCATATACTGCACATACTGAAATAGAGGGAAATCACTGAATGTAAAGAGGTTTCATCT ATGCCCCCTGCAGTTGGGGAAATACTAGTAGCTTTACCTTGTTTGACTTCATTAATGTCAGT TTAGGGGATGCCAAAAATGCAGTTACTCATCATGGTGTCTGTCACTGGTTAGGGGTAAGAT GAGGGGATAAGGAAAGAGACTTTTCAATAAGTTGTGAATGCCAACAGTGGGTTTAATGCA AATTTTTTTTCCTGTGAGGTATGACAGTTTGCTCAAACTTCAGCCAACAGGGGTGTCTGCTT CTGCTGCACTACACAGGCCAGGAGTGGCATTCCATGCCACTAGTTGGCATCCTTTTGAACT TTTGTCTCCTTTGCAAACAGTGGTCCTAAAATACGAGGTCTTCACTTGCTGTGAATGACGT ATCCCCAGTCAGGGACTTAAGAGAGGCACTGTGATATACTTGGGACCCTTTAAATTAAAA AGTGAAGATAGTCACCAGGGCCAGAAAGCTCATGGAGTGGCCGTAATGAGAATATGTTTG AAGATCAAAGAGTTAGACCAATGCTTGAATAAGTAGACCCCAAGCATCCTTTTCTAAAAA GTGACTTAAAATAAGCCAACAGACTCTCCCAGACCACACAACTAGTGGAATGATTCCTCCT TTTTCCATTACTTACTTAATCACAGTTTAGTTTTTTTCTTAACCTCGTCAGGCCCAGAGTTCA CTTCTTTGTTTCTCTGTTTCTTTTGTCTTGTCTTAGAGATGAGGGGGCTACAGCAGCATCAT GCAAAGAGGGAAAGATGAAGGGATAGAAGAAGAGAAAATCCCCCTGTTCTGATAGGAAC GGCCTGTTCCATTGTTAAATGGCAAATGGCCCAATTTAAGGGCTTTGGATCTAATTTGCCT CTGATGTTTCCTTTGGAAACATTTAGGAATATTTTTCTCCCCCTACCCCATAAATTGTGTAG CAC11111ATTCCATTTGCTTTCAAATGACTACACTAAGCCTAATAATACAAGCTCCAGTGT TATACAATAACCCATCAGTGATTGGGGAATCAAACATTTTGGTTTAAAAAACATGATTATT TAAAACTGGAAACTAAAAAGAATCAAATTGAATTAAAGCTATATAAACACAGTTAACCCT TGTAAATGAGTAAACAAATTTTTACATGTAAGATTCTCTAATTGTCATATTTTACTTTTTAG GATTCCCTAATAGTGGACTGTTTATTTGCAGTGTATTTGCTTCTCATGAACTATTTCTCGTA CAAATCATTAAATAGTTCATTGGATGAGGCTGGGTGACATTTCCCAGGACAGCATGGTGA ACATTACCAGGCATGCTAGCTGGCCCGTGTAATCCCAAGACAAGGAAAACATTCGTTTTCC TCATGGGTCTTCCAAGAAATGAGCTATTTTATTGATGCCATTAAAAAGCAAGTTGCGATGG TTTTGTATAGCCAGGAGTTTATTGTGATTAAACATCAAAGAAACAGGTAGAAAGCCTGGGT TTCTGGCTGCTAGCGTTATAGCATCCATGACACAGAACTCATTACGGACATTCCACAACTT CCAGGGTGCACATGGTAAAATCTGAAGCCCAGAATTTTCTCTCAAGCTGCGTGGTTTACTG GAGAGAAGGAGTTGGATAAGCACAGGCTCGGGTATTTTGGTAGGGACTGTAGGCATGCTC ATAAATCCTTGCTGTTGTCACAGTACGCTGAAAACCCGTTTGATTCTATACCCAATCAAGA ATAGACCCTTCACACAGGAAATGTGAACAATTGTTATATATGAACACTCAAATCTTTTACT GTAACGAAACCAAGAAACTTGTTTAGAATGTGATAGGCAGCTAAAACTGTTATGCCCACT GTGCTCAATTTGAAGCAGAATTTAGTGAAAAATTATTTTTCCACATTGAAACACTTTGCAG ACACAAATATCTATGAAAAGATGCTTTGTCAGCCACTGTGCCTTTTTTTCTGTGAAGACTC AACGGATGTGTGTGTTTGTATGTTTGTTAACAGTTACATATGTTTGTATGAGTGTATATATA TATCTGTGTGTGTGTATCTCTAACGTCAGTGTATAAGTAAGTTGGGTTTATGGTGGGCTTTG ACTATGTCATTAGGTGGGTACAAAACCCAACTGATGTGGAGAAAAATTGATGTTTGATGTT GATAGATATGCTTATACCTAATTTTTAGTTTTTAAACTATTTTAAAATATACTATGATTTTA TATGTATATTTCCTATAGACTCTTTAAGACGTATTTATAATGTTTCTAATATGAAATCACTA AACTCTAGTACATTATAGCAGGTGCTTTGTAATCTGGAATGGAGAAGAGGTAGGGGCATTT GGGGATTCCTGTTTACTTGCTGCTGCCACACCTTTTCCGACTGATCTGTCCTGGTAGGTGTT TATTAGCAAAAGTCAGTATCACCAGCTCTTTGGCACCTTTCTGTTTCTGCTTGTGAATTCAT AATGTTTTCAACTAAATTTTTTTTTTCTTTCTCAGAATTACCTAAATGTTTTGTAGAGTTTTG ACTAGTAATCAATCAAAATTATATAAAGTCTTCTCCAGTAATTAAGAAATACATATGCAAA TTCTTTTGTGATTGAGTAAAAGCAGCTTAAATTACTTTTCTTTTCTACATTAAGAAATATAT TCTCAACATTTTCAGTGAGAATTTCTTGTAATGGCACCTCAAATTTTATACTCTTAAAAAAA |
| | AACAATAATTTGTGAATTACCACCAAAAGGCAATGGCAGTCCTACATTTAAGAATAGAGC TATGCAAACTCTGTTAAAAACTATGAGGAAAACTTATATTAGAACTTTTGATATATACTAA AATACTGATTATCTTAATCACATTTTCCCCAGAGATAAACATTGAGAGAACGAAAGCCAA AGTGTCATTTAAGAGAGATATATATGAAAAAGTAACATTAATATATAGAACTTTACCATCA CCAGCCGTAGTTGATAGAAAATATTAGTTTCAGAATTACCCTCCTTTAAAAAATAAGAGAC TATTTGTTTTCTTTTAATTTCTATGAATAAAAGAAATTTTTAAAAACTTTAAAATTTTAAAT ATTAGTCAAAATACTTTTTAAGTCCTGAGTGCTTACAGGTAGTTGTTAAAAAAATTTTAAG GCCAGGCATGGTGGCTCGCTCACACCTATAATCCTAGGATCTTGGGAGGTCGAGGCAAGC TGATCGCTTGAGCCCAGGAGTTTAAGACCGGCCTGAGTAGCATAGCAAGACCCTGTCTCTA CAAAAAAAACAAAAATTAGCTGGGCATGGTGGCATGCACATGTAGTCAGAGCTACTGGGG GTGCTGAGGTGGGAGGATCGCTTGAGCCCAGGAGAGTGAGGCTGCAGTGAGCTGAGATTA CGCCACTGCACTCTAGCCTGGGCAACGGTGAGACCCTGCCTCAAAAAAAATAAAAATAAA AATAAAACACTTTAATTAGAATCTATTTTTACCTATTTTCTAAATTTATTTAAATGCTTAGC AGGAAGCATAAGGAAAAGCCATCGGCCTCCAATACCCATGATGACAGAGGGAGCACTTGA GCCTTGCCTTCCCTCCTCTTAAATCAGGGTGTGTTCCGAGATTACAGAACATCACACCTTG GCGTGATGAAATCATGCCAAGATTCTGACTCTCCCTTTCCGGTGATACTGCTCATGATTTCT CCTAATACGCTTCAAGCAACTGTTACCACAAAAAATACAGTTTCCGCAGGGCTTTAAAGGA TTGAGTTTAGCATGTATATCATGCGTTATTAAAGTTCACGTGATTCATGTGAAATTAACTGT CCTTTTTGCTAGTGCCAAAACAGTGCCTTCTCTGCACACTTTACTTGTTTATAAAGTTCTCC CACATGTCCTTAAATATCAAGGGGGAAAGTATGGATATTCGCGTAGCAATAATGCCAGCA AAGGTCATTTTCATTTTTTAGTCATATAGATATGAAAATAAGTTCATATAGATATGAAATT GCTTGACTTTATTGTTTTGGGGAGATTTTTTTTCCTTACATGATTATATTAAACACTTTAAA ATAGCCTTCCGGTTTCTGGATTTTGAGAAGCCTGATCTGTTATTGTTGTGGTTGTTGGTGTT TGTAATATTCATTATTGTTTGTATATACACGGTTTAGTCTTACTGATTTCAAATGCATTTTGT TATTGCTCAACCCAACTGGTAACACTGTTTGCTGGGAGCATTATACTTAACTTTGATTCACC ATGGTTGATGCCACTGCCATGATCGCTGGGTCTTAAAGAGCTTTCCCTAGCCACTGACAGC CCCGTGGAGATCATAATCAGGGCCCCAGGCTGGTTCCAGGATCAGGCAGCCTATAGAGTG TGAGCATCTATGTGTAGCTACCCTTGTTGGGTGGGCTCTTAGACTGATGGGGTAGGATATG AAGTGAAAGACTTCAAATGCAAGTAAGGTAGTTTGGGCTCCTTAATTCCAAACATCCCATG AGTATATCAAGATGAATAAGGACCAAGGGACCTCTGTGACTCATAGAAGGGCTGGCTGAA TCCTGAAGTAGCATAGTGGGACCTGGTCTACAATTTATGCACATGCACTGACAGCCTTGCT GTGCCACGTGTCTCACCAAGACCCAGTTGGGAAAGAGCGTCATATTGCCAACAGGTTGGG TTTCTCTGGCCTACACCTGATTAATGGGCCCTTTATCTTTGGTGTCCCCTAGGAGTGTCCAG TTGTTTTATTGCTGTATTTTGTTATTGCAGTACTTAATAAAAATTGTTGATAGGGCCCAAAA CCCTACAGAAATTCTATGTCTGTAAAAACCAACAAAGGCATTGGACTTGTGTGAATGTACA GGGTTTTTTTAGTAGTAATTTTAAATTTAAATGTTTTAAGTGATCATCAGTGTTCCTTTTTAC TTATAAAGTTGGATTCTTTTTTAGAATTTGTAATAAATAAAAACTGCTGCTTTACCACTGTA AAATATGCTTTCTGATGTGGTGTATTTTTAAAATAAATTTTAATATGTAATAA |
| 185 | TCCCAAAACAGAAAGAGCAGATGTCTCACCACGAAACTAGCAACTGGAATGAAGATAGA AACAAGTGGTTATAACTCAGACAAACTAATTTGTCGAGGGTTTATTGGAACACCTGCCCCA CCGGTTTGCGACAGCAAGTTTCTCCTGTCGCCTTCGTCAGACGTCAGAATGATTCCTTGCA GAGCCGCGCTGACCTTTGCCCGATGTCTGATCCGGAGAAAAATCGTGACCCTGGACAGTCT AGAAGACACCAAATTATGCCGCTGCTTATCCACCATGGACCTCATTGCCCTGGGCGTTGGA AGCACCCTTGGGGCCGGGGTTTATGTCCTCGCTGGGGAGGTGGCCAAGGCAGACTCGGGC CCCAGCATCGTGGTGTCCTTCCTCATTGCTGCCCTGGCTTCAGTGATGGCTGGCCTCTGCTA TGCCGAATTTGGGGCCCGTGTTCCCAAGACGGGGTCTGCATATTTGTACACCTACGTGACT GTCGGAGAGCTGTGGGCCTTCATCACTGGCTGGAATCTCATTTTATCGTATGTGATAGGTA CATCAAGTGTTGCAAGAGCCTGGAGTGGCACCTTTGATGAACTTCTTAGCAAACAGATTGG TCAGTTTTTGAGGACATACTTCAGAATGAATTACACTGGTCTTGCAGAATATCCCGATTTTT TTGCTGTGTGCCTTATATTACTTCTAGCAGGTCTTTTGTCTTTTGGAGTAAAAGAGTCTGCT TGGGTGAATAAAGTCTTCACAGCTGTTAATATTCTCGTCCTTCTGTTTGTGATGGTTGCTGG GTTTGTGAAAGGAAATGTGGCAAACTGGAAGATTAGTGAAGAGTTTCTCAAAAATATATC |
| | AGCAAGTGCCAGAGAGCCACCTTCTGAAAACGGAACAAGTATCTATGGGGCTGGTGGCTT TATGCCTTATGGCTTTACGGGAACGTTGGCTGGTGCTGCAACTTGCTTTTATGCCTTTGTGG GATTTGACTGCATTGCAACAACTGGTGAAGAAGTTCGGAATCCCCAGAAAGCTATTCCCAT TGGAATTGTGACGTCTTTGCTTGTTTGCTTTATGGCCTATTTTGGGGTCTCTGCAGCTTTAA CACTTATGATGCCGTACTACCTCCTCGATGAAAAAAGCCCCCTTCCTGTAGCGTTTGAATA TGTGGGATGGGGTCCTGCCAAATATGTCGTCGCAGCTGGTTCTCTCTGCGCCTTGTCAACA AGTCTTCTTGGATCCATTTTCCCAATGCCTCGTGTAATCTATGCTATGGCGGAGGATGGGTT GCTTTTCAAATGTCTAGCTCAAATCAATTCCAAAACGAAGACACCAATAATTGCTACTTTA TCATCGGGTGCAGTGGCAGCTTTGATGGCCTTTCTGTTTGACCTGAAGGCGCTTGTGGACA TGATGTCCATTGGCACACTCATGGCCTACTCTCTGGTGGCAGCCTGTGTTCTCATCCTCAGG TACCAGCCTGGCTTATCTTACGACCAGCCCAAATGTTCTCCTGAGAAAGATGGTCTGGGAT CGTCTCCCAGGGTAACCTCGAAGAGTGAGTCCCAGGTCACCATGCTGCAGAGACAGGGCT TCAGCATGCGGACCCTCTTCTGCCCCTCCCTTCTGCCAACACAGCAGTCAGCTTCTCTCGTG AGCTTTCTGGTAGGATTCCTAGCTTTCCTCGTGTTGGGCCTGAGTGTCTTGACCACTTACGG AGTTCATGCCATCACCAGGCTGGAGGCCTGGAGCCTCGCTCTCCTCGCGCTGTTTCTTGTTC TCTTCGTTGCCATCGTTCTCACCATCTGGAGGCAGCCCCAGAATCAGCAAAAAGTAGCCTT CATGGTTCCATTCTTACCATTTTTGCCAGCGTTCAGCATCTTGGTGAACATTTACTTGATGG TCCAGTTAAGTGCAGACACTTGGGTCAGATTCAGCATTTGGATGGCAATTGGCTTCCTGAT TTACTTTTCTTATGGCATTAGACACAGCCTGGAGGGTCATCTGAGAGATGAAAACAATGAA GAAGATGCTTATCCAGACAACGTTCATGCAGCAGCAGAAGAAAAATCTGCCATTCAAGCA AATGACCATCACCCAAGAAATCTCAGTTCACCTTTCATATTCCATGAAAAGACAAGTGAAT TCTAACACTTGCAGGAGCAGAGCTGGTCATCGTCTTAGCATACATATCCTACACTGAGTAA ACCGTAACGGGATGTCATCAGCATGCTGGGTTGTCATGGGTTTGCTGCATACATAGTTCAC CCTAATTTATACTTACTCATCTGGACAGCATCTCCTCAGATGGTGAATTATGTGCACGGGG AAACCTCCTGAGTGGAAGTTTCATTCATCAGTGATGAATAGCCCCCAAACAGTGGGAGTGT GTATGTATGTGTGTATGTATGTATCTATGTATATGCTTGGGAACATGAGTGTTACAAGTTA GCTGGTGTTTTACTATTATTGTGTTACATTTTTCCAGTGTCGTCATTAATCGGTGGCATATA CTGCACATACTGAAATAGAGGGAAATCACTGAATGTAAAGAGGTTTCATCTATGCCCCCTG CAGTTGGGGAAATACTAGTAGCTTTACCTTGTTTGACTTCATTAATGTCAGTTTAGGGGAT GCCAAAAATGCAGTTACTCATCATGGTGTCTGTCACTGGTTAGGGGTAAGATGAGGGGAT AAGGAAAGAGACTTTTCAATAAGTTGTGAATGCCAACAGTGGGTTTAATGCAAATTTTTTT TCCTGTGAGGTATGACAGTTTGCTCAAACTTCAGCCAACAGGGGTGTCTGCTTCTGCTGCA CTACACAGGCCAGGAGTGGCATTCCATGCCACTAGTTGGCATCCTTTTGAACTTTTGTCTCC TTTGCAAACAGTGGTCCTAAAATACGAGGTCTTCACTTGCTGTGAATGACGTATCCCCAGT CAGGGACTTAAGAGAGGCACTGTGATATACTTGGGACCCTTTAAATTAAAAAGTGAAGAT AGTCACCAGGGCCAGAAAGCTCATGGAGTGGCCGTAATGAGAATATGTTTGAAGATCAAA GAGTTAGACCAATGCTTGAATAAGTAGACCCCAAGCATCCTTTTCTAAAAAGTGACTTAAA ATAAGCCAACAGACTCTCCCAGACCACACAACTAGTGGAATGATTCCTCCTTTTTCCATTA CTTACTTAATCACAGTTTAGTTTTTTTCTTAACCTCGTCAGGCCCAGAGTTCACTTCTTTGTT TCTCTGTTTCTTTTGTCTTGTCTTAGAGATGAGGGGGCTACAGCAGCATCATGCAAAGAGG GAAAGATGAAGGGATAGAAGAAGAGAAAATCCCCCTGTTCTGATAGGAACGGCCTGTTCC ATTGTTAAATGGCAAATGGCCCAATTTAAGGGCTTTGGATCTAATTTGCCTCTGATGTTTCC TTTGGAAACATTTAGGAATATTTTTTCTCCCCCTACCCCATAAATTGTGTAGCACTTTTTATT CCATTTGCTTTCAAATGACTACACTAAGCCTAATAATACAAGCTCCAGTGTTATACAATAA CCCATCAGTGATTGGGGAATCAAACATTTTGGTTTAAAAAACATGATTATTTAAAACTGGA AACTAAAAAGAATCAAATTGAATTAAAGCTATATAAACACAGTTAACCCTTGTAAATGAG TAAACAAATTTTTACATGTAAGATTCTCTAATTGTCATATTTTACTTTTTAGGATTCCCTAA TAGTGGACTGTTTATTTGCAGTGTATTTGCTTCTCATGAACTATTTCTCGTACAAATCATTA AATAGTTCATTGGATGAGGCTGGGTGACATTTCCCAGGACAGCATGGTGAACATTACCAG GCATGCTAGCTGGCCCGTGTAATCCCAAGACAAGGAAAACATTCGTTTTCCTCATGGGTCT TCCAAGAAATGAGCTATTTTATTGATGCCATTAAAAAGCAAGTTGCGATGGTTTTGTATAG CCAGGAGTTTATTGTGATTAAACATCAAAGAAACAGGTAGAAAGCCTGGGTTTCTGGCTG CTAGCGTTATAGCATCCATGACACAGAACTCATTACGGACATTCCACAACTTCCAGGGTGC |
| | ACATGGTAAAATCTGAAGCCCAGAATTTTCTCTCAAGCTGCGTGGTTTACTGGAGAGAAGG AGTTGGATAAGCACAGGCTCGGGTATTTTGGTAGGGACTGTAGGCATGCTCATAAATCCTT GCTGTTGTCACAGTACGCTGAAAACCCGTTTGATTCTATACCCAATCAAGAATAGACCCTT CACACAGGAAATGTGAACAATTGTTATATATGAACACTCAAATCTTTTACTGTAACGAAAC CAAGAAACTTGTTTAGAATGTGATAGGCAGCTAAAACTGTTATGCCCACTGTGCTCAATTT GAAGCAGAATTTAGTGAAAAATTATTTTTCCACATTGAAACACTTTGCAGACACAAATATC TATGAAAAGATGCTTTGTCAGCCACTGTGCCTTTTTTTCTGTGAAGACTCAACGGATGTGT GTGTTTGTATGTTTGTTAACAGTTACATATGTTTGTATGAGTGTATATATATATCTGTGTGT GTGTATCTCTAACGTCAGTGTATAAGTAAGTTGGGTTTATGGTGGGCTTTGACTATGTCATT AGGTGGGTACAAAACCCAACTGATGTGGAGAAAAATTGATGTTTGATGTTGATAGATATG CTTATACCTAATTTTTAGTTTTTAAACTATTTTAAAATATACTATGATTTTATATGTATATTT CCTATAGACTCTTTAAGACGTATTTATAATGTTTCTAATATGAAATCACTAAACTCTAGTAC ATTATAGCAGGTGCTTTGTAATCTGGAATGGAGAAGAGGTAGGGGCATTTGGGGATTCCT GTTTACTTGCTGCTGCCACACCTTTTCCGACTGATCTGTCCTGGTAGGTGTTTATTAGCAAA AGTCAGTATCACCAGCTCTTTGGCACCTTTCTGTTTCTGCTTGTGAATTCATAATGTTTTCA ACTAAATTTTTTTTTTCTTTCTCAGAATTACCTAAATGTTTTGTAGAGTTTTGACTAGTAATC AATCAAAATTATATAAAGTCTTCTCCAGTAATTAAGAAATACATATGCAAATTCTTTTGTG ATTGAGTAAAAGCAGCTTAAATTACTTTTCTTTTCTACATTAAGAAATATATTCTCAACATT TTCAGTGAGAATTTCTTGTAATGGCACCTCAAATTTTATACTCTTAAAAAAAAACAATAAT TTGTGAATTACCACCAAAAGGCAATGGCAGTCCTACATTTAAGAATAGAGCTATGCAAAC TCTGTTAAAAACTATGAGGAAAACTTATATTAGAACTTTTGATATATACTAAAATACTGAT TATCTTAATCACATTTTCCCCAGAGATAAACATTGAGAGAACGAAAGCCAAAGTGTCATTT AAGAGAGATATATATGAAAAAGTAACATTAATATATAGAACTTTACCATCACCAGCCGTA GTTGATAGAAAATATTAGTTTCAGAATTACCCTCCTTTAAAAAATAAGAGACTATTTGTTT TCTTTTAATTTCTATGAATAAAAGAAATTTTTAAAAACTTTAAAATTTTAAATATTAGTCAA AATACTTTTTAAGTCCTGAGTGCTTACAGGTAGTTGTTAAAAAAATTTTAAGGCCAGGCAT GGTGGCTCGCTCACACCTATAATCCTAGGATCTTGGGAGGTCGAGGCAAGCTGATCGCTTG AGCCCAGGAGTTTAAGACCGGCCTGAGTAGCATAGCAAGACCCTGTCTCTACAAAAAAAA CAAAAATTAGCTGGGCATGGTGGCATGCACATGTAGTCAGAGCTACTGGGGGTGCTGAGG TGGGAGGATCGCTTGAGCCCAGGAGAGTGAGGCTGCAGTGAGCTGAGATTACGCCACTGC ACTCTAGCCTGGGCAACGGTGAGACCCTGCCTCAAAAAAAATAAAAATAAAAATAAAACA CTTTAATTAGAATCTATTTTTACCTATTTTCTAAATTTATTTAAATGCTTAGCAGGAAGCAT AAGGAAAAGCCATCGGCCTCCAATACCCATGATGACAGAGGGAGCACTTGAGCCTTGCCT TCCCTCCTCTTAAATCAGGGTGTGTTCCGAGATTACAGAACATCACACCTTGGCGTGATGA AATCATGCCAAGATTCTGACTCTCCCTTTCCGGTGATACTGCTCATGATTTCTCCTAATACG CTTCAAGCAACTGTTACCACAAAAAATACAGTTTCCGCAGGGCTTTAAAGGATTGAGTTTA GCATGTATATCATGCGTTATTAAAGTTCACGTGATTCATGTGAAATTAACTGTCCTTTTTGC TAGTGCCAAAACAGTGCCTTCTCTGCACACTTTACTTGTTTATAAAGTTCTCCCACATGTCC TTAAATATCAAGGGGGAAAGTATGGATATTCGCGTAGCAATAATGCCAGCAAAGGTCATT TTCATTTTTTAGTCATATAGATATGAAAATAAGTTCATATAGATATGAAATTGCTTGACTTT ATTGTTTTGGGGAGATTTTTTTTCCTTACATGATTATATTAAACACTTTAAAATAGCCTTCC GGTTTCTGGATTTTGAGAAGCCTGATCTGTTATTGTTGTGGTTGTTGGTGTTTGTAATATTC ATTATTGTTTGTATATACACGGTTTAGTCTTACTGATTTCAAATGCATTTTGTTATTGCTCA ACCCAACTGGTAACACTGTTTGCTGGGAGCATTATACTTAACTTTGATTCACCATGGTTGA TGCCACTGCCATGATCGCTGGGTCTTAAAGAGCTTTCCCTAGCCACTGACAGCCCCGTGGA GATCATAATCAGGGCCCCAGGCTGGTTCCAGGATCAGGCAGCCTATAGAGTGTGAGCATC TATGTGTAGCTACCCTTGTTGGGTGGGCTCTTAGACTGATGGGGTAGGATATGAAGTGAAA GACTTCAAATGCAAGTAAGGTAGTTTGGGCTCCTTAATTCCAAACATCCCATGAGTATATC AAGATGAATAAGGACCAAGGGACCTCTGTGACTCATAGAAGGGCTGGCTGAATCCTGAAG TAGCATAGTGGGACCTGGTCTACAATTTATGCACATGCACTGACAGCCTTGCTGTGCCACG TGTCTCACCAAGACCCAGTTGGGAAAGAGCGTCATATTGCCAACAGGTTGGGTTTCTCTGG CCTACACCTGATTAATGGGCCCTTTATCTTTGGTGTCCCCTAGGAGTGTCCAGTTGTTTTAT TGCTGTATTTTGTTATTGCAGTACTTAATAAAAATTGTTGATAGGGCCCAAAACCCTACAG |
| | AAATTCTATGTCTGTAAAAACCAACAAAGGCATTGGACTTGTGTGAATGTACAGGGTTTTT TTAGTAGTAATTTTAAATTTAAATGTTTTAAGTGATCATCAGTGTTCC11111ACTTATAAA GTTGGATTCTTTTTTAGAATTTGTAATAAATAAAAACTGCTGCTTTACCACTGTAAAATATG CTTTCTGATGTGGTGTATTTTTAAAATAAATTTTAATATGTAATAA |
| 186 | CTCCTTCTGCAGCGCGGCCGGCGGGCGCTCCTCTTCGCGGGACCAGCGAGGCGGCGGCCG CTGCTCCAGCGTCCCCCAGCCGCGGGCCCCCGACGCGCTGCAGCCGGCAGCCCACCGCCG CCTTCTTGGCGCGACCCCAACCCAGCCCCACAGGAGACTCTCTGAAGACCAGCAGGAAAG CAGTGAGCCCTTACAGGTCGCCTTCGTCAGACGTCAGAATGATTCCTTGCAGAGCCGCGCT GACCTTTGCCCGATGTCTGATCCGGAGAAAAATCGTGACCCTGGACAGTCTAGAAGACAC CAAATTATGCCGCTGCTTATCCACCATGGACCTCATTGCCCTGGGCGTTGGAAGCACCCTT GGGGCCGGGGTTTATGTCCTCGCTGGGGAGGTGGCCAAGGCAGACTCGGGCCCCAGCATC GTGGTGTCCTTCCTCATTGCTGCCCTGGCTTCAGTGATGGCTGGCCTCTGCTATGCCGAATT TGGGGCCCGTGTTCCCAAGACGGGGTCTGCATATTTGTACACCTACGTGACTGTCGGAGAG CTGTGGGCCTTCATCACTGGCTGGAATCTCATTTTATCGTATGTGATAGGTACATCAAGTGT TGCAAGAGCCTGGAGTGGCACCTTTGATGAACTTCTTAGCAAACAGATTGGTCAGTTTTTG AGGACATACTTCAGAATGAATTACACTGGTCTTGCAGAATATCCCGATTTTTTTGCTGTGT GCCTTATATTACTTCTAGCAGGTCTTTTGTCTTTTGGAGTAAAAGAGTCTGCTTGGGTGAAT AAAGTCTTCACAGCTGTTAATATTCTCGTCCTTCTGTTTGTGATGGTTGCTGGGTTTGTGAA AGGAAATGTGGCAAACTGGAAGATTAGTGAAGAGTTTCTCAAAAATATATCAGCAAGTGC CAGAGAGCCACCTTCTGAAAACGGAACAAGTATCTATGGGGCTGGTGGCTTTATGCCTTAT GGCTTTACGGGAACGTTGGCTGGTGCTGCAACTTGCTTTTATGCCTTTGTGGGATTTGACTG CATTGCAACAACTGGTGAAGAAGTTCGGAATCCCCAGAAAGCTATTCCCATTGGAATTGTG ACGTCTTTGCTTGTTTGCTTTATGGCCTATTTTGGGGTCTCTGCAGCTTTAACACTTATGAT GCCGTACTACCTCCTCGATGAAAAAAGCCCCCTTCCTGTAGCGTTTGAATATGTGGGATGG GGTCCTGCCAAATATGTCGTCGCAGCTGGTTCTCTCTGCGCCTTGTCAACAAGTCTTCTGGG CTCTATGTTTCCTTTACCCCGAATTCTGTTTGCCATGGCCCGGGATGGCTTACTGTTTAGAT TTCTTGCCAGAGTGAGTAAGAGGCAGTCACCAGTTGCTGCCACGTTGACTGCAGGGGTCAT TTCTGCTTTGATGGCCTTTCTGTTTGACCTGAAGGCGCTTGTGGACATGATGTCCATTGGCA CACTCATGGCCTACTCTCTGGTGGCAGCCTGTGTTCTCATCCTCAGGTACCAGCCTGGCTTA TCTTACGACCAGCCCAAATGTTCTCCTGAGAAAGATGGTCTGGGATCGTCTCCCAGGGTAA CCTCGAAGAGTGAGTCCCAGGTCACCATGCTGCAGAGACAGGGCTTCAGCATGCGGACCC TCTTCTGCCCCTCCCTTCTGCCAACACAGCAGTCAGCTTCTCTCGTGAGCTTTCTGGTAGGA TTCCTAGCTTTCCTCGTGTTGGGCCTGAGTGTCTTGACCACTTACGGAGTTCATGCCATCAC CAGGCTGGAGGCCTGGAGCCTCGCTCTCCTCGCGCTGTTTCTTGTTCTCTTCGTTGCCATCG TTCTCACCATCTGGAGGCAGCCCCAGAATCAGCAAAAAGTAGCCTTCATGGTTCCATTCTT ACCATTTTTGCCAGCGTTCAGCATCTTGGTGAACATTTACTTGATGGTCCAGTTAAGTGCA GACACTTGGGTCAGATTCAGCATTTGGATGGCAATTGGCTTCCTGATTTACTTTTCTTATGG CATTAGACACAGCCTGGAGGGTCATCTGAGAGATGAAAACAATGAAGAAGATGCTTATCC AGACAACGTTCATGCAGCAGCAGAAGAAAAATCTGCCATTCAAGCAAATGACCATCACCC AAGAAATCTCAGTTCACCTTTCATATTCCATGAAAAGACAAGTGAATTCTAACACTTGCAG GAGCAGAGCTGGTCATCGTCTTAGCATACATATCCTACACTGAGTAAACCGTAACGGGAT GTCATCAGCATGCTGGGTTGTCATGGGTTTGCTGCATACATAGTTCACCCTAATTTATACTT ACTCATCTGGACAGCATCTCCTCAGATGGTGAATTATGTGCACGGGGAAACCTCCTGAGTG GAAGTTTCATTCATCAGTGATGAATAGCCCCCAAACAGTGGGAGTGTGTATGTATGTGTGT ATGTATGTATCTATGTATATGCTTGGGAACATGAGTGTTACAAGTTAGCTGGTGTTTTACTA TTATTGTGTTACATTTTTCCAGTGTCGTCATTAATCGGTGGCATATACTGCACATACTGAAA TAGAGGGAAATCACTGAATGTAAAGAGGTTTCATCTATGCCCCCTGCAGTTGGGGAAATA CTAGTAGCTTTACCTTGTTTGACTTCATTAATGTCAGTTTAGGGGATGCCAAAAATGCAGTT ACTCATCATGGTGTCTGTCACTGGTTAGGGGTAAGATGAGGGGATAAGGAAAGAGACTTT TCAATAAGTTGTGAATGCCAACAGTGGGTTTAATGCAAATTTTTTTTCCTGTGAGGTATGA CAGTTTGCTCAAACTTCAGCCAACAGGGGTGTCTGCTTCTGCTGCACTACACAGGCCAGGA GTGGCATTCCATGCCACTAGTTGGCATCCTTTTGAACTTTTGTCTCCTTTGCAAACAGTGGT |
| | CCTAAAATACGAGGTCTTCACTTGCTGTGAATGACGTATCCCCAGTCAGGGACTTAAGAGA GGCACTGTGATATACTTGGGACCCTTTAAATTAAAAAGTGAAGATAGTCACCAGGGCCAG AAAGCTCATGGAGTGGCCGTAATGAGAATATGTTTGAAGATCAAAGAGTTAGACCAATGC TTGAATAAGTAGACCCCAAGCATCCTTTTCTAAAAAGTGACTTAAAATAAGCCAACAGACT CTCCCAGACCACACAACTAGTGGAATGATTCCTCCTTTTTCCATTACTTACTTAATCACAGT TTAGTTTTTTTCTTAACCTCGTCAGGCCCAGAGTTCACTTCTTTGTTTCTCTGTTTCTTTTGT CTTGTCTTAGAGATGAGGGGGCTACAGCAGCATCATGCAAAGAGGGAAAGATGAAGGGAT AGAAGAAGAGAAAATCCCCCTGTTCTGATAGGAACGGCCTGTTCCATTGTTAAATGGCAA ATGGCCCAATTTAAGGGCTTTGGATCTAATTTGCCTCTGATGTTTCCTTTGGAAACATTTAG GAATATTTTTCTCCCCCTACCCCATAAATTGTGTAGCACTTTTTATTCCATTTGCTTTCAAAT GACTACACTAAGCCTAATAATACAAGCTCCAGTGTTATACAATAACCCATCAGTGATTGGG GAATCAAACATTTTGGTTTAAAAAACATGATTATTTAAAACTGGAAACTAAAAAGAATCA AATTGAATTAAAGCTATATAAACACAGTTAACCCTTGTAAATGAGTAAACAAATTTTTACA TGTAAGATTCTCTAATTGTCATATTTTACTTTTTAGGATTCCCTAATAGTGGACTGTTTATTT GCAGTGTATTTGCTTCTCATGAACTATTTCTCGTACAAATCATTAAATAGTTCATTGGATGA GGCTGGGTGACATTTCCCAGGACAGCATGGTGAACATTACCAGGCATGCTAGCTGGCCCG TGTAATCCCAAGACAAGGAAAACATTCGTTTTCCTCATGGGTCTTCCAAGAAATGAGCTAT TTTATTGATGCCATTAAAAAGCAAGTTGCGATGGTTTTGTATAGCCAGGAGTTTATTGTGA TTAAACATCAAAGAAACAGGTAGAAAGCCTGGGTTTCTGGCTGCTAGCGTTATAGCATCC ATGACACAGAACTCATTACGGACATTCCACAACTTCCAGGGTGCACATGGTAAAATCTGA AGCCCAGAATTTTCTCTCAAGCTGCGTGGTTTACTGGAGAGAAGGAGTTGGATAAGCACA GGCTCGGGTATTTTGGTAGGGACTGTAGGCATGCTCATAAATCCTTGCTGTTGTCACAGTA CGCTGAAAACCCGTTTGATTCTATACCCAATCAAGAATAGACCCTTCACACAGGAAATGTG AACAATTGTTATATATGAACACTCAAATCTTTTACTGTAACGAAACCAAGAAACTTGTTTA GAATGTGATAGGCAGCTAAAACTGTTATGCCCACTGTGCTCAATTTGAAGCAGAATTTAGT GAAAAATTATTTTTCCACATTGAAACACTTTGCAGACACAAATATCTATGAAAAGATGCTT TGTCAGCCACTGTGCCTTTTTTTCTGTGAAGACTCAACGGATGTGTGTGTTTGTATGTTTGT TAACAGTTACATATGTTTGTATGAGTGTATATATATATCTGTGTGTGTGTATCTCTAACGTC AGTGTATAAGTAAGTTGGGTTTATGGTGGGCTTTGACTATGTCATTAGGTGGGTACAAAAC CCAACTGATGTGGAGAAAAATTGATGTTTGATGTTGATAGATATGCTTATACCTAATTTTT AGTTTTTAAACTATTTTAAAATATACTATGATTTTATATGTATATTTCCTATAGACTCTTTA AGACGTATTTATAATGTTTCTAATATGAAATCACTAAACTCTAGTACATTATAGCAGGTGC TTTGTAATCTGGAATGGAGAAGAGGTAGGGGCATTTGGGGATTCCTGTTTACTTGCTGCTG CCACACCTTTTCCGACTGATCTGTCCTGGTAGGTGTTTATTAGCAAAAGTCAGTATCACCA GCTCTTTGGCACCTTTCTGTTTCTGCTTGTGAATTCATAATGTTTTCAACTAAATTTTTTTTT TCTTTCTCAGAATTACCTAAATGTTTTGTAGAGTTTTGACTAGTAATCAATCAAAATTATAT AAAGTCTTCTCCAGTAATTAAGAAATACATATGCAAATTCTTTTGTGATTGAGTAAAAGCA GCTTAAATTACTTTTCTTTTCTACATTAAGAAATATATTCTCAACATTTTCAGTGAGAATTT CTTGTAATGGCACCTCAAATTTTATACTCTTAAAAAAAAACAATAATTTGTGAATTACCAC CAAAAGGCAATGGCAGTCCTACATTTAAGAATAGAGCTATGCAAACTCTGTTAAAAACTA TGAGGAAAACTTATATTAGAACTTTTGATATATACTAAAATACTGATTATCTTAATCACAT TTTCCCCAGAGATAAACATTGAGAGAACGAAAGCCAAAGTGTCATTTAAGAGAGATATAT ATGAAAAAGTAACATTAATATATAGAACTTTACCATCACCAGCCGTAGTTGATAGAAAAT ATTAGTTTCAGAATTACCCTCCTTTAAAAAATAAGAGACTATTTGTTTTCTTTTAATTTCTA TGAATAAAAGAAATTTTTTAAAAACTTTAAAATTTTAAATATTAGTCAAAATACTTTTTAAG TCCTGAGTGCTTACAGGTAGTTGTTAAAAAAATTTTAAGGCCAGGCATGGTGGCTCGCTCA CACCTATAATCCTAGGATCTTGGGAGGTCGAGGCAAGCTGATCGCTTGAGCCCAGGAGTTT AAGACCGGCCTGAGTAGCATAGCAAGACCCTGTCTCTACAAAAAAAACAAAAATTAGCTG GGCATGGTGGCATGCACATGTAGTCAGAGCTACTGGGGGTGCTGAGGTGGGAGGATCGCT TGAGCCCAGGAGAGTGAGGCTGCAGTGAGCTGAGATTACGCCACTGCACTCTAGCCTGGG CAACGGTGAGACCCTGCCTCAAAAAAAATAAAAATAAAAATAAAACACTTTAATTAGAAT CTATTTTTACCTATTTTCTAAATTTATTTAAATGCTTAGCAGGAAGCATAAGGAAAAGCCA TCGGCCTCCAATACCCATGATGACAGAGGGAGCACTTGAGCCTTGCCTTCCCTCCTCTTAA |
| | ATCAGGGTGTGTTCCGAGATTACAGAACATCACACCTTGGCGTGATGAAATCATGCCAAG ATTCTGACTCTCCCTTTCCGGTGATACTGCTCATGATTTCTCCTAATACGCTTCAAGCAACT GTTACCACAAAAAATACAGTTTCCGCAGGGCTTTAAAGGATTGAGTTTAGCATGTATATCA TGCGTTATTAAAGTTCACGTGATTCATGTGAAATTAACTGTCCTTTTTGCTAGTGCCAAAAC AGTGCCTTCTCTGCACACTTTACTTGTTTATAAAGTTCTCCCACATGTCCTTAAATATCAAG GGGGAAAGTATGGATATTCGCGTAGCAATAATGCCAGCAAAGGTCATTTTCATTTTTTAGT CATATAGATATGAAAATAAGTTCATATAGATATGAAATTGCTTGACTTTATTGTTTTGGGG AGATTTTTTTTCCTTACATGATTATATTAAACACTTTAAAATAGCCTTCCGGTTTCTGGATT TTGAGAAGCCTGATCTGTTATTGTTGTGGTTGTTGGTGTTTGTAATATTCATTATTGTTTGT ATATACACGGTTTAGTCTTACTGATTTCAAATGCATTTTGTTATTGCTCAACCCAACTGGTA ACACTGTTTGCTGGGAGCATTATACTTAACTTTGATTCACCATGGTTGATGCCACTGCCATG ATCGCTGGGTCTTAAAGAGCTTTCCCTAGCCACTGACAGCCCCGTGGAGATCATAATCAGG GCCCCAGGCTGGTTCCAGGATCAGGCAGCCTATAGAGTGTGAGCATCTATGTGTAGCTACC CTTGTTGGGTGGGCTCTTAGACTGATGGGGTAGGATATGAAGTGAAAGACTTCAAATGCA AGTAAGGTAGTTTGGGCTCCTTAATTCCAAACATCCCATGAGTATATCAAGATGAATAAGG ACCAAGGGACCTCTGTGACTCATAGAAGGGCTGGCTGAATCCTGAAGTAGCATAGTGGGA CCTGGTCTACAATTTATGCACATGCACTGACAGCCTTGCTGTGCCACGTGTCTCACCAAGA CCCAGTTGGGAAAGAGCGTCATATTGCCAACAGGTTGGGTTTCTCTGGCCTACACCTGATT AATGGGCCCTTTATCTTTGGTGTCCCCTAGGAGTGTCCAGTTGTTTTATTGCTGTATTTTGTT ATTGCAGTACTTAATAAAAATTGTTGATAGGGCCCAAAACCCTACAGAAATTCTATGTCTG TAAAAACCAACAAAGGCATTGGACTTGTGTGAATGTACAGGGTTTTTTTAGTAGTAATTTT AAATTTAAATGTTTTAAGTGATCATCAGTGTTCCTTTTTACTTATAAAGTTGGATTCTTTTTT AGAATTTGTAATAAATAAAAACTGCTGCTTTACCACTGTAAAATATGCTTTCTGATGTGGT GTATTTTTAAAATAAATTTTAATATGTAATAA |
| 187 | CTCCTTCTGCAGCGCGGCCGGCGGGCGCTCCTCTTCGCGGGACCAGCGAGGCGGCGGCCG CTGCTCCAGCGTCCCCCAGCCGCGGGCCCCCGACGCGCTGCAGCCGGCAGCCCACCGCCG CCTTCTTGGCGCGACCCCAACCCAGCCCCACAGGAGACTCTCTGAAGACCAGCAGGAAAG CAGTGAGCCCTTACAGGTCGCCTTCGTCAGACGTCAGAATGATTCCTTGCAGAGCCGCGCT GACCTTTGCCCGATGTCTGATCCGGAGAAAAATCGTGACCCTGGACAGTCTAGAAGACAC CAAATTATGCCGCTGCTTATCCACCATGGACCTCATTGCCCTGGGCGTTGGAAGCACCCTT GGGGCCGGGGTTTATGTCCTCGCTGGGGAGGTGGCCAAGGCAGACTCGGGCCCCAGCATC GTGGTGTCCTTCCTCATTGCTGCCCTGGCTTCAGTGATGGCTGGCCTCTGCTATGCCGAATT TGGGGCCCGTGTTCCCAAGACGGGGTCTGCATATTTGTACACCTACGTGACTGTCGGAGAG CTGTGGGCCTTCATCACTGGCTGGAATCTCATTTTATCGTATGTGATAGGTACATCAAGTGT TGCAAGAGCCTGGAGTGGCACCTTTGATGAACTTCTTAGCAAACAGATTGGTCAGTTTTTG AGGACATACTTCAGAATGAATTACACTGGTCTTGCAGAATATCCCGATTTTTTTGCTGTGT GCCTTATATTACTTCTAGCAGGTCTTTTGTCTTTTGGAGTAAAAGAGTCTGCTTGGGTGAAT AAAGTCTTCACAGCTGTTAATATTCTCGTCCTTCTGTTTGTGATGGTTGCTGGGTTTGTGAA AGGAAATGTGGCAAACTGGAAGATTAGTGAAGAGTTTCTCAAAAATATATCAGCAAGTGC CAGAGAGCCACCTTCTGAAAACGGAACAAGTATCTATGGGGCTGGTGGCTTTATGCCTTAT GGCTTTACGGGAACGTTGGCTGGTGCTGCAACTTGCTTTTATGCCTTTGTGGGATTTGACTG CATTGCAACAACTGGTGAAGAAGTTCGGAATCCCCAGAAAGCTATTCCCATTGGAATTGTG ACGTCTTTGCTTGTTTGCTTTATGGCCTATTTTGGGGTCTCTGCAGCTTTAACACTTATGAT GCCGTACTACCTCCTCGATGAAAAAAGCCCCCTTCCTGTAGCGTTTGAATATGTGGGATGG GGTCCTGCCAAATATGTCGTCGCAGCTGGTTCTCTCTGCGCCTTGTCAACAAGTCTTCTTGG ATCCATTTTCCCAATGCCTCGTGTAATCTATGCTATGGCGGAGGATGGGTTGCTTTTCAAAT GTCTAGCTCAAATCAATTCCAAAACGAAGACACCAATAATTGCTACTTTATCATCGGGTGC AGTGGCAGCTTTGATGGCCTTTCTGTTTGACCTGAAGGCGCTTGTGGACATGATGTCCATT GGCACACTCATGGCCTACTCTCTGGTGGCAGCCTGTGTTCTCATCCTCAGGTACCAGCCTG GCTTATCTTACGACCAGCCCAAATGTTCTCCTGAGAAAGATGGTCTGGGATCGTCTCCCAG GGTAACCTCGAAGAGTGAGTCCCAGGTCACCATGCTGCAGAGACAGGGCTTCAGCATGCG GACCCTCTTCTGCCCCTCCCTTCTGCCAACACAGCAGTCAGCTTCTCTCGTGAGCTTTCTGG |
| | TAGGATTCCTAGCTTTCCTCGTGTTGGGCCTGAGTGTCTTGACCACTTACGGAGTTCATGCC ATCACCAGGCTGGAGGCCTGGAGCCTCGCTCTCCTCGCGCTGTTTCTTGTTCTCTTCGTTGC CATCGTTCTCACCATCTGGAGGCAGCCCCAGAATCAGCAAAAAGTAGCCTTCATGGTTCCA TTCTTACCATTTTTGCCAGCGTTCAGCATCTTGGTGAACATTTACTTGATGGTCCAGTTAAG TGCAGACACTTGGGTCAGATTCAGCATTTGGATGGCAATTGGCTTCCTGATTTACTTTTCTT ATGGCATTAGACACAGCCTGGAGGGTCATCTGAGAGATGAAAACAATGAAGAAGATGCTT ATCCAGACAACGTTCATGCAGCAGCAGAAGAAAAATCTGCCATTCAAGCAAATGACCATC ACCCAAGAAATCTCAGTTCACCTTTCATATTCCATGAAAAGACAAGTGAATTCTAACACTT GCAGGAGCAGAGCTGGTCATCGTCTTAGCATACATATCCTACACTGAGTAAACCGTAACG GGATGTCATCAGCATGCTGGGTTGTCATGGGTTTGCTGCATACATAGTTCACCCTAATTTAT ACTTACTCATCTGGACAGCATCTCCTCAGATGGTGAATTATGTGCACGGGGAAACCTCCTG AGTGGAAGTTTCATTCATCAGTGATGAATAGCCCCCAAACAGTGGGAGTGTGTATGTATGT GTGTATGTATGTATCTATGTATATGCTTGGGAACATGAGTGTTACAAGTTAGCTGGTGTTTT ACTATTATTGTGTTACATTTTTCCAGTGTCGTCATTAATCGGTGGCATATACTGCACATACT GAAATAGAGGGAAATCACTGAATGTAAAGAGGTTTCATCTATGCCCCCTGCAGTTGGGGA AATACTAGTAGCTTTACCTTGTTTGACTTCATTAATGTCAGTTTAGGGGATGCCAAAAATG CAGTTACTCATCATGGTGTCTGTCACTGGTTAGGGGTAAGATGAGGGGATAAGGAAAGAG ACTTTTCAATAAGTTGTGAATGCCAACAGTGGGTTTAATGCAAATTTTTTTTCCTGTGAGGT ATGACAGTTTGCTCAAACTTCAGCCAACAGGGGTGTCTGCTTCTGCTGCACTACACAGGCC AGGAGTGGCATTCCATGCCACTAGTTGGCATCCTTTTGAACTTTTGTCTCCTTTGCAAACAG TGGTCCTAAAATACGAGGTCTTCACTTGCTGTGAATGACGTATCCCCAGTCAGGGACTTAA GAGAGGCACTGTGATATACTTGGGACCCTTTAAATTAAAAAGTGAAGATAGTCACCAGGG CCAGAAAGCTCATGGAGTGGCCGTAATGAGAATATGTTTGAAGATCAAAGAGTTAGACCA ATGCTTGAATAAGTAGACCCCAAGCATCCTTTTCTAAAAAGTGACTTAAAATAAGCCAACA GACTCTCCCAGACCACACAACTAGTGGAATGATTCCTCCTTTTTCCATTACTTACTTAATCA CAGTTTAGTTTTTTTCTTAACCTCGTCAGGCCCAGAGTTCACTTCTTTGTTTCTCTGTTTCTT TTGTCTTGTCTTAGAGATGAGGGGGCTACAGCAGCATCATGCAAAGAGGGAAAGATGAAG GGATAGAAGAAGAGAAAATCCCCCTGTTCTGATAGGAACGGCCTGTTCCATTGTTAAATG GCAAATGGCCCAATTTAAGGGCTTTGGATCTAATTTGCCTCTGATGTTTCCTTTGGAAACAT TTAGGAATATTTTTCTCCCCCTACCCCATAAATTGTGTAGCACTTTTTATTCCATTTGCTTTC AAATGACTACACTAAGCCTAATAATACAAGCTCCAGTGTTATACAATAACCCATCAGTGAT TGGGGAATCAAACATTTTGGTTTAAAAAACATGATTATTTAAAACTGGAAACTAAAAAGA ATCAAATTGAATTAAAGCTATATAAACACAGTTAACCCTTGTAAATGAGTAAACAAATTTT TACATGTAAGATTCTCTAATTGTCATATTTTACTTTTTAGGATTCCCTAATAGTGGACTGTT TATTTGCAGTGTATTTGCTTCTCATGAACTATTTCTCGTACAAATCATTAAATAGTTCATTG GATGAGGCTGGGTGACATTTCCCAGGACAGCATGGTGAACATTACCAGGCATGCTAGCTG GCCCGTGTAATCCCAAGACAAGGAAAACATTCGTTTTCCTCATGGGTCTTCCAAGAAATGA GCTATTTTATTGATGCCATTAAAAAGCAAGTTGCGATGGTTTTGTATAGCCAGGAGTTTAT TGTGATTAAACATCAAAGAAACAGGTAGAAAGCCTGGGTTTCTGGCTGCTAGCGTTATAG CATCCATGACACAGAACTCATTACGGACATTCCACAACTTCCAGGGTGCACATGGTAAAAT CTGAAGCCCAGAATTTTCTCTCAAGCTGCGTGGTTTACTGGAGAGAAGGAGTTGGATAAGC ACAGGCTCGGGTATTTTGGTAGGGACTGTAGGCATGCTCATAAATCCTTGCTGTTGTCACA GTACGCTGAAAACCCGTTTGATTCTATACCCAATCAAGAATAGACCCTTCACACAGGAAAT GTGAACAATTGTTATATATGAACACTCAAATCTTTTACTGTAACGAAACCAAGAAACTTGT TTAGAATGTGATAGGCAGCTAAAACTGTTATGCCCACTGTGCTCAATTTGAAGCAGAATTT AGTGAAAAATTATTTTTCCACATTGAAACACTTTGCAGACACAAATATCTATGAAAAGATG CTTTGTCAGCCACTGTGCCTTTTTTTCTGTGAAGACTCAACGGATGTGTGTGTTTGTATGTT TGTTAACAGTTACATATGTTTGTATGAGTGTATATATATATCTGTGTGTGTGTATCTCTAAC GTCAGTGTATAAGTAAGTTGGGTTTATGGTGGGCTTTGACTATGTCATTAGGTGGGTACAA AACCCAACTGATGTGGAGAAAAATTGATGTTTGATGTTGATAGATATGCTTATACCTAATT TTTAGTTTTTAAACTATTTTAAAATATACTATGATTTTATATGTATATTTCCTATAGACTCTT TAAGACGTATTTATAATGTTTCTAATATGAAATCACTAAACTCTAGTACATTATAGCAGGT GCTTTGTAATCTGGAATGGAGAAGAGGTAGGGGCATTTGGGGATTCCTGTTTACTTGCTGC |
| | TGCCACACCTTTTCCGACTGATCTGTCCTGGTAGGTGTTTATTAGCAAAAGTCAGTATCACC AGCTCTTTGGCACCTTTCTGTTTCTGCTTGTGAATTCATAATGTTTTCAACTAAATTTTTTTT TTCTTTCTCAGAATTACCTAAATGTTTTGTAGAGTTTTGACTAGTAATCAATCAAAATTATA TAAAGTCTTCTCCAGTAATTAAGAAATACATATGCAAATTCTTTTGTGATTGAGTAAAAGC AGCTTAAATTACTTTTCTTTTCTACATTAAGAAATATATTCTCAACATTTTCAGTGAGAATT TCTTGTAATGGCACCTCAAATTTTATACTCTTAAAAAAAAACAATAATTTGTGAATTACCA CCAAAAGGCAATGGCAGTCCTACATTTAAGAATAGAGCTATGCAAACTCTGTTAAAAACT ATGAGGAAAACTTATATTAGAACTTTTGATATATACTAAAATACTGATTATCTTAATCACA TTTTCCCCAGAGATAAACATTGAGAGAACGAAAGCCAAAGTGTCATTTAAGAGAGATATA TATGAAAAAGTAACATTAATATATAGAACTTTACCATCACCAGCCGTAGTTGATAGAAAAT ATTAGTTTCAGAATTACCCTCCTTTAAAAAATAAGAGACTATTTGTTTTCTTTTAATTTCTA TGAATAAAAGAAATTTTTAAAAACTTTAAAATTTTAAATATTAGTCAAAATACTTTTTAAG TCCTGAGTGCTTACAGGTAGTTGTTAAAAAAATTTTAAGGCCAGGCATGGTGGCTCGCTCA CACCTATAATCCTAGGATCTTGGGAGGTCGAGGCAAGCTGATCGCTTGAGCCCAGGAGTTT AAGACCGGCCTGAGTAGCATAGCAAGACCCTGTCTCTACAAAAAAAACAAAAATTAGCTG GGCATGGTGGCATGCACATGTAGTCAGAGCTACTGGGGGTGCTGAGGTGGGAGGATCGCT TGAGCCCAGGAGAGTGAGGCTGCAGTGAGCTGAGATTACGCCACTGCACTCTAGCCTGGG CAACGGTGAGACCCTGCCTCAAAAAAAATAAAAATAAAAATAAAACACTTTAATTAGAAT CTATTTTTACCTATTTTCTAAATTTATTTAAATGCTTAGCAGGAAGCATAAGGAAAAGCCA TCGGCCTCCAATACCCATGATGACAGAGGGAGCACTTGAGCCTTGCCTTCCCTCCTCTTAA ATCAGGGTGTGTTCCGAGATTACAGAACATCACACCTTGGCGTGATGAAATCATGCCAAG ATTCTGACTCTCCCTTTCCGGTGATACTGCTCATGATTTCTCCTAATACGCTTCAAGCAACT GTTACCACAAAAAATACAGTTTCCGCAGGGCTTTAAAGGATTGAGTTTAGCATGTATATCA TGCGTTATTAAAGTTCACGTGATTCATGTGAAATTAACTGTCCTTTTTGCTAGTGCCAAAAC AGTGCCTTCTCTGCACACTTTACTTGTTTATAAAGTTCTCCCACATGTCCTTAAATATCAAG GGGGAAAGTATGGATATTCGCGTAGCAATAATGCCAGCAAAGGTCATTTTCATTTTTAGT CATATAGATATGAAAATAAGTTCATATAGATATGAAATTGCTTGACTTTATTGTTTTGGGG AGATTTTTTTTCCTTACATGATTATATTAAACACTTTAAAATAGCCTTCCGGTTTCTGGATT TTGAGAAGCCTGATCTGTTATTGTTGTGGTTGTTGGTGTTTGTAATATTCATTATTGTTTGT ATATACACGGTTTAGTCTTACTGATTTCAAATGCATTTTGTTATTGCTCAACCCAACTGGTA ACACTGTTTGCTGGGAGCATTATACTTAACTTTGATTCACCATGGTTGATGCCACTGCCATG ATCGCTGGGTCTTAAAGAGCTTTCCCTAGCCACTGACAGCCCCGTGGAGATCATAATCAGG GCCCCAGGCTGGTTCCAGGATCAGGCAGCCTATAGAGTGTGAGCATCTATGTGTAGCTACC CTTGTTGGGTGGGCTCTTAGACTGATGGGGTAGGATATGAAGTGAAAGACTTCAAATGCA AGTAAGGTAGTTTGGGCTCCTTAATTCCAAACATCCCATGAGTATATCAAGATGAATAAGG ACCAAGGGACCTCTGTGACTCATAGAAGGGCTGGCTGAATCCTGAAGTAGCATAGTGGGA CCTGGTCTACAATTTATGCACATGCACTGACAGCCTTGCTGTGCCACGTGTCTCACCAAGA CCCAGTTGGGAAAGAGCGTCATATTGCCAACAGGTTGGGTTTCTCTGGCCTACACCTGATT AATGGGCCCTTTATCTTTGGTGTCCCCTAGGAGTGTCCAGTTGTTTTATTGCTGTATTTTGTT ATTGCAGTACTTAATAAAAATTGTTGATAGGGCCCAAAACCCTACAGAAATTCTATGTCTG TAAAAACCAACAAAGGCATTGGACTTGTGTGAATGTACAGGGTTTTTTTAGTAGTAATTTT AAATTTAAATGTTTTAAGTGATCATCAGTGTTCCTTTTTACTTATAAAGTTGGATTCTTTTTT AGAATTTGTAATAAATAAAAACTGCTGCTTTACCACTGTAAAATATGCTTTCTGATGTGGT GTATTTTTAAAATAAATTTTAATATGTAATAA |
| 188 | TCCCAAAACAGAAAGAGCAGATGTCTCACCACGAAACTAGCAACTGGAATGAAGATAGA AACAAGTGGTTATAACTCAGACAAACTAATTTGTCGAGGGTTTATTGGAACACCTGCCCCA CCGGTTTGCGACAGCAAGTTTCTCCTGTCGCCTTCGTCAGACGTCAGAATGATTCCTTGCA GAGCCGCGCTGACCTTTGCCCGATGTCTGATCCGGAGAAAAATCGTGACCCTGGACAGTCT AGAAGACACCAAATTATGCCGCTGCTTATCCACCATGGACCTCATTGCCCTGGGCGTTGGA AGCACCCTTGGGGCCGGGGTTTATGTCCTCGCTGGGGAGGTGGCCAAGGCAGACTCGGGC CCCAGCATCGTGGTGTCCTTCCTCATTGCTGCCCTGGCTTCAGTGATGGCTGGCCTCTGCTA TGCCGAATTTGGGGCCCGTGTTCCCAAGACGGGGTCTGCATATTTGTACACCTACGTGACT |
| | GTCGGAGAGCTGTGGGCCTTCATCACTGGCTGGAATCTCATTTTATCGTATGTGATAGGTA CATCAAGTGTTGCAAGAGCCTGGAGTGGCACCTTTGATGAACTTCTTAGCAAACAGATTGG TCAGTTTTTGAGGACATACTTCAGAATGAATTACACTGGTCTTGCAGAATATCCCGATTTTT TTGCTGTGTGCCTTATATTACTTCTAGCAGGTCTTTTGTCTTTTGGAGTAAAAGAGTCTGCT TGGGTGAATAAAGTCTTCACAGCTGTTAATATTCTCGTCCTTCTGTTTGTGATGGTTGCTGG GTTTGTGAAAGGAAATGTGGCAAACTGGAAGATTAGTGAAGAGTTTCTCAAAAATATATC AGCAAGTGCCAGAGAGCCACCTTCTGAAAACGGAACAAGTATCTATGGGGCTGGTGGCTT TATGCCTTATGGCTTTACGGGAACGTTGGCTGGTGCTGCAACTTGCTTTTATGCCTTTGTGG GATTTGACTGCATTGCAACAACTGGTGAAGAAGTTCGGAATCCCCAGAAAGCTATTCCCAT TGGAATTGTGACGTCTTTGCTTGTTTGCTTTATGGCCTATTTTGGGGTCTCTGCAGCTTTAA CACTTATGATGCCGTACTACCTCCTCGATGAAAAAAGCCCCCTTCCTGTAGCGTTTGAATA TGTGGGATGGGGTCCTGCCAAATATGTCGTCGCAGCTGGTTCTCTCTGCGCCTTGTCAACA AGTCTTCTGGGCTCTATGTTTCCTTTACCCCGAATTCTGTTTGCCATGGCCCGGGATGGCTT ACTGTTTAGATTTCTTGCCAGAGTGAGTAAGAGGCAGTCACCAGTTGCTGCCACGTTGACT GCAGGGGTCATTTCTGCTTTGATGGCCTTTCTGTTTGACCTGAAGGCGCTTGTGGACATGAT GTCCATTGGCACACTCATGGCCTACTCTCTGGTGGCAGCCTGTGTTCTCATCCTCAGGTACC AGCCTGGCTTATCTTACGACCAGCCCAAATGTTCTCCTGAGAAAGATGGTCTGGGATCGTC TCCCAGGGTAACCTCGAAGAGTGAGTCCCAGGTCACCATGCTGCAGAGACAGGGCTTCAG CATGCGGACCCTCTTCTGCCCCTCCCTTCTGCCAACACAGCAGTCAGCTTCTCTCGTGAGCT TTCTGGTAGGATTCCTAGCTTTCCTCGTGTTGGGCCTGAGTGTCTTGACCACTTACGGAGTT CATGCCATCACCAGGCTGGAGGCCTGGAGCCTCGCTCTCCTCGCGCTGTTTCTTGTTCTCTT CGTTGCCATCGTTCTCACCATCTGGAGGCAGCCCCAGAATCAGCAAAAAGTAGCCTTCATG GTTCCATTCTTACCATTTTTGCCAGCGTTCAGCATCTTGGTGAACATTTACTTGATGGTCCA GTTAAGTGCAGACACTTGGGTCAGATTCAGCATTTGGATGGCAATTGGCTTCCTGATTTAC TTTTCTTATGGCATTAGACACAGCCTGGAGGGTCATCTGAGAGATGAAAACAATGAAGAA GATGCTTATCCAGACAACGTTCATGCAGCAGCAGAAGAAAAATCTGCCATTCAAGCAAAT GACCATCACCCAAGAAATCTCAGTTCACCTTTCATATTCCATGAAAAGACAAGTGAATTCT AACACTTGCAGGAGCAGAGCTGGTCATCGTCTTAGCATACATATCCTACACTGAGTAAACC GTAACGGGATGTCATCAGCATGCTGGGTTGTCATGGGTTTGCTGCATACATAGTTCACCCT AATTTATACTTACTCATCTGGACAGCATCTCCTCAGATGGTGAATTATGTGCACGGGGAAA CCTCCTGAGTGGAAGTTTCATTCATCAGTGATGAATAGCCCCCAAACAGTGGGAGTGTGTA TGTATGTGTGTATGTATGTATCTATGTATATGCTTGGGAACATGAGTGTTACAAGTTAGCT GGTGTTTTACTATTATTGTGTTACATTTTTCCAGTGTCGTCATTAATCGGTGGCATATACTG CACATACTGAAATAGAGGGAAATCACTGAATGTAAAGAGGTTTCATCTATGCCCCCTGCA GTTGGGGAAATACTAGTAGCTTTACCTTGTTTGACTTCATTAATGTCAGTTTAGGGGATGC CAAAAATGCAGTTACTCATCATGGTGTCTGTCACTGGTTAGGGGTAAGATGAGGGGATAA GGAAAGAGACTTTTCAATAAGTTGTGAATGCCAACAGTGGGTTTAATGCAAATTTTTTTTC CTGTGAGGTATGACAGTTTGCTCAAACTTCAGCCAACAGGGGTGTCTGCTTCTGCTGCACT ACACAGGCCAGGAGTGGCATTCCATGCCACTAGTTGGCATCCTTTTGAACTTTTGTCTCCTT TGCAAACAGTGGTCCTAAAATACGAGGTCTTCACTTGCTGTGAATGACGTATCCCCAGTCA GGGACTTAAGAGAGGCACTGTGATATACTTGGGACCCTTTAAATTAAAAAGTGAAGATAG TCACCAGGGCCAGAAAGCTCATGGAGTGGCCGTAATGAGAATATGTTTGAAGATCAAAGA GTTAGACCAATGCTTGAATAAGTAGACCCCAAGCATCCTTTTCTAAAAAGTGACTTAAAAT AAGCCAACAGACTCTCCCAGACCACACAACTAGTGGAATGATTCCTCCTTTTTCCATTACT TACTTAATCACAGTTTAGTTTTTTTCTTAACCTCGTCAGGCCCAGAGTTCACTTCTTTGTTTC TCTGTTTCTTTTGTCTTGTCTTAGAGATGAGGGGGCTACAGCAGCATCATGCAAAGAGGGA AAGATGAAGGGATAGAAGAAGAGAAAATCCCCCTGTTCTGATAGGAACGGCCTGTTCCAT TGTTAAATGGCAAATGGCCCAATTTAAGGGCTTTGGATCTAATTTGCCTCTGATGTTTCCTT TGGAAACATTTAGGAATATTTTTCTCCCCCTACCCCATAAATTGTGTAGCACTTTTTATTCC ATTTGCTTTCAAATGACTACACTAAGCCTAATAATACAAGCTCCAGTGTTATACAATAACC CATCAGTGATTGGGGAATCAAACATTTTGGTTTAAAAAACATGATTATTTAAAACTGGAAA CTAAAAAGAATCAAATTGAATTAAAGCTATATAAACACAGTTAACCCTTGTAAATGAGTA AACAAATTTTTACATGTAAGATTCTCTAATTGTCATATTTTACTTTTTAGGATTCCCTAATA |
| | GTGGACTGTTTATTTGCAGTGTATTTGCTTCTCATGAACTATTTCTCGTACAAATCATTAAA TAGTTCATTGGATGAGGCTGGGTGACATTTCCCAGGACAGCATGGTGAACATTACCAGGC ATGCTAGCTGGCCCGTGTAATCCCAAGACAAGGAAAACATTCGTTTTCCTCATGGGTCTTC CAAGAAATGAGCTATTTTATTGATGCCATTAAAAAGCAAGTTGCGATGGTTTTGTATAGCC AGGAGTTTATTGTGATTAAACATCAAAGAAACAGGTAGAAAGCCTGGGTTTCTGGCTGCT AGCGTTATAGCATCCATGACACAGAACTCATTACGGACATTCCACAACTTCCAGGGTGCAC ATGGTAAAATCTGAAGCCCAGAATTTTCTCTCAAGCTGCGTGGTTTACTGGAGAGAAGGA GTTGGATAAGCACAGGCTCGGGTATTTTGGTAGGGACTGTAGGCATGCTCATAAATCCTTG CTGTTGTCACAGTACGCTGAAAACCCGTTTGATTCTATACCCAATCAAGAATAGACCCTTC ACACAGGAAATGTGAACAATTGTTATATATGAACACTCAAATCTTTTACTGTAACGAAACC AAGAAACTTGTTTAGAATGTGATAGGCAGCTAAAACTGTTATGCCCACTGTGCTCAATTTG AAGCAGAATTTAGTGAAAAATTATTTTTCCACATTGAAACACTTTGCAGACACAAATATCT ATGAAAAGATGCTTTGTCAGCCACTGTGCCTTTTTTTCTGTGAAGACTCAACGGATGTGTG TGTTTGTATGTTTGTTAACAGTTACATATGTTTGTATGAGTGTATATATATATCTGTGTGTG TGTATCTCTAACGTCAGTGTATAAGTAAGTTGGGTTTATGGTGGGCTTTGACTATGTCATTA GGTGGGTACAAAACCCAACTGATGTGGAGAAAAATTGATGTTTGATGTTGATAGATATGC TTATACCTAATTTTTAGTTTTTAAACTATTTTAAAATATACTATGATTTTATATGTATATTTC CTATAGACTCTTTAAGACGTATTTATAATGTTTCTAATATGAAATCACTAAACTCTAGTACA TTATAGCAGGTGCTTTGTAATCTGGAATGGAGAAGAGGTAGGGGCATTTGGGGATTCCTGT TTACTTGCTGCTGCCACACCTTTTCCGACTGATCTGTCCTGGTAGGTGTTTATTAGCAAAAG TCAGTATCACCAGCTCTTTGGCACCTTTCTGTTTCTGCTTGTGAATTCATAATGTTTTCAACT AAATTTTTTTTTTCTTTCTCAGAATTACCTAAATGTTTTGTAGAGTTTTGACTAGTAATCAAT CAAAATTATATAAAGTCTTCTCCAGTAATTAAGAAATACATATGCAAATTCTTTTGTGATT GAGTAAAAGCAGCTTAAATTACTTTTCTTTTCTACATTAAGAAATATATTCTCAACATTTTC AGTGAGAATTTCTTGTAATGGCACCTCAAATTTTATACTCTTAAAAAAAAACAATAATTTG TGAATTACCACCAAAAGGCAATGGCAGTCCTACATTTAAGAATAGAGCTATGCAAACTCT GTTAAAAACTATGAGGAAAACTTATATTAGAACTTTTGATATATACTAAAATACTGATTAT CTTAATCACATTTTCCCCAGAGATAAACATTGAGAGAACGAAAGCCAAAGTGTCATTTAA GAGAGATATATATGAAAAAGTAACATTAATATATAGAACTTTACCATCACCAGCCGTAGTT GATAGAAAATATTAGTTTCAGAATTACCCTCCTTTAAAAAATAAGAGACTATTTGTTTTCTT TTAATTTCTATGAATAAAAGAAATTTTTAAAAACTTTAAAATTTTAAATATTAGTCAAAAT ACTTTTTAAGTCCTGAGTGCTTACAGGTAGTTGTTAAAAAAATTTTAAGGCCAGGCATGGT GGCTCGCTCACACCTATAATCCTAGGATCTTGGGAGGTCGAGGCAAGCTGATCGCTTGAGC CCAGGAGTTTAAGACCGGCCTGAGTAGCATAGCAAGACCCTGTCTCTACAAAAAAAACAA AAATTAGCTGGGCATGGTGGCATGCACATGTAGTCAGAGCTACTGGGGGTGCTGAGGTGG GAGGATCGCTTGAGCCCAGGAGAGTGAGGCTGCAGTGAGCTGAGATTACGCCACTGCACT CTAGCCTGGGCAACGGTGAGACCCTGCCTCAAAAAAAATAAAAATAAAAATAAAACACTT TAATTAGAATCTATTTTTACCTATTTTCTAAATTTATTTAAATGCTTAGCAGGAAGCATAAG GAAAAGCCATCGGCCTCCAATACCCATGATGACAGAGGGAGCACTTGAGCCTTGCCTTCC CTCCTCTTAAATCAGGGTGTGTTCCGAGATTACAGAACATCACACCTTGGCGTGATGAAAT CATGCCAAGATTCTGACTCTCCCTTTCCGGTGATACTGCTCATGATTTCTCCTAATACGCTT CAAGCAACTGTTACCACAAAAAATACAGTTTCCGCAGGGCTTTAAAGGATTGAGTTTAGC ATGTATATCATGCGTTATTAAAGTTCACGTGATTCATGTGAAATTAACTGTCCTTTTTGCTA GTGCCAAAACAGTGCCTTCTCTGCACACTTTACTTGTTTATAAAGTTCTCCCACATGTCCTT AAATATCAAGGGGGAAAGTATGGATATTCGCGTAGCAATAATGCCAGCAAAGGTCATTTT CATTTTTTAGTCATATAGATATGAAAATAAGTTCATATAGATATGAAATTGCTTGACTTTAT TGTTTTGGGGAGATTTTTTTTCCTTACATGATTATATTAAACACTTTAAAATAGCCTTCCGG TTTCTGGATTTTGAGAAGCCTGATCTGTTATTGTTGTGGTTGTTGGTGTTTGTAATATTCATT ATTGTTTGTATATACACGGTTTAGTCTTACTGATTTCAAATGCATTTTGTTATTGCTCAACC CAACTGGTAACACTGTTTGCTGGGAGCATTATACTTAACTTTGATTCACCATGGTTGATGC CACTGCCATGATCGCTGGGTCTTAAAGAGCTTTCCCTAGCCACTGACAGCCCCGTGGAGAT CATAATCAGGGCCCCAGGCTGGTTCCAGGATCAGGCAGCCTATAGAGTGTGAGCATCTAT GTGTAGCTACCCTTGTTGGGTGGGCTCTTAGACTGATGGGGTAGGATATGAAGTGAAAGA |
| | CTTCAAATGCAAGTAAGGTAGTTTGGGCTCCTTAATTCCAAACATCCCATGAGTATATCAA GATGAATAAGGACCAAGGGACCTCTGTGACTCATAGAAGGGCTGGCTGAATCCTGAAGTA GCATAGTGGGACCTGGTCTACAATTTATGCACATGCACTGACAGCCTTGCTGTGCCACGTG TCTCACCAAGACCCAGTTGGGAAAGAGCGTCATATTGCCAACAGGTTGGGTTTCTCTGGCC TACACCTGATTAATGGGCCCTTTATCTTTGGTGTCCCCTAGGAGTGTCCAGTTGTTTTATTG CTGTATTTTGTTATTGCAGTACTTAATAAAAATTGTTGATAGGGCCCAAAACCCTACAGAA ATTCTATGTCTGTAAAAACCAACAAAGGCATTGGACTTGTGTGAATGTACAGGGTTTTTTT AGTAGTAATTTTAAATTTAAATGTTTTAAGTGATCATCAGTGTTCC11111ACTTATAAAGT TGGATTCTTTTTTAGAATTTGTAATAAATAAAAACTGCTGCTTTACCACTGTAAAATATGCT TTCTGATGTGGTGTATTTTTAAAATAAATTTTAATATGTAATAA |
| 204 | AAGCCGCAGCTTTGAAGCCTGAGCGGCCGAACTCGGCAGCTCCAACCCAACTCGGCTTAA CTCCGCCTCACCGAGCCCAGTCCAAGACTCTGTGCTCCCTAGGTTTGCAACAGCTCTCTGG ATGCCGTGGCAAGCATTTCGCAGATTTGGTCAAAAGCTGGTACGCAGACGTACACTGGAG TCAGGCATGGCTGAGACTCGCCTTGCCAGATGCCTAAGCACCCTGGATTTAGTGGCCCTGG GTGTGGGCAGCACATTGGGTGCAGGCGTGTATGTCCTAGCTGGCGAGGTGGCCAAAGATA AAGCAGGGCCATCCATTGTGATCTGCTTTTTGGTGGCTGCCCTGTCTTCTGTGTTGGCTGGG CTGTGCTATGCGGAGTTTGGTGCCCGGGTTCCCCGTTCTGGTTCGGCATATCTCTACAGCTA TGTCACTGTGGGTGAACTCTGGGCCTTCACCACTGGCTGGAACCTCATCCTCTCCTATGTCA TTGGTACAGCCAGTGTGGCCCGGGCCTGGAGCTCTGCTTTTGACAACCTGATTGGGAACCA CATCTCTAAGACTCTGCAGGGGTCCATTGCACTGCACGTGCCCCATGTCCTTGCAGAATAT CCAGATTTCTTTGCTTTGGGCCTCGTGTTGCTGCTCACTGGATTGTTGGCTCTCGGGGCTAG TGAGTCGGCCCTGGTTACCAAAGTGTTCACAGGCGTGAACCTTTTGGTTCTTGGGTTCGTC ATGATCTCTGGCTTCGTTAAGGGGGACGTGCACAACTGGAAGCTCACAGAAGAGGACTAC GAATTGGCCATGGCTGAACTCAATGACACCTATAGCTTGGGTCCTCTGGGCTCTGGAGGAT TTGTGCCTTTCGGCTTCGAGGGAATTCTCCGTGGAGCAGCGACCTGTTTCTATGCATTTGTT GGTTTCGACTGTATTGCTACCACTGGAGAAGAAGCCCAGAATCCCCAGCGTTCCATCCCGA TGGGCATTGTGATCTCACTGTCTGTCTGCTTTT TGGCGTATTTTGCTGTCTCTTCTGCACTCA CCCTGATGATGCCTTACTACCAGCTTCAGCCTGAGAGCCCTTTGCCTGAGGCATTTCTCTAC ATTGGATGGGCTCCTGCCCGCTATGTTGTGGCTGTTGGCTCCCTCTGTGCTCTTTCTACCAG CCTCCTGGGCTCCATGTTCCCCATGCCTCGGGTGATCTACGCGATGGCAGAGGATGGCCTC CTGTTCCGTGTACTTGCTCGGATCCACACCGGCACACGCACCCCAATCATAGCCACCGTGG TCTCTGGCATTATTGCAGCATTCATGGCATTCCTCTTCAAACTCACTGATCTTGTGGACCTC ATGTCAATTGGGACCCTGCTTGCTTACTCCCTGGTGTCGATTTGTGTTCTCATCCTCAGGTA TCAACCTGATCAGGAGACAAAGACTGGGGAAGAAGTGGAGTTGCAGGAGGAGGCAATAA CTACTGAATCAGAGAAGTTGACCCTATGGGGACTATTTTTCCCACTCAACTCCATCCCCAC TCCACTCTCTGGCCAAATTGTCTATGTTTGTTCCTCATTGCTTGCTGTCCTGCTGACTGCTCT TTGCCTGGTGCTGGCCCAGTGGTCAGTTCCATTGCTTTCTGGAGACCTGCTGTGGACTGCA GTGGTTGTGCTGCTCCTGCTGCTCATTATTGGGATCATTGTGGTCATCTGGAGACAGCCAC AGAGTTCCACTCCCCTTCACTTTAAGGTGCCTGCTTTGCCTCTCCTCCCACTAATGAGCATC TTTGTGAATATTTACCTTATGATGCAGATGACAGCTGGTACCTGGGCCCGATTTGGGGTCT GGATGCTGATTGGCTTTGCTATCTACTTCGGCTATGGGATCCAGCACAGCCTGGAAGAGAT TAAGAGTAACCAACCCTCACGCAAGTCTAGAGCCAAAACTGTAGACCTTGATCCCGGCAC TCTCTATGTCCACTCAGTTTGACATCGTCACACCTAAATGCTGTCTGGTCCCCTGCACAATA ATGGAGAGTACTCCTGACCCCAGTGACAGCTAGCCCTCCCCTGTGATGGTGGTGGTGGATA CTAATACAGTTCTGTACGATGTGAAGGATGTGTCTTTGCTATTTCTTGTCTATTTTAACCCG TCTGCTTCTAAATGATGTCTAGCTGCTTACCAACTTTAAAAAATGATATTAAAAGAAAGTA GAAAAATAAA |
| 205 | AAGCCGCAGCTTTGAAGCCTGAGCGGCCGAACTCGGCAGCTCCAACCCAACTCGGCTTAA CTCCGCCTCACCGAGCCCAGTCCAAGACTCTGTGCTCCCTAGGTTTGCAACAGCTCTCTGA TCATCTTCTTCAATTCCTGCTAGGATGCCGTGGCAAGCATTTCGCAGATTTGGTCAAAAGC TGGTACGCAGACGTACACTGGAGTCAGGCATGGCTGAGACTCGCCTTGCCAGATGCCTAA |
| | GCACCCTGGATTTAGTGGCCCTGGGTGTGGGCAGCACATTGGGTGCAGGCGTGTATGTCCT AGCTGGCGAGGTGGCCAAAGATAAAGCAGGGCCATCCATTGTGATCTGCTTTTTGGTGGCT GCCCTGTCTTCTGTGTTGGCTGGGCTGTGCTATGCGGAGTTTGGTGCCCGGGTTCCCCGTTC TGGTTCGGCATATCTCTACAGCTATGTCACTGTGGGTGAACTCTGGGCCTTCACCACTGGC TGGAACCTCATCCTCTCCTATGTCATTGGTACAGCCAGTGTGGCCCGGGCCTGGAGCTCTG CTTTTGACAACCTGATTGGGAACCACATCTCTAAGACTCTGCAGGGGTCCATTGCACTGCA CGTGCCCCATGTCCTTGCAGAATATCCAGATTTCTTTGCTTTGGGCCTCGTGTTGCTGCTCA CTGGATTGTTGGCTCTCGGGGCTAGTGAGTCGGCCCTGGTTACCAAAGTGTTCACAGGCGT GAACCTTTTGGTTCTTGGGTTCGTCATGATCTCTGGCTTCGTTAAGGGGGACGTGCACAAC TGGAAGCTCACAGAAGAGGACTACGAATTGGCCATGGCTGAACTCAATGACACCTATAGC TTGGGTCCTCTGGGCTCTGGAGGATTTGTGCCTTTCGGCTTCGAGGGAATTCTCCGTGGAG CAGCGACCTGTTTCTATGCATTTGTTGGTTTCGACTGTATTGCTACCACTGGAGAAGAAGC CCAGAATCCCCAGCGTTCCATCCCGATGGGCATTGTGATCTCACTGTCTGTCTGCTTTTTGG CGTATTTTGCTGTCTCTTCTGCACTCACCCTGATGATGCCTTACTACCAGCTTCAGCCTGAG AGCCCTTTGCCTGAGGCATTTCTCTACATTGGATGGGCTCCTGCCCGCTATGTTGTGGCTGT TGGCTCCCTCTGTGCTCTTTCTACCAGCCTCCTGGGCTCCATGTTCCCCATGCCTCGGGTGA TCTACGCGATGGCAGAGGATGGCCTCCTGTTCCGTGTACTTGCTCGGATCCACACCGGCAC ACGCACCCCAATCATAGCCACCGTGGTCTCTGGCATTATTGCAGCATTCATGGCATTCCTC TTCAAACTCACTGATCTTGTGGACCTCATGTCAATTGGGACCCTGCTTGCTTACTCCCTGGT GTCGATTTGTGTTCTCATCCTCAGGTATCAACCTGATCAGGAGACAAAGACTGGGGAAGA AGTGGAGTTGCAGGAGGAGGCAATAACTACTGAATCAGAGAAGTTGACCCTATGGGGACT ATTTTTCCCACTCAACTCCATCCCCACTCCACTCTCTGGCCAAATTGTCTATGTTTGTTCCTC ATTGCTTGCTGTCCTGCTGACTGCTCTTTGCCTGGTGCTGGCCCAGTGGTCAGTTCCATTGC TTTCTGGAGACCTGCTGTGGACTGCAGTGGTTGTGCTGCTCCTGCTGCTCATTATTGGGATC ATTGTGGTCATCTGGAGACAGCCACAGAGTTCCACTCCCCTTCACTTTAAGGTGCCTGCTTT GCCTCTCCTCCCACTAATGAGCATCTTTGTGAATATTTACCTTATGATGCAGATGACAGCTG GTACCTGGGCCCGATTTGGGGTCTGGATGCTGATTGGCTTTGCTATCTACTTCGGCTATGG GATCCAGCACAGCCTGGAAGAGATTAAGAGTAACCAACCCTCACGCAAGTCTAGAGCCAA AACTGTAGACCTTGATCCCGGCACTCTCTATGTCCACTCAGTTTGACATCGTCACACCTAA ATGCTGTCTGGTCCCCTGCACAATAATGGAGAGTACTCCTGACCCCAGTGACAGCTAGCCC TCCCCTGTGATGGTGGTGGTGGATACTAATACAGTTCTGTACGATGTGAAGGATGTGTCTT TGCTATTTCTTGTCTATTTTAACCCGTCTGCTTCTAAATGATGTCTAGCTGCTTACCAACTTT AAAAAATGATATTAAAAGAAAGTAGAAAAATAAA |
| 210 | AGAGCGGAGGCAGCGGCTGCGGCAGCAGCAGGTTCCAGTAGCTGGCTCGGTGCTCTTCTC GGCCACCTGCCATGGCCCGGGGGCTGCCCACCATTGCTAGCCTGGCACGCTTATGCCAGAA GCTGAACCGCCTGAAGCCGCTGGAGGACTCCACCATGGAGACGTCACTGCGGCGCTGCCT GTCCACGCTGGACCTGACTCTTCTGGGCGTGGGTGGCATGGTGGGCTCGGGTCTCTACGTG CTCACAGGTGCCGTGGCCAAGGAGGTGGCTGGCCCTGCTGTGCTCTTGTCCTTCGGTGTGG CCGCTGTGGCCTCCCTGCTGGCAGCCCTATGCTATGCAGAATTTGGGGCACGTGTGCCACG CACGGGCTCTGCCTACCTGTTCACCTACGTATCCATGGGCGAGCTGTGGGCCTTCCTCATC GGCTGGAATGTTCTCCTCGAATACATCATCGGTGGCGCCGCCGTGGCCCGTGCCTGGAGTG GCTACCTGGACTCTATGTTCAGCCACAGCATCCGCAACTTCACTGAGACCCACGTGGGTTC TTGGCAGGTGCCCCTCCTGGGCCACTACCCGGACTTCCTGGCTGCTGGCATCATCCTCCTG GCCTCTGCCTTTGTCTCCTGTGGAGCCCGCGTGTCCTCCTGGCTCAATCACACCTTCTCGGC CATCAGCCTGCTTGTCATTCTCTTCATTGTCATCCTGGGCTTCATCCTGGCCCAGCCTCACA ACTGGAGCGCTGACGAAGGCGGCTTTGCACCCTTCGGCTTCTCCGGCGTCATGGCCGGCAC TGCCTCCTGCTTCTATGCTTTCGTGGGCTTCGACGTCATTGCCGCCTCCAGTGAGGAGGCCC AGAACCCACGGCGGTCTGTGCCTCTGGCCATCGCCATCTCGCTTGCCATTGCAGCTGGTGC CTACATCCTTGTCTCCACCGTGCTAACCCTCATGGTGCCCTGGCACAGCCTGGACCCCGAC TCAGCGCTTGCAGATGCCTTCTACCAGCGGGGCTACAGGTGGGCTGGCTTCATCGTGGCAG CTGGCTCCATCTGCGCCATGAACACCGTCCTGCTCAGCCTCCTCTTCTCCCTGCCACGCATT GTCTATGCCATGGCCGCCGATGGGCTCTTCTTCCAGGTGTTTGCCCATGTGCACCCCCGGA |
| | CACAGGTGCCTGTGGCGGGCACCCTGGCGTTCGGGCTCCTCACGGCCTTCCTGGCACTGCT GCTGGACCTGGAGTCGCTGGTTCAGTTCCTGTCCCTTGGCACACTCCTGGCCTACACATTC GTGGCCACCAGTATCATTGTGCTGCGCTTCCAGAAGTCTTCCCCGCCCAGCTCCCCAGGCC CAGCCAGCCCTGGCCCCCTGACCAAGCAGCAGAGCTCCTTCTCAGACCACCTACAGCTGGT GGGCACTGTACACGCCTCCGTCCCTGAGCCAGGGGAGCTGAAGCCAGCCCTGAGGCCCTA CCTGGGCTTCTTGGATGGGTACAGCCCTGGAGCAGTGGTGACTTGGGCGCTTGGCGTTATG TTGGCCTCAGCCATCACCATAGGCTGCGTGCTTGTCTTTGGGAACTCGACCCTGCACCTCC CACACTGGGGTTACATCCTGCTGCTCCTGCTCACCAGTGTCATGTTTCTGCTCAGCCTCCTT GTCCTGGGGGCTCACCAGCAACAGTATCGGGAAGACTTATTTCAGATCCCCATGGTTCCCC TGATTCCAGCCCTGAGCATCGTCCTCAACATCTGCCTCATGCTGAAACTTAGCTATCTGAC CTGGGTGCGCTTCTCCATCTGGCTGCTGATGGGACTTGCAGTGTATTTCGGCTATGGCATCC GGCATAGCAAGGAGAACCAGCGGGAGCTGCCAGGGCTGAACTCCACACACTACGTGGTAT TCCCCAGGGGCAGCCTGGAGGAGACAGTGCAGGCTATGCAGCCCCCCAGCCAGGCACCAG CACAGGACCCTGGCCATATGGAGTAGCTGATCAGCCCACACTTGCCCCGCCCTCCCACACC TGCTTGGGAGGCCAGAGAGGCCAGACAAGCCGAGAGCCCCTTCTGTTGTGGGCAGCCTGG GTTTGCAGGCCTGCACAGGCTGGGGAGTCCTCAGGACCTTAGGACCTTCATCCAGGGGCTG GGCTTCGGGTCTTCAGGAGTGGGCCTTGGCTGGTGCTGGTGCCATGGACTCTGCCCAGAGC CTTCTTGTTTATGATCAGCTCCAGCTACCTGGGCAGTTGTGGTGGGGTGGATGGGAAGGCC CACAGCCCAAGGGATCCATAATAATAATTGCTTGGCCAGCCATGTGGCCTGCTGGCGTA |
| 214 | GCGGCGGCGGCGGCGCGACCGAGCATCCTGGCGGCGCCGGGCCACTGGGAGAGTTTATGT GGCCGAGGCAGACAAGTGGAATTAGGCCTTGCTGCAGGGGACTTCATTTCCTTCTCAGTAC TGGACCCATTTATGAGGAGGTGGCTTATGAAAGTGTGATGTTCGCGTATTTCTTGACAGGC AGTGGCGTGATCTTGGCTCACTGCAACCTCCGACTCCCTGGTTCAAGCGATTCTCCTGCCTC AGCCTCCTGAGTGGGGATTACAGGCCACAGCAAACACAGGTGTGCAGGAACCGTTTGTCA TGGAAGCCAGGGAGCCTGGGAGGCCCACACCCACCTACCATCTTGTCCCTAACACCAGCC AGTCCCAGGTGGAAGAAGATGTCAGCTCGCCACCTCAAAGGTCCTCCGAAACTATGCAGC TGAAGAAGGAGATCTCCCTGCTGAATGGGGTCAGCCTGGTGGTGGGCAACATGATCGGCT CAGGGATCTTTGTCTCACCCAAGGGTGTGCTGGTACACACTGCCTCCTATGGGATGTCACT GATTGTGTGGGCCATTGGTGGGCTCTTCTCTGTTGTGGGTGCCCTTTGTTATGCAGAGCTGG GGACCACCATCACCAAGTCGGGAGCCAGCTACGCTTATATTCTAGAGGCCTTTGGGGGCTT CATTGCCTTCATCCGCCTGTGGGTCTCACTGCTAGTTGTTGAGCCCACCGGTCAGGCCATC ATCGCCATCACCTTTGCCAACTACATCATCCAGCCGTCCTTCCCCAGCTGTGATCCCCCATA CCTGGCCTGCCGTCTCCTGGCTGCTGCTTGCATATGTCTGCTGACATTTGTGAACTGTGCCT ATGTCAAGTGGGGCACACGTGTGCAGGACACGTTCACTTACGCCAAGGTCGTAGCGCTCA TTGCCATCATTGTCATGGGCCTTGTTAAACTGTGCCAGGGACACTCTGAGCACTTTCAGGA CGCCTTTGAGGGTTCCTCCTGGGACATGGGAAACCTCTCTCTTGCCCTCTACTCTGCCCTCT TCTCTTACTCAGGTTGGGACACCCTTAATTTTGTAACAGAAGAAATCAAAAACCCAGAAAG AAATTTGCCCTTGGCCATTGGGATTTCTATGCCAATTGTGACGCTCATCTACATCCTGACCA ATGTGGCCTATTACACAGTGCTGAACATTTCAGATGTCCTTAGCAGTGATGCTGTGGCTGT GACATTTGCTGACCAGACGTTTGGCATGTTCAGCTGGACCATCCCCATTGCTGTTGCCCTGT CCTGCTTTGGGGGCCTCAATGCATCCATCTTTGCTTCATCAAGGTTGTTCTTCGTGGGCTCC CGGGAGGGCCACCTACCGGACCTTCTGTCCATGATCCACATTGAGCGTTTTACACCTATCC CTGCTTTACTGTTCAATTGCACCATGGCACTCATCTACCTCATCGTGGAGGATGTTTTCCAG CTTATCAACTACTTCAGCTTCAGCTACTGGTTCTTCGTGGGCCTGTCTGTTGTTGGACAGCT CTACCTCCGCTGGAAGGAGCCCAAGCGGCCCCGGCCTCTCAAGCTGAGCGTGTTTTTCCCC ATCGTGTTCTGCATATGCTCCGTGTTTCTGGTGATAGTGCCCCTCTTCACTGACACCATTAA TTCCCTCATTGGCATCGGGATTGCCCTTTCTGGAGTCCCTTTCTACTTCATGGGTGTTTACC TGCCAGAGTCCCGGAGGCCATTGTTTATTCGGAATGTCCTGGCTGCTATCACCAGAGGCAC CCAGCAGCTTTGCTTTTGTGTCCTGACTGAGCTTGATGTAGCCGAAGAAAAAAAGGATGAG AGGAAAACTGACTAGAGGTCAGAGGTGGCTTTCTGAGGCCTGGAAGGCAGGCCAACCAGC AAAATCCTGATAACAAGACTCTGTGGGCCCAACTCTCCTGAATTAAAGGAGCCTTTTGACC CAATCATATAGTGGGGCTCAGGGCCAGTGCTCACTCTTATTGGTAAGCTATAGGAGACTCA |
| | GGATCTGGGCCAACCTCAAGGTGGGGGCTTCAGAGGGTGGGGGGAAGATTGGGGAACGG GGGGAATGGTCATTTAGTTTTACTCCTGATAGGTAGATGCAGCTCTTACAGATATTTACTT GGTAAAGTGCAGTGGGGAAGAGGGAATGCTAGGTTGATAGGGCTGGTGGCTTCTGAATTT GGTATTTGAACTAGGAGTCCCTATAGAGGGGCTGCTTTATGGGAAGTTTTTCTCTGACCAG GTACAACACCTGACTTTAAAGGCCTGAAATGCTACCATTTCTTCCTCTGGCTCAAAATTCTT CCCTGGGGAGAGAGTTATATTCCCTTATTTATTGATATTTAGTCCAGAACACCAGTTCTAA CGAAGCATGCGTGTCTCTTCATCTACAGGATGCAATAGGCTGATTGTATTTAAAAATCAAA GTACCCAAAACTGAGTCCCTTTGGGCTCAGAAATGTCTGTGGTATTGGGTCAGACTCTGAC CACAGGTTTTATGCTGTTTAGCACAATTTCTATTGAGTCTTACCTGCAACAATGAACCTTAA AGATTTTTTTACTCACGTACCTGTTACACTTTAGCATACAGATAGATCATAGATCACGTTAC AAGCACTTGGCTCAGGTCCAGCAAGGACAGATGAACAAATTCCTGAGTCAGAAGTCTGTT AATATTGCTGTTTTGAAGGACAATCCTTTATTTTACTTGAGACCTTACATCTTTGTTCTAGC TGACAGTAAATCTCTGGGTTTCTGTTACGAACTCTAAGAGGGCTGAAACTTCTGATATTCA GGTGGATCACCTGAATTCTCTCAGCTGTCAATGGCTTGGAGAACATCTCATGGGCCCAAGT CATCAAATAACCTGTTCCTCTCTGTAAGGGCAGTGTGAGGGACTGCTGTGCAGACCCAAGC AATCCCAACCTGGTGCTAGGTCATTTCACTTTTCTGAAAACCTCACATCAGGCTGCATCCTC TTCTGTCCCTGGCACCAGGCTTTGTTTACACTTGGAGCCACCTTGGTGTGGGTCACCGGGA CAGTGTACTCCTCTCCTGCCAGCCTCCCCTTCCCCGAGGTGTGGTGGCTGCAGTCTCAGGA AGAGCTTGGTACTTGTGGGGACTTCTGTTTTCTCCCTGTGGAGATCAGTGAAGACTGGGAG GAAAGCTGCTTCAACCTGAGTCCGGCTCTTCAGCAGGCTGCACAAGTGGAAGCAACTAAT TCTGGTGCTCAGGCTGGGCTCTCCACCCAAGTTAGGCCTGCTCTGGCCTAATGGATCTTAC TGTATGAGCAGGACGGCTGCATTGGATTGTACAACTGTTTTGTGATGCCCCCAGACACTGT CATCCTGGGCCGAGAAGAACCTGCTAGCTTGACATACCCCATGGGCTTATCCTTAGGTTTT GGAATTGGTCAACAGTGAGGCAGTCTCCCTTCCTGACCATTCTTCTCCACCCAGTCACAGA TAAGGGAATAACCTTGGCCATATATTTGCTCAATAAAGATTGAAGGAAGCATGGTCATAG TTGCCCTGGGTTCAGAGCATAATGCATATGTGAAGCATGGGGTGACATTCCTACTGTCATG GGTTTGGGATTTGTAACGGCAAATTCCTGCCCGACGACAGGGTGTCTTATGCAAAGGCTGA CTTGCCTGAACGCTAAGAACATGACTTCTGTCTGAGCTAAGCTGGCACCCATCCCAGGGCT CCTCTGGAGCTAATCCTTTAAGCAAAATGTGCTTGCCTTTTAAAGATCCCTGACCCCAGCTT TAGCTTTCTCCACCAGATAACCAGCTAATCCCAGGAATTTGCTGCCCCCCACCAGTGGCTT CTAGGGAAAGCAAGGACCTCACATGCCAGGTGCCCTAGTACTTGCTTAGTGAGCCATGTC ATCCTCCTTTCATTTTTGGATGGTGACAGCATTTTTCCCCTCTGTGCTGGATACAGACTTCT CCCAGGATCCTCTCTTTGGGAGCGAAGCCAGAGGATCCCTACAGCACTCAAGCTTCATGGT GGAATTAATTTCTGCCAGCTCTTTGTTGTCTGTCTCCTTAAATCCTTTTCCTGGTGTGCTTAT TATCCCTTTTGCAGTGAGTACAGTTTATTAAGTTGTCAGCCCTTTAATATTGGGGAAACTTA ATGAGTATAAATAGCAGGGAGCACATTGTAACAGCACAGTGTTTTGTTTTTTTCACCCGGT TGCTGTATGAGAATGGCTTTCAATCCTTTGTTTCTATGCCTACAGACAGAAAGCAAGATGT CTAATATTAGACATACAAGTTGCTGCCTGTTATAACGGTGAATTATACCTTTGTGCATGCCT AGGATGTTTGTTGTTTTAATTAGCTGCAATATATACGGCCTGTGTACACAGAATTTAATCA CTTCGGCAGGTTGAACAACTCCATGTAGATAAGAGCAAGTGTAGGCAAAGGTTTAGAAAA TGGACATAAAGTCAAAGAATGATGGCAGGTAGGATGAAGGAGAGATACTTAGGAAATCCT AAAAGAGGCGGCAAGAAGGTACCTCCCTGTGTAACTCACCTTCCCCCATGACAGTGAGTA AGAGACACTCACAGGCTATGAGGGTACACCCCTAGCTGAATGTTCTGTGTTGTTTCCTTAG ACCTGTGGTGTCCGCTGCAACAGCTACTAGCCACGTGTAGCTAATTACATTAAAATGAAAT AAAATTAAAAGCTCAGTTTCTCAGTTGCGCTAATCACATTTCAAGTGCTCAGCAGCCACCC GTGTCTACTACTACACAGTGCAGACACAGAACATATCATCACTGCAGATAGTTCTACTGGA CAATGTTACGCTAGAATAAACACCAAGGCAGTCAGTTAAGGCAGCTATGGTTTGGAAAGG CATACGGACAGAGTCTGCTTAGAAGAGATACAAGTTGTTAATAAAATTGATCCTGTTGATA GTAGTTTGTTTTTGTGGTGGGTGCTGTGAAGAGTAAACATTACTCAGTGGAAAGCTAAGTT CAGAAGGTACTTTGTTTTTCCTCCCTTGCCTTAAGTCCTTGGTATTTATAATCAATGCTGAA CCTTCTATTTCACTACCGCTCCCTGTTTTAGATATTCAGATTTAAAAGGTTTTCAAAGAATT ACTTTCTTCCATGTTCAAAGCTAGATTTTACTAAACACATGTATCACATTCATATATATTGT TTCTTGGCCCCACTGCCAAAGGAAGTCAGTCAGTAATTTCACAACCGTTATCAGAGTTTGG |
| | AAGCAGAAATAGCTGTTAACTAAAATCTCCCACTGCTCAGACTACTTTCTGCCCTAATGGC CATTACTATCCAGTCTGTATTGCTACAAGGGACCCACTGGTACCCCTTTTAGATTCTATCAA AAGGAACAGGGTTTTCCTAGAGGCAGGCAGCCTGGTGGTATGGCACAGCAGAAGCTTACT GCTAATGAAATGGGAACCTCCCCCTCCCTTGTGGTTTCAGCACAGAACCTGAATGCCAGGA AAAATTCCTGGGCCAAGAAGCTAAAGCTAAAGAAACCTTCCTTTTTTCAACGTTTTTTTTTC TTTCAAACTGTAGGGTCACTTTTGATTGAGGCAAAGGGGTCCTACTGTAAGTGGAAAAGAC TCACTCCCCTAACATAAGTTTTCACTGTGGTGGGATGGTGCCGCCCGATATGCTTGATATG CTTTTCCTTCCACATGTTAAGCTAGGAAACCTAACAGGATGTCAGCAGGGCAGTTAACTCT GGACTCAGAGCCCTCAAGGGCATGTGGCAGAACCTCATGGACATCACAAGACCATCAGTC TGAATCCAGGTCGTGGGGGCTGTCATAGCCGAACTCCTTCTGCACATCCAGAGGGTACTTG CTCCACATCCGCTGTCTGCTGCTGCCTCTTTCCTCCTCACTCAGGCTGTTGTAGTCAGCAGA GCCTAGAATGACATCCCGGGAGTGGATTCTAAATGTGATTTTCCTAGGCTACTGCAGGAGC CCCTTCTCTTCTCAGAAAGGTCTGTTTTTGTTCCCGATTGTAATGCAAAATCCTTGCTCAAT AAATAAAAAAGAATATAGAATTCTTTTTTTTTTAAAGAAGGAATCACTTTCCTATCATCTA AACCAAGTTCCTTCACACTGGAGTATTTTGTCACTTCTCCCCTCCGTGGAGTATTTTGTCAC TTCTCCCCTCCGTATAGGATTTTTTGTTGTTGTAAGAGTTGTAGTCATATTGTAAATATTTTT GTACCTTTCTCCTTTTAACGTGTTATTGACAAACCTCCCCAAAAGAATATGCAATTGTTTGA TTCATTTCTCTGTTATCAGACACCAATAAATTCTTTTTGTTGGGC |
| 215 | GCGGCGGCGGCGGCGCGACCGAGCATCCTGGCGGCGCCGGGCCACTGGGAGAGTTTATGT GGCCGAGGCAGACAAGTGGAATTAGGCCTTGCTGCAGGGGACTTCATTTCCTTCTCAGTAC TGGACCCATTTATGAGGAGGTGGCTTATGAAAGTGTGATGTTCGCGTATTTCTTGACAGGC CACAGCAAACACAGGTGTGCAGGAACCGTTTGTCATGGAAGCCAGGGAGCCTGGGAGGCC CACACCCACCTACCATCTTGTCCCTAACACCAGCCAGTCCCAGGTGGAAGAAGATGTCAGC TCGCCACCTCAAAGGTCCTCCGAAACTATGCAGCTGAAGAAGGAGATCTCCCTGCTGAAT GGGGTCAGCCTGGTGGTGGGCAACATGATCGGCTCAGGGATCTTTGTCTCACCCAAGGGT GTGCTGGTACACACTGCCTCCTATGGGATGTCACTGATTGTGTGGGCCATTGGTGGGCTCT TCTCTGTTGTGGGTGCCCTTTGTTATGCAGAGCTGGGGACCACCATCACCAAGTCGGGAGC CAGCTACGCTTATATTCTAGAGGCCTTTGGGGGCTTCATTGCCTTCATCCGCCTGTGGGTCT CACTGCTAGTTGTTGAGCCCACCGGTCAGGCCATCATCGCCATCACCTTTGCCAACTACAT CATCCAGCCGTCCTTCCCCAGCTGTGATCCCCCATACCTGGCCTGCCGTCTCCTGGCTGCTG CTTGCATATGTCTGCTGACATTTGTGAACTGTGCCTATGTCAAGTGGGGCACACGTGTGCA GGACACGTTCACTTACGCCAAGGTCGTAGCGCTCATTGCCATCATTGTCATGGGCCTTGTT AAACTGTGCCAGGGACACTCTGAGCACTTTCAGGACGCCTTTGAGGGTTCCTCCTGGGACA TGGGAAACCTCTCTCTTGCCCTCTACTCTGCCCTCTTCTCTTACTCAGGTTGGGACACCCTT AATTTTGTAACAGAAGAAATCAAAAACCCAGAAAGAAATTTGCCCTTGGCCATTGGGATT TCTATGCCAATTGTGACGCTCATCTACATCCTGACCAATGTGGCCTATTACACAGTGCTGA ACATTTCAGATGTCCTTAGCAGTGATGCTGTGGCTGTGACATTTGCTGACCAGACGTTTGG CATGTTCAGCTGGACCATCCCCATTGCTGTTGCCCTGTCCTGCTTTGGGGGCCTCAATGCAT CCATCTTTGCTTCATCAAGGTTGTTCTTCGTGGGCTCCCGGGAGGGCCACCTACCGGACCTT CTGTCCATGATCCACATTGAGCGTTTTACACCTATCCCTGCTTTACTGTTCAATTGCACCAT GGCACTCATCTACCTCATCGTGGAGGATGTTTTCCAGCTTATCAACTACTTCAGCTTCAGCT ACTGGTTCTTCGTGGGCCTGTCTGTTGTTGGACAGCTCTACCTCCGCTGGAAGGAGCCCAA GCGGCCCCGGCCTCTCAAGCTGAGCGTGTTTTTCCCCATCGTGTTCTGCATATGCTCCGTGT TTCTGGTGATAGTGCCCCTCTTCACTGACACCATTAATTCCCTCATTGGCATCGGGATTGCC CTTTCTGGAGTCCCTTTCTACTTCATGGGTGTTTACCTGCCAGAGTCCCGGAGGCCATTGTT TATTCGGAATGTCCTGGCTGCTATCACCAGAGGCACCCAGCAGCTTTGCTTTTGTGTCCTG ACTGAGCTTGATGTAGCCGAAGAAAAAAAGGATGAGAGGAAAACTGACTAGAGGTCAGA GGTGGCTTTCTGAGGCCTGGAAGGCAGGCCAACCAGCAAAATCCTGATAACAAGACTCTG TGGGCCCAACTCTCCTGAATTAAAGGAGCCTTTTGACCCAATCATATAGTGGGGCTCAGGG CCAGTGCTCACTCTTATTGGTAAGCTATAGGAGACTCAGGATCTGGGCCAACCTCAAGGTG GGGGCTTCAGAGGGTGGGGGGAAGATTGGGGAACGGGGGGAATGGTCATTTAGTTTTACT CCTGATAGGTAGATGCAGCTCTTACAGATATTTACTTGGTAAAGTGCAGTGGGGAAGAGG |
| | GAATGCTAGGTTGATAGGGCTGGTGGCTTCTGAATTTGGTATTTGAACTAGGAGTCCCTAT AGAGGGGCTGCTTTATGGGAAGTTTT 1CTCTGACCAGGTACAACACCTGACTTTAAAGGCC TGAAATGCTACCATTTCTTCCTCTGGCTCAAAATTCTTCCCTGGGGAGAGAGTTATATTCCC TTATTTATTGATATTTAGTCCAGAACACCAGTTCTAACGAAGCATGCGTGTCTCTTCATCTA CAGGATGCAATAGGCTGATTGTATTTAAAAATCAAAGTACCCAAAACTGAGTCCCTTTGGG CTCAGAAATGTCTGTGGTATTGGGTCAGACTCTGACCACAGGTTTTATGCTGTTTAGCACA ATTTCTATTGAGTCTTACCTGCAACAATGAACCTTAAAGATTTTTTTACTCACGTACCTGTT ACACTTTAGCATACAGATAGATCATAGATCACGTTACAAGCACTTGGCTCAGGTCCAGCAA GGACAGATGAACAAATTCCTGAGTCAGAAGTCTGTTAATATTGCTGTTTTGAAGGACAATC CTTTATTTTACTTGAGACCTTACATCTTTGTTCTAGCTGACAGTAAATCTCTGGGTTTCTGTT ACGAACTCTAAGAGGGCTGAAACTTCTGATATTCAGGTGGATCACCTGAATTCTCTCAGCT GTCAATGGCTTGGAGAACATCTCATGGGCCCAAGTCATCAAATAACCTGTTCCTCTCTGTA AGGGCAGTGTGAGGGACTGCTGTGCAGACCCAAGCAATCCCAACCTGGTGCTAGGTCATT TCACTTTTCTGAAAACCTCACATCAGGCTGCATCCTCTTCTGTCCCTGGCACCAGGCTTTGT TTACACTTGGAGCCACCTTGGTGTGGGTCACCGGGACAGTGTACTCCTCTCCTGCCAGCCT CCCCTTCCCCGAGGTGTGGTGGCTGCAGTCTCAGGAAGAGCTTGGTACTTGTGGGGACTTC TGTTTTCTCCCTGTGGAGATCAGTGAAGACTGGGAGGAAAGCTGCTTCAACCTGAGTCCGG CTCTTCAGCAGGCTGCACAAGTGGAAGCAACTAATTCTGGTGCTCAGGCTGGGCTCTCCAC CCAAGTTAGGCCTGCTCTGGCCTAATGGATCTTACTGTATGAGCAGGACGGCTGCATTGGA TTGTACAACTGTTTTGTGATGCCCCCAGACACTGTCATCCTGGGCCGAGAAGAACCTGCTA GCTTGACATACCCCATGGGCTTATCCTTAGGTTTTGGAATTGGTCAACAGTGAGGCAGTCT CCCTTCCTGACCATTCTTCTCCACCCAGTCACAGATAAGGGAATAACCTTGGCCATATATTT GCTCAATAAAGATTGAAGGAAGCATGGTCATAGTTGCCCTGGGTTCAGAGCATAATGCAT ATGTGAAGCATGGGGTGACATTCCTACTGTCATGGGTTTGGGATTTGTAACGGCAAATTCC TGCCCGACGACAGGGTGTCTTATGCAAAGGCTGACTTGCCTGAACGCTAAGAACATGACTT CTGTCTGAGCTAAGCTGGCACCCATCCCAGGGCTCCTCTGGAGCTAATCCTTTAAGCAAAA TGTGCTTGCCTTTTAAAGATCCCTGACCCCAGCTTTAGCTTTCTCCACCAGATAACCAGCTA ATCCCAGGAATTTGCTGCCCCCCACCAGTGGCTTCTAGGGAAAGCAAGGACCTCACATGCC AGGTGCCCTAGTACTTGCTTAGTGAGCCATGTCATCCTCCTTTCATTTTTGGATGGTGACAG CATTTTTCCCCTCTGTGCTGGATACAGACTTCTCCCAGGATCCTCTCTTTGGGAGCGAAGCC AGAGGATCCCTACAGCACTCAAGCTTCATGGTGGAATTAATTTCTGCCAGCTCTTTGTTGT CTGTCTCCTTAAATCCTTTTCCTGGTGTGCTTATTATCCCTTTTGCAGTGAGTACAGTTTATT AAGTTGTCAGCCCTTTAATATTGGGGAAACTTAATGAGTATAAATAGCAGGGAGCACATT GTAACAGCACAGTGTTTTGTTTTTTTCACCCGGTTGCTGTATGAGAATGGCTTTCAATCCTT TGTTTCTATGCCTACAGACAGAAAGCAAGATGTCTAATATTAGACATACAAGTTGCTGCCT GTTATAACGGTGAATTATACCTTTGTGCATGCCTAGGATGTTTGTTGTTTTAATTAGCTGCA ATATATACGGCCTGTGTACACAGAATTTAATCACTTCGGCAGGTTGAACAACTCCATGTAG ATAAGAGCAAGTGTAGGCAAAGGTTTAGAAAATGGACATAAAGTCAAAGAATGATGGCA GGTAGGATGAAGGAGAGATACTTAGGAAATCCTAAAAGAGGCGGCAAGAAGGTACCTCC CTGTGTAACTCACCTTCCCCCATGACAGTGAGTAAGAGACACTCACAGGCTATGAGGGTAC ACCCCTAGCTGAATGTTCTGTGTTGTTTCCTTAGACCTGTGGTGTCCGCTGCAACAGCTACT AGCCACGTGTAGCTAATTACATTAAAATGAAATAAAATTAAAAGCTCAGTTTCTCAGTTGC GCTAATCACATTTCAAGTGCTCAGCAGCCACCCGTGTCTACTACTACACAGTGCAGACACA GAACATATCATCACTGCAGATAGTTCTACTGGACAATGTTACGCTAGAATAAACACCAAG GCAGTCAGTTAAGGCAGCTATGGTTTGGAAAGGCATACGGACAGAGTCTGCTTAGAAGAG ATACAAGTTGTTAATAAAATTGATCCTGTTGATAGTAGTTTGTTTTTGTGGTGGGTGCTGTG AAGAGTAAACATTACTCAGTGGAAAGCTAAGTTCAGAAGGTACTTTGTTTTTCCTCCCTTG CCTTAAGTCCTTGGTATTTATAATCAATGCTGAACCTTCTATTTCACTACCGCTCCCTGTTTT AGATATTCAGATTTAAAAGGTTTTCAAAGAATTACTTTCTTCCATGTTCAAAGCTAGATTTT ACTAAACACATGTATCACATTCATATATATTGTTTCTTGGCCCCACTGCCAAAGGAAGTCA GTCAGTAATTTCACAACCGTTATCAGAGTTTGGAAGCAGAAATAGCTGTTAACTAAAATCT CCCACTGCTCAGACTACTTTCTGCCCTAATGGCCATTACTATCCAGTCTGTATTGCTACAAG GGACCCACTGGTACCCCTTTTAGATTCTATCAAAAGGAACAGGGTTTTCCTAGAGGCAGGC |
| | AGCCTGGTGGTATGGCACAGCAGAAGCTTACTGCTAATGAAATGGGAACCTCCCCCTCCCT TGTGGTTTCAGCACAGAACCTGAATGCCAGGAAAAATTCCTGGGCCAAGAAGCTAAAGCT AAAGAAACCTTCCTTTTTTCAACGTTTTTTTTTCTTTCAAACTGTAGGGTCACTTTTGATTGA GGCAAAGGGGTCCTACTGTAAGTGGAAAAGACTCACTCCCCTAACATAAGTTTTCACTGTG GTGGGATGGTGCCGCCCGATATGCTTGATATGCTTTTCCTTCCACATGTTAAGCTAGGAAA CCTAACAGGATGTCAGCAGGGCAGTTAACTCTGGACTCAGAGCCCTCAAGGGCATGTGGC AGAACCTCATGGACATCACAAGACCATCAGTCTGAATCCAGGTCGTGGGGGCTGTCATAG CCGAACTCCTTCTGCACATCCAGAGGGTACTTGCTCCACATCCGCTGTCTGCTGCTGCCTCT TTCCTCCTCACTCAGGCTGTTGTAGTCAGCAGAGCCTAGAATGACATCCCGGGAGTGGATT CTAAATGTGATTTTCCTAGGCTACTGCAGGAGCCCCTTCTCTTCTCAGAAAGGTCTGTTTTT GTTCCCGATTGTAATGCAAAATCCTTGCTCAATAAATAAAAAAGAATATAGAATTCTTTTT TTTTTAAAGAAGGAATCACTTTCCTATCATCTAAACCAAGTTCCTTCACACTGGAGTATTTT GTCACTTCTCCCCTCCGTGGAGTATTTTGTCACTTCTCCCCTCCGTATAGGATTTTTTGTTGT TGTAAGAGTTGTAGTCATATTGTAAATATTTTTGTACCTTTCTCCTTTTAACGTGTTATTGA CAAACCTCCCCAAAAGAATATGCAATTGTTTGATTCATTTCTCTGTTATCAGACACCAATA AATTCTTTTTGTTGGGC |
| 220 | GTCACACTGTGCAACCTTCCTCCCTTTCTTAAATGCTTGGGGCATTTGTCTGGCCTTCCCTT TTACTGCTGGCTGGGAAGGAGGAGCATCAGACCACAGATCCTGGAAGGCACTTCTCTCCCT GACTGCTGCTCACACTGCCGTGAGAACCTGCTTATATCCAGGACCAAGGAGGCAATGCCA GGAAGCTGGTGAAGGGTTTCCTCTCCTCCACCATGGTTGACAGCACTGAGTATGAAGTGGC CTCCCAGCCTGAGGTGGAAACCTCCCCTTTGGGTGATGGGGCCAGCCCAGGGCCGGAGCA GGTGAAGCTGAAGAAGGAGATCTCACTGCTTAACGGCGTGTGCCTGATTGTGGGGAACAT GATCGGCTCGGGCATCTTTGTTTCCCCCAAGGGTGTGCTCATATACAGTGCCTCCTTTGGTC TCTCTCTGGTCATCTGGGCTGTCGGGGGCCTCTTCTCCGTCTTTGGGGCCCTTTGTTATGCG GAACTGGGCACCACCATTAAGAAATCTGGGGCCAGCTATGCCTATATCCTGGAGGCCTTTG GAGGATTCCTTGCTTTCATCAGACTCTGGACCTCCCTGCTCATCATTGAGCCCACCAGCCA GGCCATCATTGCCATCACCTTTGCCAACTACATGGTACAGCCTCTCTTCCCGAGCTGCTTCG CCCCTTATGCTGCCAGCCGCCTGCTGGCTGCTGCCTGCATTTGTCTCTTAACCTTCATTAAC TGTGCCTATGTCAAATGGGGAACCCTGGTACAAGATATTTTCACCTATGCTAAAGTATTGG CACTGATCGCGGTCATCGTTGCAGGCATTGTTAGACTTGGCCAGGGAGCCTCTACTCATTT TGAGAATTCCTTTGAGGGTTCATCATTTGCAGTGGGTGACATTGCCCTGGCACTGTACTCA GCTCTGTTCTCCTACTCAGGCTGGGACACCCTCAACTATGTCACTGAAGAGATCAAGAATC CTGAGAGGAACCTGCCCCTCTCCATTGGCATCTCCATGCCCATTGTCACCATCATCTATATC TTGACCAATGTGGCCTATTATACTGTGCTAGACATGAGAGACATCTTGGCCAGTGATGCTG TTGCTGTGACTTTTGCAGATCAGATATTTGGAATATTTAACTGGATAATTCCACTGTCAGTT GCATTATCCTGTTTTGGTGGCCTCAATGCCTCCATTGTGGCTGCTTCTAGGCTTTTCTTTGTG GGCTCAAGAGAAGGCCATCTCCCTGATGCCATCTGCATGATCCATGTTGAGCGGTTCACAC CAGTGCCTTCTCTGCTCTTCAATGGTATCATGGCATTGATCTACTTGTGCGTGGAAGACATC TTCCAGCTCATTAACTACTACAGCTTCAGCTACTGGTTCTTTGTGGGGCTTTCTATTGTGGG TCAGCTTTATCTGCGCTGGAAGGAGCCTGATCGACCTCGTCCCCTCAAGCTCAGCGTTTTCT TCCCGATTGTCTTCTGCCTCTGCACCATCTTCCTGGTGGCTGTTCCACTTTACAGTGATACT ATCAACTCCCTCATCGGCATTGCCATTGCCCTCTCAGGCCTGCCCTTTTACTTCCTCATCAT CAGAGTGCCAGAACATAAGCGACCGCTTTACCTCCGAAGGATCGTGGGGTCTGCCACAAG GTACCTCCAGGTCCTGTGTATGTCAGTTGCTGCAGAAATGGATTTGGAAGATGGAGGAGA GATGCCCAAGCAACGGGATCCCAAATCTAACTAAACACCATCTGGAATCCTGATGTGGAA AGCAGGGGTTTCTGGTCTACTGGCTAGAGCTAAGGAAGTTGAAAAGGAAAGCTCACTTCT TTGGAGGCACCTGTCCAGAAGCCTGGCCTAGGCAGCTTCAACCTTTGAACTTACTTTTTGA AATGAAAAGTAATTTATTTGTTTTGCTACATACTGTTCCAGACTTTTAAAGGGGACAATGA AGGTGACTGTGGGGAGGAGCATGTCAGGTTTGGGCTTGGTTGTTTTAGAAGCACCTGGGTG TGCCTACCTACTCCTCTTTTCTTTTAAAAGGGCCCACAATGCTCCAATTTCCTGTCTCCTTTA GAGAGACATGAAACTATCACAGGTGCTGGATGACAATAAAAGTTTATGTTCCTAAA |
| 221 | CCCTTTTACTGCTGGCTGGGAAGGAGGAGCATCAGACCACAGATCCTGGAAGGCACTTCTC TCCCTGACTGCTGCTCACACTGCCGTGAGAACCTGCTTATATCCAGGACCAAGGAGGCAAT GCCAGGAAGCTGGTGAAGGGTTTCCTCTCCTCCACCATGGTTGACAGCACTGAGTATGAAG TGGCCTCCCAGCCTGAGGTGGAAACCTCCCCTTTGGGTGATGGGGCCAGCCCAGGGCCGG AGCAGGTGAAGCTGAAGAAGGAGATCTCACTGCTTAACGGCGTGTGCCTGATTGTGGGGA ACATGATCGGCTCGGGCATCTTTGTTTCCCCCAAGGGTGTGCTCATATACAGTGCCTCCTTT GGTCTCTCTCTGGTCATCTGGGCTGTCGGGGGCCTCTTCTCCGTCTTTGGGGCCCTTTGTTA TGCGGAACTGGGCACCACCATTAAGAAATCTGGGGCCAGCTATGCCTATATCCTGGAGGC CTTTGGAGGATTCCTTGCTTTCATCAGACTCTGGACCTCCCTGCTCATCATTGAGCCCACCA GCCAGGCCATCATTGCCATCACCTTTGCCAACTACATGGTACAGCCTCTCTTCCCGAGCTG CTTCGCCCCTTATGCTGCCAGCCGCCTGCTGGCTGCTGCCTGCATTTGTCTCTTAACCTTCA TTAACTGTGCCTATGTCAAATGGGGAACCCTGGTACAAGATATTTTCACCTATGCTAAAGT ATTGGCACTGATCGCGGTCATCGTTGCAGGCATTGTTAGACTTGGCCAGGGAGCCTCTACT CATTTTGAGAATTCCTTTGAGGGTTCATCATTTGCAGTGGGTGACATTGCCCTGGCACTGTA CTCAGCTCTGTTCTCCTACTCAGGCTGGGACACCCTCAACTATGTCACTGAAGAGATCAAG AATCCTGAGAGGAACCTGCCCCTCTCCATTGGCATCTCCATGCCCATTGTCACCATCATCT ATATCTTGACCAATGTGGCCTATTATACTGTGCTAGACATGAGAGACATCTTGGCCAGTGA TGCTGTTGCTGTGACTTTTGCAGATCAGATATTTGGAATATTTAACTGGATAATTCCACTGT CAGTTGCATTATCCTGTTTTGGTGGCCTCAATGCCTCCATTGTGGCTGCTTCTAGGCTTTTC TTTGTGGGCTCAAGAGAAGGCCATCTCCCTGATGCCATCTGCATGATCCATGTTGAGCGGT TCACACCAGTGCCTTCTCTGCTCTTCAATGGTATCATGGCATTGATCTACTTGTGCGTGGAA GACATCTTCCAGCTCATTAACTACTACAGCTTCAGCTACTGGTTCTTTGTGGGGCTTTCTAT TGTGGGTCAGCTTTATCTGCGCTGGAAGGAGCCTGATCGACCTCGTCCCCTCAAGCTCAGC GTTTTCTTCCCGATTGTCTTCTGCCTCTGCACCATCTTCCTGGTGGCTGTTCCACTTTACAGT GATACTATCAACTCCCTCATCGGCATTGCCATTGCCCTCTCAGGCCTGCCCTTTTACTTCCT CATCATCAGAGTGCCAGAACATAAGCGACCGCTTTACCTCCGAAGGATCGTGGGGTCTGC CACAAGGTACCTCCAGGTCCTGTGTATGTCAGTTGCTGCAGAAATGGATTTGGAAGATGGA GGAGAGATGCCCAAGCAACGGGATCCCAAATCTAACTAAACACCATCTGGAATCCTGATG TGGAAAGCAGGGGTTTCTGGTCTACTGGCTAGAGCTAAGGAAGTTGAAAAGGAAAGCTCA CTTCTTTGGAGGCACCTGTCCAGAAGCCTGGCCTAGGCAGCTTCAACCTTTGAACTTACTTT TTGAAATGAAAAGTAATTTATTTGTTTTGCTACATACTGTTCCAGACTTTTAAAGGGGACA ATGAAGGTGACTGTGGGGAGGAGCATGTCAGGTTTGGGCTTGGTTGTTTTAGAAGCACCTG GGTGTGCCTACCTACTCCTCTTTTCTTTTAAAAGGGCCCACAATGCTCCAATTTCCTGTCTC CTTTAGAGAGACATGAAACTATCACAGGTGCTGGATGACAATAAAAGTTTATGTTCCTAAA |
| 222 | AGTCCCCGTTACCCTCTGC11111CTGCTCCTCAGAGTCAACAGCTGTTGCAGCATGAGCGA TACGCTTGGTTCTCCTAACTAGCACCTTCCCCTCTCCCCTGACTCAGCTGGTAGCCCCTCCT CCCCGCACCTGCCCAAAGGTCACTGGACAGGCATTTGTCTGGCCTTCCCTTTTACTGCTGG CTGGGAAGGAGGAGCATCAGACCACAGATCCTGGAAGGCACTTCTCTCCCTGACTGCTGC TCACACTGCCGTGAGAACCTGCTTATATCCAGGACCAAGGAGGCAATGCCAGGAAGCTGG TGAAGGGTTTCCTCTCCTCCACCATGGTTGACAGCACTGAGTATGAAGTGGCCTCCCAGCC TGAGGTGGAAACCTCCCCTTTGGGTGATGGGGCCAGCCCAGGGCCGGAGCAGGTGAAGCT GAAGAAGGAGATCTCACTGCTTAACGGCGTGTGCCTGATTGTGGGGAACATGATCGGCTC GGGCATCTTTGTTTCCCCCAAGGGTGTGCTCATATACAGTGCCTCCTTTGGTCTCTCTCTGG TCATCTGGGCTGTCGGGGGCCTCTTCTCCGTCTTTGGGGCCCTTTGTTATGCGGAACTGGGC ACCACCATTAAGAAATCTGGGGCCAGCTATGCCTATATCCTGGAGGCCTTTGGAGGATTCC TTGCTTTCATCAGACTCTGGACCTCCCTGCTCATCATTGAGCCCACCAGCCAGGCCATCATT GCCATCACCTTTGCCAACTACATGGTACAGCCTCTCTTCCCGAGCTGCTTCGCCCCTTATGC TGCCAGCCGCCTGCTGGCTGCTGCCTGCATTTGTCTCTTAACCTTCATTAACTGTGCCTATG TCAAATGGGGAACCCTGGTACAAGATATTTTCACCTATGCTAAAGTATTGGCACTGATCGC GGTCATCGTTGCAGGCATTGTTAGACTTGGCCAGGGAGCCTCTACTCATTTTGAGAATTCC TTTGAGGGTTCATCATTTGCAGTGGGTGACATTGCCCTGGCACTGTACTCAGCTCTGTTCTC CTACTCAGGCTGGGACACCCTCAACTATGTCACTGAAGAGATCAAGAATCCTGAGAGGAA |
| | CCTGCCCCTCTCCATTGGCATCTCCATGCCCATTGTCACCATCATCTATATCTTGACCAATG TGGCCTATTATACTGTGCTAGACATGAGAGACATCTTGGCCAGTGATGCTGTTGCTGTGAC TTTTGCAGATCAGATATTTGGAATATTTAACTGGATAATTCCACTGTCAGTTGCATTATCCT GTTTTGGTGGCCTCAATGCCTCCATTGTGGCTGCTTCTAGGCTTTTCTTTGTGGGCTCAAGA GAAGGCCATCTCCCTGATGCCATCTGCATGATCCATGTTGAGCGGTTCACACCAGTGCCTT CTCTGCTCTTCAATGGTATCATGGCATTGATCTACTTGTGCGTGGAAGACATCTTCCAGCTC ATTAACTACTACAGCTTCAGCTACTGGTTCTTTGTGGGGCTTTCTATTGTGGGTCAGCTTTA TCTGCGCTGGAAGGAGCCTGATCGACCTCGTCCCCTCAAGCTCAGCGTTTTCTTCCCGATT GTCTTCTGCCTCTGCACCATCTTCCTGGTGGCTGTTCCACTTTACAGTGATACTATCAACTC CCTCATCGGCATTGCCATTGCCCTCTCAGGCCTGCCCTTTTACTTCCTCATCATCAGAGTGC CAGAACATAAGCGACCGCTTTACCTCCGAAGGATCGTGGGGTCTGCCACAAGGTACCTCC AGGTCCTGTGTATGTCAGTTGCTGCAGAAATGGATTTGGAAGATGGAGGAGAGATGCCCA AGCAACGGGATCCCAAATCTAACTAAACACCATCTGGAATCCTGATGTGGAAAGCAGGGG TTTCTGGTCTACTGGCTAGAGCTAAGGAAGTTGAAAAGGAAAGCTCACTTCTTTGGAGGCA CCTGTCCAGAAGCCTGGCCTAGGCAGCTTCAACCTTTGAACTTACTTTTTGAAATGAAAAG TAATTTATTTGTTTTGCTACATACTGTTCCAGACTTTTAAAGGGGACAATGAAGGTGACTGT GGGGAGGAGCATGTCAGGTTTGGGCTTGGTTGTTTTAGAAGCACCTGGGTGTGCCTACCTA CTCCTCTTTTCTTTTAAAAGGGCCCACAATGCTCCAATTTCCTGTCTCCTTTAGAGAGACAT GAAACTATCACAGGTGCTGGATGACAATAAAAGTTTATGTTCCTAAA |
| 227 | GCATTGCGGCTTGGTTTTCTCACCCAGTGCATGTGGCAGGAGCGGTGAGATCACTGCCTCA CGGCGATCCTGGACTGACGGTCACGACTGCCTACCCTCTAACCCTGTTCTGAGCTGCCCCT TGCCCACACACCCCAAACCTGTGTGCAGGATCCGCCTCCATGGAGCTACAGCCTCCTGAAG CCTCGATCGCCGTCGTGTCGATTCCGCGCCAGTTGCCTGGCTCACATTCGGAGGCTGGTGT CCAGGGTCTCAGCGCGGGGGACGACTCAGAGACGGGGTCTGACTGTGTTACCCAGGCTGG TCTTCAACTCTTGGCCTCAAGTGATCCTCCTGCCTTAGCTTCCAAGAATGCTGAGGTTACAG TAGAAACGGGGTTTCACCATGTTAGCCAGGCTGATATTGAATTCCTGACCTCAATTGATCC GACTGCCTCGGCCTCCGGAAGTGCTGGGATTACAGGCACCATGAGCCAGGACACCGAGGT GGATATGAAGGAGGTGGAGCTGAATGAGTTAGAGCCCGAGAAGCAGCCGATGAACGCGG CGTCTGGGGCGGCCATGTCCCTGGCGGGAGCCGAGAAGAATGGTCTGGTGAAGATCAAGG TGGCGGAAGACGAGGCGGAGGCGGCAGCCGCGGCTAAGTTCACGGGCCTGTCCAAGGAG GAGCTGCTGAAGGTGGCAGGCAGCCCCGGCTGGGTACGCACCCGCTGGGCACTGCTGCTG CTCTTCTGGCTCGGCTGGCTCGGCATGCTTGCTGGTGCCGTGGTCATAATCGTGCGAGCGC CGCGTTGTCGCGAGCTACCGGCGCAGAAGTGGTGGCACACGGGCGCCCTCTACCGCATCG GCGACCTTCAGGCCTTCCAGGGCCACGGCGCGGGCAACCTGGCGGGTCTGAAGGGGCGTC TCGATTACCTGAGCTCTCTGAAGGTGAAGGGCCTTGTGCTGGGTCCAATTCACAAGAACCA GAAGGATGATGTCGCTCAGACTGACTTGCTGCAGATCGACCCCAATTTTGGCTCCAAGGAA GATTTTGACAGTCTCTTGCAATCGGCTAAAAAAAAGAGCATCCGTGTCATTCTGGACCTTA CTCCCAACTACCGGGGTGAGAACTCGTGGTTCTCCACTCAGGTTGACACTGTGGCCACCAA GGTGAAGGATGCTCTGGAGTTTTGGCTGCAAGCTGGCGTGGATGGGTTCCAGGTTCGGGA CATAGAGAATCTGAAGGATGCATCCTCATTCTTGGCTGAGTGGCAAAATATCACCAAGGG CTTCAGTGAAGACAGGCTCTTGATTGCGGGGACTAACTCCTCCGACCTTCAGCAGATCCTG AGCCTACTCGAATCCAACAAAGACTTGCTGTTGACTAGCTCATACCTGTCTGATTCTGGTT CTACTGGGGAGCATACAAAATCCCTAGTCACACAGTATTTGAATGCCACTGGCAATCGCTG GTGCAGCTGGAGTTTGTCTCAGGCAAGGCTCCTGACTTCCTTCTTGCCGGCTCAACTTCTCC GACTCTACCAGCTGATGCTCTTCACCCTGCCAGGGACCCCTGTTTTCAGCTACGGGGATGA GATTGGCCTGGATGCAGCTGCCCTTCCTGGACAGCCTATGGAGGCTCCAGTCATGCTGTGG GATGAGTCCAGCTTCCCTGACATCCCAGGGGCTGTAAGTGCCAACATGACTGTGAAGGGC CAGAGTGAAGACCCTGGCTCCCTCCTTTCCTTGTTCCGGCGGCTGAGTGACCAGCGGAGTA AGGAGCGCTCCCTACTGCATGGGGACTTCCACGCGTTCTCCGCTGGGCCTGGACTCTTCTC CTATATCCGCCACTGGGACCAGAATGAGCGTTTTCTGGTAGTGCTTAACTTTGGGGATGTG GGCCTCTCGGCTGGACTGCAGGCCTCCGACCTGCCTGCCAGCGCCAGCCTGCCAGCCAAG GCTGACCTCCTGCTCAGCACCCAGCCAGGCCGTGAGGAGGGCTCCCCTCTTGAGCTGGAAC |
| | GCCTGAAACTGGAGCCTCACGAAGGGCTGCTGCTCCGCTTCCCCTACGCGGCCTGACTTCA GCCTGACATGGACCCACTACCCTTCTCCTTTCCTTCCCAGGCCCTTTGGCTTCTGATTTTTCT CTTTTTTAAAAACAAACAAACAAACTGTTGCAGATTATGAGTGAACCCCCAAATAGGGTGT TTTCTGCCTTCAAATAAAAGTCACCCCTGCATGGTGAA |
| 228 | GCATTGCGGCTTGGTTTTCTCACCCAGTGCATGTGGCAGGAGCGGTGAGATCACTGCCTCA CGGCGATCCTGGACTGACGGTCACGACTGCCTACCCTCTAACCCTGTTCTGAGCTGCCCCT TGCCCACACACCCCAAACCTGTGTGCAGGATCCGCCTCCATGGAGCTACAGCCTCCTGAAG CCTCGATCGCCGTCGTGTCGATTCCGCGCCAGTTGCCTGGCTCACATTCGGAGGCTGGTGT CCAGGGTCTCAGCGCGGGGGACGACTCAGGCACCATGAGCCAGGACACCGAGGTGGATAT GAAGGAGGTGGAGCTGAATGAGTTAGAGCCCGAGAAGCAGCCGATGAACGCGGCGTCTG GGGCGGCCATGTCCCTGGCGGGAGCCGAGAAGAATGGTCTGGTGAAGATCAAGGTGGCGG AAGACGAGGCGGAGGCGGCAGCCGCGGCTAAGTTCACGGGCCTGTCCAAGGAGGAGCTG CTGAAGGTGGCAGGCAGCCCCGGCTGGGTACGCACCCGCTGGGCACTGCTGCTGCTCTTCT GGCTCGGCTGGCTCGGCATGCTTGCTGGTGCCGTGGTCATAATCGTGCGAGCGCCGCGTTG TCGCGAGCTACCGGCGCAGAAGTGGTGGCACACGGGCGCCCTCTACCGCATCGGCGACCT TCAGGCCTTCCAGGGCCACGGCGCGGGCAACCTGGCGGGTCTGAAGGGGCGTCTCGATTA CCTGAGCTCTCTGAAGGTGAAGGGCCTTGTGCTGGGTCCAATTCACAAGAACCAGAAGGA TGATGTCGCTCAGACTGACTTGCTGCAGATCGACCCCAATTTTGGCTCCAAGGAAGATTTT GACAGTCTCTTGCAATCGGCTAAAAAAAAGAGCATCCGTGTCATTCTGGACCTTACTCCCA ACTACCGGGGTGAGAACTCGTGGTTCTCCACTCAGGTTGACACTGTGGCCACCAAGGTGA AGGATGCTCTGGAGTTTTGGCTGCAAGCTGGCGTGGATGGGTTCCAGGTTCGGGACATAG AGAATCTGAAGGATGCATCCTCATTCTTGGCTGAGTGGCAAAATATCACCAAGGGCTTCAG TGAAGACAGGCTCTTGATTGCGGGGACTAACTCCTCCGACCTTCAGCAGATCCTGAGCCTA CTCGAATCCAACAAAGACTTGCTGTTGACTAGCTCATACCTGTCTGATTCTGGTTCTACTGG GGAGCATACAAAATCCCTAGTCACACAGTATTTGAATGCCACTGGCAATCGCTGGTGCAG CTGGAGTTTGTCTCAGGCAAGGCTCCTGACTTCCTTCTTGCCGGCTCAACTTCTCCGACTCT ACCAGCTGATGCTCTTCACCCTGCCAGGGACCCCTGTTTTCAGCTACGGGGATGAGATTGG CCTGGATGCAGCTGCCCTTCCTGGACAGCCTATGGAGGCTCCAGTCATGCTGTGGGATGAG TCCAGCTTCCCTGACATCCCAGGGGCTGTAAGTGCCAACATGACTGTGAAGGGCCAGAGT GAAGACCCTGGCTCCCTCCTTTCCTTGTTCCGGCGGCTGAGTGACCAGCGGAGTAAGGAGC GCTCCCTACTGCATGGGGACTTCCACGCGTTCTCCGCTGGGCCTGGACTCTTCTCCTATATC CGCCACTGGGACCAGAATGAGCGTTTTCTGGTAGTGCTTAACTTTGGGGATGTGGGCCTCT CGGCTGGACTGCAGGCCTCCGACCTGCCTGCCAGCGCCAGCCTGCCAGCCAAGGCTGACC TCCTGCTCAGCACCCAGCCAGGCCGTGAGGAGGGCTCCCCTCTTGAGCTGGAACGCCTGA AACTGGAGCCTCACGAAGGGCTGCTGCTCCGCTTCCCCTACGCGGCCTGACTTCAGCCTGA CATGGACCCACTACCCTTCTCCTTTCCTTCCCAGGCCCTTTGGCTTCTGATTTTTCTCTTTTT TAAAAACAAACAAACAAACTGTTGCAGATTATGAGTGAACCCCCAAATAGGGTGTTTTCT GCCTTCAAATAAAAGTCACCCCTGCATGGTGAA |
| 229 | AGATGCAGTAGCCGAAACTGCGCGGAGGCACAGAGGCCGGGGAGAGCGTTCTGGGTCCG AGGGTCCAGGTAGGGGTTGAGCCACCATCTGACCGCAAGCTGCGTCGTGTCGCCGGTTCTG CAGGCACCATGAGCCAGGACACCGAGGTGGATATGAAGGAGGTGGAGCTGAATGAGTTA GAGCCCGAGAAGCAGCCGATGAACGCGGCGTCTGGGGCGGCCATGTCCCTGGCGGGAGCC GAGAAGAATGGTCTGGTGAAGATCAAGGTGGCGGAAGACGAGGCGGAGGCGGCAGCCGC GGCTAAGTTCACGGGCCTGTCCAAGGAGGAGCTGCTGAAGGTGGCAGGCAGCCCCGGCTG GGTACGCACCCGCTGGGCACTGCTGCTGCTCTTCTGGCTCGGCTGGCTCGGCATGCTTGCT GGTGCCGTGGTCATAATCGTGCGAGCGCCGCGTTGTCGCGAGCTACCGGCGCAGAAGTGG TGGCACACGGGCGCCCTCTACCGCATCGGCGACCTTCAGGCCTTCCAGGGCCACGGCGCG GGCAACCTGGCGGGTCTGAAGGGGCGTCTCGATTACCTGAGCTCTCTGAAGGTGAAGGGC CTTGTGCTGGGTCCAATTCACAAGAACCAGAAGGATGATGTCGCTCAGACTGACTTGCTGC AGATCGACCCCAATTTTGGCTCCAAGGAAGATTTTGACAGTCTCTTGCAATCGGCTAAAAA AAAGAGCATCCGTGTCATTCTGGACCTTACTCCCAACTACCGGGGTGAGAACTCGTGGTTC |
| | TCCACTCAGGTTGACACTGTGGCCACCAAGGTGAAGGATGCTCTGGAGTTTTGGCTGCAAG CTGGCGTGGATGGGTTCCAGGTTCGGGACATAGAGAATCTGAAGGATGCATCCTCATTCTT GGCTGAGTGGCAAAATATCACCAAGGGCTTCAGTGAAGACAGGCTCTTGATTGCGGGGAC TAACTCCTCCGACCTTCAGCAGATCCTGAGCCTACTCGAATCCAACAAAGACTTGCTGTTG ACTAGCTCATACCTGTCTGATTCTGGTTCTACTGGGGAGCATACAAAATCCCTAGTCACAC AGTATTTGAATGCCACTGGCAATCGCTGGTGCAGCTGGAGTTTGTCTCAGGCAAGGCTCCT GACTTCCTTCTTGCCGGCTCAACTTCTCCGACTCTACCAGCTGATGCTCTTCACCCTGCCAG GGACCCCTGTTTTCAGCTACGGGGATGAGATTGGCCTGGATGCAGCTGCCCTTCCTGGACA GCCTATGGAGGCTCCAGTCATGCTGTGGGATGAGTCCAGCTTCCCTGACATCCCAGGGGCT GTAAGTGCCAACATGACTGTGAAGGGCCAGAGTGAAGACCCTGGCTCCCTCCTTTCCTTGT TCCGGCGGCTGAGTGACCAGCGGAGTAAGGAGCGCTCCCTACTGCATGGGGACTTCCACG CGTTCTCCGCTGGGCCTGGACTCTTCTCCTATATCCGCCACTGGGACCAGAATGAGCGTTTT CTGGTAGTGCTTAACTTTGGGGATGTGGGCCTCTCGGCTGGACTGCAGGCCTCCGACCTGC CTGCCAGCGCCAGCCTGCCAGCCAAGGCTGACCTCCTGCTCAGCACCCAGCCAGGCCGTG AGGAGGGCTCCCCTCTTGAGCTGGAACGCCTGAAACTGGAGCCTCACGAAGGGCTGCTGC TCCGCTTCCCCTACGCGGCCTGACTTCAGCCTGACATGGACCCACTACCCTTCTCCTTTCCT TCCCAGGCCCTTTGGCTTCTGATTTTTCTCTTTTTTAAAAACAAACAAACAAACTGTTGCAG ATTATGAGTGAACCCCCAAATAGGGTGTTTTCTGCCTTCAAATAAAAGTCACCCCTGCATG GTGAA |
| 230 | GCATTGCGGCTTGGTTTTCTCACCCAGTGCATGTGGCAGGAGCGGTGAGATCACTGCCTCA CGGCGATCCTGGACTGACGGTCACGACTGCCTACCCTCTAACCCTGTTCTGAGCTGCCCCT TGCCCACACACCCCAAACCTGTGTGCAGGATCCGCCTCCATGGAGCTACAGCCTCCTGAAG CCTCGATCGCCGTCGTGTCGATTCCGCGCCAGTTGCCTGGCTCACATTCGGAGGCTGGTGT CCAGGGTCTCAGCGCGGGGGACGACTCAGAGTTGGGGTCTCACTGTGTTGCCCAGACTGG TCTCGAACTCTTGGCCTCAGGTGATCCTCTTCCCTCAGCTTCCCAGAATGCCGAGATGATA GAGACGGGGTCTGACTGTGTTACCCAGGCTGGTCTTCAACTCTTGGCCTCAAGTGATCCTC CTGCCTTAGCTTCCAAGAATGCTGAGGTTACAGGCACCATGAGCCAGGACACCGAGGTGG ATATGAAGGAGGTGGAGCTGAATGAGTTAGAGCCCGAGAAGCAGCCGATGAACGCGGCG TCTGGGGCGGCCATGTCCCTGGCGGGAGCCGAGAAGAATGGTCTGGTGAAGATCAAGGTG GCGGAAGACGAGGCGGAGGCGGCAGCCGCGGCTAAGTTCACGGGCCTGTCCAAGGAGGA GCTGCTGAAGGTGGCAGGCAGCCCCGGCTGGGTACGCACCCGCTGGGCACTGCTGCTGCT CTTCTGGCTCGGCTGGCTCGGCATGCTTGCTGGTGCCGTGGTCATAATCGTGCGAGCGCCG CGTTGTCGCGAGCTACCGGCGCAGAAGTGGTGGCACACGGGCGCCCTCTACCGCATCGGC GACCTTCAGGCCTTCCAGGGCCACGGCGCGGGCAACCTGGCGGGTCTGAAGGGGCGTCTC GATTACCTGAGCTCTCTGAAGGTGAAGGGCCTTGTGCTGGGTCCAATTCACAAGAACCAG AAGGATGATGTCGCTCAGACTGACTTGCTGCAGATCGACCCCAATTTTGGCTCCAAGGAAG ATTTTGACAGTCTCTTGCAATCGGCTAAAAAAAAGAGCATCCGTGTCATTCTGGACCTTAC TCCCAACTACCGGGGTGAGAACTCGTGGTTCTCCACTCAGGTTGACACTGTGGCCACCAAG GTGAAGGATGCTCTGGAGTTTTGGCTGCAAGCTGGCGTGGATGGGTTCCAGGTTCGGGAC ATAGAGAATCTGAAGGATGCATCCTCATTCTTGGCTGAGTGGCAAAATATCACCAAGGGC TTCAGTGAAGACAGGCTCTTGATTGCGGGGACTAACTCCTCCGACCTTCAGCAGATCCTGA GCCTACTCGAATCCAACAAAGACTTGCTGTTGACTAGCTCATACCTGTCTGATTCTGGTTCT ACTGGGGAGCATACAAAATCCCTAGTCACACAGTATTTGAATGCCACTGGCAATCGCTGGT GCAGCTGGAGTTTGTCTCAGGCAAGGCTCCTGACTTCCTTCTTGCCGGCTCAACTTCTCCGA CTCTACCAGCTGATGCTCTTCACCCTGCCAGGGACCCCTGTTTTCAGCTACGGGGATGAGA TTGGCCTGGATGCAGCTGCCCTTCCTGGACAGCCTATGGAGGCTCCAGTCATGCTGTGGGA TGAGTCCAGCTTCCCTGACATCCCAGGGGCTGTAAGTGCCAACATGACTGTGAAGGGCCA GAGTGAAGACCCTGGCTCCCTCCTTTCCTTGTTCCGGCGGCTGAGTGACCAGCGGAGTAAG GAGCGCTCCCTACTGCATGGGGACTTCCACGCGTTCTCCGCTGGGCCTGGACTCTTCTCCT ATATCCGCCACTGGGACCAGAATGAGCGTTTTCTGGTAGTGCTTAACTTTGGGGATGTGGG CCTCTCGGCTGGACTGCAGGCCTCCGACCTGCCTGCCAGCGCCAGCCTGCCAGCCAAGGCT GACCTCCTGCTCAGCACCCAGCCAGGCCGTGAGGAGGGCTCCCCTCTTGAGCTGGAACGC |
| | CTGAAACTGGAGCCTCACGAAGGGCTGCTGCTCCGCTTCCCCTACGCGGCCTGACTTCAGC CTGACATGGACCCACTACCCTTCTCCTTTCCTTCCCAGGCCCTTTGGCTTCTGATTTTTCTCT TTTTTAAAAACAAACAAACAAACTGTTGCAGATTATGAGTGAACCCCCAAATAGGGTGTTT TCTGCCTTCAAATAAAAGTCACCCCTGCATGGTGAA |
| 234 | GCCATTTCTAGGGTTGGACCGTGCAGGCACGGGCGGTCAGCTGGGCCGCAGCTCCTCCGG CTCTGCAGGGTCACGGAGGAAGTCTCCTGGAACCAGCAGGAGGAAACATGGGGGATACTG GCCTGAGAAAGCGGAGAGAGGATGAGAAGTCGATCCAGAGCCAAGAGCCTAAGACCACC AGTCTCCAAAAGGAGCTGGGCCTCATCAGTGGCATCTCCATCATCGTGGGCACCATCATTG GCTCTGGGATCTTCGTTTCCCCCAAGTCTGTGCTCAGCAACACGGAAGCTGTGGGGCCCTG CCTCATCATATGGGCGGCTTGCGGGGTCCTCGCGACGCTGGGTGCCCTGTGCTTTGCGGAG CTTGGCACAATGATCACCAAGTCAGGGGGAGAGTATCCCTACCTGATGGAGGCCTACGGG CCCATCCCCGCCTACCTCTTCTCCTGGGCCAGCCTGATCGTCATTAAGCCCACGTCCTTCGC CATCATCTGCCTCAGCTTCTCCGAGTATGTGTGTGCGCCCTTCTATGTGGGCTGCAAGCCTC CTCAAATCGTTGTGAAATGCCTGGCCGCCGCCGCCATCTTGTTCATCTCGACAGTGAACTC ACTGAGCGTGCGGCTGGGAAGCTACGTCCAGAACATCTTCACCGCGGCCAAGCTGGTGAT CGTGGCCATCATCATCATCAGCGGGCTGGTGCTCCTGGCCCAAGGAAACACAAAGAATTTT GATAATTCTTTCGAGGGCGCCCAGCTGTCTGTGGGAGCCATCAGCCTGGCGTTTTACAATG GACTCTGGGCCTATGATGGATGGAATCAACTCAATTACATCACAGAAGAACTTAGAAACC CTTACAGAAACCTGCCTTTGGCCATTATCATCGGGATCCCCCTGGTGACGGCGTGCTACAT CCTCATGAACGTGTCCTACTTCACCGTGATGACTGCCACCGAACTCCTGCAGTCCCAGGCG GTGGCTGTGACATTTGGTGACCGTGTTCTCTATCCTGCTTCTTGGATCGTTCCACIIIIIGT GGCATTTTCAACCATCGGTGCTGCTAACGGGACCTGCTTCACAGCGGGCAGACTCATTTAC GTGGCGGGCCGGGAGGGTCACATGCTCAAAGTGCTTTCTTACATCAGCGTCAGGCGCCTCA CTCCAGCCCCCGCCATCATCTTTTATGGTATCATAGCAACGATTTATATCATCCCTGGTGAC ATAAACTCGTTAGTCAATTATTTCAGCTTTGCCGCATGGCTGTTTTATGGCCTGACGATTCT AGGACTCATCGTGATGAGATTTACAAGGAAAGAGCTGGAAAGGCCTATCAAGGTGCCCGT AGTCATTCCCGTCTTGATGACACTCATCTCTGTGTTTTTGGTTCTGGCTCCAATCATCAGCA AGCCCACCTGGGAGTACCTCTACTGTGTGCTGTTTATATTAAGCGGCCTTTTATTTTACTTC CTGTTTGTCCACTACAAGTTTGGATGGGCTCAGAAAATCTCAAAGCCGATTACCATGCACC TTCAGATGCTAATGGAAGTGGTCCCACCGGAGGAAGACCCTGAGTAACAAGCTCCGTCTC TTGTAGCCAAGTCAGCTGAATTTATTTTCTTAAGCAATATTTGTGGTTATTTCTTCCTTTTTT TCTTACGAATAAAATATACTCAGATGTTTAAAA |
| 235 | GCCATTTCTAGGGTTGGACCGTGCAGGCACGGGCGGTCAGCTGGGCCGCAGCTCCTCCGG CTCTGCAGGGTCACGGAGGAAGGTAAGTAAGCCAGCTCCCCTAGTCCAGGCCGAGCTTGC ACTTGCGTCTTGTCTGCTGCTGCTGAACCAAGATTTAGCTGTGCGCCCTCCTTGCAGTCTCC TGGAACCAGCAGGAGGAAACATGGGGGATACTGGCCTGAGAAAGCGGAGAGAGGATGAG AAGTCGATCCAGAGCCAAGAGCCTAAGACCACCAGTCTCCAAAAGGAGCTGGGCCTCATC AGTGGCATCTCCATCATCGTGGGCACCATCATTGGCTCTGGGATCTTCGTTTCCCCCAAGTC TGTGCTCAGCAACACGGAAGCTGTGGGGCCCTGCCTCATCATATGGGCGGCTTGCGGGGTC CTCGCGACGCTGGGTGCCCTGTGCTTTGCGGAGCTTGGCACAATGATCACCAAGTCAGGGG GAGAGTATCCCTACCTGATGGAGGCCTACGGGCCCATCCCCGCCTACCTCTTCTCCTGGGC CAGCCTGATCGTCATTAAGCCCACGTCCTTCGCCATCATCTGCCTCAGCTTCTCCGAGTATG TGTGTGCGCCCTTCTATGTGGGCTGCAAGCCTCCTCAAATCGTTGTGAAATGCCTGGCCGC CGCCGCCATCTTGTTCATCTCGACAGTGAACTCACTGAGCGTGCGGCTGGGAAGCTACGTC CAGAACATCTTCACCGCGGCCAAGCTGGTGATCGTGGCCATCATCATCATCAGCGGGCTGG TGCTCCTGGCCCAAGGAAACACAAAGAATTTTGATAATTCTTTCGAGGGCGCCCAGCTGTC TGTGGGAGCCATCAGCCTGGCGTTTTACAATGGACTCTGGGCCTATGATGGATGGAATCAA CTCAATTACATCACAGAAGAACTTAGAAACCCTTACAGAAACCTGCCTTTGGCCATTATCA TCGGGATCCCCCTGGTGACGGCGTGCTACATCCTCATGAACGTGTCCTACTTCACCGTGAT GACTGCCACCGAACTCCTGCAGTCCCAGGCGGTGGCTGTGACATTTGGTGACCGTGTTCTC TATCCTGCTTCTTGGATCGTTCCACTTTTTGTGGCATTTTCAACCATCGGTGCTGCTAACGG |
| | GACCTGCTTCACAGCGGGCAGACTCATTTACGTGGCGGGCCGGGAGGGTCACATGCTCAA AGTGCTTTCTTACATCAGCGTCAGGCGCCTCACTCCAGCCCCCGCCATCATCTTTTATGGTA TCATAGCAACGATTTATATCATCCCTGGTGACATAAACTCGTTAGTCAATTATTTCAGCTTT GCCGCATGGCTGTTTTATGGCCTGACGATTCTAGGACTCATCGTGATGAGATTTACAAGGA AAGAGCTGGAAAGGCCTATCAAGGTGCCCGTAGTCATTCCCGTCTTGATGACACTCATCTC TGTGTTTTTGGTTCTGGCTCCAATCATCAGCAAGCCCACCTGGGAGTACCTCTACTGTGTGC TGTTTATATTAAGCGGCCTTTTATTTTACTTCCTGTTTGTCCACTACAAGTTTGGATGGGCT CAGAAAATCTCAAAGCCGATTACCATGCACCTTCAGATGCTAATGGAAGTGGTCCCACCG GAGGAAGACCCTGAGTAACAAGCTCCGTCTCTTGTAGCCAAGTCAGCTGAATTTATTTTCT TAAGCAATATTTGTGGTTATTTCTTCCTTTTTTTCTTACGAATAAAATATACTCAGATGTTT AAAA |
| 236 | GCCATTTCTAGGGTTGGACCGTGCAGGCACGGGCGGTCAGCTGGGCCGCAGCTCCTCCGG CTCTGCAGGGTCACGGAGGAAGCCAGCTCCCCTAGTCCAGGCCGAGCTTGCACTTGCGTCT TGTCTGCTGCTGCTGAACCAAGATTTAGCTGTGCGCCCTCCTTGCAGTCTCCTGGAACCAG CAGGAGGAAACATGGGGGATACTGGCCTGAGAAAGCGGAGAGAGGATGAGAAGTCGATC CAGAGCCAAGAGCCTAAGACCACCAGTCTCCAAAAGGAGCTGGGCCTCATCAGTGGCATC TCCATCATCGTGGGCACCATCATTGGCTCTGGGATCTTCGTTTCCCCCAAGTCTGTGCTCAG CAACACGGAAGCTGTGGGGCCCTGCCTCATCATATGGGCGGCTTGCGGGGTCCTCGCGAC GCTGGGTGCCCTGTGCTTTGCGGAGCTTGGCACAATGATCACCAAGTCAGGGGGAGAGTA TCCCTACCTGATGGAGGCCTACGGGCCCATCCCCGCCTACCTCTTCTCCTGGGCCAGCCTG ATCGTCATTAAGCCCACGTCCTTCGCCATCATCTGCCTCAGCTTCTCCGAGTATGTGTGTGC GCCCTTCTATGTGGGCTGCAAGCCTCCTCAAATCGTTGTGAAATGCCTGGCCGCCGCCGCC ATCTTGTTCATCTCGACAGTGAACTCACTGAGCGTGCGGCTGGGAAGCTACGTCCAGAACA TCTTCACCGCGGCCAAGCTGGTGATCGTGGCCATCATCATCATCAGCGGGCTGGTGCTCCT GGCCCAAGGAAACACAAAGAATTTTGATAATTCTTTCGAGGGCGCCCAGCTGTCTGTGGG AGCCATCAGCCTGGCGTTTTACAATGGACTCTGGGCCTATGATGGATGGAATCAACTCAAT TACATCACAGAAGAACTTAGAAACCCTTACAGAAACCTGCCTTTGGCCATTATCATCGGGA TCCCCCTGGTGACGGCGTGCTACATCCTCATGAACGTGTCCTACTTCACCGTGATGACTGC CACCGAACTCCTGCAGTCCCAGGCGGTGGCTGTGACATTTGGTGACCGTGTTCTCTATCCT GCTTCTTGGATCGTTCCACTTTTTGTGGCATTTTCAACCATCGGTGCTGCTAACGGGACCTG CTTCACAGCGGGCAGACTCATTTACGTGGCGGGCCGGGAGGGTCACATGCTCAAAGTGCT TTCTTACATCAGCGTCAGGCGCCTCACTCCAGCCCCCGCCATCATCTTTTATGGTATCATAG CAACGATTTATATCATCCCTGGTGACATAAACTCGTTAGTCAATTATTTCAGCTTTGCCGCA TGGCTGTTTTATGGCCTGACGATTCTAGGACTCATCGTGATGAGATTTACAAGGAAAGAGC TGGAAAGGCCTATCAAGGTGCCCGTAGTCATTCCCGTCTTGATGACACTCATCTCTGTGTTT TTGGTTCTGGCTCCAATCATCAGCAAGCCCACCTGGGAGTACCTCTACTGTGTGCTGTTTAT ATTAAGCGGCCTTTTATTTTACTTCCTGTTTGTCCACTACAAGTTTGGATGGGCTCAGAAAA TCTCAAAGCCGATTACCATGCACCTTCAGATGCTAATGGAAGTGGTCCCACCGGAGGAAG ACCCTGAGTAACAAGCTCCGTCTCTTGTAGCCAAGTCAGCTGAATTTATTTTCTTAAGCAA TATTTGTGGTTATTTCTTCCTTTTTTTCTTACGAATAAAATATACTCAGATGTTTAAAA |
| 242 | ACTCTTCCACCTCCCTTACTGCAGGAAGGCACTCCGAAGACATAAGTCGGTGAGACATGGC TGAAGATAAAAGCAAGAGAGACTCCATCGAGATGAGTATGAAGGGATGCCAGACAAACA ACGGGTTTGTCCATAATGAAGACATTCTGGAGCAGACCCCGGATCCAGGAAGCTCAACAG ACAACCTGAAGCACAGCACCAGGGGCATCCTTGGCTCCCAGGAGCCCGACTTCAAGGGCG TCCAGCCCTATGCGGGGATGCCCAAGGAGGTGCTGTTCCAGTTCTCTGGCCAGGCCCGCTA CCGCATACCTCGGGAGATCCTCTTCTGGCTCACAGTGGCTTCTGTGCTGGTGCTCATCGCG GCCACCATAGCCATCATTGCCCTCTCTCCAAAGTGCCTAGACTGGTGGCAGGAGGGGCCCA TGTACCAGATCTACCCAAGGTCTTTCAAGGACAGTAACAAGGATGGGAACGGAGATCTGA AAGGTATTCAAGATAAACTGGACTACATCACAGCTTTAAATATAAAAACTGTTTGGATTAC TTCATTTTATAAATCGTCCCTTAAAGATTTCAGATATGGTGTTGAAGATTTCCGGGAAGTTG ATCCCATTTTTGGAACGATGGAAGATTTTGAGAATCTGGTTGCAGCCATACATGATAAAGG |
| | TTTAAAATTAATCATCGATTTCATACCAAACCACACGAGTGATAAACATATTTGGTTTCAA TTGAGTCGGACACGGACAGGAAAATATACTGATTATTATATCTGGCATGACTGTACCCATG AAAATGGCAAAACCATTCCACCCAACAACTGGTTAAGTGTGTATGGAAACTCCAGTTGGC ACTTTGACGAAGTGCGAAACCAATGTTATTTTCATCAGTTTATGAAAGAGCAACCTGATTT AAATTTCCGCAATCCTGATGTTCAAGAAGAAATAAAAGAAATTTTACGGTTCTGGCTCACA AAGGGTGTTGATGGTTTTAGTTTGGATGCTGTTAAATTCCTCCTAGAAGCAAAGCACCTGA GAGATGAGATCCAAGTAAATAAGACCCAAATCCCGGACACGGTCACACAATACTCGGAGC TGTACCATGACTTCACCACCACGCAGGTGGGAATGCACGACATTGTCCGCAGCTTCCGGCA GACCATGGACCAATACAGCACGGAGCCCGGCAGATACAGGTTCATGGGGACTGAAGCCTA TGCAGAGAGTATTGACAGGACCGTGATGTACTATGGATTGCCATTTATCCAAGAAGCTGAT TTTCCCTTCAACAATTACCTCAGCATGCTAGACACTGTTTCTGGGAACAGCGTGTATGAGG TTATCACATCCTGGATGGAAAACATGCCAGAAGGAAAATGGCCTAACTGGATGATTGGTG GACCAGACAGTTCACGGCTGACTTCGCGTTTGGGGAATCAGTATGTCAACGTGATGAACAT GCTTCTTTTCACACTCCCTGGAACTCCTATAACTTACTATGGAGAAGAAATTGGAATGGGA AATATTGTAGCCGCAAATCTCAATGAAAGCTATGATATTAATACCCTTCGCTCAAAGTCAC CAATGCAGTGGGACAATAGTTCAAATGCTGGTTTTTCTGAAGCTAGTAACACCTGGTTACC TACCAATTCAGATTACCACACTGTGAATGTTGATGTCCAAAAGACTCAGCCCAGATCGGCT TTGAAGTTATATCAAGATTTAAGTCTACTTCATGCCAATGAGCTACTCCTCAACAGGGGCT GGTTTTGCCATTTGAGGAATGACAGCCACTATGTTGTGTACACAAGAGAGCTGGATGGCAT CGACAGAATCTTTATCGTGGTTCTGAATTTTGGAGAATCAACACTGTTAAATCTACATAAT ATGATTTCGGGCCTTCCCGCTAAAATGAGAATAAGGTTAAGTACCAATTCTGCCGACAAAG GCAGTAAAGTTGATACAAGTGGCATTTTTCTGGACAAGGGAGAGGGACTCATCTTTGAAC ACAACACGAAGAATCTCCTTCATCGCCAAACAGCTTTCAGAGATAGATGCTTTGTTTCCAA TCGAGCATGCTATTCCAGTGTACTGAACATACTGTATACCTCGTGTTAGGCACCTTTATGA AGAGATGAAGACACTGGCATTTCAGTGGGATTGTAAGCATTTGTAATAGCTTCATGTACAG CATGCTGCTTGGTGAACAATCATTAATTCTTCGATATTTCTGTAGCTTGAATGTAACTGCTT TAAGAAAGGTTCTCAAATGTTTTGAAAAAAATAAAATGTTTAAAAGTAAATTATGGCTTAT AGGAGCTTATAACTTTATTCAGATAGCATCAATCAGGGATGACCAGAACACATTAGGACC CCAGATTATTCAAAAACTTTAACGAATTTTAAGGGGAAGAATTTTATCTTTTCCCTTAAAA TGCAGTCATAGAAATTAGAGGATGACTCACTGCCACAGTGTCTAAAAGCATTTGCTAGCA AAGAGGCAGGACACTAATTTGTAAACTGCTCAACTGTTCTGACTGGAAGGGAGGCCTGGA GCTCTGCTATCACCAATCCTTCCCTTCCCTCTACTCCACATCCTTCTAAGGAGCATGATTTG AAAATTACTTTCCTAGGTTAATGGGCATGTGCATCAATGGAGAGAATAGTATAAGCAAGT GAGATGTAGACTAAGCAAAATTTAGATGGAGAAGCACATTTTAAAAAATTAATAACTTAA AAGTCTCAAGTTATTAATTTTTTTTTTGCTAACTCAATTGGAAGTAAGACTATGAAATATTT CAGTGTGTTTCCAATTCCCAGTTGAATGCAGTGTTTCAGAATTTCAGGTATTTCTTAAGATC CTCGAAAACACTGGTGCTGTCAAGTCCAAGTTCCTCGTACAGGAATTTAATTTGGGCTGTA ATCTAAAAGAAACACATTAAAAAAATTAAATAGAAGGCCTTTGTAGTAAAA |
| 246 | GGCTCACTCTGGCAGGTAGGAACAGGGGAGAGTGCACCTGCTACCAGTCAAGCTCAGCCA GACTGCAAGAGGAGGCGAGGCGGAGCCAGCCGAGGGAGTGAACCATGGACAAGTTGAAA TGCCCGAGTTTCTTCAAGTGCAGGGAGAAGGAGAAAGTGTCGGCTTCATCAGAGAATTTC CATGTTGGTGAAAATGATGAGAATCAGGACCGTGGTAACTGGTCCAAAAAATCGGATTAT CTTCTATCTATGATTGGATACGCAGTGGGATTAGGAAATGTGTGGAGATTTCCATATCTGA CCTACAGCAATGGTGGAGGCGCCTTCTTGATACCTTATGCAATTATGTTAGCATTGGCTGG TTTACCTTTGTTCTTTCTGGAGTGTTCACTGGGACAATTTGCTAGCTTAGGTCCAGTTTCAG TTTGGAGGATTCTTCCATTGTTTCAAGGTGTGGGAATTACAATGGTCCTGATCTCCATTTTT GTGACAATCTATTACAATGTCATAATTGCCTATAGTCTTTACTACATGTTTGCTTCTTTTCA AAGTGAACTACCATGGAAAAATTGTTCTTCGTGGTCAGATAAAAACTGTAGCAGATCACC AATAGTAACTCACTGTAATGTGAGTACAGTGAATAAAGGAATACAAGAGATCATCCAAAT GAATAAAAGCTGGGTAGACATCAACAATTTTACCTGCATCAACGGCAGTGAAATTTATCA GCCAGGGCAGCTTCCCAGTGAACAATATTGGAATAAAGTGGCGCTCCAACGGTCAAGTGG AATGAATGAGACTGGAGTAATTGTTTGGTATTTAGCACTTTGTCTTCTTCTGGCTTGGCTCA |
| | TAGTTGGAGCAGCACTATTTAAAGGAATCAAATCGTCTGGCAAGGTGGTATATTTTACAGC TCTTTTCCCCTATGTGGTCCTACTCATCCTGTTAGTACGAGGTGCAACTCTGGAGGGTGCTT CAAAAGGCATTTCATACTATATTGGAGCCCAGTCAAATTTTACAAAACTTAAGGAAGCTGA GGTATGGAAAGATGCTGCCACTCAGATATTTTACTCCCTTTCAGTGGCTTGGGGTGGCTTA GTTGCTCTATCATCTTACAATAAGTTCAAAAACAACTGCTTCTCTGATGCCATTGTGGTTTG TTTGACAAACTGTCTCACTAGCGTGTTTGCTGGATTTGCTATTTTTTCTATATTGGGACACA TGGCCCATATATCTGGAAAGGAAGTTTCTCAAGTTGTAAAATCAGGTTTTGATTTGGCATT CATTGCCTATCCAGAGGCTCTAGCCCAACTCCCAGGTGGTCCATTTTGGTCCATATTATTTT TTTTCATGCTTTTAACTTTGGGTCTCGATTCTCAGTTTGCTTCGATTGAAACGATCACAACA ACAATTCAAGATTTATTTCCCAAAGTGATGAAGAAAATGAGGGTTCCCATAACTTTGGGCT GCTGCTTGGTTTTGTTTCTCCTTGGTCTCGTCTGTGTGACTCAGGCTGGAATTTACTGGGTT CATCTGATTGACCACTTCTGTGCTGGATGGGGCATTTTAATTGCAGCTATACTGGAGCTAG TTGGAATCATCTGGATTTATGGAGGGAACAGATTCATTGAGGATACAGAAATGATGATTG GAGCAAAGAGGTGGATATTCTGGCTATGGTGGAGAGCTTGCTGGTTTGTAATTACGCCTAT CCTTTTGATTGCAATATTTATCTGGTCATTGGTGCAATTTCATAGACCTAATTATGGCGCAA TTCCATACCCTGACTGGGGAGTTGCTTTAGGCTGGTGTATGATTGTTTTCTGCATTATTTGG ATTCCAATTATGGCTATCATAAAAATAATTCAGGCTAAAGGAAACATCTTTCAACGCCTTA TAAGTTGCTGCAGACCAGCTTCTAACTGGGGTCCATACCTGGAACAACATCGTGGGGAAA GATATAAAGACATGGTAGATCCTAAAAAAGAGGCTGACCATGAAATACCTACTGTTAGTG GCAGCAGAAAACCGGAATGAGATCTCATTGAAAAAAATATATGATTGTATAATGTGATTT TTTTTAGAATAGGGGGAACCTTATTTATTTGTGTGTTAACTGAATAGGAAAATGTACATAC TATGTTCATGATAGTGTGATTTTTTTCACATTTAAGCAGGAATGCAATATAAAAATGTGAA TCTCTTAATTCTCAGCCATGTGCTTATTATATTTCTTTTTAGATTGTCTATCTGTATAACACA CACACACACACCTAAGAGTCTCTATTTCACAATTATATTTTTGTAAATAGTATATGCATTTT TAATACATTGGAGGCTTTATTTTGAACTAATTTCTTAGAGAATAGTTATATTTTCTATTACA CAAGTTTAAAAATATTATTAACTTGTATTTTCTTAATATACAATCTATCTTTTCCACAAATA TGAGTGGGAAATAAATCAGCACATTTGAAAGAAAGTGTTAAAACTGAAGGCCTCACTTAA TTAGAAACGTGATAAATATATGGACAAATGGACTATACATACTATAAGAGGACTGTAGTT TAATACTTTTTACCCAAATATGTTTAAAAACTTCGTGCATTTGTTACAGCTCATGTTTTCTA TATGAACTTAGTCATTAATGTTCTTTATAAAAAGTGAAATAAGATGGAAAAATAAGGATCC TACAGCCAGTAAGTGATAAATCTAGAAAATTGAGTTTTGAGTACCTCTTTTCCCATATACA ATCTTCCTTCCTTAGGTAATTTGGAAGAAAACTATGACCCATTTAATTTCTATTGTGTTTCA CAAAATTAAGTGTTGTTCATTATACTCTCTGAAATATAGGTTTAATTTCAAATAGAATATG GACTTAAATGTTAATGAGAAAATGGCTTTAATCAATTCTAGCATTTTATTACTGTAATACA GGGCTGATAGAGTGATTTTGTCTTATATGAGTAAGTTACTACTTACAGGTGATAACTTGCA TACTATTGGAAGATAAACTTGTCAAACTTGTCAAGAATGAGAAAAGCCAAATTAGAAAAT CCTATGTCCTAGTTTCCTTACCAAGGATAATTAAATATATCACTAAGAGCTTTATATATTGA TTATATATTGTTGACAACTGGTTTAAGCATCATAGCCTATGATGATAAACACTGCCTATAT ATGTAAATAGCTTTTCATCAATTCTTAAATTTCTTAACCTAGGCTTCAGGGAGCATATGAA ACCAAAATTATATGGAACATTTTCTGTGTGTACATGTACATGCATTTTTCTAGGGAGAGAG TCCGTAGGTTTATCAGAATATCAAGGAAAACTGTGACCCAAAGAAGTTTAAGAATCACAT ACACTGCTGCTGGC11111GTGCTTGGCAAATGAGTGACAATAGAAGAAATAA11111CTT ACACATTTTAAAACGTTTTCTCTTCCTTGTGATTGAAGATGAAAGGAGTAAGAAATTAAGG CATTTGTTTAATTTATACTGGTAACTTATTTAGGGGGGAGGGGACATGAAGGTAGGTAAAT AGGTAGGCCTCTAATTGAACCACCTCTCTAAGTTATGTACGTATATATAAGCTGAAATTGT GTTTGACATTCTGAGGGTTTTCTTTTTCTTTTTCCTTTTTTTTTTTTTTGGTGGGGGGCTGGG GGTCAGAGTCTTGTTCTGTTGCCCGGGCTGGAGTGCAGTGGCATGATCTCAGCTCACTGCA ACCTCTGCCTTCTGGATTCAAGTGATTCTCCTGCCTCAGCCTCTTGAGTAGCTGGGACTACA GGTGCCCGCCACCACACCAGCTAATTTTTGTATTTTTAGTAGAGGCGAAGTTTCCCCATGTT GGCCAGGCTGGTCTTGAACTCCCGACCTCAAGTGATCTGTCTACCTCGGCCTCCTAAAGTG CTGAGATTACAGGTGTGAGCCACCGTGCCCGGCCCATTCTAAGGGTTTTCTTTGAAGACAG GTCAAATGCTGTTAGTAAGTTTCAGGAGATTGTTAATTCCTCAGTTATACCAGATTTTATAA AATATTTGAGAATAGATGGCTAACAAGAGGTTAGAAATACTTTTCCTTAATTTTAATCCAC |
| | AGTATGTTACATGCATTCTACCACTACATTTTGGTGCTATTTAAGGTGTGCAATTTTCTATA GGTGACTTTTGCAATTCAGGGAAGATTTGGGCATATTAAATGAAAGAATATCTAATTGGGG GAGGTGTGAAGGGAAAGAAATTCTTTTCAAAAGCTGACCACAAAGAGTAGTTAAAAGTTT TTGTCACTATCTTCACAAGTGTGTAAAGCACAGATTTCAACAGAGTGCTTGGCATATTGTA GGGTGCTCAATGGTGGTTTTTATTATTATTACTCAGATTCCACAGTGGCAAGAAACATCAT TCTACATAATGGAAAACATTTACATCAAATCCCACTTACTTTAATGCGAACTTGGAGATAA TTTATGGTATTGTATTGTAAACCATTAATGAAAACTTTTTCACAGTTGAGTGAAATTAAAA TCACTATATCTCAA |

What is claimed is:

1. A genetically modified T-cell genetically modified to express:
a) a recombinant arginine transporter and
b) a chimeric antigen receptor having at least one antigen-specific targeting region that specifically binds a cell surface antigen present on a target cell population, a transmembrane domain, and an intracellular signaling domain.

2. An expression vector comprising an isolated nucleic acid encoding a) an antigen-specific targeting region, b) a transmembrane domain, c) optionally at least one co-stimulatory domain, d) an intracellular signaling domain and e) an arginine transporter.

3. A genetically modified T-cell modified to express a chimeric antigen receptor encoded by the expression vector of claim 2.

4. The genetically modified T-cell of claim 1 or 3, wherein the arginine transporter is selected from the group consisting of CAT-1, CAT-2, CAT-3, CAT-4, y⁺LAT1 and 4F2hc, y⁺LAT2 and 4F2hc, b^{0,+}AT and rBAT, and ATB^{0,+}.

5. The genetically modified T-cell of any one of claims 1, 3, or 4, wherein the genetically modified T-cell comprises a nucleic acid sequence selected from the group consisting of: SEQ ID NO: 180, 184-188, 204, 205, 210, 214, 215, 220-222, 227-230, 234-236, 242, and 246, or a fragment or a variant thereof.

6. The genetically modified T-cell of claims 1, 3, or 4, wherein the genetically modified T-cell comprises a nucleic acid expressing a sequence having about 90%, 95%, or 99% percent identity to one of SEQ ID NO: 180, 184-188, 204, 205, 210, 214, 215, 220-222, 227-230, 234-236, 242, and 246.

7. A pharmaceutically acceptable composition comprising the genetically modified T-cell of any one of claims 1 or 3-6, and a pharmaceutically acceptable excipient.

8. A priming medium comprising the genetically modified T-cell of any one of claims 1 or 3-6, and L-arginine.

9. A pharmaceutical composition comprising a chimeric antigen receptor T cell (CAR-T cell) which expresses a recombinant arginine transporter and a chimeric antigen receptor protein.

10. The pharmaceutical composition of claim 9, wherein the arginine transporter is CAT-1.

11. The pharmaceutical composition of claim 9, wherein the arginine transporter is CAT-2.

12. The pharmaceutical composition of claim 9, wherein the arginine transporter is CAT-3.

13. The pharmaceutical composition of claim 9, wherein the arginine transporter is CAT-4.

14. The pharmaceutical composition of claim 9, wherein the arginine transporter is y⁺LAT1 and 4F2hc.

15. The pharmaceutical composition of claim 9, wherein the arginine transporter is y⁺LAT2 and 4F2hc.

16. The pharmaceutical composition of claim 9, wherein the arginine transporter is b^{0,+}AT and rBAT.

17. The pharmaceutical composition of claim 9, wherein the arginine transporter is ATB^{0,+}.

18. The pharmaceutical composition of claim 9, wherein the pharmaceutical composition is packaged as a kit.

19. A method of treating a solid tumor cancer in a patient in need thereof, comprising administering to the patient an effective amount of the pharmaceutical composition of claim 7 or 9.

20. A method of treating a hematological cancer in a patient in need thereof, comprising administering to the patient an effective amount of the pharmaceutical composition of claim 7 or 9.

21. A method of modulating intracellular arginine levels to effect a T cell-mediated immune response in a patient in need thereof, comprising administering to the patient an effective amount of the pharmaceutical composition of claim 7 or 9.

22. A method for treating a condition in a human patient in need thereof, comprising: administering to the human patient a therapeutically effective amount of a composition comprising a chimeric antigen receptor T cell (CAR-T cell) which expresses a recombinant arginine transporter and a chimeric antigen receptor protein.

23. A method of modulating a T cell-mediated immune response to a target cell population expressing a cell surface antigen in a patient in need thereof, comprising administering to the patient a therapeutically effective amount of T-cells that are a) genetically modified to express a chimeric antigen receptor wherein the chimeric antigen receptor comprises: at least one antigen-specific targeting region that specifically binds the cell surface antigen present on the target cell population, a transmembrane domain, an intracellular signaling domain, and b) genetically modified to express a recombinant arginine transporter.

24. The method of claim 22 or 23, wherein the T-cells are cultured in a culture medium comprising arginine before administering.

25. The method according to any one of claims 22–24, wherein the arginine transporter is CAT-1.

26. The method according to any one of claims 22–24, wherein the arginine transporter is CAT-2.

27. The method according to any one of claims 22–24, wherein the arginine transporter is CAT-3.

28. The method according to any one of claims 22–24, wherein the arginine transporter is CAT-4.

29. The method according to any one of claims 22–24, wherein the arginine transporter is y⁺LAT1 and 4F2hc.

30. The method according to any one of claims 22–24, wherein the arginine transporter is y⁺LAT2 and 4F2hc.

31. The method according to any one of claims 22–24, wherein the arginine transporter is b^{0,+}AT and rBAT.

32. The method according to any one of claims 22–24, wherein the arginine transporter is ATB^{0,+}.

33. The method of any one of claims 22–24, comprising administering the T-cell of any one of claims 1 and 3-6.

34. The method according to any one of claims 22-33, further comprising administering a second therapeutic agent to the human patient.

35. The method according to claim 34, wherein the second therapeutic agent an anti-PD-1, anti-PD-L1, or an anti-CTLA-4 antibody.

36. The method according to claim 34 or 35, wherein the administering of the second therapeutic agent is performed before, during or after the administering of the composition comprising the CAR-T cell.

37. The method according to claim 33 or 34, wherein the administering of the second therapeutic agent is performed before, during or after the administering of the therapeutically effective amount of T-cells.

38. The method according to any one of claims 22-37, wherein the composition comprising the CAR-T cells is administered to the human patient once every week, once every 2 weeks, once every 3 weeks, or once every 4 weeks.

39. The method according to any one of claims 22-37, comprising administering 10⁷ to 10¹⁰ CAR-T cells per kilogram of the patient.

40. A method of making a genetically modified CAR-T cell that expresses a recombinant arginine transporter, comprising:
transfecting a T-cell with a DNA construct comprising a nucleotide sequence for a specific chimeric antigen receptor and for an arginine transporter thereby producing a genetically modified CAR-T cell that expresses both the chimeric antigen receptor and the arginine transporter; and
culturing the genetically modified CAR-T cell in a culture medium comprising arginine.

41. The method of claim 40, wherein said culturing comprises culturing the genetically modified CAR-T cell in the culture medium until the intracellular arginine level of the CAR-T cell accumulates to a certain level.

42. A genetically modified T-cell genetically modified to express a recombinant arginine transporter.

43. The genetically modified T-cell of claim 42, wherein the arginine transporter is selected from the group consisting of CAT-1, CAT-2, CAT-3, CAT-4, y⁺LAT1 and 4F2hc, y⁺LAT2 and 4F2hc, b^{0,+}AT and rBAT, and ATB^{0,+}.

44. The genetically modified T-cell of claim 42 or 43, wherein the genetically modified T-cell comprises a nucleic acid sequence selected from the group consisting of: SEQ ID NO: 180, 184-188, 204, 205, 210, 214, 215, 220-222, 227-230, 234-236, 242, and 246, or a fragment or a variant thereof.

45. The genetically modified T-cell of claim 42 or 43, wherein the genetically modified T-cell comprises a nucleic acid expressing a sequence having about 90%, 95%, or 99% percent identity to one of SEQ ID NO: 180, 184-188, 204, 205, 210, 214, 215, 220-222, 227-230, 234-236, 242, and 246.

46. A pharmaceutically acceptable composition comprising the genetically modified T-cell of any one of claims 42-45, and a pharmaceutically acceptable excipient.

47. A priming medium comprising the genetically modified T-cell of any one of claims 42-45, and L-arginine.

48. A pharmaceutical composition comprising a T cell which expresses a recombinant arginine transporter protein.

49. The pharmaceutical composition of claim 48, wherein the arginine transporter is CAT-1.

50. The pharmaceutical composition of claim 48, wherein the arginine transporter is CAT-2.

51. The pharmaceutical composition of claim 48, wherein the arginine transporter is CAT-3.

52. The pharmaceutical composition of claim 48, wherein the arginine transporter is CAT-4.

53. The pharmaceutical composition of claim 48, wherein the arginine transporter is y⁺LAT1 and 4F2hc.

54. The pharmaceutical composition of claim 48, wherein the arginine transporter is y⁺LAT2 and 4F2hc.

55. The pharmaceutical composition of claim 48, wherein the arginine transporter is b^{0,+}AT and rBAT.

56. The pharmaceutical composition of claim 48, wherein the arginine transporter is ATB^{0,+}.

57. The pharmaceutical composition of claim 48, wherein the pharmaceutical composition is packaged as a kit.

58. A method of treating a solid tumor cancer in a patient in need thereof, comprising administering to the patient an effective amount of the pharmaceutical composition of claim 46 or 48.

59. A method of treating a hematological cancer in a patient in need thereof, comprising administering to the patient an effective amount of the pharmaceutical composition of claim 46 or 48.

60. A method of modulating intracellular arginine levels to effect a T cell-mediated immune response in a patient in need thereof, comprising administering to the patient an effective amount of the pharmaceutical composition of claim 46 or 48.

61. A method for treating a condition in a human patient in need thereof, comprising: administering to the human patient a therapeutically effective amount of a composition comprising a T cell which expresses a recombinant arginine transporter.

62. The method of claim 61, wherein the T-cells are cultured in a culture medium comprising arginine before administering.

63. The method of claim 61 or 62, wherein the arginine transporter is CAT-1.

64. The method of claim 61 or 62, wherein the arginine transporter is CAT-2.

65. The method of claim 61 or 62, wherein the arginine transporter is CAT-3.

66. The method of claim 61 or 62, wherein the arginine transporter is CAT-4.

67. The method of claim 61 or 62, wherein the arginine transporter is y⁺LAT1 and 4F2hc.

68. The method of claim 61 or 62, wherein the arginine transporter is y⁺LAT2 and 4F2hc.

69. The method of claim 61 or 62, wherein the arginine transporter is b^{0,+}AT and rBAT.

70. The method of claim 61 or 62, wherein the arginine transporter is ATB^{0,+}.

71. The method of claim 61 or 62, comprising administering the T-cell of any one of claims 42-45.

72. The method according to any one of claims 58-71, further comprising administering a second therapeutic agent to the human patient.

73. The method according to claim 72, wherein the second therapeutic agent an anti-PD-1, anti-PD-L1, or an anti-CTLA-4 antibody.

74. The method according to claim 72 or 73, wherein the administering of the second therapeutic agent is performed before, during or after the administering of the composition comprising the T cell.

75. The method according to claim 72 or 73, wherein the administering of the second therapeutic agent is performed before, during or after the administering of the therapeutically effective amount of T-cells.

76. A method of making a genetically modified T cell that expresses a recombinant arginine transporter, comprising:
transfecting a T-cell with a DNA construct comprising a nucleotide sequence for an arginine transporter thereby producing a genetically modified T cell that expresses the arginine transporter; and
culturing the genetically modified T cell in a culture medium comprising arginine.

77. The method of claim 76, wherein said culturing comprises culturing the genetically modified T cell in the culture medium until the intracellular arginine level of the T cell accumulates to a certain level.

78. A method of increasing T cell survival in a low arginine environment, the method comprising: administering a T cell comprising a recombinant arginine transporter to a low arginine environment.

79. The method of claim 78, wherein prior to the administering step, the method comprises transfecting the T cell with a DNA construct comprising a nucleotide sequence encoding the recombinant arginine transporter.

80. The method of claim 78 or claim 79, wherein the T-cell comprises a chimeric antigen receptor and/or a DNA construct comprising a nucleotide sequence encoding a chimeric antigen receptor.

81. The method of any one of claims 78-80, wherein prior to the administering step, the method comprises culturing the T cell in a culture medium comprising arginine.

82. The method of claim 81, wherein the culturing comprises culturing the T cell in the culture medium until the intracellular arginine level of the T cell accumulates to a certain level.

83. The method of any one of claims 78-82, wherein the arginine transporter is selected from the group consisting of CAT-1, CAT-2, CAT-3, CAT-4, y⁺LAT1 and 4F2hc, y⁺LAT2 and 4F2hc, b^{0,+}AT and rBAT, and ATB^{0,+}.

84. The method of any one of claims 78-82, wherein the DNA construct comprising a nucleotide sequence encoding the recombinant arginine transporter comprises a nucleic acid sequence selected from the group consisting of: SEQ ID NO: 180, 184-188, 204, 205, 210, 214, 215, 220-222, 227-230, 234-236, 242, and 246, or a fragment or a variant thereof.

85. The method of any one of claims 78-82, wherein the DNA construct comprising a nucleotide sequence encoding the recombinant arginine transporter comprises a nucleic acid expressing a sequence having about 90%, 95%, or 99% percent identity to one of SEQ ID NO: 180, 184-188, 204, 205, 210, 214, 215, 220-222, 227-230, 234-236, 242, and 246.

86. The method of any one of claims 78-85, wherein the low arginine environment is a cell culture medium.

87. The method of any one of claims 78-85, wherein the low arginine environment is a tumor microenvironment.


SEQUENCE LISTING (278 sequences)

SEQ ID NO: 1 - DNA, 348 bp, Unknown
  Description of Unknown: CD33 antigen-recognition domain
gaagtgcagctggtgcagagcggagcagaagtgaagaagcccggaagcagcgtgaaggtgtcttgcaaggccagcggctacaccatcaccgacagcaacatccattgggtccggcaggctccaggacagtctctggagtggatcggctacatctacccctacaacggcggcaccgactacaaccagaagttcaagaaccgggccaccctgaccgtggataaccccaccaacaccgcctacatggagctgagcagcctgagaagcgaggacaccgccttctactattgcgtgaacggcaacccttggctggcctattggggacagggaacactggtgaccgtgtcctct

SEQ ID NO: 2 - DNA, 342 bp, Unknown
  Description of Unknown: CD33 antigen-recognition domain
gacatccagctgacccagtctcctagcaccctgagcgctagcgtgggagatagagtgaccatcacttgcagagccagcgagagcctggacaactacggcatccggttcctgacttggttccagcagaaacccggcaaggcccctaaactgctgatgtacgccgcctctaaccagggaagcggagtgcctagcagattcagcggcagcggaagcggaaccgagttcaccctgaccatcagctctctgcagccagacgacttcgccacctactactgccagcagaccaaggaggtgccttggagcttcggccagggaaccaaggtggaagtgaagcggacagtg

SEQ ID NO: 3 - protein, 116 aa, Unknown [sequence omitted]

SEQ ID NO: 4 - protein, 114 aa, Unknown
  Description of Unknown: anti-CD33 light chain variable domain sequence
DIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTV

SEQ ID NO: 5 - DNA, 135 bp, Unknown
  Description of Unknown: CD8alpha-derived hinge region sequence
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 6 - DNA, 144 bp, Unknown
  Description of Unknown: CD8alpha-derived hinge region sequence
gcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 7 - protein, 45 aa, Unknown
  Description of Unknown: CD8alpha-derived hinge region sequence
TTTPAPRPPTPAPTIASQPLSLRPEACRPAAGGAVHTRGLDFACD

SEQ ID NO: 8 - protein, 48 aa, Unknown
  Description of Unknown: CD8alpha-derived hinge region sequence
AKPTTTPAPRPPTPAPTIASQPLSLRPEACRPAAGGAVHTRGLDFACD

SEQ ID NO: 9 - DNA, 117 bp, Unknown
  Description of Unknown: CD28-derived hinge region sequence
attgaagttatgtatcctcctccttacctagacaatgagaagagcaatggaaccattatccatgtgaaagggaaacacctttgtccaagtcccctatttcccggaccttctaagccc

SEQ ID NO: 10 - protein, 39 aa, Unknown
  Description of Unknown: CD28-derived hinge region sequence
IEVMYPPPYLDNEKSNGTIIHVKGKHLCPSPLFPGPSKP

SEQ ID NO: 11 - DNA, 45 bp, Unknown
  Description of Unknown: IgG1-derived hinge region sequence
gagcccaagagctgcgacaagacccacacctgccccccctgcccc

SEQ ID NO: 12 - protein, 15 aa, Unknown
  Description of Unknown: IgG1-derived hinge region sequence
EPKSCDKTHTCPPCP

SEQ ID NO: 13 - DNA, 117 bp, Unknown; identical to SEQ ID NO: 9
  Description of Unknown: IgG2-derived hinge region sequence

SEQ ID NO: 14 - protein, 12 aa, Unknown
  Description of Unknown: IgG2-derived hinge region sequence
ERKCCVECPPCP

SEQ ID NO: 15 - DNA, 186 bp, Unknown
  Description of Unknown: IgG3-derived hinge region sequence
gagctcaaaaccccacttggtgacacaactcacacatgcccacggtgcccagagcccaaatcttgtgacacacctcccccgtgcccacggtgcccagagcccaaatcttgtgacacacctcccccatgcccacggtgcccagagcccaaatcttgtgacacacctcccccgtgcccaaggtgccca

SEQ ID NO: 16 - protein, 62 aa, Unknown
  Description of Unknown: IgG3-derived hinge region sequence
ELKTPLGDTTHTCPRCPEPKSCDTPPPCPRCPEPKSCDTPPPCPRCPEPKSCDTPPPCPRCP

SEQ ID NO: 17 - DNA, 36 bp, Unknown
  Description of Unknown: IgG4-derived hinge region sequence
gagtccaaatatggtcccccatgcccatcatgccca

SEQ ID NO: 18 - DNA, 39 bp, Unknown
  Description of Unknown: IgG4-derived hinge region sequence
gagtccaaatatggtcccccatgcccatcatgcccagca

SEQ ID NO: 19 - DNA, 321 bp, Unknown
  Description of Unknown: IgG4-derived hinge region sequence
gggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctctgggtaaa

SEQ ID NO: 20 - DNA, 387 bp, Unknown
  Description of Unknown: IgG4-derived hinge region sequence
gagagcaagtacggccccccctgccccccctgccccggcggcggcagcagcggcggcggcagcggcggccagcccagagagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaag

SEQ ID NO: 21 - DNA, 687 bp, Unknown
  Description of Unknown: IgG4-derived hinge region sequence
gagagcaagtacggccccccctgccccagctgccccgcccccgagttcgagggcggccccagcgtgttcctgttcccccccaagcccaaggacaccctgatgatcagcagaacccccgaggtgacctgcgtggtggtggacgtgagccaggaggaccccgaggtgcagttcaactggtacgtggacggcgtggaggtgcaccaggccaagaccaagcccagagaggagcagttcaacagcacctacagagtggtgagcgtgctgaccgtgctgcaccaggactggctgaacggcaaggagtacaagtgcaaggtgagcaacaagggcctgcccagcagcatcgagaagaccatcagcaaggccaagggccagcccagagagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaag

SEQ ID NO: 22 - DNA, 687 bp, Unknown
  Description of Unknown: IgG4-derived hinge region sequence
gagagcaagtacggccccccctgccccccctgccccgcccccgagttcgagggcggccccagcgtgttcctgttcccccccaagcccaaggacaccctgatgatcagcagaacccccgaggtgacctgcgtggtggtggacgtgagccaggaggaccccgaggtgcagttcaactggtacgtggacggcgtggaggtgcaccaggccaagaccaagcccagagaggagcagttcaacagcacctacagagtggtgagcgtgctgaccgtgctgcaccaggactggctgaacggcaaggagtacaagtgcaaggtgagcaacaagggcctgcccagcagcatcgagaagaccatcagcaaggccaagggccagcccagagagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaag

SEQ ID NO: 23 - protein, 12 aa, Unknown
  Description of Unknown: IgG4-derived hinge region sequence
ESKYGPPCPSCP

SEQ ID NO: 24 - protein, 13 aa, Unknown
  Description of Unknown: IgG4-derived hinge region sequence
ESKYGPPCPSCPA

SEQ ID NO: 25 - protein, 107 aa, Unknown
  Description of Unknown: IgG4-derived hinge region sequence
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - protein, 129 aa, Unknown
  Description of Unknown: IgG4-derived hinge region sequence
ESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 229 aa, Unknown
  Description of Unknown: IgG4-derived hinge region sequence
ESKYGPPCPSCPAPEFEGGPSVFLFPPKPKDTLMISRTPEVTCVVVDVSQEDPEVQFNWYVDGVEVHQAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 28 - protein, 229 aa, Unknown
  Description of Unknown: IgG4-derived hinge region sequence
ESKYGPPCPPCPAPEFEGGPSVFLFPPKPKDTLMISRTPEVTCVVVDVSQEDPEVQFNWYVDGVEVHQAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 29 - DNA, 45 bp, Artificial Sequence
  Description of Artificial Sequence: Synthetic oligonucleotide
ggcggcggaggatctggcggaggtggaagcggaggcggtggaagc

SEQ ID NO: 30 - protein, 15 aa, Artificial Sequence
  Description of Artificial Sequence: Synthetic peptide
GGGGSGGGGSGGGGS

SEQ ID NO: 31-41 - skipped (11 entries)

SEQ ID NO: 42 - DNA, 66 bp, Homo sapiens
atggccctgattgtgctggggggcgtcgccggcctcctgcttttcattgggctaggcatcttcttc

SEQ ID NO: 43-48 - skipped (6 entries)

SEQ ID NO: 49 - protein, 21 aa, Homo sapiens
IYIWAPLAGTCGVLLLSLVIT

SEQ ID NO: 50 - protein, 23 aa, Homo sapiens
IYIWAPLAGTCGVLLLSLVITLY

SEQ ID NO: 51 - protein, 24 aa, Homo sapiens
IYIWAPLAGTCGVLLLSLVITLYC

SEQ ID NO: 52 - DNA, 63 bp, Homo sapiens
atctacatctgggcgcccttggccgggacttgtggggtccttctcctgtcactggttatcacc

SEQ ID NO: 53 - DNA, 69 bp, Homo sapiens
atctacatctgggcgcccttggccgggacttgtggggtccttctcctgtcactggttatcaccctttac

SEQ ID NO: 54 - DNA, 72 bp, Homo sapiens
atctacatctgggcgcccttggccgggacttgtggggtccttctcctgtcactggttatcaccctttactgc

SEQ ID NO: 55-61 - skipped (7 entries)

SEQ ID NO: 62 - protein, 27 aa, Homo sapiens
FWVLVVVGGVLACYSLLVTVAFIIFWV

SEQ ID NO: 63 - DNA, 81 bp, Homo sapiens
ttttgggtgctggtggtggttggtggagtcctggcttgctatagcttgctagtaacagtggcctttattattttctgggtg

SEQ ID NO: 64-67 - skipped (4 entries)

SEQ ID NO: 68 - protein, 21 aa, Homo sapiens
LCYLLDGILFIYGVILTALFL

SEQ ID NO: 69 - DNA, 63 bp, Homo sapiens
ctctgctacctgctggatggaatcctcttcatctatggtgtcattctcactgccttgttcctg

SEQ ID NO: 70 - skipped

SEQ ID NO: 71 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 72 - protein, 179 aa, Homo sapiens
EINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 73 - protein, 21 aa, Homo sapiens
FWLPIGCAAFVVVCILGCILI

SEQ ID NO: 74 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - DNA, 336 bp, Homo sapiens
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 76-78 - skipped (3 entries)

SEQ ID NO: 79 - protein, 42 aa, Homo sapiens
CSPCPPNSFSSAGGQRTCDICRQCKGVFRTRKECSSTSNAEC

SEQ ID NO: 80 - protein, 112 aa, Unknown
  Description of Unknown: CD3zeta sequence
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 81-83 - skipped (3 entries)

SEQ ID NO: 84 - protein, 27 aa, Unknown
  Description of Unknown: 4-1BB transmembrance region sequence
IISFFLALTSTALLFLLFFLTLRFSVV

SEQ ID NO: 85 - DNA, 81 bp, Unknown
  Description of Unknown: 4-1BB transmembrance region sequence
atcatctccttctttcttgcgctgacgtcgactgcgttgctcttcctgctgttcttcctcacgctccgtttctctgttgtt

SEQ ID NO: 86 - protein, 42 aa, Homo sapiens
KRGRKKLLYIFKQPFMRPVQTTQEEDGCSCRFPEEEEGGCEL

SEQ ID NO: 87 - DNA, 126 bp, Homo sapiens
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 88-90 - skipped (3 entries)

SEQ ID NO: 91 - protein, 48 aa, Homo sapiens
QRRKYRSNKGESPVEPAEPCRYSCPREEEGSTIPIQEDYRKPEPACSP

SEQ ID NO: 92 - protein, 41 aa, Homo sapiens
RSKRSRLLHSDYMNMTPRRPGPTRKHYQPYAPPRDFAAYRS

SEQ ID NO: 93 - protein, 41 aa, Unknown
  Description of Unknown: CD28 co-stimulatory domain
RSKRSRGGHSDYMNMTPRRPGPTRKHYQPYAPPRDFAAYRS

SEQ ID NO: 94 - DNA, 123 bp, Unknown
  Description of Unknown: CD28 co-stimulatory domain
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 95 - DNA, 123 bp, Unknown
  Description of Unknown: CD28 co-stimulatory domain
aggagtaagaggagcaggggcggccacagtgactacatgaacatgactccccgccgccccgggcccacccgcaagcattaccagccctatgccccaccacgcgacttcgcagcctatcgctcc

SEQ ID NO: 96-108 - skipped (13 entries)

SEQ ID NO: 109 - protein, 62 aa, Homo sapiens
KKVAKKPTNKAPHPKQEPQEINFPDDLPGSNTAAPVQETLHGCQPVTQEDGKESRISVQERQ

SEQ ID NO: 110-111 - skipped (2 entries)

SEQ ID NO: 112 - protein, 22 aa, Homo sapiens
MIETYNQTSPRSAATGLPISMK

SEQ ID NO: 113-129 - skipped (17 entries)

SEQ ID NO: 130 - protein, 174 aa, Homo sapiens
HRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKEWCKYELDFSHFRLFDENNDAAILILLEPIEKKAIPQRFCKLRKIMNTKTYLEWPMDEAQREGFWVNLRAAIKS

SEQ ID NO: 131 - protein, 145 aa, Homo sapiens
CYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 132-136 - skipped (5 entries)

SEQ ID NO: 137 - protein, 22 aa, Homo sapiens
CARPRRSPAQDGKVYINMPGRG

SEQ ID NO: 138-140 - skipped (3 entries)

SEQ ID NO: 141 - protein, 42 aa, Homo sapiens
ALYLLRRDQRLPPDAHKPPGGGSFRTPIQEEQADAHSTLAKI

SEQ ID NO: 142 - DNA, 126 bp, Homo sapiens
gccctgtacctgctccggagggaccagaggctgccccccgatgcccacaagccccctgggggaggcagtttccggacccccatccaagaggagcaggccgacgcccactccaccctggccaagatc

SEQ ID NO: 143 - protein, 35 aa, Homo sapiens
TKKKYSSSYHDPNGEYMFMRAVNTAKKSRLTDVTL

SEQ ID NO: 144 - DNA, 105 bp, Homo sapiens
acaaaaaagaagtattcatccagtgtgcacgaccctaacggtgaatacatgttcatgagagcagtgaacacagccaaaaaatctagactcacagatgtgacccta

SEQ ID NO: 145-151 - skipped (7 entries)

SEQ ID NO: 152 - protein, 286 aa, Homo sapiens
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 153-178 - skipped (26 entries)

SEQ ID NO: 179 - protein, 145 aa, Homo sapiens
RFDAFICYCPSDIQFVQEMIRQLEQTNYRLKLCVSDRDVLPGTCVWSIASELIEKRLARRPRGGCRRMVVVVSDDYLQSKECDFQTKFALSLSPGAHQKRLIPIKYKAMKKEFPSILRFITVCDYTNPCTKSWFWTRLAKALSLP

SEQ ID NO: 180 - DNA, 7343 bp, Homo sapiens
gcactgctgatgaaacctggcgccggaacccgccagccctcggcgcccattcagtccgcgcaggcaggtgtgagcagcgggtcaactacctggcaggcgcgcacgcggccgcgggctcccgctaaccgcagcctccactcctctccccgcgcgccgcgcccccgccccgccccgccccgcccggtctcgccggccgagcgtccgttggtccttgagcgcgtccgacagtctgtctgttcgcgatcctgccggagccccgccgccgccggcttggattctgaaaccttccttgtatccctcctgagacatctttgctgcaagatcgaggctgtcctctggtgagaaggtggtgaggcttcccgtcatattccagctctgaacagcaacatggggtgcaaagtcctgctcaacattgggcagcagatgctgcggcggaaggtggtggactgtagccgggaggagacgcggctgtctcgctgcctgaacacttttgatctggtggccctcggggtgggcagcacactgggtgctggtgtctacgtcctggctggagctgtggcccgtgagaatgcaggccctgccattgtcatctccttcctgatcgctgcgctggcctcagtgctggctggcctgtgctatggcgagtttggtgctcgggtccccaagacgggctcagcttacctctacagctatgtcaccgttggagagctctgggccttcatcaccggctggaacttaatcctctcctacatcatcggtacttcaagcgtagcgagggcctggagcgccaccttcgacgagctgataggcagacccatcggggagttctcacggacacacatgactctgaacgcccccggcgtgctggctgaaaaccccgacatattcgcagtgatcataattctcatcttgacaggacttttaactcttggtgtgaaagagtcggccatggtcaacaaaatattcacttgtattaacgtcctggtcctgggcttcataatggtgtcaggatttgtgaaaggatcggttaaaaactggcagctcacggaggaggattttgggaacacatcaggccgtctctgtttgaacaatgacacaaaagaagggaagcccggtgttggtggattcatgcccttcgggttctctggtgtcctgtcgggggcagcgacttgcttctatgccttcgtgggctttgactgcatcgccaccacaggtgaagaggtgaagaacccacagaaggccatccccgtggggatcgtggcgtccctcttgatctgcttcatcgcctactttggggtgtcggctgccctcacgctcatgatgccctacttctgcctggacaataacagccccctgcccgacgcctttaagcacgtgggctgggaaggtgccaagtacgcagtggccgtgggctccctctgcgctctttccgccagtcttctaggttccatgtttcccatgcctcgggttatctatgccatggctgaggatggactgctatttaaattcttagccaacgtcaatgataggaccaaaacaccaataatcgccacattagcctcgggtgccgttgctgctgtgatggccttcctctttgacctgaaggacttggtggacctcatgtccattggcactctcctggcttactcgttggtggctgcctgtgtgttggtcttacggtaccagccagagcagcctaacctggtataccagatggccagtacttccgacgagttagatccagcagaccaaaatgaattggcaagcaccaatgattcccagctgggctttttaccagaggcagagatgttctctttgaaaaccatactctcacccaaaaacatggagccttccaaaatctctgggctaattgtgaacatttcaaccagcctcatagctgttctcatcatcaccttctgcattgtgaccgtgcttggaagggaggctctcaccaaaggggcgctgtgggcagtctttctgctcgcagggtctgccctcctctgtgccgtggtcacgggcgtcatctggaggcagcccgagagcaagaccaagctctcatttaaggttcccttcctgccagtgctccccatcctgagcatcttcgtgaacgtctatctcatgatgcagctggaccagggcacctgggtccggtttgctgtgtggatgctgataggcttcatcatctactttggctatggcctgtggcacagcgaggaggcgtccctggatgccgaccaagcaaggactcctgacggcaacttggaccagtgcaagtgacgcacagccccgccccccggaggtggcagcagccccgagggacgcccccagaggaccgggaggcaccccaccctccccaccagtgcaacagaaaccacctgcgtccacaccctcactgcagccaaaggtgcaattacttgacctgcagccccagcccaccctcggctctgcagccggttctccgggccctggtcacctccagacagctgcctggccggggccactaggctgcggctggccactgtgtctcctcacttctctgaacaaagcagttcctcccctaccagctcagccccgagctgccgcagcctcaggcagaacggaggtcaccttctctccttatcttgggaaccaggccttcctcccggggactgttctgggattgaaattgtgcatactccaaactttcgcagccatcttcccgctcagccccagacacccagcaatcaagccagatgagtaccacaaaacagtgtgtccccagcagctccccaccccagagccaaatgacagtagtgcacttaaaaaggaaaatcaggcctgttgtccttctccggttgcattcagatgggtcattagggccggaccctgcctgccccttggcttctcagggctttgctctgacaccatgacagctgcccggggctgagggcagctggctccactcaaatgaggaagaagggatcactcccattagggcctgctttgcttatgcatgtgtgtgcacatgcatgtaaaccagggaccttcagctcacggcctccaggcctgggccagttcttgctgctcctgccgtctcccccgactggctgtgtcctgagtaactggaacatgagacagtatctgcaggactggccccatggtggccgagtcagaagtctgtttcctgtgagtcgccaccgttcactcagtcttgccctcccatgctttggagccagtctggtggctcctgtaaggttctcaaggctggtggcagctcagtctggggtcaggacatgtcggggtcatgcgtttctggccctgacataagctgtctggcctctctgtgacatgatgaaattgaaatcaatccacagtccatgaaattgtgacactccaccagattaagttagggcataacattaacttggaaatggccatgtcatcacccctgcggctgtcctatagctgagatgcgtgggtcgcaggggaggtgatttctaggcatattgctgtcccttttgtgtatctgtcatccggatgcttcggaccccacgcctctgcaagtgggagagacccgagcatcctccccacccccatagctccagtgcacgccacccccgtcttgcctgggtcggggcctgcggccagcaccatttcacacacactccttgtagatgggagccagaggaaacctgaacgtgggtggagcgttccactgagtctacttcaggagacagaaggcccatgctgatgggggaggaggagggatgtgggcattttggacaccaggggaaatggaaatgctgctttcaaaacttagtttcctttccatttcttcctagtctggcctttgacacaaatctggtagaaagaagcctgataaattgagggcacttgtaccctccctgtgcccccagaaggttcttggagagaagtgcaagaatttgtgaacacggcggtggagggcgggtggatggccatgggctgagcctccgtatcaggcctgctcaccttgctgggagctttattctgatctcattttgaatgttccagagggagcatcataagagcccagagctccgatttccaaagagtgatattgacatttatggagattggtgttgtaacatattttgataaatactaacttattttgttggggttttggttgtctcttgtcttaggacctggtagttatttgcttgatttttttttccgttattttctacataggcaaagagaattcgagggatagacagtctccaagaaaagtgaagtggtgggagagaattgcttttttcttttttttcttttctctagtttttctttctggctgagatttccgtgcaagacagcacccaatagactatttagagttgacatttgacattttaatgggcgccatggctcattttgtagattgagaaggtgcgtctcccctgctccaagtctcatcatgacagcgtgctgacagctgggagtctgtggccttcctcacgcagaggccttaaagctggacacagaagcacgcctaggctgggcagggatgggacccatgccccctccttagaggacgggcttcctggttaggaaaggacacgtgggggtgccttgcataatagttcactggtcaccgtgcttttatgagtagtgtttttgtgcacttgccaggggttttctctctgtgtgagaggggagtgatttaagcaatggtgtctggagtaagccttacaattttaatagactttttcttatcatatccctcatttctttccctgaaataaaaatacacacaagcaaaaaaaaaatgatagtttcacatctcttagttcccttgcccaaacaagaatattcttagttccactggccaggattttcctacatagtcagaacttacacattactagaggcacacccaccaaggagtattgtgtctacttttatctgtgcaccagccacaaatacccacattggaaagacccatttgtgatgggtaaacatcccttcctgtctcccacaacccctgtgactgccctgcatgtgttcatgacctccgaaggcccaaattcatgaagcagcaaacccagcagatctccacccccctgcctcaggacctctgctgaagagggggatgaagtgggtctccagggaggcagtgggggccttgttggcagctggctcgggagccggcttacaggagggcagctctgcagttgggaggggcaccgtccggaggagaccaggcctctacacaccccccactctacttatcatccctgctcacacacccttgtccaaggctttatgcatcggatttatttttccaaatcaagaggacagtgatagatgcattttccccaggctgtctcagaaaggtcgctaaatgtatactgttgtcagaattgctgagatctccccccacttttggtttttgcagcagtaaaaactctttccactgtgacttattttctctctcaggcagccagccacctggtcccttgtgctgactctagcacagtggccaggatccaatacgagtccaggggtgaccgcaggatggtgggggcagcgggcttctccacctaccccagccaccaaggccctgacgcactgcctcctgcaccttcagcacatccctgtgcacagctggaagggtgcatggcccgctcacctttgttcagatgggtggaaacgctgatgataccagctcctccctgccgtgcccctgccacggagcaggcattgtgaactggctggtgtttgcagtcccacgtggcatggcctccagcccaacccacagtggagactggagacagggcaatgagtctggtggggggcacgtggacatgccccataggggccccacccagacttaacaggcaaggtcctgggcattgcgcgacgcaggactcaatgctaaagcaagcctgcctggctctgtgccagggcccctcttctgattcacacatcccatttttacacagacccttccttcttaataaaggctgacagttctgttggcagccaagaacccacaccatgaagacagggagtgaggggcctttgtgcccaactccagcacagctgcgttctggggtgtgtgagaggcatgttcgtgtctgtgcgctggtggtctcgtgagacagttccgaggacggggaaattgcagggtggtgggggcgtgaggcttatatgtggaactgatgcagagttcgcctgcagacggatctggatatacactatgtataattgttacgtgtaatttaaaatatatctgtttgccatcgtcatgagaagattatatgtaaggctctgaagggagagggagatgtacattctgccaggctcctggggaccttatccgagtcatgaaattgattactgttgatccagtggtgcaagaagctacactccatgtgtcatcacgcttatgactcctaatgtatttttaaggcaaaaaatgtcagccgactccatcttcacccctcgattcctcgagtccagcctttctgtgccagtgcttcactgagccacaacgctctcgccatcgggacccggctgggcctggagtctcggggcacagttgccatggagccctcctgggtcattctacaaatgtgctgagtgccagctgaaaaccccacaggagatggagtaccttggccaagcttaaagagaagattttctcagggtatttattagtgtgtccagcagggtcaggaagcaggatggaaagatgcactcagactgttaatttattaacaaggcaaatgattttgtgtttcttgatgacagactattaagtttgggacttattttcccatttgagaagttataatatatatttaagatgataagtttcctgcttaagttgtgcctttcagcttcaatgagtttaaggagcactaagggtaatgataccaatgagggttggtttattatcaaacctgaatagctgtggtttctccagtaaatattttcttctactgaacatggagccattattaagagttgtgtgttttttattatgtacatttgtatatttttttgcttgtttgatgttctatttttctaatagttttcttttagtttcttaaagttgtgatactagatttagattctgatgctaactgcaaatcaggttggtctctgctgggtctctcctgcttttattttactttaaggacaagtgtagttgtcgtccaccacctttcaaaaaatgtgaaactgccctgcctcccctttttgctgacaacactgtgtacattgaccacttcctaccatactttatgttgtaaaatcaaactcttttgtggtacattatctcatgcttctgcaaattcgaataaattctatggcttcca

SEQ ID NO: 181 - skipped

SEQ ID NO: 182 - protein, 629 aa, Unknown
  Description of Unknown: CAT-1 sequence
MGCKVLLNIGQQMLRRKVVDCSREETRLSRCLNTFDLVALGVGSTLGAGVYVLAGAVARENAGPAIVISFLIAALASVLAGLCYGEFGARVPKTGSAYLYSYVTVGELWAFITGWNLILSYIIGTSSVARAWSATFDELIGRPIGEFSRTHMTLNAPGVLAENPDIFAVIIILILTGLLTLGVKESAMVNKIFTCINVLVLGFIMVSGFVKGSVKNWQLTEEDFGNTSGRLCLNNDTKEGKPGVGGFMPFGFSGVLSGAATCFYAFVGFDCIATTGEEVKNPQKAIPVGIVASLLICFIAYFGVSAALTLMMPYFCLDNNSPLPDAFKHVGWEGAKYAVAVGSLCALSASLLGSMFPMPRVIYAMAEDGLLFKFLANVNDRTKTPIIATLASGAVAAVMAFLFDLKDLVDLMSIGTLLAYSLVAACVLVLRYQPEQPNLVYQMASTSDELDPADQNELASTNDSQLGFLPEAEMFSLKTILSPKNMEPSKISGLIVNISTSLIAVLIITFCIVTVLGREALTKGALWAVFLLAGSALLCAVVTGVIWRQPESKTKLSFKVPFLPVLPILSIFVNVYLMMQLDQGTWVRFAVWMLIGFIIYFGYGLWHSEEASLDADQARTPDGNLDQCK

SEQ ID NO: 183 - DNA, 1890 bp, Unknown
  Description of Unknown: CAT-1 sequence
atggggtgcaaagtcctgctcaacattgggcagcagatgctgcggcggaaggtggtggactgtagccgggaggagacgcggctgtctcgctgcctgaacacttttgatctggtggccctcggggtgggcagcacactgggtgctggtgtctacgtcctggctggagctgtggcccgtgagaatgcaggccctgccattgtcatctccttcctgatcgctgcgctggcctcagtgctggctggcctgtgctatggcgagtttggtgctcgggtccccaagacgggctcagcttacctctacagctatgtcaccgttggagagctctgggccttcatcaccggctggaacttaatcctctcctacatcatcggtacttcaagcgtagcgagggcctggagcgccaccttcgacgagctgataggcagacccatcggggagttctcacggacacacatgactctgaacgcccccggcgtgctggctgaaaaccccgacatattcgcagtgatcataattctcatcttgacaggacttttaactcttggtgtgaaagagtcggccatggtcaacaaaatattcacttgtattaacgtcctggtcctgggcttcataatggtgtcaggatttgtgaaaggatcggttaaaaactggcagctcacggaggaggattttgggaacacatcaggccgtctctgtttgaacaatgacacaaaagaagggaagcccggtgttggtggattcatgcccttcgggttctctggtgtcctgtcgggggcagcgacttgcttctatgccttcgtgggctttgactgcatcgccaccacaggtgaagaggtgaagaacccacagaaggccatccccgtggggatcgtggcgtccctcttgatctgcttcatcgcctactttggggtgtcggctgccctcacgctcatgatgccctacttctgcctggacaataacagccccctgcccgacgcctttaagcacgtgggctgggaaggtgccaagtacgcagtggccgtgggctccctctgcgctctttccgccagtcttctaggttccatgtttcccatgcctcgggttatctatgccatggctgaggatggactgctatttaaattcttagccaacgtcaatgataggaccaaaacaccaataatcgccacattagcctcgggtgccgttgctgctgtgatggccttcctctttgacctgaaggacttggtggacctcatgtccattggcactctcctggcttactcgttggtggctgcctgtgtgttggtcttacggtaccagccagagcagcctaacctggtataccagatggccagtacttccgacgagttagatccagcagaccaaaatgaattggcaagcaccaatgattcccagctgggctttttaccagaggcagagatgttctctttgaaaaccatactctcacccaaaaacatggagccttccaaaatctctgggctaattgtgaacatttcaaccagcctcatagctgttctcatcatcaccttctgcattgtgaccgtgcttggaagggaggctctcaccaaaggggcgctgtgggcagtctttctgctcgcagggtctgccctcctctgtgccgtggtcacgggcgtcatctggaggcagcccgagagcaagaccaagctctcatttaaggttcccttcctgccagtgctccccatcctgagcatcttcgtgaacgtctatctcatgatgcagctggaccagggcacctgggtccggtttgctgtgtggatgctgataggcttcatcatctactttggctatggcctgtggcacagcgaggaggcgtccctggatgccgaccaagcaaggactcctgacggcaacttggaccagtgcaagtga

SEQ ID NO: 184 - DNA, 7569 bp, Homo sapiens
ctccttctgcagcgcggccggcgggcgctcctcttcgcgggaccagcgaggcggcggccgctgctccagcgtcccccagccgcgggcccccgacgcgctgcagccggcagcccaccgccgccttcttggcgcgaccccaacccagccccagtcgccttcgtcagacgtcagaatgattccttgcagagccgcgctgacctttgcccgatgtctgatccggagaaaaatcgtgaccctggacagtctagaagacaccaaattatgccgctgcttatccaccatggacctcattgccctgggcgttggaagcacccttggggccggggtttatgtcctcgctggggaggtggccaaggcagactcgggccccagcatcgtggtgtccttcctcattgctgccctggcttcagtgatggctggcctctgctatgccgaatttggggcccgtgttcccaagacggggtctgcatatttgtacacctacgtgactgtcggagagctgtgggccttcatcactggctggaatctcattttatcgtatgtgataggtacatcaagtgttgcaagagcctggagtggcacctttgatgaacttcttagcaaacagattggtcagtttttgaggacatacttcagaatgaattacactggtcttgcagaatatcccgatttttttgctgtgtgccttatattacttctagcaggtcttttgtcttttggagtaaaagagtctgcttgggtgaataaagtcttcacagctgttaatattctcgtccttctgtttgtgatggttgctgggtttgtgaaaggaaatgtggcaaactggaagattagtgaagagtttctcaaaaatatatcagcaagtgccagagagccaccttctgaaaacggaacaagtatctatggggctggtggctttatgccttatggctttacgggaacgttggctggtgctgcaacttgcttttatgcctttgtgggatttgactgcattgcaacaactggtgaagaagttcggaatccccagaaagctattcccattggaattgtgacgtctttgcttgtttgctttatggcctattttggggtctctgcagctttaacacttatgatgccgtactacctcctcgatgaaaaaagcccccttcctgtagcgtttgaatatgtgggatggggtcctgccaaatatgtcgtcgcagctggttctctctgcgccttgtcaacaagtcttcttggatccattttcccaatgcctcgtgtaatctatgctatggcggaggatgggttgcttttcaaatgtctagctcaaatcaattccaaaacgaagacaccaataattgctactttatcatcgggtgcagtggcagctttgatggcctttctgtttgacctgaaggcgcttgtggacatgatgtccattggcacactcatggcctactctctggtggcagcctgtgttctcatcctcaggtaccagcctggcttatcttacgaccagcccaaatgttctcctgagaaagatggtctgggatcgtctcccagggtaacctcgaagagtgagtcccaggtcaccatgctgcagagacagggcttcagcatgcggaccctcttctgcccctcccttctgccaacacagcagtcagcttctctcgtgagctttctggtaggattcctagctttcctcgtgttgggcctgagtgtcttgaccacttacggagttcatgccatcaccaggctggaggcctggagcctcgctctcctcgcgctgtttcttgttctcttcgttgccatcgttctcaccatctggaggcagccccagaatcagcaaaaagtagccttcatggttccattcttaccatttttgccagcgttcagcatcttggtgaacatttacttgatggtccagttaagtgcagacacttgggtcagattcagcatttggatggcaattggcttcctgatttacttttcttatggcattagacacagcctggagggtcatctgagagatgaaaacaatgaagaagatgcttatccagacaacgttcatgcagcagcagaagaaaaatctgccattcaagcaaatgaccatcacccaagaaatctcagttcacctttcatattccatgaaaagacaagtgaattctaacacttgcaggagcagagctggtcatcgtcttagcatacatatcctacactgagtaaaccgtaacgggatgtcatcagcatgctgggttgtcatgggtttgctgcatacatagttcaccctaatttatacttactcatctggacagcatctcctcagatggtgaattatgtgcacggggaaacctcctgagtggaagtttcattcatcagtgatgaatagcccccaaacagtgggagtgtgtatgtatgtgtgtatgtatgtatctatgtatatgcttgggaacatgagtgttacaagttagctggtgttttactattattgtgttacatttttccagtgtcgtcattaatcggtggcatatactgcacatactgaaatagagggaaatcactgaatgtaaagaggtttcatctatgccccctgcagttggggaaatactagtagctttaccttgtttgacttcattaatgtcagtttaggggatgccaaaaatgcagttactcatcatggtgtctgtcactggttaggggtaagatgaggggataaggaaagagacttttcaataagttgtgaatgccaacagtgggtttaatgcaaattttttttcctgtgaggtatgacagtttgctcaaacttcagccaacaggggtgtctgcttctgctgcactacacaggccaggagtggcattccatgccactagttggcatccttttgaacttttgtctcctttgcaaacagtggtcctaaaatacgaggtcttcacttgctgtgaatgacgtatccccagtcagggacttaagagaggcactgtgatatacttgggaccctttaaattaaaaagtgaagatagtcaccagggccagaaagctcatggagtggccgtaatgagaatatgtttgaagatcaaagagttagaccaatgcttgaataagtagaccccaagcatccttttctaaaaagtgacttaaaataagccaacagactctcccagaccacacaactagtggaatgattcctcctttttccattacttacttaatcacagtttagtttttttcttaacctcgtcaggcccagagttcacttctttgtttctctgtttcttttgtcttgtcttagagatgagggggctacagcagcatcatgcaaagagggaaagatgaagggatagaagaagagaaaatccccctgttctgataggaacggcctgttccattgttaaatggcaaatggcccaatttaagggctttggatctaatttgcctctgatgtttcctttggaaacatttaggaatatttttctccccctaccccataaattgtgtagcactttttattccatttgctttcaaatgactacactaagcctaataatacaagctccagtgttatacaataacccatcagtgattggggaatcaaacattttggtttaaaaaacatgattatttaaaactggaaactaaaaagaatcaaattgaattaaagctatataaacacagttaacccttgtaaatgagtaaacaaatttttacatgtaagattctctaattgtcatattttactttttaggattccctaatagtggactgtttatttgcagtgtatttgcttctcatgaactatttctcgtacaaatcattaaatagttcattggatgaggctgggtgacatttcccaggacagcatggtgaacattaccaggcatgctagctggcccgtgtaatcccaagacaaggaaaacattcgttttcctcatgggtcttccaagaaatgagctattttattgatgccattaaaaagcaagttgcgatggttttgtatagccaggagtttattgtgattaaacatcaaagaaacaggtagaaagcctgggtttctggctgctagcgttatagcatccatgacacagaactcattacggacattccacaacttccagggtgcacatggtaaaatctgaagcccagaattttctctcaagctgcgtggtttactggagagaaggagttggataagcacaggctcgggtattttggtagggactgtaggcatgctcataaatccttgctgttgtcacagtacgctgaaaacccgtttgattctatacccaatcaagaatagacccttcacacaggaaatgtgaacaattgttatatatgaacactcaaatcttttactgtaacgaaaccaagaaacttgtttagaatgtgataggcagctaaaactgttatgcccactgtgctcaatttgaagcagaatttagtgaaaaattatttttccacattgaaacactttgcagacacaaatatctatgaaaagatgctttgtcagccactgtgcctttttttctgtgaagactcaacggatgtgtgtgtttgtatgtttgttaacagttacatatgtttgtatgagtgtatatatatatctgtgtgtgtgtatctctaacgtcagtgtataagtaagttgggtttatggtgggctttgactatgtcattaggtgggtacaaaacccaactgatgtggagaaaaattgatgtttgatgttgatagatatgcttatacctaatttttagtttttaaactattttaaaatatactatgattttatatgtatatttcctatagactctttaagacgtatttataatgtttctaatatgaaatcactaaactctagtacattatagcaggtgctttgtaatctggaatggagaagaggtaggggcatttggggattcctgtttacttgctgctgccacaccttttccgactgatctgtcctggtaggtgtttattagcaaaagtcagtatcaccagctctttggcacctttctgtttctgcttgtgaattcataatgttttcaactaaattttttttttctttctcagaattacctaaatgttttgtagagttttgactagtaatcaatcaaaattatataaagtcttctccagtaattaagaaatacatatgcaaattcttttgtgattgagtaaaagcagcttaaattacttttcttttctacattaagaaatatattctcaacattttcagtgagaatttcttgtaatggcacctcaaattttatactcttaaaaaaaaacaataatttgtgaattaccaccaaaaggcaatggcagtcctacatttaagaatagagctatgcaaactctgttaaaaactatgaggaaaacttatattagaacttttgatatatactaaaatactgattatcttaatcacattttccccagagataaacattgagagaacgaaagccaaagtgtcatttaagagagatatatatgaaaaagtaacattaatatatagaactttaccatcaccagccgtagttgatagaaaatattagtttcagaattaccctcctttaaaaaataagagactatttgttttcttttaatttctatgaataaaagaaatttttaaaaactttaaaattttaaatattagtcaaaatactttttaagtcctgagtgcttacaggtagttgttaaaaaaattttaaggccaggcatggtggctcgctcacacctataatcctaggatcttgggaggtcgaggcaagctgatcgcttgagcccaggagtttaagaccggcctgagtagcatagcaagaccctgtctctacaaaaaaaacaaaaattagctgggcatggtggcatgcacatgtagtcagagctactgggggtgctgaggtgggaggatcgcttgagcccaggagagtgaggctgcagtgagctgagattacgccactgcactctagcctgggcaacggtgagaccctgcctcaaaaaaaataaaaataaaaataaaacactttaattagaatctatttttacctattttctaaatttatttaaatgcttagcaggaagcataaggaaaagccatcggcctccaatacccatgatgacagagggagcacttgagccttgccttccctcctcttaaatcagggtgtgttccgagattacagaacatcacaccttggcgtgatgaaatcatgccaagattctgactctccctttccggtgatactgctcatgatttctcctaatacgcttcaagcaactgttaccacaaaaaatacagtttccgcagggctttaaaggattgagtttagcatgtatatcatgcgttattaaagttcacgtgattcatgtgaaattaactgtcctttttgctagtgccaaaacagtgccttctctgcacactttacttgtttataaagttctcccacatgtccttaaatatcaagggggaaagtatggatattcgcgtagcaataatgccagcaaaggtcattttcattttttagtcatatagatatgaaaataagttcatatagatatgaaattgcttgactttattgttttggggagattttttttccttacatgattatattaaacactttaaaatagccttccggtttctggattttgagaagcctgatctgttattgttgtggttgttggtgtttgtaatattcattattgtttgtatatacacggtttagtcttactgatttcaaatgcattttgttattgctcaacccaactggtaacactgtttgctgggagcattatacttaactttgattcaccatggttgatgccactgccatgatcgctgggtcttaaagagctttccctagccactgacagccccgtggagatcataatcagggccccaggctggttccaggatcaggcagcctatagagtgtgagcatctatgtgtagctacccttgttgggtgggctcttagactgatggggtaggatatgaagtgaaagacttcaaatgcaagtaaggtagtttgggctccttaattccaaacatcccatgagtatatcaagatgaataaggaccaagggacctctgtgactcatagaagggctggctgaatcctgaagtagcatagtgggacctggtctacaatttatgcacatgcactgacagccttgctgtgccacgtgtctcaccaagacccagttgggaaagagcgtcatattgccaacaggttgggtttctctggcctacacctgattaatgggccctttatctttggtgtcccctaggagtgtccagttgttttattgctgtattttgttattgcagtacttaataaaaattgttgatagggcccaaaaccctacagaaattctatgtctgtaaaaaccaacaaaggcattggacttgtgtgaatgtacagggtttttttagtagtaattttaaatttaaatgttttaagtgatcatcagtgttcctttttacttataaagttggattcttttttagaatttgtaataaataaaaactgctgctttaccactgtaaaatatgctttctgatgtggtgtatttttaaaataaattttaatatgtaataa

SEQ ID NO: 185 - DNA, 7565 bp, Homo sapiens
tcccaaaacagaaagagcagatgtctcaccacgaaactagcaactggaatgaagatagaaacaagtggttataactcagacaaactaatttgtcgagggtttattggaacacctgccccaccggtttgcgacagcaagtttctcctgtcgccttcgtcagacgtcagaatgattccttgcagagccgcgctgacctttgcccgatgtctgatccggagaaaaatcgtgaccctggacagtctagaagacaccaaattatgccgctgcttatccaccatggacctcattgccctgggcgttggaagcacccttggggccggggtttatgtcctcgctggggaggtggccaaggcagactcgggccccagcatcgtggtgtccttcctcattgctgccctggcttcagtgatggctggcctctgctatgccgaatttggggcccgtgttcccaagacggggtctgcatatttgtacacctacgtgactgtcggagagctgtgggccttcatcactggctggaatctcattttatcgtatgtgataggtacatcaagtgttgcaagagcctggagtggcacctttgatgaacttcttagcaaacagattggtcagtttttgaggacatacttcagaatgaattacactggtcttgcagaatatcccgatttttttgctgtgtgccttatattacttctagcaggtcttttgtcttttggagtaaaagagtctgcttgggtgaataaagtcttcacagctgttaatattctcgtccttctgtttgtgatggttgctgggtttgtgaaaggaaatgtggcaaactggaagattagtgaagagtttctcaaaaatatatcagcaagtgccagagagccaccttctgaaaacggaacaagtatctatggggctggtggctttatgccttatggctttacgggaacgttggctggtgctgcaacttgcttttatgcctttgtgggatttgactgcattgcaacaactggtgaagaagttcggaatccccagaaagctattcccattggaattgtgacgtctttgcttgtttgctttatggcctattttggggtctctgcagctttaacacttatgatgccgtactacctcctcgatgaaaaaagcccccttcctgtagcgtttgaatatgtgggatggggtcctgccaaatatgtcgtcgcagctggttctctctgcgccttgtcaacaagtcttcttggatccattttcccaatgcctcgtgtaatctatgctatggcggaggatgggttgcttttcaaatgtctagctcaaatcaattccaaaacgaagacaccaataattgctactttatcatcgggtgcagtggcagctttgatggcctttctgtttgacctgaaggcgcttgtggacatgatgtccattggcacactcatggcctactctctggtggcagcctgtgttctcatcctcaggtaccagcctggcttatcttacgaccagcccaaatgttctcctgagaaagatggtctgggatcgtctcccagggtaacctcgaagagtgagtcccaggtcaccatgctgcagagacagggcttcagcatgcggaccctcttctgcccctcccttctgccaacacagcagtcagcttctctcgtgagctttctggtaggattcctagctttcctcgtgttgggcctgagtgtcttgaccacttacggagttcatgccatcaccaggctggaggcctggagcctcgctctcctcgcgctgtttcttgttctcttcgttgccatcgttctcaccatctggaggcagccccagaatcagcaaaaagtagccttcatggttccattcttaccatttttgccagcgttcagcatcttggtgaacatttacttgatggtccagttaagtgcagacacttgggtcagattcagcatttggatggcaattggcttcctgatttacttttcttatggcattagacacagcctggagggtcatctgagagatgaaaacaatgaagaagatgcttatccagacaacgttcatgcagcagcagaagaaaaatctgccattcaagcaaatgaccatcacccaagaaatctcagttcacctttcatattccatgaaaagacaagtgaattctaacacttgcaggagcagagctggtcatcgtcttagcatacatatcctacactgagtaaaccgtaacgggatgtcatcagcatgctgggttgtcatgggtttgctgcatacatagttcaccctaatttatacttactcatctggacagcatctcctcagatggtgaattatgtgcacggggaaacctcctgagtggaagtttcattcatcagtgatgaatagcccccaaacagtgggagtgtgtatgtatgtgtgtatgtatgtatctatgtatatgcttgggaacatgagtgttacaagttagctggtgttttactattattgtgttacatttttccagtgtcgtcattaatcggtggcatatactgcacatactgaaatagagggaaatcactgaatgtaaagaggtttcatctatgccccctgcagttggggaaatactagtagctttaccttgtttgacttcattaatgtcagtttaggggatgccaaaaatgcagttactcatcatggtgtctgtcactggttaggggtaagatgaggggataaggaaagagacttttcaataagttgtgaatgccaacagtgggtttaatgcaaattttttttcctgtgaggtatgacagtttgctcaaacttcagccaacaggggtgtctgcttctgctgcactacacaggccaggagtggcattccatgccactagttggcatccttttgaacttttgtctcctttgcaaacagtggtcctaaaatacgaggtcttcacttgctgtgaatgacgtatccccagtcagggacttaagagaggcactgtgatatacttgggaccctttaaattaaaaagtgaagatagtcaccagggccagaaagctcatggagtggccgtaatgagaatatgtttgaagatcaaagagttagaccaatgcttgaataagtagaccccaagcatccttttctaaaaagtgacttaaaataagccaacagactctcccagaccacacaactagtggaatgattcctcctttttccattacttacttaatcacagtttagtttttttcttaacctcgtcaggcccagagttcacttctttgtttctctgtttcttttgtcttgtcttagagatgagggggctacagcagcatcatgcaaagagggaaagatgaagggatagaagaagagaaaatccccctgttctgataggaacggcctgttccattgttaaatggcaaatggcccaatttaagggctttggatctaatttgcctctgatgtttcctttggaaacatttaggaatatttttctccccctaccccataaattgtgtagcactttttattccatttgctttcaaatgactacactaagcctaataatacaagctccagtgttatacaataacccatcagtgattggggaatcaaacattttggtttaaaaaacatgattatttaaaactggaaactaaaaagaatcaaattgaattaaagctatataaacacagttaacccttgtaaatgagtaaacaaatttttacatgtaagattctctaattgtcatattttactttttaggattccctaatagtggactgtttatttgcagtgtatttgcttctcatgaactatttctcgtacaaatcattaaatagttcattggatgaggctgggtgacatttcccaggacagcatggtgaacattaccaggcatgctagctggcccgtgtaatcccaagacaaggaaaacattcgttttcctcatgggtcttccaagaaatgagctattttattgatgccattaaaaagcaagttgcgatggttttgtatagccaggagtttattgtgattaaacatcaaagaaacaggtagaaagcctgggtttctggctgctagcgttatagcatccatgacacagaactcattacggacattccacaacttccagggtgcacatggtaaaatctgaagcccagaattttctctcaagctgcgtggtttactggagagaaggagttggataagcacaggctcgggtattttggtagggactgtaggcatgctcataaatccttgctgttgtcacagtacgctgaaaacccgtttgattctatacccaatcaagaatagacccttcacacaggaaatgtgaacaattgttatatatgaacactcaaatcttttactgtaacgaaaccaagaaacttgtttagaatgtgataggcagctaaaactgttatgcccactgtgctcaatttgaagcagaatttagtgaaaaattatttttccacattgaaacactttgcagacacaaatatctatgaaaagatgctttgtcagccactgtgcctttttttctgtgaagactcaacggatgtgtgtgtttgtatgtttgttaacagttacatatgtttgtatgagtgtatatatatatctgtgtgtgtgtatctctaacgtcagtgtataagtaagttgggtttatggtgggctttgactatgtcattaggtgggtacaaaacccaactgatgtggagaaaaattgatgtttgatgttgatagatatgcttatacctaatttttagtttttaaactattttaaaatatactatgattttatatgtatatttcctatagactctttaagacgtatttataatgtttctaatatgaaatcactaaactctagtacattatagcaggtgctttgtaatctggaatggagaagaggtaggggcatttggggattcctgtttacttgctgctgccacaccttttccgactgatctgtcctggtaggtgtttattagcaaaagtcagtatcaccagctctttggcacctttctgtttctgcttgtgaattcataatgttttcaactaaattttttttttctttctcagaattacctaaatgttttgtagagttttgactagtaatcaatcaaaattatataaagtcttctccagtaattaagaaatacatatgcaaattcttttgtgattgagtaaaagcagcttaaattacttttcttttctacattaagaaatatattctcaacattttcagtgagaatttcttgtaatggcacctcaaattttatactcttaaaaaaaaacaataatttgtgaattaccaccaaaaggcaatggcagtcctacatttaagaatagagctatgcaaactctgttaaaaactatgaggaaaacttatattagaacttttgatatatactaaaatactgattatcttaatcacattttccccagagataaacattgagagaacgaaagccaaagtgtcatttaagagagatatatatgaaaaagtaacattaatatatagaactttaccatcaccagccgtagttgatagaaaatattagtttcagaattaccctcctttaaaaaataagagactatttgttttcttttaatttctatgaataaaagaaatttttaaaaactttaaaattttaaatattagtcaaaatactttttaagtcctgagtgcttacaggtagttgttaaaaaaattttaaggccaggcatggtggctcgctcacacctataatcctaggatcttgggaggtcgaggcaagctgatcgcttgagcccaggagtttaagaccggcctgagtagcatagcaagaccctgtctctacaaaaaaaacaaaaattagctgggcatggtggcatgcacatgtagtcagagctactgggggtgctgaggtgggaggatcgcttgagcccaggagagtgaggctgcagtgagctgagattacgccactgcactctagcctgggcaacggtgagaccctgcctcaaaaaaaataaaaataaaaataaaacactttaattagaatctatttttacctattttctaaatttatttaaatgcttagcaggaagcataaggaaaagccatcggcctccaatacccatgatgacagagggagcacttgagccttgccttccctcctcttaaatcagggtgtgttccgagattacagaacatcacaccttggcgtgatgaaatcatgccaagattctgactctccctttccggtgatactgctcatgatttctcctaatacgcttcaagcaactgttaccacaaaaaatacagtttccgcagggctttaaaggattgagtttagcatgtatatcatgcgttattaaagttcacgtgattcatgtgaaattaactgtcctttttgctagtgccaaaacagtgccttctctgcacactttacttgtttataaagttctcccacatgtccttaaatatcaagggggaaagtatggatattcgcgtagcaataatgccagcaaaggtcattttcattttttagtcatatagatatgaaaataagttcatatagatatgaaattgcttgactttattgttttggggagattttttttccttacatgattatattaaacactttaaaatagccttccggtttctggattttgagaagcctgatctgttattgttgtggttgttggtgtttgtaatattcattattgtttgtatatacacggtttagtcttactgatttcaaatgcattttgttattgctcaacccaactggtaacactgtttgctgggagcattatacttaactttgattcaccatggttgatgccactgccatgatcgctgggtcttaaagagctttccctagccactgacagccccgtggagatcataatcagggccccaggctggttccaggatcaggcagcctatagagtgtgagcatctatgtgtagctacccttgttgggtgggctcttagactgatggggtaggatatgaagtgaaagacttcaaatgcaagtaaggtagtttgggctccttaattccaaacatcccatgagtatatcaagatgaataaggaccaagggacctctgtgactcatagaagggctggctgaatcctgaagtagcatagtgggacctggtctacaatttatgcacatgcactgacagccttgctgtgccacgtgtctcaccaagacccagttgggaaagagcgtcatattgccaacaggttgggtttctctggcctacacctgattaatgggccctttatctttggtgtcccctaggagtgtccagttgttttattgctgtattttgttattgcagtacttaataaaaattgttgatagggcccaaaaccctacagaaattctatgtctgtaaaaaccaacaaaggcattggacttgtgtgaatgtacagggtttttttagtagtaattttaaatttaaatgttttaagtgatcatcagtgttcctttttacttataaagttggattcttttttagaatttgtaataaataaaaactgctgctttaccactgtaaaatatgctttctgatgtggtgtatttttaaaataaattttaatatgtaataa

SEQ ID NO: 186 - DNA, 7612 bp, Homo sapiens
ctccttctgcagcgcggccggcgggcgctcctcttcgcgggaccagcgaggcggcggccgctgctccagcgtcccccagccgcgggcccccgacgcgctgcagccggcagcccaccgccgccttcttggcgcgaccccaacccagccccacaggagactctctgaagaccagcaggaaagcagtgagcccttacaggtcgccttcgtcagacgtcagaatgattccttgcagagccgcgctgacctttgcccgatgtctgatccggagaaaaatcgtgaccctggacagtctagaagacaccaaattatgccgctgcttatccaccatggacctcattgccctgggcgttggaagcacccttggggccggggtttatgtcctcgctggggaggtggccaaggcagactcgggccccagcatcgtggtgtccttcctcattgctgccctggcttcagtgatggctggcctctgctatgccgaatttggggcccgtgttcccaagacggggtctgcatatttgtacacctacgtgactgtcggagagctgtgggccttcatcactggctggaatctcattttatcgtatgtgataggtacatcaagtgttgcaagagcctggagtggcacctttgatgaacttcttagcaaacagattggtcagtttttgaggacatacttcagaatgaattacactggtcttgcagaatatcccgatttttttgctgtgtgccttatattacttctagcaggtcttttgtcttttggagtaaaagagtctgcttgggtgaataaagtcttcacagctgttaatattctcgtccttctgtttgtgatggttgctgggtttgtgaaaggaaatgtggcaaactggaagattagtgaagagtttctcaaaaatatatcagcaagtgccagagagccaccttctgaaaacggaacaagtatctatggggctggtggctttatgccttatggctttacgggaacgttggctggtgctgcaacttgcttttatgcctttgtgggatttgactgcattgcaacaactggtgaagaagttcggaatccccagaaagctattcccattggaattgtgacgtctttgcttgtttgctttatggcctattttggggtctctgcagctttaacacttatgatgccgtactacctcctcgatgaaaaaagcccccttcctgtagcgtttgaatatgtgggatggggtcctgccaaatatgtcgtcgcagctggttctctctgcgccttgtcaacaagtcttctgggctctatgtttcctttaccccgaattctgtttgccatggcccgggatggcttactgtttagatttcttgccagagtgagtaagaggcagtcaccagttgctgccacgttgactgcaggggtcatttctgctttgatggcctttctgtttgacctgaaggcgcttgtggacatgatgtccattggcacactcatggcctactctctggtggcagcctgtgttctcatcctcaggtaccagcctggcttatcttacgaccagcccaaatgttctcctgagaaagatggtctgggatcgtctcccagggtaacctcgaagagtgagtcccaggtcaccatgctgcagagacagggcttcagcatgcggaccctcttctgcccctcccttctgccaacacagcagtcagcttctctcgtgagctttctggtaggattcctagctttcctcgtgttgggcctgagtgtcttgaccacttacggagttcatgccatcaccaggctggaggcctggagcctcgctctcctcgcgctgtttcttgttctcttcgttgccatcgttctcaccatctggaggcagccccagaatcagcaaaaagtagccttcatggttccattcttaccatttttgccagcgttcagcatcttggtgaacatttacttgatggtccagttaagtgcagacacttgggtcagattcagcatttggatggcaattggcttcctgatttacttttcttatggcattagacacagcctggagggtcatctgagagatgaaaacaatgaagaagatgcttatccagacaacgttcatgcagcagcagaagaaaaatctgccattcaagcaaatgaccatcacccaagaaatctcagttcacctttcatattccatgaaaagacaagtgaattctaacacttgcaggagcagagctggtcatcgtcttagcatacatatcctacactgagtaaaccgtaacgggatgtcatcagcatgctgggttgtcatgggtttgctgcatacatagttcaccctaatttatacttactcatctggacagcatctcctcagatggtgaattatgtgcacggggaaacctcctgagtggaagtttcattcatcagtgatgaatagcccccaaacagtgggagtgtgtatgtatgtgtgtatgtatgtatctatgtatatgcttgggaacatgagtgttacaagttagctggtgttttactattattgtgttacatttttccagtgtcgtcattaatcggtggcatatactgcacatactgaaatagagggaaatcactgaatgtaaagaggtttcatctatgccccctgcagttggggaaatactagtagctttaccttgtttgacttcattaatgtcagtttaggggatgccaaaaatgcagttactcatcatggtgtctgtcactggttaggggtaagatgaggggataaggaaagagacttttcaataagttgtgaatgccaacagtgggtttaatgcaaattttttttcctgtgaggtatgacagtttgctcaaacttcagccaacaggggtgtctgcttctgctgcactacacaggccaggagtggcattccatgccactagttggcatccttttgaacttttgtctcctttgcaaacagtggtcctaaaatacgaggtcttcacttgctgtgaatgacgtatccccagtcagggacttaagagaggcactgtgatatacttgggaccctttaaattaaaaagtgaagatagtcaccagggccagaaagctcatggagtggccgtaatgagaatatgtttgaagatcaaagagttagaccaatgcttgaataagtagaccccaagcatccttttctaaaaagtgacttaaaataagccaacagactctcccagaccacacaactagtggaatgattcctcctttttccattacttacttaatcacagtttagtttttttcttaacctcgtcaggcccagagttcacttctttgtttctctgtttcttttgtcttgtcttagagatgagggggctacagcagcatcatgcaaagagggaaagatgaagggatagaagaagagaaaatccccctgttctgataggaacggcctgttccattgttaaatggcaaatggcccaatttaagggctttggatctaatttgcctctgatgtttcctttggaaacatttaggaatatttttctccccctaccccataaattgtgtagcactttttattccatttgctttcaaatgactacactaagcctaataatacaagctccagtgttatacaataacccatcagtgattggggaatcaaacattttggtttaaaaaacatgattatttaaaactggaaactaaaaagaatcaaattgaattaaagctatataaacacagttaacccttgtaaatgagtaaacaaatttttacatgtaagattctctaattgtcatattttactttttaggattccctaatagtggactgtttatttgcagtgtatttgcttctcatgaactatttctcgtacaaatcattaaatagttcattggatgaggctgggtgacatttcccaggacagcatggtgaacattaccaggcatgctagctggcccgtgtaatcccaagacaaggaaaacattcgttttcctcatgggtcttccaagaaatgagctattttattgatgccattaaaaagcaagttgcgatggttttgtatagccaggagtttattgtgattaaacatcaaagaaacaggtagaaagcctgggtttctggctgctagcgttatagcatccatgacacagaactcattacggacattccacaacttccagggtgcacatggtaaaatctgaagcccagaattttctctcaagctgcgtggtttactggagagaaggagttggataagcacaggctcgggtattttggtagggactgtaggcatgctcataaatccttgctgttgtcacagtacgctgaaaacccgtttgattctatacccaatcaagaatagacccttcacacaggaaatgtgaacaattgttatatatgaacactcaaatcttttactgtaacgaaaccaagaaacttgtttagaatgtgataggcagctaaaactgttatgcccactgtgctcaatttgaagcagaatttagtgaaaaattatttttccacattgaaacactttgcagacacaaatatctatgaaaagatgctttgtcagccactgtgcctttttttctgtgaagactcaacggatgtgtgtgtttgtatgtttgttaacagttacatatgtttgtatgagtgtatatatatatctgtgtgtgtgtatctctaacgtcagtgtataagtaagttgggtttatggtgggctttgactatgtcattaggtgggtacaaaacccaactgatgtggagaaaaattgatgtttgatgttgatagatatgcttatacctaatttttagtttttaaactattttaaaatatactatgattttatatgtatatttcctatagactctttaagacgtatttataatgtttctaatatgaaatcactaaactctagtacattatagcaggtgctttgtaatctggaatggagaagaggtaggggcatttggggattcctgtttacttgctgctgccacaccttttccgactgatctgtcctggtaggtgtttattagcaaaagtcagtatcaccagctctttggcacctttctgtttctgcttgtgaattcataatgttttcaactaaattttttttttctttctcagaattacctaaatgttttgtagagttttgactagtaatcaatcaaaattatataaagtcttctccagtaattaagaaatacatatgcaaattcttttgtgattgagtaaaagcagcttaaattacttttcttttctacattaagaaatatattctcaacattttcagtgagaatttcttgtaatggcacctcaaattttatactcttaaaaaaaaacaataatttgtgaattaccaccaaaaggcaatggcagtcctacatttaagaatagagctatgcaaactctgttaaaaactatgaggaaaacttatattagaacttttgatatatactaaaatactgattatcttaatcacattttccccagagataaacattgagagaacgaaagccaaagtgtcatttaagagagatatatatgaaaaagtaacattaatatatagaactttaccatcaccagccgtagttgatagaaaatattagtttcagaattaccctcctttaaaaaataagagactatttgttttcttttaatttctatgaataaaagaaatttttaaaaactttaaaattttaaatattagtcaaaatactttttaagtcctgagtgcttacaggtagttgttaaaaaaattttaaggccaggcatggtggctcgctcacacctataatcctaggatcttgggaggtcgaggcaagctgatcgcttgagcccaggagtttaagaccggcctgagtagcatagcaagaccctgtctctacaaaaaaaacaaaaattagctgggcatggtggcatgcacatgtagtcagagctactgggggtgctgaggtgggaggatcgcttgagcccaggagagtgaggctgcagtgagctgagattacgccactgcactctagcctgggcaacggtgagaccctgcctcaaaaaaaataaaaataaaaataaaacactttaattagaatctatttttacctattttctaaatttatttaaatgcttagcaggaagcataaggaaaagccatcggcctccaatacccatgatgacagagggagcacttgagccttgccttccctcctcttaaatcagggtgtgttccgagattacagaacatcacaccttggcgtgatgaaatcatgccaagattctgactctccctttccggtgatactgctcatgatttctcctaatacgcttcaagcaactgttaccacaaaaaatacagtttccgcagggctttaaaggattgagtttagcatgtatatcatgcgttattaaagttcacgtgattcatgtgaaattaactgtcctttttgctagtgccaaaacagtgccttctctgcacactttacttgtttataaagttctcccacatgtccttaaatatcaagggggaaagtatggatattcgcgtagcaataatgccagcaaaggtcattttcattttttagtcatatagatatgaaaataagttcatatagatatgaaattgcttgactttattgttttggggagattttttttccttacatgattatattaaacactttaaaatagccttccggtttctggattttgagaagcctgatctgttattgttgtggttgttggtgtttgtaatattcattattgtttgtatatacacggtttagtcttactgatttcaaatgcattttgttattgctcaacccaactggtaacactgtttgctgggagcattatacttaactttgattcaccatggttgatgccactgccatgatcgctgggtcttaaagagctttccctagccactgacagccccgtggagatcataatcagggccccaggctggttccaggatcaggcagcctatagagtgtgagcatctatgtgtagctacccttgttgggtgggctcttagactgatggggtaggatatgaagtgaaagacttcaaatgcaagtaaggtagtttgggctccttaattccaaacatcccatgagtatatcaagatgaataaggaccaagggacctctgtgactcatagaagggctggctgaatcctgaagtagcatagtgggacctggtctacaatttatgcacatgcactgacagccttgctgtgccacgtgtctcaccaagacccagttgggaaagagcgtcatattgccaacaggttgggtttctctggcctacacctgattaatgggccctttatctttggtgtcccctaggagtgtccagttgttttattgctgtattttgttattgcagtacttaataaaaattgttgatagggcccaaaaccctacagaaattctatgtctgtaaaaaccaacaaaggcattggacttgtgtgaatgtacagggtttttttagtagtaattttaaatttaaatgttttaagtgatcatcagtgttcctttttacttataaagttggattcttttttagaatttgtaataaataaaaactgctgctttaccactgtaaaatatgctttctgatgtggtgtatttttaaaataaattttaatatgtaataa

SEQ ID NO: 187 - DNA, 7615 bp, Homo sapiens
ctccttctgcagcgcggccggcgggcgctcctcttcgcgggaccagcgaggcggcggccgctgctccagcgtcccccagccgcgggcccccgacgcgctgcagccggcagcccaccgccgccttcttggcgcgaccccaacccagccccacaggagactctctgaagaccagcaggaaagcagtgagcccttacaggtcgccttcgtcagacgtcagaatgattccttgcagagccgcgctgacctttgcccgatgtctgatccggagaaaaatcgtgaccctggacagtctagaagacaccaaattatgccgctgcttatccaccatggacctcattgccctgggcgttggaagcacccttggggccggggtttatgtcctcgctggggaggtggccaaggcagactcgggccccagcatcgtggtgtccttcctcattgctgccctggcttcagtgatggctggcctctgctatgccgaatttggggcccgtgttcccaagacggggtctgcatatttgtacacctacgtgactgtcggagagctgtgggccttcatcactggctggaatctcattttatcgtatgtgataggtacatcaagtgttgcaagagcctggagtggcacctttgatgaacttcttagcaaacagattggtcagtttttgaggacatacttcagaatgaattacactggtcttgcagaatatcccgatttttttgctgtgtgccttatattacttctagcaggtcttttgtcttttggagtaaaagagtctgcttgggtgaataaagtcttcacagctgttaatattctcgtccttctgtttgtgatggttgctgggtttgtgaaaggaaatgtggcaaactggaagattagtgaagagtttctcaaaaatatatcagcaagtgccagagagccaccttctgaaaacggaacaagtatctatggggctggtggctttatgccttatggctttacgggaacgttggctggtgctgcaacttgcttttatgcctttgtgggatttgactgcattgcaacaactggtgaagaagttcggaatccccagaaagctattcccattggaattgtgacgtctttgcttgtttgctttatggcctattttggggtctctgcagctttaacacttatgatgccgtactacctcctcgatgaaaaaagcccccttcctgtagcgtttgaatatgtgggatggggtcctgccaaatatgtcgtcgcagctggttctctctgcgccttgtcaacaagtcttcttggatccattttcccaatgcctcgtgtaatctatgctatggcggaggatgggttgcttttcaaatgtctagctcaaatcaattccaaaacgaagacaccaataattgctactttatcatcgggtgcagtggcagctttgatggcctttctgtttgacctgaaggcgcttgtggacatgatgtccattggcacactcatggcctactctctggtggcagcctgtgttctcatcctcaggtaccagcctggcttatcttacgaccagcccaaatgttctcctgagaaagatggtctgggatcgtctcccagggtaacctcgaagagtgagtcccaggtcaccatgctgcagagacagggcttcagcatgcggaccctcttctgcccctcccttctgccaacacagcagtcagcttctctcgtgagctttctggtaggattcctagctttcctcgtgttgggcctgagtgtcttgaccacttacggagttcatgccatcaccaggctggaggcctggagcctcgctctcctcgcgctgtttcttgttctcttcgttgccatcgttctcaccatctggaggcagccccagaatcagcaaaaagtagccttcatggttccattcttaccatttttgccagcgttcagcatcttggtgaacatttacttgatggtccagttaagtgcagacacttgggtcagattcagcatttggatggcaattggcttcctgatttacttttcttatggcattagacacagcctggagggtcatctgagagatgaaaacaatgaagaagatgcttatccagacaacgttcatgcagcagcagaagaaaaatctgccattcaagcaaatgaccatcacccaagaaatctcagttcacctttcatattccatgaaaagacaagtgaattctaacacttgcaggagcagagctggtcatcgtcttagcatacatatcctacactgagtaaaccgtaacgggatgtcatcagcatgctgggttgtcatgggtttgctgcatacatagttcaccctaatttatacttactcatctggacagcatctcctcagatggtgaattatgtgcacggggaaacctcctgagtggaagtttcattcatcagtgatgaatagcccccaaacagtgggagtgtgtatgtatgtgtgtatgtatgtatctatgtatatgcttgggaacatgagtgttacaagttagctggtgttttactattattgtgttacatttttccagtgtcgtcattaatcggtggcatatactgcacatactgaaatagagggaaatcactgaatgtaaagaggtttcatctatgccccctgcagttggggaaatactagtagctttaccttgtttgacttcattaatgtcagtttaggggatgccaaaaatgcagttactcatcatggtgtctgtcactggttaggggtaagatgaggggataaggaaagagacttttcaataagttgtgaatgccaacagtgggtttaatgcaaattttttttcctgtgaggtatgacagtttgctcaaacttcagccaacaggggtgtctgcttctgctgcactacacaggccaggagtggcattccatgccactagttggcatccttttgaacttttgtctcctttgcaaacagtggtcctaaaatacgaggtcttcacttgctgtgaatgacgtatccccagtcagggacttaagagaggcactgtgatatacttgggaccctttaaattaaaaagtgaagatagtcaccagggccagaaagctcatggagtggccgtaatgagaatatgtttgaagatcaaagagttagaccaatgcttgaataagtagaccccaagcatccttttctaaaaagtgacttaaaataagccaacagactctcccagaccacacaactagtggaatgattcctcctttttccattacttacttaatcacagtttagtttttttcttaacctcgtcaggcccagagttcacttctttgtttctctgtttcttttgtcttgtcttagagatgagggggctacagcagcatcatgcaaagagggaaagatgaagggatagaagaagagaaaatccccctgttctgataggaacggcctgttccattgttaaatggcaaatggcccaatttaagggctttggatctaatttgcctctgatgtttcctttggaaacatttaggaatatttttctccccctaccccataaattgtgtagcactttttattccatttgctttcaaatgactacactaagcctaataatacaagctccagtgttatacaataacccatcagtgattggggaatcaaacattttggtttaaaaaacatgattatttaaaactggaaactaaaaagaatcaaattgaattaaagctatataaacacagttaacccttgtaaatgagtaaacaaatttttacatgtaagattctctaattgtcatattttactttttaggattccctaatagtggactgtttatttgcagtgtatttgcttctcatgaactatttctcgtacaaatcattaaatagttcattggatgaggctgggtgacatttcccaggacagcatggtgaacattaccaggcatgctagctggcccgtgtaatcccaagacaaggaaaacattcgttttcctcatgggtcttccaagaaatgagctattttattgatgccattaaaaagcaagttgcgatggttttgtatagccaggagtttattgtgattaaacatcaaagaaacaggtagaaagcctgggtttctggctgctagcgttatagcatccatgacacagaactcattacggacattccacaacttccagggtgcacatggtaaaatctgaagcccagaattttctctcaagctgcgtggtttactggagagaaggagttggataagcacaggctcgggtattttggtagggactgtaggcatgctcataaatccttgctgttgtcacagtacgctgaaaacccgtttgattctatacccaatcaagaatagacccttcacacaggaaatgtgaacaattgttatatatgaacactcaaatcttttactgtaacgaaaccaagaaacttgtttagaatgtgataggcagctaaaactgttatgcccactgtgctcaatttgaagcagaatttagtgaaaaattatttttccacattgaaacactttgcagacacaaatatctatgaaaagatgctttgtcagccactgtgcctttttttctgtgaagactcaacggatgtgtgtgtttgtatgtttgttaacagttacatatgtttgtatgagtgtatatatatatctgtgtgtgtgtatctctaacgtcagtgtataagtaagttgggtttatggtgggctttgactatgtcattaggtgggtacaaaacccaactgatgtggagaaaaattgatgtttgatgttgatagatatgcttatacctaatttttagtttttaaactattttaaaatatactatgattttatatgtatatttcctatagactctttaagacgtatttataatgtttctaatatgaaatcactaaactctagtacattatagcaggtgctttgtaatctggaatggagaagaggtaggggcatttggggattcctgtttacttgctgctgccacaccttttccgactgatctgtcctggtaggtgtttattagcaaaagtcagtatcaccagctctttggcacctttctgtttctgcttgtgaattcataatgttttcaactaaattttttttttctttctcagaattacctaaatgttttgtagagttttgactagtaatcaatcaaaattatataaagtcttctccagtaattaagaaatacatatgcaaattcttttgtgattgagtaaaagcagcttaaattacttttcttttctacattaagaaatatattctcaacattttcagtgagaatttcttgtaatggcacctcaaattttatactcttaaaaaaaaacaataatttgtgaattaccaccaaaaggcaatggcagtcctacatttaagaatagagctatgcaaactctgttaaaaactatgaggaaaacttatattagaacttttgatatatactaaaatactgattatcttaatcacattttccccagagataaacattgagagaacgaaagccaaagtgtcatttaagagagatatatatgaaaaagtaacattaatatatagaactttaccatcaccagccgtagttgatagaaaatattagtttcagaattaccctcctttaaaaaataagagactatttgttttcttttaatttctatgaataaaagaaatttttaaaaactttaaaattttaaatattagtcaaaatactttttaagtcctgagtgcttacaggtagttgttaaaaaaattttaaggccaggcatggtggctcgctcacacctataatcctaggatcttgggaggtcgaggcaagctgatcgcttgagcccaggagtttaagaccggcctgagtagcatagcaagaccctgtctctacaaaaaaaacaaaaattagctgggcatggtggcatgcacatgtagtcagagctactgggggtgctgaggtgggaggatcgcttgagcccaggagagtgaggctgcagtgagctgagattacgccactgcactctagcctgggcaacggtgagaccctgcctcaaaaaaaataaaaataaaaataaaacactttaattagaatctatttttacctattttctaaatttatttaaatgcttagcaggaagcataaggaaaagccatcggcctccaatacccatgatgacagagggagcacttgagccttgccttccctcctcttaaatcagggtgtgttccgagattacagaacatcacaccttggcgtgatgaaatcatgccaagattctgactctccctttccggtgatactgctcatgatttctcctaatacgcttcaagcaactgttaccacaaaaaatacagtttccgcagggctttaaaggattgagtttagcatgtatatcatgcgttattaaagttcacgtgattcatgtgaaattaactgtcctttttgctagtgccaaaacagtgccttctctgcacactttacttgtttataaagttctcccacatgtccttaaatatcaagggggaaagtatggatattcgcgtagcaataatgccagcaaaggtcattttcattttttagtcatatagatatgaaaataagttcatatagatatgaaattgcttgactttattgttttggggagattttttttccttacatgattatattaaacactttaaaatagccttccggtttctggattttgagaagcctgatctgttattgttgtggttgttggtgtttgtaatattcattattgtttgtatatacacggtttagtcttactgatttcaaatgcattttgttattgctcaacccaactggtaacactgtttgctgggagcattatacttaactttgattcaccatggttgatgccactgccatgatcgctgggtcttaaagagctttccctagccactgacagccccgtggagatcataatcagggccccaggctggttccaggatcaggcagcctatagagtgtgagcatctatgtgtagctacccttgttgggtgggctcttagactgatggggtaggatatgaagtgaaagacttcaaatgcaagtaaggtagtttgggctccttaattccaaacatcccatgagtatatcaagatgaataaggaccaagggacctctgtgactcatagaagggctggctgaatcctgaagtagcatagtgggacctggtctacaatttatgcacatgcactgacagccttgctgtgccacgtgtctcaccaagacccagttgggaaagagcgtcatattgccaacaggttgggtttctctggcctacacctgattaatgggccctttatctttggtgtcccctaggagtgtccagttgttttattgctgtattttgttattgcagtacttaataaaaattgttgatagggcccaaaaccctacagaaattctatgtctgtaaaaaccaacaaaggcattggacttgtgtgaatgtacagggtttttttagtagtaattttaaatttaaatgttttaagtgatcatcagtgttcctttttacttataaagttggattcttttttagaatttgtaataaataaaaactgctgctttaccactgtaaaatatgctttctgatgtggtgtatttttaaaataaattttaatatgtaataa

SEQ ID NO: 188 - DNA, 7562 bp, Homo sapiens
tcccaaaacagaaagagcagatgtctcaccacgaaactagcaactggaatgaagatagaaacaagtggttataactcagacaaactaatttgtcgagggtttattggaacacctgccccaccggtttgcgacagcaagtttctcctgtcgccttcgtcagacgtcagaatgattccttgcagagccgcgctgacctttgcccgatgtctgatccggagaaaaatcgtgaccctggacagtctagaagacaccaaattatgccgctgcttatccaccatggacctcattgccctgggcgttggaagcacccttggggccggggtttatgtcctcgctggggaggtggccaaggcagactcgggccccagcatcgtggtgtccttcctcattgctgccctggcttcagtgatggctggcctctgctatgccgaatttggggcccgtgttcccaagacggggtctgcatatttgtacacctacgtgactgtcggagagctgtgggccttcatcactggctggaatctcattttatcgtatgtgataggtacatcaagtgttgcaagagcctggagtggcacctttgatgaacttcttagcaaacagattggtcagtttttgaggacatacttcagaatgaattacactggtcttgcagaatatcccgatttttttgctgtgtgccttatattacttctagcaggtcttttgtcttttggagtaaaagagtctgcttgggtgaataaagtcttcacagctgttaatattctcgtccttctgtttgtgatggttgctgggtttgtgaaaggaaatgtggcaaactggaagattagtgaagagtttctcaaaaatatatcagcaagtgccagagagccaccttctgaaaacggaacaagtatctatggggctggtggctttatgccttatggctttacgggaacgttggctggtgctgcaacttgcttttatgcctttgtgggatttgactgcattgcaacaactggtgaagaagttcggaatccccagaaagctattcccattggaattgtgacgtctttgcttgtttgctttatggcctattttggggtctctgcagctttaacacttatgatgccgtactacctcctcgatgaaaaaagcccccttcctgtagcgtttgaatatgtgggatggggtcctgccaaatatgtcgtcgcagctggttctctctgcgccttgtcaacaagtcttctgggctctatgtttcctttaccccgaattctgtttgccatggcccgggatggcttactgtttagatttcttgccagagtgagtaagaggcagtcaccagttgctgccacgttgactgcaggggtcatttctgctttgatggcctttctgtttgacctgaaggcgcttgtggacatgatgtccattggcacactcatggcctactctctggtggcagcctgtgttctcatcctcaggtaccagcctggcttatcttacgaccagcccaaatgttctcctgagaaagatggtctgggatcgtctcccagggtaacctcgaagagtgagtcccaggtcaccatgctgcagagacagggcttcagcatgcggaccctcttctgcccctcccttctgccaacacagcagtcagcttctctcgtgagctttctggtaggattcctagctttcctcgtgttgggcctgagtgtcttgaccacttacggagttcatgccatcaccaggctggaggcctggagcctcgctctcctcgcgctgtttcttgttctcttcgttgccatcgttctcaccatctggaggcagccccagaatcagcaaaaagtagccttcatggttccattcttaccatttttgccagcgttcagcatcttggtgaacatttacttgatggtccagttaagtgcagacacttgggtcagattcagcatttggatggcaattggcttcctgatttacttttcttatggcattagacacagcctggagggtcatctgagagatgaaaacaatgaagaagatgcttatccagacaacgttcatgcagcagcagaagaaaaatctgccattcaagcaaatgaccatcacccaagaaatctcagttcacctttcatattccatgaaaagacaagtgaattctaacacttgcaggagcagagctggtcatcgtcttagcatacatatcctacactgagtaaaccgtaacgggatgtcatcagcatgctgggttgtcatgggtttgctgcatacatagttcaccctaatttatacttactcatctggacagcatctcctcagatggtgaattatgtgcacggggaaacctcctgagtggaagtttcattcatcagtgatgaatagcccccaaacagtgggagtgtgtatgtatgtgtgtatgtatgtatctatgtatatgcttgggaacatgagtgttacaagttagctggtgttttactattattgtgttacatttttccagtgtcgtcattaatcggtggcatatactgcacatactgaaatagagggaaatcactgaatgtaaagaggtttcatctatgccccctgcagttggggaaatactagtagctttaccttgtttgacttcattaatgtcagtttaggggatgccaaaaatgcagttactcatcatggtgtctgtcactggttaggggtaagatgaggggataaggaaagagacttttcaataagttgtgaatgccaacagtgggtttaatgcaaattttttttcctgtgaggtatgacagtttgctcaaacttcagccaacaggggtgtctgcttctgctgcactacacaggccaggagtggcattccatgccactagttggcatccttttgaacttttgtctcctttgcaaacagtggtcctaaaatacgaggtcttcacttgctgtgaatgacgtatccccagtcagggacttaagagaggcactgtgatatacttgggaccctttaaattaaaaagtgaagatagtcaccagggccagaaagctcatggagtggccgtaatgagaatatgtttgaagatcaaagagttagaccaatgcttgaataagtagaccccaagcatccttttctaaaaagtgacttaaaataagccaacagactctcccagaccacacaactagtggaatgattcctcctttttccattacttacttaatcacagtttagtttttttcttaacctcgtcaggcccagagttcacttctttgtttctctgtttcttttgtcttgtcttagagatgagggggctacagcagcatcatgcaaagagggaaagatgaagggatagaagaagagaaaatccccctgttctgataggaacggcctgttccattgttaaatggcaaatggcccaatttaagggctttggatctaatttgcctctgatgtttcctttggaaacatttaggaatatttttctccccctaccccataaattgtgtagcactttttattccatttgctttcaaatgactacactaagcctaataatacaagctccagtgttatacaataacccatcagtgattggggaatcaaacattttggtttaaaaaacatgattatttaaaactggaaactaaaaagaatcaaattgaattaaagctatataaacacagttaacccttgtaaatgagtaaacaaatttttacatgtaagattctctaattgtcatattttactttttaggattccctaatagtggactgtttatttgcagtgtatttgcttctcatgaactatttctcgtacaaatcattaaatagttcattggatgaggctgggtgacatttcccaggacagcatggtgaacattaccaggcatgctagctggcccgtgtaatcccaagacaaggaaaacattcgttttcctcatgggtcttccaagaaatgagctattttattgatgccattaaaaagcaagttgcgatggttttgtatagccaggagtttattgtgattaaacatcaaagaaacaggtagaaagcctgggtttctggctgctagcgttatagcatccatgacacagaactcattacggacattccacaacttccagggtgcacatggtaaaatctgaagcccagaattttctctcaagctgcgtggtttactggagagaaggagttggataagcacaggctcgggtattttggtagggactgtaggcatgctcataaatccttgctgttgtcacagtacgctgaaaacccgtttgattctatacccaatcaagaatagacccttcacacaggaaatgtgaacaattgttatatatgaacactcaaatcttttactgtaacgaaaccaagaaacttgtttagaatgtgataggcagctaaaactgttatgcccactgtgctcaatttgaagcagaatttagtgaaaaattatttttccacattgaaacactttgcagacacaaatatctatgaaaagatgctttgtcagccactgtgcctttttttctgtgaagactcaacggatgtgtgtgtttgtatgtttgttaacagttacatatgtttgtatgagtgtatatatatatctgtgtgtgtgtatctctaacgtcagtgtataagtaagttgggtttatggtgggctttgactatgtcattaggtgggtacaaaacccaactgatgtggagaaaaattgatgtttgatgttgatagatatgcttatacctaatttttagtttttaaactattttaaaatatactatgattttatatgtatatttcctatagactctttaagacgtatttataatgtttctaatatgaaatcactaaactctagtacattatagcaggtgctttgtaatctggaatggagaagaggtaggggcatttggggattcctgtttacttgctgctgccacaccttttccgactgatctgtcctggtaggtgtttattagcaaaagtcagtatcaccagctctttggcacctttctgtttctgcttgtgaattcataatgttttcaactaaattttttttttctttctcagaattacctaaatgttttgtagagttttgactagtaatcaatcaaaattatataaagtcttctccagtaattaagaaatacatatgcaaattcttttgtgattgagtaaaagcagcttaaattacttttcttttctacattaagaaatatattctcaacattttcagtgagaatttcttgtaatggcacctcaaattttatactcttaaaaaaaaacaataatttgtgaattaccaccaaaaggcaatggcagtcctacatttaagaatagagctatgcaaactctgttaaaaactatgaggaaaacttatattagaacttttgatatatactaaaatactgattatcttaatcacattttccccagagataaacattgagagaacgaaagccaaagtgtcatttaagagagatatatatgaaaaagtaacattaatatatagaactttaccatcaccagccgtagttgatagaaaatattagtttcagaattaccctcctttaaaaaataagagactatttgttttcttttaatttctatgaataaaagaaatttttaaaaactttaaaattttaaatattagtcaaaatactttttaagtcctgagtgcttacaggtagttgttaaaaaaattttaaggccaggcatggtggctcgctcacacctataatcctaggatcttgggaggtcgaggcaagctgatcgcttgagcccaggagtttaagaccggcctgagtagcatagcaagaccctgtctctacaaaaaaaacaaaaattagctgggcatggtggcatgcacatgtagtcagagctactgggggtgctgaggtgggaggatcgcttgagcccaggagagtgaggctgcagtgagctgagattacgccactgcactctagcctgggcaacggtgagaccctgcctcaaaaaaaataaaaataaaaataaaacactttaattagaatctatttttacctattttctaaatttatttaaatgcttagcaggaagcataaggaaaagccatcggcctccaatacccatgatgacagagggagcacttgagccttgccttccctcctcttaaatcagggtgtgttccgagattacagaacatcacaccttggcgtgatgaaatcatgccaagattctgactctccctttccggtgatactgctcatgatttctcctaatacgcttcaagcaactgttaccacaaaaaatacagtttccgcagggctttaaaggattgagtttagcatgtatatcatgcgttattaaagttcacgtgattcatgtgaaattaactgtcctttttgctagtgccaaaacagtgccttctctgcacactttacttgtttataaagttctcccacatgtccttaaatatcaagggggaaagtatggatattcgcgtagcaataatgccagcaaaggtcattttcattttttagtcatatagatatgaaaataagttcatatagatatgaaattgcttgactttattgttttggggagattttttttccttacatgattatattaaacactttaaaatagccttccggtttctggattttgagaagcctgatctgttattgttgtggttgttggtgtttgtaatattcattattgtttgtatatacacggtttagtcttactgatttcaaatgcattttgttattgctcaacccaactggtaacactgtttgctgggagcattatacttaactttgattcaccatggttgatgccactgccatgatcgctgggtcttaaagagctttccctagccactgacagccccgtggagatcataatcagggccccaggctggttccaggatcaggcagcctatagagtgtgagcatctatgtgtagctacccttgttgggtgggctcttagactgatggggtaggatatgaagtgaaagacttcaaatgcaagtaaggtagtttgggctccttaattccaaacatcccatgagtatatcaagatgaataaggaccaagggacctctgtgactcatagaagggctggctgaatcctgaagtagcatagtgggacctggtctacaatttatgcacatgcactgacagccttgctgtgccacgtgtctcaccaagacccagttgggaaagagcgtcatattgccaacaggttgggtttctctggcctacacctgattaatgggccctttatctttggtgtcccctaggagtgtccagttgttttattgctgtattttgttattgcagtacttaataaaaattgttgatagggcccaaaaccctacagaaattctatgtctgtaaaaaccaacaaaggcattggacttgtgtgaatgtacagggtttttttagtagtaattttaaatttaaatgttttaagtgatcatcagtgttcctttttacttataaagttggattcttttttagaatttgtaataaataaaaactgctgctttaccactgtaaaatatgctttctgatgtggtgtatttttaaaataaattttaatatgtaataa

SEQ ID NO: 189-193 - skipped (5 entries)

SEQ ID NO: 194 - protein, 697 aa, Unknown
  Description of Unknown: CAT-2A sequence
MKIETSGYNSDKLICRGFIGTPAPPVCDSKFLLSPSSDVRMIPCRAALTFARCLIRRKIVTLDSLEDTKLCRCLSTMDLIALGVGSTLGAGVYVLAGEVAKADSGPSIVVSFLIAALASVMAGLCYAEFGARVPKTGSAYLYTYVTVGELWAFITGWNLILSYVIGTSSVARAWSGTFDELLSKQIGQFLRTYFRMNYTGLAEYPDFFAVCLILLLAGLLSFGVKESAWVNKVFTAVNILVLLFVMVAGFVKGNVANWKISEEFLKNISASAREPPSENGTSIYGAGGFMPYGFTGTLAGAATCFYAFVGFDCIATTGEEVRNPQKAIPIGIVTSLLVCFMAYFGVSAALTLMMPYYLLDEKSPLPVAFEYVGWGPAKYVVAAGSLCALSTSLLGSMFPLPRILFAMARDGLLFRFLARVSKRQSPVAATLTAGVISALMAFLFDLKALVDMMSIGTLMAYSLVAACVLILRYQPGLSYDQPKCSPEKDGLGSSPRVTSKSESQVTMLQRQGFSMRTLFCPSLLPTQQSASLVSFLVGFLAFLVLGLSVLTTYGVHAITRLEAWSLALLALFLVLFVAIVLTIWRQPQNQQKVAFMVPFLPFLPAFSILVNIYLMVQLSADTWVRFSIWMAIGFLIYFSYGIRHSLEGHLRDENNEEDAYPDNVHAAAEEKSAIQANDHHPRNLSSPFIFHEKTSEF

SEQ ID NO: 195 - protein, 658 aa, Unknown
  Description of Unknown: CAT-2B sequence
MIPCRAALTFARCLIRRKIVTLDSLEDTKLCRCLSTMDLIALGVGSTLGAGVYVLAGEVAKADSGPSIVVSFLIAALASVMAGLCYAEFGARVPKTGSAYLYTYVTVGELWAFITGWNLILSYVIGTSSVARAWSGTFDELLSKQIGQFLRTYFRMNYTGLAEYPDFFAVCLILLLAGLLSFGVKESAWVNKVFTAVNILVLLFVMVAGFVKGNVANWKISEEFLKNISASAREPPSENGTSIYGAGGFMPYGFTGTLAGAATCFYAFVGFDCIATTGEEVRNPQKAIPIGIVTSLLVCFMAYFGVSAALTLMMPYYLLDEKSPLPVAFEYVGWGPAKYVVAAGSLCALSTSLLGSIFPMPRVIYAMAEDGLLFKCLAQINSKTKTPIIATLSSGAVAALMAFLFDLKALVDMMSIGTLMAYSLVAACVLILRYQPGLSYDQPKCSPEKDGLGSSPRVTSKSESQVTMLQRQGFSMRTLFCPSLLPTQQSASLVSFLVGFLAFLVLGLSVLTTYGVHAITRLEAWSLALLALFLVLFVAIVLTIWRQPQNQQKVAFMVPFLPFLPAFSILVNIYLMVQLSADTWVRFSIWMAIGFLIYFSYGIRHSLEGHLRDENNEEDAYPDNVHAAAEEKSAIQANDHHPRNLSSPFIFHEKTSEF

SEQ ID NO: 196 - DNA, 2094 bp, Unknown
  Description of Unknown: CAT-2A sequence
atgaagatagaaacaagtggttataactcagacaaactaatttgtcgagggtttattggaacacctgccccaccggtttgcgacagcaagtttctcctgtcgccttcgtcagacgtcagaatgattccttgcagagccgcgctgacctttgcccgatgtctgatccggagaaaaatcgtgaccctggacagtctagaagacaccaaattatgccgctgcttatccaccatggacctcattgccctgggcgttggaagcacccttggggccggggtttatgtcctcgctggggaggtggccaaggcagactcgggccccagcatcgtggtgtccttcctcattgctgccctggcttcagtgatggctggcctctgctatgccgaatttggggcccgtgttcccaagacggggtctgcatatttgtacacctacgtgactgtcggagagctgtgggccttcatcactggctggaatctcattttatcgtatgtgataggtacatcaagtgttgcaagagcctggagtggcacctttgatgaacttcttagcaaacagattggtcagtttttgaggacatacttcagaatgaattacactggtcttgcagaatatcccgatttttttgctgtgtgccttatattacttctagcaggtcttttgtcttttggagtaaaagagtctgcttgggtgaataaagtcttcacagctgttaatattctcgtccttctgtttgtgatggttgctgggtttgtgaaaggaaatgtggcaaactggaagattagtgaagagtttctcaaaaatatatcagcaagtgccagagagccaccttctgaaaacggaacaagtatctatggggctggtggctttatgccttatggctttacgggaacgttggctggtgctgcaacttgcttttatgcctttgtgggatttgactgcattgcaacaactggtgaagaagttcggaatccccagaaagctattcccattggaattgtgacgtctttgcttgtttgctttatggcctattttggggtctctgcagctttaacacttatgatgccgtactacctcctcgatgaaaaaagcccccttcctgtagcgtttgaatatgtgggatggggtcctgccaaatatgtcgtcgcagctggttctctctgcgccttgtcaacaagtcttctgggctctatgtttcctttaccccgaattctgtttgccatggcccgggatggcttactgtttagatttcttgccagagtgagtaagaggcagtcaccagttgctgccacgttgactgcaggggtcatttctgctttgatggcctttctgtttgacctgaaggcgcttgtggacatgatgtccattggcacactcatggcctactctctggtggcagcctgtgttctcatcctcaggtaccagcctggcttatcttacgaccagcccaaatgttctcctgagaaagatggtctgggatcgtctcccagggtaacctcgaagagtgagtcccaggtcaccatgctgcagagacagggcttcagcatgcggaccctcttctgcccctcccttctgccaacacagcagtcagcttctctcgtgagctttctggtaggattcctagctttcctcgtgttgggcctgagtgtcttgaccacttacggagttcatgccatcaccaggctggaggcctggagcctcgctctcctcgcgctgtttcttgttctcttcgttgccatcgttctcaccatctggaggcagccccagaatcagcaaaaagtagccttcatggttccattcttaccatttttgccagcgttcagcatcttggtgaacatttacttgatggtccagttaagtgcagacacttgggtcagattcagcatttggatggcaattggcttcctgatttacttttcttatggcattagacacagcctggagggtcatctgagagatgaaaacaatgaagaagatgcttatccagacaacgttcatgcagcagcagaagaaaaatctgccattcaagcaaatgaccatcacccaagaaatctcagttcacctttcatattccatgaaaagacaagtgaattctaa

SEQ ID NO: 197 - DNA, 1977 bp, Unknown
  Description of Unknown: CAT-2B sequence
atgattccttgcagagccgcgctgacctttgcccgatgtctgatccggagaaaaatcgtgaccctggacagtctagaagacaccaaattatgccgctgcttatccaccatggacctcattgccctgggcgttggaagcacccttggggccggggtttatgtcctcgctggggaggtggccaaggcagactcgggccccagcatcgtggtgtccttcctcattgctgccctggcttcagtgatggctggcctctgctatgccgaatttggggcccgtgttcccaagacggggtctgcatatttgtacacctacgtgactgtcggagagctgtgggccttcatcactggctggaatctcattttatcgtatgtgataggtacatcaagtgttgcaagagcctggagtggcacctttgatgaacttcttagcaaacagattggtcagtttttgaggacatacttcagaatgaattacactggtcttgcagaatatcccgatttttttgctgtgtgccttatattacttctagcaggtcttttgtcttttggagtaaaagagtctgcttgggtgaataaagtcttcacagctgttaatattctcgtccttctgtttgtgatggttgctgggtttgtgaaaggaaatgtggcaaactggaagattagtgaagagtttctcaaaaatatatcagcaagtgccagagagccaccttctgaaaacggaacaagtatctatggggctggtggctttatgccttatggctttacgggaacgttggctggtgctgcaacttgcttttatgcctttgtgggatttgactgcattgcaacaactggtgaagaagttcggaatccccagaaagctattcccattggaattgtgacgtctttgcttgtttgctttatggcctattttggggtctctgcagctttaacacttatgatgccgtactacctcctcgatgaaaaaagcccccttcctgtagcgtttgaatatgtgggatggggtcctgccaaatatgtcgtcgcagctggttctctctgcgccttgtcaacaagtcttcttggatccattttcccaatgcctcgtgtaatctatgctatggcggaggatgggttgcttttcaaatgtctagctcaaatcaattccaaaacgaagacaccaataattgctactttatcatcgggtgcagtggcagctttgatggcctttctgtttgacctgaaggcgcttgtggacatgatgtccattggcacactcatggcctactctctggtggcagcctgtgttctcatcctcaggtaccagcctggcttatcttacgaccagcccaaatgttctcctgagaaagatggtctgggatcgtctcccagggtaacctcgaagagtgagtcccaggtcaccatgctgcagagacagggcttcagcatgcggaccctcttctgcccctcccttctgccaacacagcagtcagcttctctcgtgagctttctggtaggattcctagctttcctcgtgttgggcctgagtgtcttgaccacttacggagttcatgccatcaccaggctggaggcctggagcctcgctctcctcgcgctgtttcttgttctcttcgttgccatcgttctcaccatctggaggcagccccagaatcagcaaaaagtagccttcatggttccattcttaccatttttgccagcgttcagcatcttggtgaacatttacttgatggtccagttaagtgcagacacttgggtcagattcagcatttggatggcaattggcttcctgatttacttttcttatggcattagacacagcctggagggtcatctgagagatgaaaacaatgaagaagatgcttatccagacaacgttcatgcagcagcagaagaaaaatctgccattcaagcaaatgaccatcacccaagaaatctcagttcacctttcatattccatgaaaagacaagtgaattctaa

SEQ ID NO: 198 - protein, 657 aa, Artificial Sequence
  Description of Artificial Sequence: Synthetic polypeptide
MIPCRAALTFARCLIRRKIVTLDSLEDTKLCRCLSTMDLIALGVGSTLGAGVYVLAGEVAKADSGPSIVVSFLIAALASVMAGLCYAEFGARVPKTGSAYLYTYVTVGELWAFITGWNLILSYVIGTSSVARAWSGTFDELLSKQIGQFLRTYFRMNYTGLAEYPDFFAVCLILLLAGLLSFGVKESAWVNKVFTAVNILVLLFVMVAGFVKGNVANWKISEEFLKNISASAREPPSENGTSIYGAGGFMPYGFTGTLAGAATCFYAFVGFDCIATTGEEVRNPQKAIPIGIVTSLLVCFMAYFGVSAALTLMMPYYLLDEKSPLPVAFEYVGWGPAKYVVAAGSLCALSTSLLGSMFPLPRILFAMAEDGLLFRFLARVSKRQSPVAATLTAGVISALMAFLFDLKALVDMMSIGTLMAYSLVAACVLILRYQPGLSYDQPKCSPEKDGLGSSPRVTSKSESQVTMLQRQGFSMRTLFCPSLLPTQQSASLVSFLVGFLAFLVLGLSVLTTYGVHAITRLEAWSLALLALFLVLFVAIVLTIWRQPQNQQKVAFMVPFLPFLPAFSILVNIYLMVQLSADTWVRFSIWMAIGFLIYFSYGIRHSLEGHLRDENNEEDAYPDNVHAAAEEKSAIQANDHHPRNLSSPFIFHEKTSEF

SEQ ID NO: 199 - protein, 658 aa, Artificial Sequence
  Description of Artificial Sequence: Synthetic polypeptide
MIPCRAALTFARCLIRRKIVTLDSLEDTKLCRCLSTMDLIALGVGSTLGAGVYVLAGEVAKADSGPSIVVSFLIAALASVMAGLCYAEFGARVPKTGSAYLYTYVTVGELWAFITGWNLILSYVIGTSSVARAWSGTFDELLSKQIGQFLRTYFRMNYTGLAEYPDFFAVCLILLLAGLLSFGVKESAWVNKVFTAVNILVLLFVMVAGFVKGNVANWKISEEFLKNISASAREPPSENGTSIYGAGGFMPYGFTGTLAGAATCFYAFVGFDCIATTGEEVRNPQKAIPIGIVTSLLVCFMAYFGVSAALTLMMPYYLLDEKSPLPVAFEYVGWGPAKYVVAAGSLCALSTSLLGSMFPLPRILFAMARDGLLFRFLARVNSKRQSPVAATLTAGVISALMAFLFDLKALVDMMSIGTLMAYSLVAACVLILRYQPGLSYDQPKCSPEKDGLGSSPRVTSKSESQVTMLQRQGFSMRTLFCPSLLPTQQSASLVSFLVGFLAFLVLGLSVLTTYGVHAITRLEAWSLALLALFLVLFVAIVLTIWRQPQNQQKVAFMVPFLPFLPAFSILVNIYLMVQLSADTWVRFSIWMAIGFLIYFSYGIRHSLEGHLRDENNEEDAYPDNVHAAAEEKSAIQANDHHPRNLSSPFIFHEKTSEF

SEQ ID NO: 200 - protein, 658 aa, Artificial Sequence
  Description of Artificial Sequence: Synthetic polypeptide
MIPCRAALTFARCLIRRKIVTLDSLEDTKLCRCLSTMDLIALGVGSTLGAGVYVLAGEVAKADSGPSIVVSFLIAALASVMAGLCYAEFGARVPKTGSAYLYTYVTVGELWAFITGWNLILSYVIGTSSVARAWSGTFDELLSKQIGQFLRTYFRMNYTGLAEYPDFFAVCLILLLAGLLSFGVKESAWVNKVFTAVNILVLLFVMVAGFVKGNVANWKISEEFLKNISASAREPPSENGTSIYGAGGFMPYGFTGTLAGAATCFYAFVGFDCIATTGEEVRNPQKAIPIGIVTSLLVCFMAYFGVSAALTLMMPYYLLDEKSPLPVAFEYVGWGPAKYVVAAGSLCALSTSLLGSMFPLPRILFAMAEDGLLFRFLARVNSKRQSPVAATLTAGVISALMAFLFDLKALVDMMSIGTLMAYSLVAACVLILRYQPGLSYDQPKCSPEKDGLGSSPRVTSKSESQVTMLQRQGFSMRTLFCPSLLPTQQSASLVSFLVGFLAFLVLGLSVLTTYGVHAITRLEAWSLALLALFLVLFVAIVLTIWRQPQNQQKVAFMVPFLPFLPAFSILVNIYLMVQLSADTWVRFSIWMAIGFLIYFSYGIRHSLEGHLRDENNEEDAYPDNVHAAAEEKSAIQANDHHPRNLSSPFIFHEKTSEF

SEQ ID NO: 201 - DNA, 1971 bp, Artificial Sequence
  Description of Artificial Sequence: Synthetic polynucleotide
atgattccctgcagagccgctctgaccttcgccagatgcctgatcagacggaagatcgtgaccctggacagcctggaagataccaagctgtgccggtgcctgagcaccatggatctgattgccctcggcgtgggctctacacttggagctggtgtttatgtgctggctggcgaggtggccaaggccgattctggaccttctatcgtggtgtccttcctgatcgccgctctggcctctgttatggccggactgtgttacgccgagttcggagccagagtgcctaagacaggcagcgcctacctgtacacctacgtgacagtgggagagctgtgggcctttatcaccggctggaacctgatcctgagctacgtgatcggcacctcctctgtggctagagcttggagcggcacctttgacgagctgctgtctaagcagatcggccagttcctgcggacctacttccggatgaattacaccggcctggccgagtatcccgacttcttcgccgtgtgtctgatcctgctgcttgccggactgctgagcttcggcgtgaaagagtctgcctgggtcaacaaggtgttcaccgccgtgaatatcctggtgctgctgttcgtgatggtggccggcttcgtgaagggcaacgtggccaattggaagatcagcgaagagttcctgaagaacatcagcgccagcgccagagagcctccttctgaaaacggcaccagcatctatggcgcaggcggctttatgccctacggctttactggaacactggcaggcgccgctacctgcttctatgccttcgtgggcttcgactgtatcgccaccactggggaagaagtgcggaaccctcagaaggctatccccatcggcatcgtgacaagcctgctcgtgtgcttcatggcctacttcggagtgtccgccgcactgaccctgatgatgccttactacctgctggacgagaagtcccctctgcctgtggcctttgagtatgttggctggggccctgccaaatacgtggtggctgctggatctctgtgcgccctgtctacatctctgctgggcagcatgttccctctgccaagaatcctgttcgccatggccgaggatggcctgctgttcagattcctggccagagtgagcaagcggcagtctcctgtggccgctacacttacagctggcgtgatctctgccctgatggctttcctgttcgacctgaaggccctggtggacatgatgagcatcggcacactgatggcctacagcctggtggcagcctgcgtgctgattctgagataccagccaggcctgtcctacgaccagcctaagtgttcccctgagaaggacggcctgggcagctctcctagagtgacaagcaagagcgagagccaagtgaccatgctgcagagacagggcttcagcatgcggaccctgttctgcccttctctgctgcctacacagcagtctgctagcctggtgtctttcctcgtgggatttctggcctttctggtgctgggcctgagcgtgctgacaacatatggggtgcacgccatcaccagactggaagcttggagtctggctctgctggccctgttcctggttctgtttgtggccatcgtgctgaccatttggcggcagccccagaaccagcagaaagtggctttcatggtgccctttctgcctttcctgccagccttcagcatcctggtcaacatctacctgatggtgcagctgagcgccgacacctgggtccgattttccatctggatggctatcggcttcctcatctacttcagctacggcatccggcactccctggaaggccatctgagagatgagaacaacgaagaggacgcttaccccgacaacgtgcacgccgctgccgaagagaaatctgccatccaggccaacgaccaccatccaagaaacctgagcagccccttcatcttccacgagaaaaccagcgagttt

SEQ ID NO: 202 - DNA, 1974 bp, Artificial Sequence
  Description of Artificial Sequence: Synthetic polynucleotide
atgattccctgcagagccgctctgaccttcgccagatgcctgatcagacggaagatcgtgaccctggacagcctggaagataccaagctgtgccggtgcctgagcaccatggatctgattgccctcggcgtgggctctacacttggagctggtgtttatgtgctggctggcgaggtggccaaggccgattctggaccttctatcgtggtgtccttcctgatcgccgctctggcctctgttatggccggactgtgttacgccgagttcggagccagagtgcctaagacaggcagcgcctacctgtacacctacgtgacagtgggagagctgtgggcctttatcaccggctggaacctgatcctgagctacgtgatcggcacctcctctgtggctagagcttggagcggcacctttgacgagctgctgtctaagcagatcggccagttcctgcggacctacttccggatgaattacaccggcctggccgagtatcccgacttcttcgccgtgtgtctgatcctgctgcttgccggactgctgagcttcggcgtgaaagagtctgcctgggtcaacaaggtgttcaccgccgtgaatatcctggtgctgctgttcgtgatggtggccggcttcgtgaagggcaacgtggccaattggaagatcagcgaagagttcctgaagaacatcagcgccagcgccagagagcctccttctgaaaacggcaccagcatctatggcgcaggcggctttatgccctacggctttactggaacactggcaggcgccgctacctgcttctatgccttcgtgggcttcgactgtatcgccaccactggggaagaagtgcggaaccctcagaaggctatccccatcggcatcgtgacaagcctgctcgtgtgcttcatggcctacttcggagtgtccgccgcactgaccctgatgatgccttactacctgctggacgagaagtcccctctgcctgtggcctttgagtatgttggctggggccctgccaaatacgtggtggctgctggatctctgtgcgccctgtctacatctctgctgggcagcatgttccctctgccaagaatcctgttcgccatggcccgggatggcctgctgttcagattcctggccagagtgaacagcaagcggcagtctcctgtggccgctacacttacagctggcgtgatctctgccctgatggctttcctgttcgacctgaaggccctggtggacatgatgagcatcggcacactgatggcctacagcctggtggcagcctgcgtgctgattctgagataccagccaggcctgtcctacgaccagcctaagtgttcccctgagaaggacggcctgggcagctctcctagagtgacaagcaagagcgagagccaagtgaccatgctgcagagacagggcttcagcatgcggaccctgttctgcccttctctgctgcctacacagcagtctgctagcctggtgtctttcctcgtgggatttctggcctttctggtgctgggcctgagcgtgctgacaacatatggggtgcacgccatcaccagactggaagcttggagtctggctctgctggccctgttcctggttctgtttgtggccatcgtgctgaccatttggcggcagccccagaaccagcagaaagtggctttcatggtgccctttctgcctttcctgccagccttcagcatcctggtcaacatctacctgatggtgcagctgagcgccgacacctgggtccgattttccatctggatggctatcggcttcctcatctacttcagctacggcatccggcactccctggaaggccatctgagagatgagaacaacgaagaggacgcttaccccgacaacgtgcacgccgctgccgaagagaaatctgccatccaggccaacgaccaccatccaagaaacctgagcagccccttcatcttccacgagaaaaccagcgagttt

SEQ ID NO: 203 - DNA, 1974 bp, Artificial Sequence
  Description of Artificial Sequence: Synthetic polynucleotide
atgattccctgcagagccgctctgaccttcgccagatgcctgatcagacggaagatcgtgaccctggacagcctggaagataccaagctgtgccggtgcctgagcaccatggatctgattgccctcggcgtgggctctacacttggagctggtgtttatgtgctggctggcgaggtggccaaggccgattctggaccttctatcgtggtgtccttcctgatcgccgctctggcctctgttatggccggactgtgttacgccgagttcggagccagagtgcctaagacaggcagcgcctacctgtacacctacgtgacagtgggagagctgtgggcctttatcaccggctggaacctgatcctgagctacgtgatcggcacctcctctgtggctagagcttggagcggcacctttgacgagctgctgtctaagcagatcggccagttcctgcggacctacttccggatgaattacaccggcctggccgagtatcccgacttcttcgccgtgtgtctgatcctgctgcttgccggactgctgagcttcggcgtgaaagagtctgcctgggtcaacaaggtgttcaccgccgtgaatatcctggtgctgctgttcgtgatggtggccggcttcgtgaagggcaacgtggccaattggaagatcagcgaagagttcctgaagaacatcagcgccagcgccagagagcctccttctgaaaacggcaccagcatctatggcgcaggcggctttatgccctacggctttactggaacactggcaggcgccgctacctgcttctatgccttcgtgggcttcgactgtatcgccaccactggggaagaagtgcggaaccctcagaaggctatccccatcggcatcgtgacaagcctgctcgtgtgcttcatggcctacttcggagtgtccgccgcactgaccctgatgatgccttactacctgctggacgagaagtcccctctgcctgtggcctttgagtatgttggctggggccctgccaaatacgtggtggctgctggatctctgtgcgccctgtctacatctctgctgggcagcatgttccctctgccaagaatcctgttcgccatggccgaggatggcctgctgttcagattcctggccagagtgaacagcaagcggcagtctcctgtggccgctacacttacagctggcgtgatctctgccctgatggctttcctgttcgacctgaaggccctggtggacatgatgagcatcggcacactgatggcctacagcctggtggcagcctgcgtgctgattctgagataccagccaggcctgtcctacgaccagcctaagtgttcccctgagaaggacggcctgggcagctctcctagagtgacaagcaagagcgagagccaagtgaccatgctgcagagacagggcttcagcatgcggaccctgttctgcccttctctgctgcctacacagcagtctgctagcctggtgtctttcctcgtgggatttctggcctttctggtgctgggcctgagcgtgctgacaacatatggggtgcacgccatcaccagactggaagcttggagtctggctctgctggccctgttcctggttctgtttgtggccatcgtgctgaccatttggcggcagccccagaaccagcagaaagtggctttcatggtgccctttctgcctttcctgccagccttcagcatcctggtcaacatctacctgatggtgcagctgagcgccgacacctgggtccgattttccatctggatggctatcggcttcctcatctacttcagctacggcatccggcactccctggaaggccatctgagagatgagaacaacgaagaggacgcttaccccgacaacgtgcacgccgctgccgaagagaaatctgccatccaggccaacgaccaccatccaagaaacctgagcagccccttcatcttccacgagaaaaccagcgagttt

SEQ ID NO: 204 - DNA, 2215 bp, Homo sapiens
aagccgcagctttgaagcctgagcggccgaactcggcagctccaacccaactcggcttaactccgcctcaccgagcccagtccaagactctgtgctccctaggtttgcaacagctctctggatgccgtggcaagcatttcgcagatttggtcaaaagctggtacgcagacgtacactggagtcaggcatggctgagactcgccttgccagatgcctaagcaccctggatttagtggccctgggtgtgggcagcacattgggtgcaggcgtgtatgtcctagctggcgaggtggccaaagataaagcagggccatccattgtgatctgctttttggtggctgccctgtcttctgtgttggctgggctgtgctatgcggagtttggtgcccgggttccccgttctggttcggcatatctctacagctatgtcactgtgggtgaactctgggccttcaccactggctggaacctcatcctctcctatgtcattggtacagccagtgtggcccgggcctggagctctgcttttgacaacctgattgggaaccacatctctaagactctgcaggggtccattgcactgcacgtgccccatgtccttgcagaatatccagatttctttgctttgggcctcgtgttgctgctcactggattgttggctctcggggctagtgagtcggccctggttaccaaagtgttcacaggcgtgaaccttttggttcttgggttcgtcatgatctctggcttcgttaagggggacgtgcacaactggaagctcacagaagaggactacgaattggccatggctgaactcaatgacacctatagcttgggtcctctgggctctggaggatttgtgcctttcggcttcgagggaattctccgtggagcagcgacctgtttctatgcatttgttggtttcgactgtattgctaccactggagaagaagcccagaatccccagcgttccatcccgatgggcattgtgatctcactgtctgtctgctttttggcgtattttgctgtctcttctgcactcaccctgatgatgccttactaccagcttcagcctgagagccctttgcctgaggcatttctctacattggatgggctcctgcccgctatgttgtggctgttggctccctctgtgctctttctaccagcctcctgggctccatgttccccatgcctcgggtgatctacgcgatggcagaggatggcctcctgttccgtgtacttgctcggatccacaccggcacacgcaccccaatcatagccaccgtggtctctggcattattgcagcattcatggcattcctcttcaaactcactgatcttgtggacctcatgtcaattgggaccctgcttgcttactccctggtgtcgatttgtgttctcatcctcaggtatcaacctgatcaggagacaaagactggggaagaagtggagttgcaggaggaggcaataactactgaatcagagaagttgaccctatggggactatttttcccactcaactccatccccactccactctctggccaaattgtctatgtttgttcctcattgcttgctgtcctgctgactgctctttgcctggtgctggcccagtggtcagttccattgctttctggagacctgctgtggactgcagtggttgtgctgctcctgctgctcattattgggatcattgtggtcatctggagacagccacagagttccactccccttcactttaaggtgcctgctttgcctctcctcccactaatgagcatctttgtgaatatttaccttatgatgcagatgacagctggtacctgggcccgatttggggtctggatgctgattggctttgctatctacttcggctatgggatccagcacagcctggaagagattaagagtaaccaaccctcacgcaagtctagagccaaaactgtagaccttgatcccggcactctctatgtccactcagtttgacatcgtcacacctaaatgctgtctggtcccctgcacaataatggagagtactcctgaccccagtgacagctagccctcccctgtgatggtggtggtggatactaatacagttctgtacgatgtgaaggatgtgtctttgctatttcttgtctattttaacccgtctgcttctaaatgatgtctagctgcttaccaactttaaaaaatgatattaaaagaaagtagaaaaataaa

SEQ ID NO: 205 - DNA, 2239 bp, Homo sapiens
aagccgcagctttgaagcctgagcggccgaactcggcagctccaacccaactcggcttaactccgcctcaccgagcccagtccaagactctgtgctccctaggtttgcaacagctctctgatcatcttcttcaattcctgctaggatgccgtggcaagcatttcgcagatttggtcaaaagctggtacgcagacgtacactggagtcaggcatggctgagactcgccttgccagatgcctaagcaccctggatttagtggccctgggtgtgggcagcacattgggtgcaggcgtgtatgtcctagctggcgaggtggccaaagataaagcagggccatccattgtgatctgctttttggtggctgccctgtcttctgtgttggctgggctgtgctatgcggagtttggtgcccgggttccccgttctggttcggcatatctctacagctatgtcactgtgggtgaactctgggccttcaccactggctggaacctcatcctctcctatgtcattggtacagccagtgtggcccgggcctggagctctgcttttgacaacctgattgggaaccacatctctaagactctgcaggggtccattgcactgcacgtgccccatgtccttgcagaatatccagatttctttgctttgggcctcgtgttgctgctcactggattgttggctctcggggctagtgagtcggccctggttaccaaagtgttcacaggcgtgaaccttttggttcttgggttcgtcatgatctctggcttcgttaagggggacgtgcacaactggaagctcacagaagaggactacgaattggccatggctgaactcaatgacacctatagcttgggtcctctgggctctggaggatttgtgcctttcggcttcgagggaattctccgtggagcagcgacctgtttctatgcatttgttggtttcgactgtattgctaccactggagaagaagcccagaatccccagcgttccatcccgatgggcattgtgatctcactgtctgtctgctttttggcgtattttgctgtctcttctgcactcaccctgatgatgccttactaccagcttcagcctgagagccctttgcctgaggcatttctctacattggatgggctcctgcccgctatgttgtggctgttggctccctctgtgctctttctaccagcctcctgggctccatgttccccatgcctcgggtgatctacgcgatggcagaggatggcctcctgttccgtgtacttgctcggatccacaccggcacacgcaccccaatcatagccaccgtggtctctggcattattgcagcattcatggcattcctcttcaaactcactgatcttgtggacctcatgtcaattgggaccctgcttgcttactccctggtgtcgatttgtgttctcatcctcaggtatcaacctgatcaggagacaaagactggggaagaagtggagttgcaggaggaggcaataactactgaatcagagaagttgaccctatggggactatttttcccactcaactccatccccactccactctctggccaaattgtctatgtttgttcctcattgcttgctgtcctgctgactgctctttgcctggtgctggcccagtggtcagttccattgctttctggagacctgctgtggactgcagtggttgtgctgctcctgctgctcattattgggatcattgtggtcatctggagacagccacagagttccactccccttcactttaaggtgcctgctttgcctctcctcccactaatgagcatctttgtgaatatttaccttatgatgcagatgacagctggtacctgggcccgatttggggtctggatgctgattggctttgctatctacttcggctatgggatccagcacagcctggaagagattaagagtaaccaaccctcacgcaagtctagagccaaaactgtagaccttgatcccggcactctctatgtccactcagtttgacatcgtcacacctaaatgctgtctggtcccctgcacaataatggagagtactcctgaccccagtgacagctagccctcccctgtgatggtggtggtggatactaatacagttctgtacgatgtgaaggatgtgtctttgctatttcttgtctattttaacccgtctgcttctaaatgatgtctagctgcttaccaactttaaaaaatgatattaaaagaaagtagaaaaataaa

SEQ ID NO: 206-207 - skipped (2 entries)

SEQ ID NO: 208 - protein, 619 aa, Unknown
  Description of Unknown: CAT-3 sequence
MPWQAFRRFGQKLVRRRTLESGMAETRLARCLSTLDLVALGVGSTLGAGVYVLAGEVAKDKAGPSIVICFLVAALSSVLAGLCYAEFGARVPRSGSAYLYSYVTVGELWAFTTGWNLILSYVIGTASVARAWSSAFDNLIGNHISKTLQGSIALHVPHVLAEYPDFFALGLVLLLTGLLALGASESALVTKVFTGVNLLVLGFVMISGFVKGDVHNWKLTEEDYELAMAELNDTYSLGPLGSGGFVPFGFEGILRGAATCFYAFVGFDCIATTGEEAQNPQRSIPMGIVISLSVCFLAYFAVSSALTLMMPYYQLQPESPLPEAFLYIGWAPARYVVAVGSLCALSTSLLGSMFPMPRVIYAMAEDGLLFRVLARIHTGTRTPIIATVVSGIIAAFMAFLFKLTDLVDLMSIGTLLAYSLVSICVLILRYQPDQETKTGEEVELQEEAITTESEKLTLWGLFFPLNSIPTPLSGQIVYVCSSLLAVLLTALCLVLAQWSVPLLSGDLLWTAVVVLLLLLIIGIIVVIWRQPQSSTPLHFKVPALPLLPLMSIFVNIYLMMQMTAGTWARFGVWMLIGFAIYFGYGIQHSLEEIKSNQPSRKSRAKTVDLDPGTLYVHSV

SEQ ID NO: 209 - DNA, 1860 bp, Unknown
  Description of Unknown: CAT-3 sequence
atgccgtggcaagcatttcgcagatttggtcaaaagctggtacgcagacgtacactggagtcaggcatggctgagactcgccttgccagatgcctaagcaccctggatttagtggccctgggtgtgggcagcacattgggtgcaggcgtgtatgtcctagctggcgaggtggccaaagataaagcagggccatccattgtgatctgctttttggtggctgccctgtcttctgtgttggctgggctgtgctatgcggagtttggtgcccgggttccccgttctggttcggcatatctctacagctatgtcactgtgggtgaactctgggccttcaccactggctggaacctcatcctctcctatgtcattggtacagccagtgtggcccgggcctggagctctgcttttgacaacctgattgggaaccacatctctaagactctgcaggggtccattgcactgcacgtgccccatgtccttgcagaatatccagatttctttgctttgggcctcgtgttgctgctcactggattgttggctctcggggctagtgagtcggccctggttaccaaagtgttcacaggcgtgaaccttttggttcttgggttcgtcatgatctctggcttcgttaagggggacgtgcacaactggaagctcacagaagaggactacgaattggccatggctgaactcaatgacacctatagcttgggtcctctgggctctggaggatttgtgcctttcggcttcgagggaattctccgtggagcagcgacctgtttctatgcatttgttggtttcgactgtattgctaccactggagaagaagcccagaatccccagcgttccatcccgatgggcattgtgatctcactgtctgtctgctttttggcgtattttgctgtctcttctgcactcaccctgatgatgccttactaccagcttcagcctgagagccctttgcctgaggcatttctctacattggatgggctcctgcccgctatgttgtggctgttggctccctctgtgctctttctaccagcctcctgggctccatgttccccatgcctcgggtgatctacgcgatggcagaggatggcctcctgttccgtgtacttgctcggatccacaccggcacacgcaccccaatcatagccaccgtggtctctggcattattgcagcattcatggcattcctcttcaaactcactgatcttgtggacctcatgtcaattgggaccctgcttgcttactccctggtgtcgatttgtgttctcatcctcaggtatcaacctgatcaggagacaaagactggggaagaagtggagttgcaggaggaggcaataactactgaatcagagaagttgaccctatggggactatttttcccactcaactccatccccactccactctctggccaaattgtctatgtttgttcctcattgcttgctgtcctgctgactgctctttgcctggtgctggcccagtggtcagttccattgctttctggagacctgctgtggactgcagtggttgtgctgctcctgctgctcattattgggatcattgtggtcatctggagacagccacagagttccactccccttcactttaaggtgcctgctttgcctctcctcccactaatgagcatctttgtgaatatttaccttatgatgcagatgacagctggtacctgggcccgatttggggtctggatgctgattggctttgctatctacttcggctatgggatccagcacagcctggaagagattaagagtaaccaaccctcacgcaagtctagagccaaaactgtagaccttgatcccggcactctctatgtccactcagtttga

SEQ ID NO: 210 - DNA, 2316 bp, Homo sapiens
agagcggaggcagcggctgcggcagcagcaggttccagtagctggctcggtgctcttctcggccacctgccatggcccgggggctgcccaccattgctagcctggcacgcttatgccagaagctgaaccgcctgaagccgctggaggactccaccatggagacgtcactgcggcgctgcctgtccacgctggacctgactcttctgggcgtgggtggcatggtgggctcgggtctctacgtgctcacaggtgccgtggccaaggaggtggctggccctgctgtgctcttgtccttcggtgtggccgctgtggcctccctgctggcagccctatgctatgcagaatttggggcacgtgtgccacgcacgggctctgcctacctgttcacctacgtatccatgggcgagctgtgggccttcctcatcggctggaatgttctcctcgaatacatcatcggtggcgccgccgtggcccgtgcctggagtggctacctggactctatgttcagccacagcatccgcaacttcactgagacccacgtgggttcttggcaggtgcccctcctgggccactacccggacttcctggctgctggcatcatcctcctggcctctgcctttgtctcctgtggagcccgcgtgtcctcctggctcaatcacaccttctcggccatcagcctgcttgtcattctcttcattgtcatcctgggcttcatcctggcccagcctcacaactggagcgctgacgaaggcggctttgcacccttcggcttctccggcgtcatggccggcactgcctcctgcttctatgctttcgtgggcttcgacgtcattgccgcctccagtgaggaggcccagaacccacggcggtctgtgcctctggccatcgccatctcgcttgccattgcagctggtgcctacatccttgtctccaccgtgctaaccctcatggtgccctggcacagcctggaccccgactcagcgcttgcagatgccttctaccagcggggctacaggtgggctggcttcatcgtggcagctggctccatctgcgccatgaacaccgtcctgctcagcctcctcttctccctgccacgcattgtctatgccatggccgccgatgggctcttcttccaggtgtttgcccatgtgcacccccggacacaggtgcctgtggcgggcaccctggcgttcgggctcctcacggccttcctggcactgctgctggacctggagtcgctggttcagttcctgtcccttggcacactcctggcctacacattcgtggccaccagtatcattgtgctgcgcttccagaagtcttccccgcccagctccccaggcccagccagccctggccccctgaccaagcagcagagctccttctcagaccacctacagctggtgggcactgtacacgcctccgtccctgagccaggggagctgaagccagccctgaggccctacctgggcttcttggatgggtacagccctggagcagtggtgacttgggcgcttggcgttatgttggcctcagccatcaccataggctgcgtgcttgtctttgggaactcgaccctgcacctcccacactggggttacatcctgctgctcctgctcaccagtgtcatgtttctgctcagcctccttgtcctgggggctcaccagcaacagtatcgggaagacttatttcagatccccatggttcccctgattccagccctgagcatcgtcctcaacatctgcctcatgctgaaacttagctatctgacctgggtgcgcttctccatctggctgctgatgggacttgcagtgtatttcggctatggcatccggcatagcaaggagaaccagcgggagctgccagggctgaactccacacactacgtggtattccccaggggcagcctggaggagacagtgcaggctatgcagccccccagccaggcaccagcacaggaccctggccatatggagtagctgatcagcccacacttgccccgccctcccacacctgcttgggaggccagagaggccagacaagccgagagccccttctgttgtgggcagcctgggtttgcaggcctgcacaggctggggagtcctcaggaccttaggaccttcatccaggggctgggcttcgggtcttcaggagtgggccttggctggtgctggtgccatggactctgcccagagccttcttgtttatgatcagctccagctacctgggcagttgtggtggggtggatgggaaggcccacagcccaagggatccataataataattgcttggccagccatgtggcctgctggcgta

SEQ ID NO: 211 - skipped

SEQ ID NO: 212 - protein, 635 aa, Unknown
  Description of Unknown: CAT-4 sequence
MARGLPTIASLARLCQKLNRLKPLEDSTMETSLRRCLSTLDLTLLGVGGMVGSGLYVLTGAVAKEVAGPAVLLSFGVAAVASLLAALCYAEFGARVPRTGSAYLFTYVSMGELWAFLIGWNVLLEYIIGGAAVARAWSGYLDSMFSHSIRNFTETHVGSWQVPLLGHYPDFLAAGIILLASAFVSCGARVSSWLNHTFSAISLLVILFIVILGFILAQPHNWSADEGGFAPFGFSGVMAGTASCFYAFVGFDVIAASSEEAQNPRRSVPLAIAISLAIAAGAYILVSTVLTLMVPWHSLDPDSALADAFYQRGYRWAGFIVAAGSICAMNTVLLSLLFSLPRIVYAMAADGLFFQVFAHVHPRTQVPVAGTLAFGLLTAFLALLLDLESLVQFLSLGTLLAYTFVATSIIVLRFQKSSPPSSPGPASPGPLTKQQSSFSDHLQLVGTVHASVPEPGELKPALRPYLGFLDGYSPGAVVTWALGVMLASAITIGCVLVFGNSTLHLPHWGYILLLLLTSVMFLLSLLVLGAHQQQYREDLFQIPMVPLIPALSIVLNICLMLKLSYLTWVRFSIWLLMGLAVYFGYGIRHSKENQRELPGLNSTHYVVFPRGSLEETVQAMQPPSQAPAQDPGHME

SEQ ID NO: 213 - DNA, 1908 bp, Unknown
  Description of Unknown: CAT-4 sequence
atggcccgggggctgcccaccattgctagcctggcacgcttatgccagaagctgaaccgcctgaagccgctggaggactccaccatggagacgtcactgcggcgctgcctgtccacgctggacctgactcttctgggcgtgggtggcatggtgggctcgggtctctacgtgctcacaggtgccgtggccaaggaggtggctggccctgctgtgctcttgtccttcggtgtggccgctgtggcctccctgctggcagccctatgctatgcagaatttggggcacgtgtgccacgcacgggctctgcctacctgttcacctacgtatccatgggcgagctgtgggccttcctcatcggctggaatgttctcctcgaatacatcatcggtggcgccgccgtggcccgtgcctggagtggctacctggactctatgttcagccacagcatccgcaacttcactgagacccacgtgggttcttggcaggtgcccctcctgggccactacccggacttcctggctgctggcatcatcctcctggcctctgcctttgtctcctgtggagcccgcgtgtcctcctggctcaatcacaccttctcggccatcagcctgcttgtcattctcttcattgtcatcctgggcttcatcctggcccagcctcacaactggagcgctgacgaaggcggctttgcacccttcggcttctccggcgtcatggccggcactgcctcctgcttctatgctttcgtgggcttcgacgtcattgccgcctccagtgaggaggcccagaacccacggcggtctgtgcctctggccatcgccatctcgcttgccattgcagctggtgcctacatccttgtctccaccgtgctaaccctcatggtgccctggcacagcctggaccccgactcagcgcttgcagatgccttctaccagcggggctacaggtgggctggcttcatcgtggcagctggctccatctgcgccatgaacaccgtcctgctcagcctcctcttctccctgccacgcattgtctatgccatggccgccgatgggctcttcttccaggtgtttgcccatgtgcacccccggacacaggtgcctgtggcgggcaccctggcgttcgggctcctcacggccttcctggcactgctgctggacctggagtcgctggttcagttcctgtcccttggcacactcctggcctacacattcgtggccaccagtatcattgtgctgcgcttccagaagtcttccccgcccagctccccaggcccagccagccctggccccctgaccaagcagcagagctccttctcagaccacctacagctggtgggcactgtacacgcctccgtccctgagccaggggagctgaagccagccctgaggccctacctgggcttcttggatgggtacagccctggagcagtggtgacttgggcgcttggcgttatgttggcctcagccatcaccataggctgcgtgcttgtctttgggaactcgaccctgcacctcccacactggggttacatcctgctgctcctgctcaccagtgtcatgtttctgctcagcctccttgtcctgggggctcaccagcaacagtatcgggaagacttatttcagatccccatggttcccctgattccagccctgagcatcgtcctcaacatctgcctcatgctgaaacttagctatctgacctgggtgcgcttctccatctggctgctgatgggacttgcagtgtatttcggctatggcatccggcatagcaaggagaaccagcgggagctgccagggctgaactccacacactacgtggtattccccaggggcagcctggaggagacagtgcaggctatgcagccccccagccaggcaccagcacaggaccctggccatatggagtag

SEQ ID NO: 214 - DNA, 6342 bp, Homo sapiens
gcggcggcggcggcgcgaccgagcatcctggcggcgccgggccactgggagagtttatgtggccgaggcagacaagtggaattaggccttgctgcaggggacttcatttccttctcagtactggacccatttatgaggaggtggcttatgaaagtgtgatgttcgcgtatttcttgacaggcagtggcgtgatcttggctcactgcaacctccgactccctggttcaagcgattctcctgcctcagcctcctgagtggggattacaggccacagcaaacacaggtgtgcaggaaccgtttgtcatggaagccagggagcctgggaggcccacacccacctaccatcttgtccctaacaccagccagtcccaggtggaagaagatgtcagctcgccacctcaaaggtcctccgaaactatgcagctgaagaaggagatctccctgctgaatggggtcagcctggtggtgggcaacatgatcggctcagggatctttgtctcacccaagggtgtgctggtacacactgcctcctatgggatgtcactgattgtgtgggccattggtgggctcttctctgttgtgggtgccctttgttatgcagagctggggaccaccatcaccaagtcgggagccagctacgcttatattctagaggcctttgggggcttcattgccttcatccgcctgtgggtctcactgctagttgttgagcccaccggtcaggccatcatcgccatcacctttgccaactacatcatccagccgtccttccccagctgtgatcccccatacctggcctgccgtctcctggctgctgcttgcatatgtctgctgacatttgtgaactgtgcctatgtcaagtggggcacacgtgtgcaggacacgttcacttacgccaaggtcgtagcgctcattgccatcattgtcatgggccttgttaaactgtgccagggacactctgagcactttcaggacgcctttgagggttcctcctgggacatgggaaacctctctcttgccctctactctgccctcttctcttactcaggttgggacacccttaattttgtaacagaagaaatcaaaaacccagaaagaaatttgcccttggccattgggatttctatgccaattgtgacgctcatctacatcctgaccaatgtggcctattacacagtgctgaacatttcagatgtccttagcagtgatgctgtggctgtgacatttgctgaccagacgtttggcatgttcagctggaccatccccattgctgttgccctgtcctgctttgggggcctcaatgcatccatctttgcttcatcaaggttgttcttcgtgggctcccgggagggccacctaccggaccttctgtccatgatccacattgagcgttttacacctatccctgctttactgttcaattgcaccatggcactcatctacctcatcgtggaggatgttttccagcttatcaactacttcagcttcagctactggttcttcgtgggcctgtctgttgttggacagctctacctccgctggaaggagcccaagcggccccggcctctcaagctgagcgtgtttttccccatcgtgttctgcatatgctccgtgtttctggtgatagtgcccctcttcactgacaccattaattccctcattggcatcgggattgccctttctggagtccctttctacttcatgggtgtttacctgccagagtcccggaggccattgtttattcggaatgtcctggctgctatcaccagaggcacccagcagctttgcttttgtgtcctgactgagcttgatgtagccgaagaaaaaaaggatgagaggaaaactgactagaggtcagaggtggctttctgaggcctggaaggcaggccaaccagcaaaatcctgataacaagactctgtgggcccaactctcctgaattaaaggagccttttgacccaatcatatagtggggctcagggccagtgctcactcttattggtaagctataggagactcaggatctgggccaacctcaaggtgggggcttcagagggtggggggaagattggggaacggggggaatggtcatttagttttactcctgataggtagatgcagctcttacagatatttacttggtaaagtgcagtggggaagagggaatgctaggttgatagggctggtggcttctgaatttggtatttgaactaggagtccctatagaggggctgctttatgggaagtttttctctgaccaggtacaacacctgactttaaaggcctgaaatgctaccatttcttcctctggctcaaaattcttccctggggagagagttatattcccttatttattgatatttagtccagaacaccagttctaacgaagcatgcgtgtctcttcatctacaggatgcaataggctgattgtatttaaaaatcaaagtacccaaaactgagtccctttgggctcagaaatgtctgtggtattgggtcagactctgaccacaggttttatgctgtttagcacaatttctattgagtcttacctgcaacaatgaaccttaaagatttttttactcacgtacctgttacactttagcatacagatagatcatagatcacgttacaagcacttggctcaggtccagcaaggacagatgaacaaattcctgagtcagaagtctgttaatattgctgttttgaaggacaatcctttattttacttgagaccttacatctttgttctagctgacagtaaatctctgggtttctgttacgaactctaagagggctgaaacttctgatattcaggtggatcacctgaattctctcagctgtcaatggcttggagaacatctcatgggcccaagtcatcaaataacctgttcctctctgtaagggcagtgtgagggactgctgtgcagacccaagcaatcccaacctggtgctaggtcatttcacttttctgaaaacctcacatcaggctgcatcctcttctgtccctggcaccaggctttgtttacacttggagccaccttggtgtgggtcaccgggacagtgtactcctctcctgccagcctccccttccccgaggtgtggtggctgcagtctcaggaagagcttggtacttgtggggacttctgttttctccctgtggagatcagtgaagactgggaggaaagctgcttcaacctgagtccggctcttcagcaggctgcacaagtggaagcaactaattctggtgctcaggctgggctctccacccaagttaggcctgctctggcctaatggatcttactgtatgagcaggacggctgcattggattgtacaactgttttgtgatgcccccagacactgtcatcctgggccgagaagaacctgctagcttgacataccccatgggcttatccttaggttttggaattggtcaacagtgaggcagtctcccttcctgaccattcttctccacccagtcacagataagggaataaccttggccatatatttgctcaataaagattgaaggaagcatggtcatagttgccctgggttcagagcataatgcatatgtgaagcatggggtgacattcctactgtcatgggtttgggatttgtaacggcaaattcctgcccgacgacagggtgtcttatgcaaaggctgacttgcctgaacgctaagaacatgacttctgtctgagctaagctggcacccatcccagggctcctctggagctaatcctttaagcaaaatgtgcttgccttttaaagatccctgaccccagctttagctttctccaccagataaccagctaatcccaggaatttgctgccccccaccagtggcttctagggaaagcaaggacctcacatgccaggtgccctagtacttgcttagtgagccatgtcatcctcctttcatttttggatggtgacagcatttttcccctctgtgctggatacagacttctcccaggatcctctctttgggagcgaagccagaggatccctacagcactcaagcttcatggtggaattaatttctgccagctctttgttgtctgtctccttaaatccttttcctggtgtgcttattatcccttttgcagtgagtacagtttattaagttgtcagccctttaatattggggaaacttaatgagtataaatagcagggagcacattgtaacagcacagtgttttgtttttttcacccggttgctgtatgagaatggctttcaatcctttgtttctatgcctacagacagaaagcaagatgtctaatattagacatacaagttgctgcctgttataacggtgaattatacctttgtgcatgcctaggatgtttgttgttttaattagctgcaatatatacggcctgtgtacacagaatttaatcacttcggcaggttgaacaactccatgtagataagagcaagtgtaggcaaaggtttagaaaatggacataaagtcaaagaatgatggcaggtaggatgaaggagagatacttaggaaatcctaaaagaggcggcaagaaggtacctccctgtgtaactcaccttcccccatgacagtgagtaagagacactcacaggctatgagggtacacccctagctgaatgttctgtgttgtttccttagacctgtggtgtccgctgcaacagctactagccacgtgtagctaattacattaaaatgaaataaaattaaaagctcagtttctcagttgcgctaatcacatttcaagtgctcagcagccacccgtgtctactactacacagtgcagacacagaacatatcatcactgcagatagttctactggacaatgttacgctagaataaacaccaaggcagtcagttaaggcagctatggtttggaaaggcatacggacagagtctgcttagaagagatacaagttgttaataaaattgatcctgttgatagtagtttgtttttgtggtgggtgctgtgaagagtaaacattactcagtggaaagctaagttcagaaggtactttgtttttcctcccttgccttaagtccttggtatttataatcaatgctgaaccttctatttcactaccgctccctgttttagatattcagatttaaaaggttttcaaagaattactttcttccatgttcaaagctagattttactaaacacatgtatcacattcatatatattgtttcttggccccactgccaaaggaagtcagtcagtaatttcacaaccgttatcagagtttggaagcagaaatagctgttaactaaaatctcccactgctcagactactttctgccctaatggccattactatccagtctgtattgctacaagggacccactggtaccccttttagattctatcaaaaggaacagggttttcctagaggcaggcagcctggtggtatggcacagcagaagcttactgctaatgaaatgggaacctccccctcccttgtggtttcagcacagaacctgaatgccaggaaaaattcctgggccaagaagctaaagctaaagaaaccttccttttttcaacgtttttttttctttcaaactgtagggtcacttttgattgaggcaaaggggtcctactgtaagtggaaaagactcactcccctaacataagttttcactgtggtgggatggtgccgcccgatatgcttgatatgcttttccttccacatgttaagctaggaaacctaacaggatgtcagcagggcagttaactctggactcagagccctcaagggcatgtggcagaacctcatggacatcacaagaccatcagtctgaatccaggtcgtgggggctgtcatagccgaactccttctgcacatccagagggtacttgctccacatccgctgtctgctgctgcctctttcctcctcactcaggctgttgtagtcagcagagcctagaatgacatcccgggagtggattctaaatgtgattttcctaggctactgcaggagccccttctcttctcagaaaggtctgtttttgttcccgattgtaatgcaaaatccttgctcaataaataaaaaagaatatagaattcttttttttttaaagaaggaatcactttcctatcatctaaaccaagttccttcacactggagtattttgtcacttctcccctccgtggagtattttgtcacttctcccctccgtataggattttttgttgttgtaagagttgtagtcatattgtaaatatttttgtacctttctccttttaacgtgttattgacaaacctccccaaaagaatatgcaattgtttgattcatttctctgttatcagacaccaataaattctttttgttgggc

SEQ ID NO: 215 - DNA, 6255 bp, Homo sapiens
gcggcggcggcggcgcgaccgagcatcctggcggcgccgggccactgggagagtttatgtggccgaggcagacaagtggaattaggccttgctgcaggggacttcatttccttctcagtactggacccatttatgaggaggtggcttatgaaagtgtgatgttcgcgtatttcttgacaggccacagcaaacacaggtgtgcaggaaccgtttgtcatggaagccagggagcctgggaggcccacacccacctaccatcttgtccctaacaccagccagtcccaggtggaagaagatgtcagctcgccacctcaaaggtcctccgaaactatgcagctgaagaaggagatctccctgctgaatggggtcagcctggtggtgggcaacatgatcggctcagggatctttgtctcacccaagggtgtgctggtacacactgcctcctatgggatgtcactgattgtgtgggccattggtgggctcttctctgttgtgggtgccctttgttatgcagagctggggaccaccatcaccaagtcgggagccagctacgcttatattctagaggcctttgggggcttcattgccttcatccgcctgtgggtctcactgctagttgttgagcccaccggtcaggccatcatcgccatcacctttgccaactacatcatccagccgtccttccccagctgtgatcccccatacctggcctgccgtctcctggctgctgcttgcatatgtctgctgacatttgtgaactgtgcctatgtcaagtggggcacacgtgtgcaggacacgttcacttacgccaaggtcgtagcgctcattgccatcattgtcatgggccttgttaaactgtgccagggacactctgagcactttcaggacgcctttgagggttcctcctgggacatgggaaacctctctcttgccctctactctgccctcttctcttactcaggttgggacacccttaattttgtaacagaagaaatcaaaaacccagaaagaaatttgcccttggccattgggatttctatgccaattgtgacgctcatctacatcctgaccaatgtggcctattacacagtgctgaacatttcagatgtccttagcagtgatgctgtggctgtgacatttgctgaccagacgtttggcatgttcagctggaccatccccattgctgttgccctgtcctgctttgggggcctcaatgcatccatctttgcttcatcaaggttgttcttcgtgggctcccgggagggccacctaccggaccttctgtccatgatccacattgagcgttttacacctatccctgctttactgttcaattgcaccatggcactcatctacctcatcgtggaggatgttttccagcttatcaactacttcagcttcagctactggttcttcgtgggcctgtctgttgttggacagctctacctccgctggaaggagcccaagcggccccggcctctcaagctgagcgtgtttttccccatcgtgttctgcatatgctccgtgtttctggtgatagtgcccctcttcactgacaccattaattccctcattggcatcgggattgccctttctggagtccctttctacttcatgggtgtttacctgccagagtcccggaggccattgtttattcggaatgtcctggctgctatcaccagaggcacccagcagctttgcttttgtgtcctgactgagcttgatgtagccgaagaaaaaaaggatgagaggaaaactgactagaggtcagaggtggctttctgaggcctggaaggcaggccaaccagcaaaatcctgataacaagactctgtgggcccaactctcctgaattaaaggagccttttgacccaatcatatagtggggctcagggccagtgctcactcttattggtaagctataggagactcaggatctgggccaacctcaaggtgggggcttcagagggtggggggaagattggggaacggggggaatggtcatttagttttactcctgataggtagatgcagctcttacagatatttacttggtaaagtgcagtggggaagagggaatgctaggttgatagggctggtggcttctgaatttggtatttgaactaggagtccctatagaggggctgctttatgggaagtttttctctgaccaggtacaacacctgactttaaaggcctgaaatgctaccatttcttcctctggctcaaaattcttccctggggagagagttatattcccttatttattgatatttagtccagaacaccagttctaacgaagcatgcgtgtctcttcatctacaggatgcaataggctgattgtatttaaaaatcaaagtacccaaaactgagtccctttgggctcagaaatgtctgtggtattgggtcagactctgaccacaggttttatgctgtttagcacaatttctattgagtcttacctgcaacaatgaaccttaaagatttttttactcacgtacctgttacactttagcatacagatagatcatagatcacgttacaagcacttggctcaggtccagcaaggacagatgaacaaattcctgagtcagaagtctgttaatattgctgttttgaaggacaatcctttattttacttgagaccttacatctttgttctagctgacagtaaatctctgggtttctgttacgaactctaagagggctgaaacttctgatattcaggtggatcacctgaattctctcagctgtcaatggcttggagaacatctcatgggcccaagtcatcaaataacctgttcctctctgtaagggcagtgtgagggactgctgtgcagacccaagcaatcccaacctggtgctaggtcatttcacttttctgaaaacctcacatcaggctgcatcctcttctgtccctggcaccaggctttgtttacacttggagccaccttggtgtgggtcaccgggacagtgtactcctctcctgccagcctccccttccccgaggtgtggtggctgcagtctcaggaagagcttggtacttgtggggacttctgttttctccctgtggagatcagtgaagactgggaggaaagctgcttcaacctgagtccggctcttcagcaggctgcacaagtggaagcaactaattctggtgctcaggctgggctctccacccaagttaggcctgctctggcctaatggatcttactgtatgagcaggacggctgcattggattgtacaactgttttgtgatgcccccagacactgtcatcctgggccgagaagaacctgctagcttgacataccccatgggcttatccttaggttttggaattggtcaacagtgaggcagtctcccttcctgaccattcttctccacccagtcacagataagggaataaccttggccatatatttgctcaataaagattgaaggaagcatggtcatagttgccctgggttcagagcataatgcatatgtgaagcatggggtgacattcctactgtcatgggtttgggatttgtaacggcaaattcctgcccgacgacagggtgtcttatgcaaaggctgacttgcctgaacgctaagaacatgacttctgtctgagctaagctggcacccatcccagggctcctctggagctaatcctttaagcaaaatgtgcttgccttttaaagatccctgaccccagctttagctttctccaccagataaccagctaatcccaggaatttgctgccccccaccagtggcttctagggaaagcaaggacctcacatgccaggtgccctagtacttgcttagtgagccatgtcatcctcctttcatttttggatggtgacagcatttttcccctctgtgctggatacagacttctcccaggatcctctctttgggagcgaagccagaggatccctacagcactcaagcttcatggtggaattaatttctgccagctctttgttgtctgtctccttaaatccttttcctggtgtgcttattatcccttttgcagtgagtacagtttattaagttgtcagccctttaatattggggaaacttaatgagtataaatagcagggagcacattgtaacagcacagtgttttgtttttttcacccggttgctgtatgagaatggctttcaatcctttgtttctatgcctacagacagaaagcaagatgtctaatattagacatacaagttgctgcctgttataacggtgaattatacctttgtgcatgcctaggatgtttgttgttttaattagctgcaatatatacggcctgtgtacacagaatttaatcacttcggcaggttgaacaactccatgtagataagagcaagtgtaggcaaaggtttagaaaatggacataaagtcaaagaatgatggcaggtaggatgaaggagagatacttaggaaatcctaaaagaggcggcaagaaggtacctccctgtgtaactcaccttcccccatgacagtgagtaagagacactcacaggctatgagggtacacccctagctgaatgttctgtgttgtttccttagacctgtggtgtccgctgcaacagctactagccacgtgtagctaattacattaaaatgaaataaaattaaaagctcagtttctcagttgcgctaatcacatttcaagtgctcagcagccacccgtgtctactactacacagtgcagacacagaacatatcatcactgcagatagttctactggacaatgttacgctagaataaacaccaaggcagtcagttaaggcagctatggtttggaaaggcatacggacagagtctgcttagaagagatacaagttgttaataaaattgatcctgttgatagtagtttgtttttgtggtgggtgctgtgaagagtaaacattactcagtggaaagctaagttcagaaggtactttgtttttcctcccttgccttaagtccttggtatttataatcaatgctgaaccttctatttcactaccgctccctgttttagatattcagatttaaaaggttttcaaagaattactttcttccatgttcaaagctagattttactaaacacatgtatcacattcatatatattgtttcttggccccactgccaaaggaagtcagtcagtaatttcacaaccgttatcagagtttggaagcagaaatagctgttaactaaaatctcccactgctcagactactttctgccctaatggccattactatccagtctgtattgctacaagggacccactggtaccccttttagattctatcaaaaggaacagggttttcctagaggcaggcagcctggtggtatggcacagcagaagcttactgctaatgaaatgggaacctccccctcccttgtggtttcagcacagaacctgaatgccaggaaaaattcctgggccaagaagctaaagctaaagaaaccttccttttttcaacgtttttttttctttcaaactgtagggtcacttttgattgaggcaaaggggtcctactgtaagtggaaaagactcactcccctaacataagttttcactgtggtgggatggtgccgcccgatatgcttgatatgcttttccttccacatgttaagctaggaaacctaacaggatgtcagcagggcagttaactctggactcagagccctcaagggcatgtggcagaacctcatggacatcacaagaccatcagtctgaatccaggtcgtgggggctgtcatagccgaactccttctgcacatccagagggtacttgctccacatccgctgtctgctgctgcctctttcctcctcactcaggctgttgtagtcagcagagcctagaatgacatcccgggagtggattctaaatgtgattttcctaggctactgcaggagccccttctcttctcagaaaggtctgtttttgttcccgattgtaatgcaaaatccttgctcaataaataaaaaagaatatagaattcttttttttttaaagaaggaatcactttcctatcatctaaaccaagttccttcacactggagtattttgtcacttctcccctccgtggagtattttgtcacttctcccctccgtataggattttttgttgttgtaagagttgtagtcatattgtaaatatttttgtacctttctccttttaacgtgttattgacaaacctccccaaaagaatatgcaattgtttgattcatttctctgttatcagacaccaataaattctttttgttgggc

SEQ ID NO: 216-217 - skipped (2 entries)

SEQ ID NO: 218 - protein, 515 aa, Unknown
  Description of Unknown: y+LAT2 sequence
MEAREPGRPTPTYHLVPNTSQSQVEEDVSSPPQRSSETMQLKKEISLLNGVSLVVGNMIGSGIFVSPKGVLVHTASYGMSLIVWAIGGLFSVVGALCYAELGTTITKSGASYAYILEAFGGFIAFIRLWVSLLVVEPTGQAIIAITFANYIIQPSFPSCDPPYLACRLLAAACICLLTFVNCAYVKWGTRVQDTFTYAKVVALIAIIVMGLVKLCQGHSEHFQDAFEGSSWDMGNLSLALYSALFSYSGWDTLNFVTEEIKNPERNLPLAIGISMPIVTLIYILTNVAYYTVLNISDVLSSDAVAVTFADQTFGMFSWTIPIAVALSCFGGLNASIFASSRLFFVGSREGHLPDLLSMIHIERFTPIPALLFNCTMALIYLIVEDVFQLINYFSFSYWFFVGLSVVGQLYLRWKEPKRPRPLKLSVFFPIVFCICSVFLVIVPLFTDTINSLIGIGIALSGVPFYFMGVYLPESRRPLFIRNVLAAITRGTQQLCFCVLTELDVAEEKKDERKTD

SEQ ID NO: 219 - DNA, 1548 bp, Unknown
  Description of Unknown: y+LAT2 sequence
atggaagccagggagcctgggaggcccacacccacctaccatcttgtccctaacaccagccagtcccaggtggaagaagatgtcagctcgccacctcaaaggtcctccgaaactatgcagctgaagaaggagatctccctgctgaatggggtcagcctggtggtgggcaacatgatcggctcagggatctttgtctcacccaagggtgtgctggtacacactgcctcctatgggatgtcactgattgtgtgggccattggtgggctcttctctgttgtgggtgccctttgttatgcagagctggggaccaccatcaccaagtcgggagccagctacgcttatattctagaggcctttgggggcttcattgccttcatccgcctgtgggtctcactgctagttgttgagcccaccggtcaggccatcatcgccatcacctttgccaactacatcatccagccgtccttccccagctgtgatcccccatacctggcctgccgtctcctggctgctgcttgcatatgtctgctgacatttgtgaactgtgcctatgtcaagtggggcacacgtgtgcaggacacgttcacttacgccaaggtcgtagcgctcattgccatcattgtcatgggccttgttaaactgtgccagggacactctgagcactttcaggacgcctttgagggttcctcctgggacatgggaaacctctctcttgccctctactctgccctcttctcttactcaggttgggacacccttaattttgtaacagaagaaatcaaaaacccagaaagaaatttgcccttggccattgggatttctatgccaattgtgacgctcatctacatcctgaccaatgtggcctattacacagtgctgaacatttcagatgtccttagcagtgatgctgtggctgtgacatttgctgaccagacgtttggcatgttcagctggaccatccccattgctgttgccctgtcctgctttgggggcctcaatgcatccatctttgcttcatcaaggttgttcttcgtgggctcccgggagggccacctaccggaccttctgtccatgatccacattgagcgttttacacctatccctgctttactgttcaattgcaccatggcactcatctacctcatcgtggaggatgttttccagcttatcaactacttcagcttcagctactggttcttcgtgggcctgtctgttgttggacagctctacctccgctggaaggagcccaagcggccccggcctctcaagctgagcgtgtttttccccatcgtgttctgcatatgctccgtgtttctggtgatagtgcccctcttcactgacaccattaattccctcattggcatcgggattgccctttctggagtccctttctacttcatgggtgtttacctgccagagtcccggaggccattgtttattcggaatgtcctggctgctatcaccagaggcacccagcagctttgcttttgtgtcctgactgagcttgatgtagccgaagaaaaaaaggatgagaggaaaactgactag

SEQ ID NO: 220 - DNA, 2139 bp, Homo sapiens
gtcacactgtgcaaccttcctccctttcttaaatgcttggggcatttgtctggccttcccttttactgctggctgggaaggaggagcatcagaccacagatcctggaaggcacttctctccctgactgctgctcacactgccgtgagaacctgcttatatccaggaccaaggaggcaatgccaggaagctggtgaagggtttcctctcctccaccatggttgacagcactgagtatgaagtggcctcccagcctgaggtggaaacctcccctttgggtgatggggccagcccagggccggagcaggtgaagctgaagaaggagatctcactgcttaacggcgtgtgcctgattgtggggaacatgatcggctcgggcatctttgtttcccccaagggtgtgctcatatacagtgcctcctttggtctctctctggtcatctgggctgtcgggggcctcttctccgtctttggggccctttgttatgcggaactgggcaccaccattaagaaatctggggccagctatgcctatatcctggaggcctttggaggattccttgctttcatcagactctggacctccctgctcatcattgagcccaccagccaggccatcattgccatcacctttgccaactacatggtacagcctctcttcccgagctgcttcgccccttatgctgccagccgcctgctggctgctgcctgcatttgtctcttaaccttcattaactgtgcctatgtcaaatggggaaccctggtacaagatattttcacctatgctaaagtattggcactgatcgcggtcatcgttgcaggcattgttagacttggccagggagcctctactcattttgagaattcctttgagggttcatcatttgcagtgggtgacattgccctggcactgtactcagctctgttctcctactcaggctgggacaccctcaactatgtcactgaagagatcaagaatcctgagaggaacctgcccctctccattggcatctccatgcccattgtcaccatcatctatatcttgaccaatgtggcctattatactgtgctagacatgagagacatcttggccagtgatgctgttgctgtgacttttgcagatcagatatttggaatatttaactggataattccactgtcagttgcattatcctgttttggtggcctcaatgcctccattgtggctgcttctaggcttttctttgtgggctcaagagaaggccatctccctgatgccatctgcatgatccatgttgagcggttcacaccagtgccttctctgctcttcaatggtatcatggcattgatctacttgtgcgtggaagacatcttccagctcattaactactacagcttcagctactggttctttgtggggctttctattgtgggtcagctttatctgcgctggaaggagcctgatcgacctcgtcccctcaagctcagcgttttcttcccgattgtcttctgcctctgcaccatcttcctggtggctgttccactttacagtgatactatcaactccctcatcggcattgccattgccctctcaggcctgcccttttacttcctcatcatcagagtgccagaacataagcgaccgctttacctccgaaggatcgtggggtctgccacaaggtacctccaggtcctgtgtatgtcagttgctgcagaaatggatttggaagatggaggagagatgcccaagcaacgggatcccaaatctaactaaacaccatctggaatcctgatgtggaaagcaggggtttctggtctactggctagagctaaggaagttgaaaaggaaagctcacttctttggaggcacctgtccagaagcctggcctaggcagcttcaacctttgaacttactttttgaaatgaaaagtaatttatttgttttgctacatactgttccagacttttaaaggggacaatgaaggtgactgtggggaggagcatgtcaggtttgggcttggttgttttagaagcacctgggtgtgcctacctactcctcttttcttttaaaagggcccacaatgctccaatttcctgtctcctttagagagacatgaaactatcacaggtgctggatgacaataaaagtttatgttcctaaa

SEQ ID NO: 221 - DNA, 2082 bp, Homo sapiens
cccttttactgctggctgggaaggaggagcatcagaccacagatcctggaaggcacttctctccctgactgctgctcacactgccgtgagaacctgcttatatccaggaccaaggaggcaatgccaggaagctggtgaagggtttcctctcctccaccatggttgacagcactgagtatgaagtggcctcccagcctgaggtggaaacctcccctttgggtgatggggccagcccagggccggagcaggtgaagctgaagaaggagatctcactgcttaacggcgtgtgcctgattgtggggaacatgatcggctcgggcatctttgtttcccccaagggtgtgctcatatacagtgcctcctttggtctctctctggtcatctgggctgtcgggggcctcttctccgtctttggggccctttgttatgcggaactgggcaccaccattaagaaatctggggccagctatgcctatatcctggaggcctttggaggattccttgctttcatcagactctggacctccctgctcatcattgagcccaccagccaggccatcattgccatcacctttgccaactacatggtacagcctctcttcccgagctgcttcgccccttatgctgccagccgcctgctggctgctgcctgcatttgtctcttaaccttcattaactgtgcctatgtcaaatggggaaccctggtacaagatattttcacctatgctaaagtattggcactgatcgcggtcatcgttgcaggcattgttagacttggccagggagcctctactcattttgagaattcctttgagggttcatcatttgcagtgggtgacattgccctggcactgtactcagctctgttctcctactcaggctgggacaccctcaactatgtcactgaagagatcaagaatcctgagaggaacctgcccctctccattggcatctccatgcccattgtcaccatcatctatatcttgaccaatgtggcctattatactgtgctagacatgagagacatcttggccagtgatgctgttgctgtgacttttgcagatcagatatttggaatatttaactggataattccactgtcagttgcattatcctgttttggtggcctcaatgcctccattgtggctgcttctaggcttttctttgtgggctcaagagaaggccatctccctgatgccatctgcatgatccatgttgagcggttcacaccagtgccttctctgctcttcaatggtatcatggcattgatctacttgtgcgtggaagacatcttccagctcattaactactacagcttcagctactggttctttgtggggctttctattgtgggtcagctttatctgcgctggaaggagcctgatcgacctcgtcccctcaagctcagcgttttcttcccgattgtcttctgcctctgcaccatcttcctggtggctgttccactttacagtgatactatcaactccctcatcggcattgccattgccctctcaggcctgcccttttacttcctcatcatcagagtgccagaacataagcgaccgctttacctccgaaggatcgtggggtctgccacaaggtacctccaggtcctgtgtatgtcagttgctgcagaaatggatttggaagatggaggagagatgcccaagcaacgggatcccaaatctaactaaacaccatctggaatcctgatgtggaaagcaggggtttctggtctactggctagagctaaggaagttgaaaaggaaagctcacttctttggaggcacctgtccagaagcctggcctaggcagcttcaacctttgaacttactttttgaaatgaaaagtaatttatttgttttgctacatactgttccagacttttaaaggggacaatgaaggtgactgtggggaggagcatgtcaggtttgggcttggttgttttagaagcacctgggtgtgcctacctactcctcttttcttttaaaagggcccacaatgctccaatttcctgtctcctttagagagacatgaaactatcacaggtgctggatgacaataaaagtttatgttcctaaa

SEQ ID NO: 222 - DNA, 2252 bp, Homo sapiens
agtccccgttaccctctgctttttctgctcctcagagtcaacagctgttgcagcatgagcgatacgcttggttctcctaactagcaccttcccctctcccctgactcagctggtagcccctcctccccgcacctgcccaaaggtcactggacaggcatttgtctggccttcccttttactgctggctgggaaggaggagcatcagaccacagatcctggaaggcacttctctccctgactgctgctcacactgccgtgagaacctgcttatatccaggaccaaggaggcaatgccaggaagctggtgaagggtttcctctcctccaccatggttgacagcactgagtatgaagtggcctcccagcctgaggtggaaacctcccctttgggtgatggggccagcccagggccggagcaggtgaagctgaagaaggagatctcactgcttaacggcgtgtgcctgattgtggggaacatgatcggctcgggcatctttgtttcccccaagggtgtgctcatatacagtgcctcctttggtctctctctggtcatctgggctgtcgggggcctcttctccgtctttggggccctttgttatgcggaactgggcaccaccattaagaaatctggggccagctatgcctatatcctggaggcctttggaggattccttgctttcatcagactctggacctccctgctcatcattgagcccaccagccaggccatcattgccatcacctttgccaactacatggtacagcctctcttcccgagctgcttcgccccttatgctgccagccgcctgctggctgctgcctgcatttgtctcttaaccttcattaactgtgcctatgtcaaatggggaaccctggtacaagatattttcacctatgctaaagtattggcactgatcgcggtcatcgttgcaggcattgttagacttggccagggagcctctactcattttgagaattcctttgagggttcatcatttgcagtgggtgacattgccctggcactgtactcagctctgttctcctactcaggctgggacaccctcaactatgtcactgaagagatcaagaatcctgagaggaacctgcccctctccattggcatctccatgcccattgtcaccatcatctatatcttgaccaatgtggcctattatactgtgctagacatgagagacatcttggccagtgatgctgttgctgtgacttttgcagatcagatatttggaatatttaactggataattccactgtcagttgcattatcctgttttggtggcctcaatgcctccattgtggctgcttctaggcttttctttgtgggctcaagagaaggccatctccctgatgccatctgcatgatccatgttgagcggttcacaccagtgccttctctgctcttcaatggtatcatggcattgatctacttgtgcgtggaagacatcttccagctcattaactactacagcttcagctactggttctttgtggggctttctattgtgggtcagctttatctgcgctggaaggagcctgatcgacctcgtcccctcaagctcagcgttttcttcccgattgtcttctgcctctgcaccatcttcctggtggctgttccactttacagtgatactatcaactccctcatcggcattgccattgccctctcaggcctgcccttttacttcctcatcatcagagtgccagaacataagcgaccgctttacctccgaaggatcgtggggtctgccacaaggtacctccaggtcctgtgtatgtcagttgctgcagaaatggatttggaagatggaggagagatgcccaagcaacgggatcccaaatctaactaaacaccatctggaatcctgatgtggaaagcaggggtttctggtctactggctagagctaaggaagttgaaaaggaaagctcacttctttggaggcacctgtccagaagcctggcctaggcagcttcaacctttgaacttactttttgaaatgaaaagtaatttatttgttttgctacatactgttccagacttttaaaggggacaatgaaggtgactgtggggaggagcatgtcaggtttgggcttggttgttttagaagcacctgggtgtgcctacctactcctcttttcttttaaaagggcccacaatgctccaatttcctgtctcctttagagagacatgaaactatcacaggtgctggatgacaataaaagtttatgttcctaaa

SEQ ID NO: 223-224 - skipped (2 entries)

SEQ ID NO: 225 - protein, 511 aa, Unknown
  Description of Unknown: y+LAT1 sequence
MVDSTEYEVASQPEVETSPLGDGASPGPEQVKLKKEISLLNGVCLIVGNMIGSGIFVSPKGVLIYSASFGLSLVIWAVGGLFSVFGALCYAELGTTIKKSGASYAYILEAFGGFLAFIRLWTSLLIIEPTSQAIIAITFANYMVQPLFPSCFAPYAASRLLAAACICLLTFINCAYVKWGTLVQDIFTYAKVLALIAVIVAGIVRLGQGASTHFENSFEGSSFAVGDIALALYSALFSYSGWDTLNYVTEEIKNPERNLPLSIGISMPIVTIIYILTNVAYYTVLDMRDILASDAVAVTFADQIFGIFNWIIPLSVALSCFGGLNASIVAASRLFFVGSREGHLPDAICMIHVERFTPVPSLLFNGIMALIYLCVEDIFQLINYYSFSYWFFVGLSIVGQLYLRWKEPDRPRPLKLSVFFPIVFCLCTIFLVAVPLYSDTINSLIGIAIALSGLPFYFLIIRVPEHKRPLYLRRIVGSATRYLQVLCMSVAAEMDLEDGGEMPKQRDPKSN

SEQ ID NO: 226 - DNA, 1536 bp, Unknown
  Description of Unknown: y+LAT1 sequence
atggttgacagcactgagtatgaagtggcctcccagcctgaggtggaaacctcccctttgggtgatggggccagcccagggccggagcaggtgaagctgaagaaggagatctcactgcttaacggcgtgtgcctgattgtggggaacatgatcggctcgggcatctttgtttcccccaagggtgtgctcatatacagtgcctcctttggtctctctctggtcatctgggctgtcgggggcctcttctccgtctttggggccctttgttatgcggaactgggcaccaccattaagaaatctggggccagctatgcctatatcctggaggcctttggaggattccttgctttcatcagactctggacctccctgctcatcattgagcccaccagccaggccatcattgccatcacctttgccaactacatggtacagcctctcttcccgagctgcttcgccccttatgctgccagccgcctgctggctgctgcctgcatttgtctcttaaccttcattaactgtgcctatgtcaaatggggaaccctggtacaagatattttcacctatgctaaagtattggcactgatcgcggtcatcgttgcaggcattgttagacttggccagggagcctctactcattttgagaattcctttgagggttcatcatttgcagtgggtgacattgccctggcactgtactcagctctgttctcctactcaggctgggacaccctcaactatgtcactgaagagatcaagaatcctgagaggaacctgcccctctccattggcatctccatgcccattgtcaccatcatctatatcttgaccaatgtggcctattatactgtgctagacatgagagacatcttggccagtgatgctgttgctgtgacttttgcagatcagatatttggaatatttaactggataattccactgtcagttgcattatcctgttttggtggcctcaatgcctccattgtggctgcttctaggcttttctttgtgggctcaagagaaggccatctccctgatgccatctgcatgatccatgttgagcggttcacaccagtgccttctctgctcttcaatggtatcatggcattgatctacttgtgcgtggaagacatcttccagctcattaactactacagcttcagctactggttctttgtggggctttctattgtgggtcagctttatctgcgctggaaggagcctgatcgacctcgtcccctcaagctcagcgttttcttcccgattgtcttctgcctctgcaccatcttcctggtggctgttccactttacagtgatactatcaactccctcatcggcattgccattgccctctcaggcctgcccttttacttcctcatcatcagagtgccagaacataagcgaccgctttacctccgaaggatcgtggggtctgccacaaggtacctccaggtcctgtgtatgtcagttgctgcagaaatggatttggaagatggaggagagatgcccaagcaacgggatcccaaatctaactaa

SEQ ID NO: 227 - DNA, 2224 bp, Homo sapiens
gcattgcggcttggttttctcacccagtgcatgtggcaggagcggtgagatcactgcctcacggcgatcctggactgacggtcacgactgcctaccctctaaccctgttctgagctgccccttgcccacacaccccaaacctgtgtgcaggatccgcctccatggagctacagcctcctgaagcctcgatcgccgtcgtgtcgattccgcgccagttgcctggctcacattcggaggctggtgtccagggtctcagcgcgggggacgactcagagacggggtctgactgtgttacccaggctggtcttcaactcttggcctcaagtgatcctcctgccttagcttccaagaatgctgaggttacagtagaaacggggtttcaccatgttagccaggctgatattgaattcctgacctcaattgatccgactgcctcggcctccggaagtgctgggattacaggcaccatgagccaggacaccgaggtggatatgaaggaggtggagctgaatgagttagagcccgagaagcagccgatgaacgcggcgtctggggcggccatgtccctggcgggagccgagaagaatggtctggtgaagatcaaggtggcggaagacgaggcggaggcggcagccgcggctaagttcacgggcctgtccaaggaggagctgctgaaggtggcaggcagccccggctgggtacgcacccgctgggcactgctgctgctcttctggctcggctggctcggcatgcttgctggtgccgtggtcataatcgtgcgagcgccgcgttgtcgcgagctaccggcgcagaagtggtggcacacgggcgccctctaccgcatcggcgaccttcaggccttccagggccacggcgcgggcaacctggcgggtctgaaggggcgtctcgattacctgagctctctgaaggtgaagggccttgtgctgggtccaattcacaagaaccagaaggatgatgtcgctcagactgacttgctgcagatcgaccccaattttggctccaaggaagattttgacagtctcttgcaatcggctaaaaaaaagagcatccgtgtcattctggaccttactcccaactaccggggtgagaactcgtggttctccactcaggttgacactgtggccaccaaggtgaaggatgctctggagttttggctgcaagctggcgtggatgggttccaggttcgggacatagagaatctgaaggatgcatcctcattcttggctgagtggcaaaatatcaccaagggcttcagtgaagacaggctcttgattgcggggactaactcctccgaccttcagcagatcctgagcctactcgaatccaacaaagacttgctgttgactagctcatacctgtctgattctggttctactggggagcatacaaaatccctagtcacacagtatttgaatgccactggcaatcgctggtgcagctggagtttgtctcaggcaaggctcctgacttccttcttgccggctcaacttctccgactctaccagctgatgctcttcaccctgccagggacccctgttttcagctacggggatgagattggcctggatgcagctgcccttcctggacagcctatggaggctccagtcatgctgtgggatgagtccagcttccctgacatcccaggggctgtaagtgccaacatgactgtgaagggccagagtgaagaccctggctccctcctttccttgttccggcggctgagtgaccagcggagtaaggagcgctccctactgcatggggacttccacgcgttctccgctgggcctggactcttctcctatatccgccactgggaccagaatgagcgttttctggtagtgcttaactttggggatgtgggcctctcggctggactgcaggcctccgacctgcctgccagcgccagcctgccagccaaggctgacctcctgctcagcacccagccaggccgtgaggagggctcccctcttgagctggaacgcctgaaactggagcctcacgaagggctgctgctccgcttcccctacgcggcctgacttcagcctgacatggacccactacccttctcctttccttcccaggccctttggcttctgatttttctcttttttaaaaacaaacaaacaaactgttgcagattatgagtgaacccccaaatagggtgttttctgccttcaaataaaagtcacccctgcatggtgaa

SEQ ID NO: 228 - DNA, 2035 bp, Homo sapiens
gcattgcggcttggttttctcacccagtgcatgtggcaggagcggtgagatcactgcctcacggcgatcctggactgacggtcacgactgcctaccctctaaccctgttctgagctgccccttgcccacacaccccaaacctgtgtgcaggatccgcctccatggagctacagcctcctgaagcctcgatcgccgtcgtgtcgattccgcgccagttgcctggctcacattcggaggctggtgtccagggtctcagcgcgggggacgactcaggcaccatgagccaggacaccgaggtggatatgaaggaggtggagctgaatgagttagagcccgagaagcagccgatgaacgcggcgtctggggcggccatgtccctggcgggagccgagaagaatggtctggtgaagatcaaggtggcggaagacgaggcggaggcggcagccgcggctaagttcacgggcctgtccaaggaggagctgctgaaggtggcaggcagccccggctgggtacgcacccgctgggcactgctgctgctcttctggctcggctggctcggcatgcttgctggtgccgtggtcataatcgtgcgagcgccgcgttgtcgcgagctaccggcgcagaagtggtggcacacgggcgccctctaccgcatcggcgaccttcaggccttccagggccacggcgcgggcaacctggcgggtctgaaggggcgtctcgattacctgagctctctgaaggtgaagggccttgtgctgggtccaattcacaagaaccagaaggatgatgtcgctcagactgacttgctgcagatcgaccccaattttggctccaaggaagattttgacagtctcttgcaatcggctaaaaaaaagagcatccgtgtcattctggaccttactcccaactaccggggtgagaactcgtggttctccactcaggttgacactgtggccaccaaggtgaaggatgctctggagttttggctgcaagctggcgtggatgggttccaggttcgggacatagagaatctgaaggatgcatcctcattcttggctgagtggcaaaatatcaccaagggcttcagtgaagacaggctcttgattgcggggactaactcctccgaccttcagcagatcctgagcctactcgaatccaacaaagacttgctgttgactagctcatacctgtctgattctggttctactggggagcatacaaaatccctagtcacacagtatttgaatgccactggcaatcgctggtgcagctggagtttgtctcaggcaaggctcctgacttccttcttgccggctcaacttctccgactctaccagctgatgctcttcaccctgccagggacccctgttttcagctacggggatgagattggcctggatgcagctgcccttcctggacagcctatggaggctccagtcatgctgtgggatgagtccagcttccctgacatcccaggggctgtaagtgccaacatgactgtgaagggccagagtgaagaccctggctccctcctttccttgttccggcggctgagtgaccagcggagtaaggagcgctccctactgcatggggacttccacgcgttctccgctgggcctggactcttctcctatatccgccactgggaccagaatgagcgttttctggtagtgcttaactttggggatgtgggcctctcggctggactgcaggcctccgacctgcctgccagcgccagcctgccagccaaggctgacctcctgctcagcacccagccaggccgtgaggagggctcccctcttgagctggaacgcctgaaactggagcctcacgaagggctgctgctccgcttcccctacgcggcctgacttcagcctgacatggacccactacccttctcctttccttcccaggccctttggcttctgatttttctcttttttaaaaacaaacaaacaaactgttgcagattatgagtgaacccccaaatagggtgttttctgccttcaaataaaagtcacccctgcatggtgaa

SEQ ID NO: 229 - DNA, 1885 bp, Homo sapiens
agatgcagtagccgaaactgcgcggaggcacagaggccggggagagcgttctgggtccgagggtccaggtaggggttgagccaccatctgaccgcaagctgcgtcgtgtcgccggttctgcaggcaccatgagccaggacaccgaggtggatatgaaggaggtggagctgaatgagttagagcccgagaagcagccgatgaacgcggcgtctggggcggccatgtccctggcgggagccgagaagaatggtctggtgaagatcaaggtggcggaagacgaggcggaggcggcagccgcggctaagttcacgggcctgtccaaggaggagctgctgaaggtggcaggcagccccggctgggtacgcacccgctgggcactgctgctgctcttctggctcggctggctcggcatgcttgctggtgccgtggtcataatcgtgcgagcgccgcgttgtcgcgagctaccggcgcagaagtggtggcacacgggcgccctctaccgcatcggcgaccttcaggccttccagggccacggcgcgggcaacctggcgggtctgaaggggcgtctcgattacctgagctctctgaaggtgaagggccttgtgctgggtccaattcacaagaaccagaaggatgatgtcgctcagactgacttgctgcagatcgaccccaattttggctccaaggaagattttgacagtctcttgcaatcggctaaaaaaaagagcatccgtgtcattctggaccttactcccaactaccggggtgagaactcgtggttctccactcaggttgacactgtggccaccaaggtgaaggatgctctggagttttggctgcaagctggcgtggatgggttccaggttcgggacatagagaatctgaaggatgcatcctcattcttggctgagtggcaaaatatcaccaagggcttcagtgaagacaggctcttgattgcggggactaactcctccgaccttcagcagatcctgagcctactcgaatccaacaaagacttgctgttgactagctcatacctgtctgattctggttctactggggagcatacaaaatccctagtcacacagtatttgaatgccactggcaatcgctggtgcagctggagtttgtctcaggcaaggctcctgacttccttcttgccggctcaacttctccgactctaccagctgatgctcttcaccctgccagggacccctgttttcagctacggggatgagattggcctggatgcagctgcccttcctggacagcctatggaggctccagtcatgctgtgggatgagtccagcttccctgacatcccaggggctgtaagtgccaacatgactgtgaagggccagagtgaagaccctggctccctcctttccttgttccggcggctgagtgaccagcggagtaaggagcgctccctactgcatggggacttccacgcgttctccgctgggcctggactcttctcctatatccgccactgggaccagaatgagcgttttctggtagtgcttaactttggggatgtgggcctctcggctggactgcaggcctccgacctgcctgccagcgccagcctgccagccaaggctgacctcctgctcagcacccagccaggccgtgaggagggctcccctcttgagctggaacgcctgaaactggagcctcacgaagggctgctgctccgcttcccctacgcggcctgacttcagcctgacatggacccactacccttctcctttccttcccaggccctttggcttctgatttttctcttttttaaaaacaaacaaacaaactgttgcagattatgagtgaacccccaaatagggtgttttctgccttcaaataaaagtcacccctgcatggtgaa

SEQ ID NO: 230 - DNA, 2221 bp, Homo sapiens
gcattgcggcttggttttctcacccagtgcatgtggcaggagcggtgagatcactgcctcacggcgatcctggactgacggtcacgactgcctaccctctaaccctgttctgagctgccccttgcccacacaccccaaacctgtgtgcaggatccgcctccatggagctacagcctcctgaagcctcgatcgccgtcgtgtcgattccgcgccagttgcctggctcacattcggaggctggtgtccagggtctcagcgcgggggacgactcagagttggggtctcactgtgttgcccagactggtctcgaactcttggcctcaggtgatcctcttccctcagcttcccagaatgccgagatgatagagacggggtctgactgtgttacccaggctggtcttcaactcttggcctcaagtgatcctcctgccttagcttccaagaatgctgaggttacaggcaccatgagccaggacaccgaggtggatatgaaggaggtggagctgaatgagttagagcccgagaagcagccgatgaacgcggcgtctggggcggccatgtccctggcgggagccgagaagaatggtctggtgaagatcaaggtggcggaagacgaggcggaggcggcagccgcggctaagttcacgggcctgtccaaggaggagctgctgaaggtggcaggcagccccggctgggtacgcacccgctgggcactgctgctgctcttctggctcggctggctcggcatgcttgctggtgccgtggtcataatcgtgcgagcgccgcgttgtcgcgagctaccggcgcagaagtggtggcacacgggcgccctctaccgcatcggcgaccttcaggccttccagggccacggcgcgggcaacctggcgggtctgaaggggcgtctcgattacctgagctctctgaaggtgaagggccttgtgctgggtccaattcacaagaaccagaaggatgatgtcgctcagactgacttgctgcagatcgaccccaattttggctccaaggaagattttgacagtctcttgcaatcggctaaaaaaaagagcatccgtgtcattctggaccttactcccaactaccggggtgagaactcgtggttctccactcaggttgacactgtggccaccaaggtgaaggatgctctggagttttggctgcaagctggcgtggatgggttccaggttcgggacatagagaatctgaaggatgcatcctcattcttggctgagtggcaaaatatcaccaagggcttcagtgaagacaggctcttgattgcggggactaactcctccgaccttcagcagatcctgagcctactcgaatccaacaaagacttgctgttgactagctcatacctgtctgattctggttctactggggagcatacaaaatccctagtcacacagtatttgaatgccactggcaatcgctggtgcagctggagtttgtctcaggcaaggctcctgacttccttcttgccggctcaacttctccgactctaccagctgatgctcttcaccctgccagggacccctgttttcagctacggggatgagattggcctggatgcagctgcccttcctggacagcctatggaggctccagtcatgctgtgggatgagtccagcttccctgacatcccaggggctgtaagtgccaacatgactgtgaagggccagagtgaagaccctggctccctcctttccttgttccggcggctgagtgaccagcggagtaaggagcgctccctactgcatggggacttccacgcgttctccgctgggcctggactcttctcctatatccgccactgggaccagaatgagcgttttctggtagtgcttaactttggggatgtgggcctctcggctggactgcaggcctccgacctgcctgccagcgccagcctgccagccaaggctgacctcctgctcagcacccagccaggccgtgaggagggctcccctcttgagctggaacgcctgaaactggagcctcacgaagggctgctgctccgcttcccctacgcggcctgacttcagcctgacatggacccactacccttctcctttccttcccaggccctttggcttctgatttttctcttttttaaaaacaaacaaacaaactgttgcagattatgagtgaacccccaaatagggtgttttctgccttcaaataaaagtcacccctgcatggtgaa

SEQ ID NO: 231 - skipped

SEQ ID NO: 232 - protein, 630 aa, Unknown
  Description of Unknown: 4F2hc sequence
MELQPPEASIAVVSIPRQLPGSHSEAGVQGLSAGDDSELGSHCVAQTGLELLASGDPLPSASQNAEMIETGSDCVTQAGLQLLASSDPPALASKNAEVTGTMSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAA

SEQ ID NO: 233 - DNA, 1893 bp, Unknown
  Description of Unknown: 4F2hc sequence
atggagctacagcctcctgaagcctcgatcgccgtcgtgtcgattccgcgccagttgcctggctcacattcggaggctggtgtccagggtctcagcgcgggggacgactcagagttggggtctcactgtgttgcccagactggtctcgaactcttggcctcaggtgatcctcttccctcagcttcccagaatgccgagatgatagagacggggtctgactgtgttacccaggctggtcttcaactcttggcctcaagtgatcctcctgccttagcttccaagaatgctgaggttacaggcaccatgagccaggacaccgaggtggatatgaaggaggtggagctgaatgagttagagcccgagaagcagccgatgaacgcggcgtctggggcggccatgtccctggcgggagccgagaagaatggtctggtgaagatcaaggtggcggaagacgaggcggaggcggcagccgcggctaagttcacgggcctgtccaaggaggagctgctgaaggtggcaggcagccccggctgggtacgcacccgctgggcactgctgctgctcttctggctcggctggctcggcatgcttgctggtgccgtggtcataatcgtgcgagcgccgcgttgtcgcgagctaccggcgcagaagtggtggcacacgggcgccctctaccgcatcggcgaccttcaggccttccagggccacggcgcgggcaacctggcgggtctgaaggggcgtctcgattacctgagctctctgaaggtgaagggccttgtgctgggtccaattcacaagaaccagaaggatgatgtcgctcagactgacttgctgcagatcgaccccaattttggctccaaggaagattttgacagtctcttgcaatcggctaaaaaaaagagcatccgtgtcattctggaccttactcccaactaccggggtgagaactcgtggttctccactcaggttgacactgtggccaccaaggtgaaggatgctctggagttttggctgcaagctggcgtggatgggttccaggttcgggacatagagaatctgaaggatgcatcctcattcttggctgagtggcaaaatatcaccaagggcttcagtgaagacaggctcttgattgcggggactaactcctccgaccttcagcagatcctgagcctactcgaatccaacaaagacttgctgttgactagctcatacctgtctgattctggttctactggggagcatacaaaatccctagtcacacagtatttgaatgccactggcaatcgctggtgcagctggagtttgtctcaggcaaggctcctgacttccttcttgccggctcaacttctccgactctaccagctgatgctcttcaccctgccagggacccctgttttcagctacggggatgagattggcctggatgcagctgcccttcctggacagcctatggaggctccagtcatgctgtgggatgagtccagcttccctgacatcccaggggctgtaagtgccaacatgactgtgaagggccagagtgaagaccctggctccctcctttccttgttccggcggctgagtgaccagcggagtaaggagcgctccctactgcatggggacttccacgcgttctccgctgggcctggactcttctcctatatccgccactgggaccagaatgagcgttttctggtagtgcttaactttggggatgtgggcctctcggctggactgcaggcctccgacctgcctgccagcgccagcctgccagccaaggctgacctcctgctcagcacccagccaggccgtgaggagggctcccctcttgagctggaacgcctgaaactggagcctcacgaagggctgctgctccgcttcccctacgcggcctga

SEQ ID NO: 234 - DNA, 1680 bp, Homo sapiens
gccatttctagggttggaccgtgcaggcacgggcggtcagctgggccgcagctcctccggctctgcagggtcacggaggaagtctcctggaaccagcaggaggaaacatgggggatactggcctgagaaagcggagagaggatgagaagtcgatccagagccaagagcctaagaccaccagtctccaaaaggagctgggcctcatcagtggcatctccatcatcgtgggcaccatcattggctctgggatcttcgtttcccccaagtctgtgctcagcaacacggaagctgtggggccctgcctcatcatatgggcggcttgcggggtcctcgcgacgctgggtgccctgtgctttgcggagcttggcacaatgatcaccaagtcagggggagagtatccctacctgatggaggcctacgggcccatccccgcctacctcttctcctgggccagcctgatcgtcattaagcccacgtccttcgccatcatctgcctcagcttctccgagtatgtgtgtgcgcccttctatgtgggctgcaagcctcctcaaatcgttgtgaaatgcctggccgccgccgccatcttgttcatctcgacagtgaactcactgagcgtgcggctgggaagctacgtccagaacatcttcaccgcggccaagctggtgatcgtggccatcatcatcatcagcgggctggtgctcctggcccaaggaaacacaaagaattttgataattctttcgagggcgcccagctgtctgtgggagccatcagcctggcgttttacaatggactctgggcctatgatggatggaatcaactcaattacatcacagaagaacttagaaacccttacagaaacctgcctttggccattatcatcgggatccccctggtgacggcgtgctacatcctcatgaacgtgtcctacttcaccgtgatgactgccaccgaactcctgcagtcccaggcggtggctgtgacatttggtgaccgtgttctctatcctgcttcttggatcgttccactttttgtggcattttcaaccatcggtgctgctaacgggacctgcttcacagcgggcagactcatttacgtggcgggccgggagggtcacatgctcaaagtgctttcttacatcagcgtcaggcgcctcactccagcccccgccatcatcttttatggtatcatagcaacgatttatatcatccctggtgacataaactcgttagtcaattatttcagctttgccgcatggctgttttatggcctgacgattctaggactcatcgtgatgagatttacaaggaaagagctggaaaggcctatcaaggtgcccgtagtcattcccgtcttgatgacactcatctctgtgtttttggttctggctccaatcatcagcaagcccacctgggagtacctctactgtgtgctgtttatattaagcggccttttattttacttcctgtttgtccactacaagtttggatgggctcagaaaatctcaaagccgattaccatgcaccttcagatgctaatggaagtggtcccaccggaggaagaccctgagtaacaagctccgtctcttgtagccaagtcagctgaatttattttcttaagcaatatttgtggttatttcttcctttttttcttacgaataaaatatactcagatgtttaaaa

SEQ ID NO: 235 - DNA, 1775 bp, Homo sapiens
gccatttctagggttggaccgtgcaggcacgggcggtcagctgggccgcagctcctccggctctgcagggtcacggaggaaggtaagtaagccagctcccctagtccaggccgagcttgcacttgcgtcttgtctgctgctgctgaaccaagatttagctgtgcgccctccttgcagtctcctggaaccagcaggaggaaacatgggggatactggcctgagaaagcggagagaggatgagaagtcgatccagagccaagagcctaagaccaccagtctccaaaaggagctgggcctcatcagtggcatctccatcatcgtgggcaccatcattggctctgggatcttcgtttcccccaagtctgtgctcagcaacacggaagctgtggggccctgcctcatcatatgggcggcttgcggggtcctcgcgacgctgggtgccctgtgctttgcggagcttggcacaatgatcaccaagtcagggggagagtatccctacctgatggaggcctacgggcccatccccgcctacctcttctcctgggccagcctgatcgtcattaagcccacgtccttcgccatcatctgcctcagcttctccgagtatgtgtgtgcgcccttctatgtgggctgcaagcctcctcaaatcgttgtgaaatgcctggccgccgccgccatcttgttcatctcgacagtgaactcactgagcgtgcggctgggaagctacgtccagaacatcttcaccgcggccaagctggtgatcgtggccatcatcatcatcagcgggctggtgctcctggcccaaggaaacacaaagaattttgataattctttcgagggcgcccagctgtctgtgggagccatcagcctggcgttttacaatggactctgggcctatgatggatggaatcaactcaattacatcacagaagaacttagaaacccttacagaaacctgcctttggccattatcatcgggatccccctggtgacggcgtgctacatcctcatgaacgtgtcctacttcaccgtgatgactgccaccgaactcctgcagtcccaggcggtggctgtgacatttggtgaccgtgttctctatcctgcttcttggatcgttccactttttgtggcattttcaaccatcggtgctgctaacgggacctgcttcacagcgggcagactcatttacgtggcgggccgggagggtcacatgctcaaagtgctttcttacatcagcgtcaggcgcctcactccagcccccgccatcatcttttatggtatcatagcaacgatttatatcatccctggtgacataaactcgttagtcaattatttcagctttgccgcatggctgttttatggcctgacgattctaggactcatcgtgatgagatttacaaggaaagagctggaaaggcctatcaaggtgcccgtagtcattcccgtcttgatgacactcatctctgtgtttttggttctggctccaatcatcagcaagcccacctgggagtacctctactgtgtgctgtttatattaagcggccttttattttacttcctgtttgtccactacaagtttggatgggctcagaaaatctcaaagccgattaccatgcaccttcagatgctaatggaagtggtcccaccggaggaagaccctgagtaacaagctccgtctcttgtagccaagtcagctgaatttattttcttaagcaatatttgtggttatttcttcctttttttcttacgaataaaatatactcagatgtttaaaa

SEQ ID NO: 236 - DNA, 1766 bp, Homo sapiens
gccatttctagggttggaccgtgcaggcacgggcggtcagctgggccgcagctcctccggctctgcagggtcacggaggaagccagctcccctagtccaggccgagcttgcacttgcgtcttgtctgctgctgctgaaccaagatttagctgtgcgccctccttgcagtctcctggaaccagcaggaggaaacatgggggatactggcctgagaaagcggagagaggatgagaagtcgatccagagccaagagcctaagaccaccagtctccaaaaggagctgggcctcatcagtggcatctccatcatcgtgggcaccatcattggctctgggatcttcgtttcccccaagtctgtgctcagcaacacggaagctgtggggccctgcctcatcatatgggcggcttgcggggtcctcgcgacgctgggtgccctgtgctttgcggagcttggcacaatgatcaccaagtcagggggagagtatccctacctgatggaggcctacgggcccatccccgcctacctcttctcctgggccagcctgatcgtcattaagcccacgtccttcgccatcatctgcctcagcttctccgagtatgtgtgtgcgcccttctatgtgggctgcaagcctcctcaaatcgttgtgaaatgcctggccgccgccgccatcttgttcatctcgacagtgaactcactgagcgtgcggctgggaagctacgtccagaacatcttcaccgcggccaagctggtgatcgtggccatcatcatcatcagcgggctggtgctcctggcccaaggaaacacaaagaattttgataattctttcgagggcgcccagctgtctgtgggagccatcagcctggcgttttacaatggactctgggcctatgatggatggaatcaactcaattacatcacagaagaacttagaaacccttacagaaacctgcctttggccattatcatcgggatccccctggtgacggcgtgctacatcctcatgaacgtgtcctacttcaccgtgatgactgccaccgaactcctgcagtcccaggcggtggctgtgacatttggtgaccgtgttctctatcctgcttcttggatcgttccactttttgtggcattttcaaccatcggtgctgctaacgggacctgcttcacagcgggcagactcatttacgtggcgggccgggagggtcacatgctcaaagtgctttcttacatcagcgtcaggcgcctcactccagcccccgccatcatcttttatggtatcatagcaacgatttatatcatccctggtgacataaactcgttagtcaattatttcagctttgccgcatggctgttttatggcctgacgattctaggactcatcgtgatgagatttacaaggaaagagctggaaaggcctatcaaggtgcccgtagtcattcccgtcttgatgacactcatctctgtgtttttggttctggctccaatcatcagcaagcccacctgggagtacctctactgtgtgctgtttatattaagcggccttttattttacttcctgtttgtccactacaagtttggatgggctcagaaaatctcaaagccgattaccatgcaccttcagatgctaatggaagtggtcccaccggaggaagaccctgagtaacaagctccgtctcttgtagccaagtcagctgaatttattttcttaagcaatatttgtggttatttcttcctttttttcttacgaataaaatatactcagatgtttaaaa

SEQ ID NO: 237-239 - skipped (3 entries)

SEQ ID NO: 240 - protein, 487 aa, Unknown
  Description of Unknown: b0,+AT sequence
MGDTGLRKRREDEKSIQSQEPKTTSLQKELGLISGISIIVGTIIGSGIFVSPKSVLSNTEAVGPCLIIWAACGVLATLGALCFAELGTMITKSGGEYPYLMEAYGPIPAYLFSWASLIVIKPTSFAIICLSFSEYVCAPFYVGCKPPQIVVKCLAAAAILFISTVNSLSVRLGSYVQNIFTAAKLVIVAIIIISGLVLLAQGNTKNFDNSFEGAQLSVGAISLAFYNGLWAYDGWNQLNYITEELRNPYRNLPLAIIIGIPLVTACYILMNVSYFTVMTATELLQSQAVAVTFGDRVLYPASWIVPLFVAFSTIGAANGTCFTAGRLIYVAGREGHMLKVLSYISVRRLTPAPAIIFYGIIATIYIIPGDINSLVNYFSFAAWLFYGLTILGLIVMRFTRKELERPIKVPVVIPVLMTLISVFLVLAPIISKPTWEYLYCVLFILSGLLFYFLFVHYKFGWAQKISKPITMHLQMLMEVVPPEEDPE

SEQ ID NO: 241 - DNA, 1464 bp, Unknown
  Description of Unknown: b0,+AT sequence
atgggggatactggcctgagaaagcggagagaggatgagaagtcgatccagagccaagagcctaagaccaccagtctccaaaaggagctgggcctcatcagtggcatctccatcatcgtgggcaccatcattggctctgggatcttcgtttcccccaagtctgtgctcagcaacacggaagctgtggggccctgcctcatcatatgggcggcttgcggggtcctcgcgacgctgggtgccctgtgctttgcggagcttggcacaatgatcaccaagtcagggggagagtatccctacctgatggaggcctacgggcccatccccgcctacctcttctcctgggccagcctgatcgtcattaagcccacgtccttcgccatcatctgcctcagcttctccgagtatgtgtgtgcgcccttctatgtgggctgcaagcctcctcaaatcgttgtgaaatgcctggccgccgccgccatcttgttcatctcgacagtgaactcactgagcgtgcggctgggaagctacgtccagaacatcttcaccgcggccaagctggtgatcgtggccatcatcatcatcagcgggctggtgctcctggcccaaggaaacacaaagaattttgataattctttcgagggcgcccagctgtctgtgggagccatcagcctggcgttttacaatggactctgggcctatgatggatggaatcaactcaattacatcacagaagaacttagaaacccttacagaaacctgcctttggccattatcatcgggatccccctggtgacggcgtgctacatcctcatgaacgtgtcctacttcaccgtgatgactgccaccgaactcctgcagtcccaggcggtggctgtgacatttggtgaccgtgttctctatcctgcttcttggatcgttccactttttgtggcattttcaaccatcggtgctgctaacgggacctgcttcacagcgggcagactcatttacgtggcgggccgggagggtcacatgctcaaagtgctttcttacatcagcgtcaggcgcctcactccagcccccgccatcatcttttatggtatcatagcaacgatttatatcatccctggtgacataaactcgttagtcaattatttcagctttgccgcatggctgttttatggcctgacgattctaggactcatcgtgatgagatttacaaggaaagagctggaaaggcctatcaaggtgcccgtagtcattcccgtcttgatgacactcatctctgtgtttttggttctggctccaatcatcagcaagcccacctgggagtacctctactgtgtgctgtttatattaagcggccttttattttacttcctgtttgtccactacaagtttggatgggctcagaaaatctcaaagccgattaccatgcaccttcagatgctaatggaagtggtcccaccggaggaagaccctgagtaa

SEQ ID NO: 242 - DNA, 2969 bp, Homo sapiens
actcttccacctcccttactgcaggaaggcactccgaagacataagtcggtgagacatggctgaagataaaagcaagagagactccatcgagatgagtatgaagggatgccagacaaacaacgggtttgtccataatgaagacattctggagcagaccccggatccaggaagctcaacagacaacctgaagcacagcaccaggggcatccttggctcccaggagcccgacttcaagggcgtccagccctatgcggggatgcccaaggaggtgctgttccagttctctggccaggcccgctaccgcatacctcgggagatcctcttctggctcacagtggcttctgtgctggtgctcatcgcggccaccatagccatcattgccctctctccaaagtgcctagactggtggcaggaggggcccatgtaccagatctacccaaggtctttcaaggacagtaacaaggatgggaacggagatctgaaaggtattcaagataaactggactacatcacagctttaaatataaaaactgtttggattacttcattttataaatcgtcccttaaagatttcagatatggtgttgaagatttccgggaagttgatcccatttttggaacgatggaagattttgagaatctggttgcagccatacatgataaaggtttaaaattaatcatcgatttcataccaaaccacacgagtgataaacatatttggtttcaattgagtcggacacggacaggaaaatatactgattattatatctggcatgactgtacccatgaaaatggcaaaaccattccacccaacaactggttaagtgtgtatggaaactccagttggcactttgacgaagtgcgaaaccaatgttattttcatcagtttatgaaagagcaacctgatttaaatttccgcaatcctgatgttcaagaagaaataaaagaaattttacggttctggctcacaaagggtgttgatggttttagtttggatgctgttaaattcctcctagaagcaaagcacctgagagatgagatccaagtaaataagacccaaatcccggacacggtcacacaatactcggagctgtaccatgacttcaccaccacgcaggtgggaatgcacgacattgtccgcagcttccggcagaccatggaccaatacagcacggagcccggcagatacaggttcatggggactgaagcctatgcagagagtattgacaggaccgtgatgtactatggattgccatttatccaagaagctgattttcccttcaacaattacctcagcatgctagacactgtttctgggaacagcgtgtatgaggttatcacatcctggatggaaaacatgccagaaggaaaatggcctaactggatgattggtggaccagacagttcacggctgacttcgcgtttggggaatcagtatgtcaacgtgatgaacatgcttcttttcacactccctggaactcctataacttactatggagaagaaattggaatgggaaatattgtagccgcaaatctcaatgaaagctatgatattaatacccttcgctcaaagtcaccaatgcagtgggacaatagttcaaatgctggtttttctgaagctagtaacacctggttacctaccaattcagattaccacactgtgaatgttgatgtccaaaagactcagcccagatcggctttgaagttatatcaagatttaagtctacttcatgccaatgagctactcctcaacaggggctggttttgccatttgaggaatgacagccactatgttgtgtacacaagagagctggatggcatcgacagaatctttatcgtggttctgaattttggagaatcaacactgttaaatctacataatatgatttcgggccttcccgctaaaatgagaataaggttaagtaccaattctgccgacaaaggcagtaaagttgatacaagtggcatttttctggacaagggagagggactcatctttgaacacaacacgaagaatctccttcatcgccaaacagctttcagagatagatgctttgtttccaatcgagcatgctattccagtgtactgaacatactgtatacctcgtgttaggcacctttatgaagagatgaagacactggcatttcagtgggattgtaagcatttgtaatagcttcatgtacagcatgctgcttggtgaacaatcattaattcttcgatatttctgtagcttgaatgtaactgctttaagaaaggttctcaaatgttttgaaaaaaataaaatgtttaaaagtaaattatggcttataggagcttataactttattcagatagcatcaatcagggatgaccagaacacattaggaccccagattattcaaaaactttaacgaattttaaggggaagaattttatcttttcccttaaaatgcagtcatagaaattagaggatgactcactgccacagtgtctaaaagcatttgctagcaaagaggcaggacactaatttgtaaactgctcaactgttctgactggaagggaggcctggagctctgctatcaccaatccttcccttccctctactccacatccttctaaggagcatgatttgaaaattactttcctaggttaatgggcatgtgcatcaatggagagaatagtataagcaagtgagatgtagactaagcaaaatttagatggagaagcacattttaaaaaattaataacttaaaagtctcaagttattaattttttttttgctaactcaattggaagtaagactatgaaatatttcagtgtgtttccaattcccagttgaatgcagtgtttcagaatttcaggtatttcttaagatcctcgaaaacactggtgctgtcaagtccaagttcctcgtacaggaatttaatttgggctgtaatctaaaagaaacacattaaaaaaattaaatagaaggcctttgtagtaaaa

SEQ ID NO: 243 - skipped

SEQ ID NO: 244 - protein, 685 aa, Unknown
  Description of Unknown: rBAT sequence
MAEDKSKRDSIEMSMKGCQTNNGFVHNEDILEQTPDPGSSTDNLKHSTRGILGSQEPDFKGVQPYAGMPKEVLFQFSGQARYRIPREILFWLTVASVLVLIAATIAIIALSPKCLDWWQEGPMYQIYPRSFKDSNKDGNGDLKGIQDKLDYITALNIKTVWITSFYKSSLKDFRYGVEDFREVDPIFGTMEDFENLVAAIHDKGLKLIIDFIPNHTSDKHIWFQLSRTRTGKYTDYYIWHDCTHENGKTIPPNNWLSVYGNSSWHFDEVRNQCYFHQFMKEQPDLNFRNPDVQEEIKEILRFWLTKGVDGFSLDAVKFLLEAKHLRDEIQVNKTQIPDTVTQYSELYHDFTTTQVGMHDIVRSFRQTMDQYSTEPGRYRFMGTEAYAESIDRTVMYYGLPFIQEADFPFNNYLSMLDTVSGNSVYEVITSWMENMPEGKWPNWMIGGPDSSRLTSRLGNQYVNVMNMLLFTLPGTPITYYGEEIGMGNIVAANLNESYDINTLRSKSPMQWDNSSNAGFSEASNTWLPTNSDYHTVNVDVQKTQPRSALKLYQDLSLLHANELLLNRGWFCHLRNDSHYVVYTRELDGIDRIFIVVLNFGESTLLNLHNMISGLPAKMRIRLSTNSADKGSKVDTSGIFLDKGEGLIFEHNTKNLLHRQTAFRDRCFVSNRACYSSVLNILYTSC

SEQ ID NO: 245 - DNA, 2058 bp, Unknown
  Description of Unknown: rBAT sequence
atggctgaagataaaagcaagagagactccatcgagatgagtatgaagggatgccagacaaacaacgggtttgtccataatgaagacattctggagcagaccccggatccaggaagctcaacagacaacctgaagcacagcaccaggggcatccttggctcccaggagcccgacttcaagggcgtccagccctatgcggggatgcccaaggaggtgctgttccagttctctggccaggcccgctaccgcatacctcgggagatcctcttctggctcacagtggcttctgtgctggtgctcatcgcggccaccatagccatcattgccctctctccaaagtgcctagactggtggcaggaggggcccatgtaccagatctacccaaggtctttcaaggacagtaacaaggatgggaacggagatctgaaaggtattcaagataaactggactacatcacagctttaaatataaaaactgtttggattacttcattttataaatcgtcccttaaagatttcagatatggtgttgaagatttccgggaagttgatcccatttttggaacgatggaagattttgagaatctggttgcagccatacatgataaaggtttaaaattaatcatcgatttcataccaaaccacacgagtgataaacatatttggtttcaattgagtcggacacggacaggaaaatatactgattattatatctggcatgactgtacccatgaaaatggcaaaaccattccacccaacaactggttaagtgtgtatggaaactccagttggcactttgacgaagtgcgaaaccaatgttattttcatcagtttatgaaagagcaacctgatttaaatttccgcaatcctgatgttcaagaagaaataaaagaaattttacggttctggctcacaaagggtgttgatggttttagtttggatgctgttaaattcctcctagaagcaaagcacctgagagatgagatccaagtaaataagacccaaatcccggacacggtcacacaatactcggagctgtaccatgacttcaccaccacgcaggtgggaatgcacgacattgtccgcagcttccggcagaccatggaccaatacagcacggagcccggcagatacaggttcatggggactgaagcctatgcagagagtattgacaggaccgtgatgtactatggattgccatttatccaagaagctgattttcccttcaacaattacctcagcatgctagacactgtttctgggaacagcgtgtatgaggttatcacatcctggatggaaaacatgccagaaggaaaatggcctaactggatgattggtggaccagacagttcacggctgacttcgcgtttggggaatcagtatgtcaacgtgatgaacatgcttcttttcacactccctggaactcctataacttactatggagaagaaattggaatgggaaatattgtagccgcaaatctcaatgaaagctatgatattaatacccttcgctcaaagtcaccaatgcagtgggacaatagttcaaatgctggtttttctgaagctagtaacacctggttacctaccaattcagattaccacactgtgaatgttgatgtccaaaagactcagcccagatcggctttgaagttatatcaagatttaagtctacttcatgccaatgagctactcctcaacaggggctggttttgccatttgaggaatgacagccactatgttgtgtacacaagagagctggatggcatcgacagaatctttatcgtggttctgaattttggagaatcaacactgttaaatctacataatatgatttcgggccttcccgctaaaatgagaataaggttaagtaccaattctgccgacaaaggcagtaaagttgatacaagtggcatttttctggacaagggagagggactcatctttgaacacaacacgaagaatctccttcatcgccaaacagctttcagagatagatgctttgtttccaatcgagcatgctattccagtgtactgaacatactgtatacctcgtgttag

SEQ ID NO: 246 - DNA, 4536 bp, Homo sapiens
ggctcactctggcaggtaggaacaggggagagtgcacctgctaccagtcaagctcagccagactgcaagaggaggcgaggcggagccagccgagggagtgaaccatggacaagttgaaatgcccgagtttcttcaagtgcagggagaaggagaaagtgtcggcttcatcagagaatttccatgttggtgaaaatgatgagaatcaggaccgtggtaactggtccaaaaaatcggattatcttctatctatgattggatacgcagtgggattaggaaatgtgtggagatttccatatctgacctacagcaatggtggaggcgccttcttgataccttatgcaattatgttagcattggctggtttacctttgttctttctggagtgttcactgggacaatttgctagcttaggtccagtttcagtttggaggattcttccattgtttcaaggtgtgggaattacaatggtcctgatctccatttttgtgacaatctattacaatgtcataattgcctatagtctttactacatgtttgcttcttttcaaagtgaactaccatggaaaaattgttcttcgtggtcagataaaaactgtagcagatcaccaatagtaactcactgtaatgtgagtacagtgaataaaggaatacaagagatcatccaaatgaataaaagctgggtagacatcaacaattttacctgcatcaacggcagtgaaatttatcagccagggcagcttcccagtgaacaatattggaataaagtggcgctccaacggtcaagtggaatgaatgagactggagtaattgtttggtatttagcactttgtcttcttctggcttggctcatagttggagcagcactatttaaaggaatcaaatcgtctggcaaggtggtatattttacagctcttttcccctatgtggtcctactcatcctgttagtacgaggtgcaactctggagggtgcttcaaaaggcatttcatactatattggagcccagtcaaattttacaaaacttaaggaagctgaggtatggaaagatgctgccactcagatattttactccctttcagtggcttggggtggcttagttgctctatcatcttacaataagttcaaaaacaactgcttctctgatgccattgtggtttgtttgacaaactgtctcactagcgtgtttgctggatttgctattttttctatattgggacacatggcccatatatctggaaaggaagtttctcaagttgtaaaatcaggttttgatttggcattcattgcctatccagaggctctagcccaactcccaggtggtccattttggtccatattattttttttcatgcttttaactttgggtctcgattctcagtttgcttcgattgaaacgatcacaacaacaattcaagatttatttcccaaagtgatgaagaaaatgagggttcccataactttgggctgctgcttggttttgtttctccttggtctcgtctgtgtgactcaggctggaatttactgggttcatctgattgaccacttctgtgctggatggggcattttaattgcagctatactggagctagttggaatcatctggatttatggagggaacagattcattgaggatacagaaatgatgattggagcaaagaggtggatattctggctatggtggagagcttgctggtttgtaattacgcctatccttttgattgcaatatttatctggtcattggtgcaatttcatagacctaattatggcgcaattccataccctgactggggagttgctttaggctggtgtatgattgttttctgcattatttggattccaattatggctatcataaaaataattcaggctaaaggaaacatctttcaacgccttataagttgctgcagaccagcttctaactggggtccatacctggaacaacatcgtggggaaagatataaagacatggtagatcctaaaaaagaggctgaccatgaaatacctactgttagtggcagcagaaaaccggaatgagatctcattgaaaaaaatatatgattgtataatgtgattttttttagaatagggggaaccttatttatttgtgtgttaactgaataggaaaatgtacatactatgttcatgatagtgtgatttttttcacatttaagcaggaatgcaatataaaaatgtgaatctcttaattctcagccatgtgcttattatatttctttttagattgtctatctgtataacacacacacacacacctaagagtctctatttcacaattatatttttgtaaatagtatatgcatttttaatacattggaggctttattttgaactaatttcttagagaatagttatattttctattacacaagtttaaaaatattattaacttgtattttcttaatatacaatctatcttttccacaaatatgagtgggaaataaatcagcacatttgaaagaaagtgttaaaactgaaggcctcacttaattagaaacgtgataaatatatggacaaatggactatacatactataagaggactgtagtttaatactttttacccaaatatgtttaaaaacttcgtgcatttgttacagctcatgttttctatatgaacttagtcattaatgttctttataaaaagtgaaataagatggaaaaataaggatcctacagccagtaagtgataaatctagaaaattgagttttgagtacctcttttcccatatacaatcttccttccttaggtaatttggaagaaaactatgacccatttaatttctattgtgtttcacaaaattaagtgttgttcattatactctctgaaatataggtttaatttcaaatagaatatggacttaaatgttaatgagaaaatggctttaatcaattctagcattttattactgtaatacagggctgatagagtgattttgtcttatatgagtaagttactacttacaggtgataacttgcatactattggaagataaacttgtcaaacttgtcaagaatgagaaaagccaaattagaaaatcctatgtcctagtttccttaccaaggataattaaatatatcactaagagctttatatattgattatatattgttgacaactggtttaagcatcatagcctatgatgataaacactgcctatatatgtaaatagcttttcatcaattcttaaatttcttaacctaggcttcagggagcatatgaaaccaaaattatatggaacattttctgtgtgtacatgtacatgcatttttctagggagagagtccgtaggtttatcagaatatcaaggaaaactgtgacccaaagaagtttaagaatcacatacactgctgctggctttttgtgcttggcaaatgagtgacaatagaagaaataatttttcttacacattttaaaacgttttctcttccttgtgattgaagatgaaaggagtaagaaattaaggcatttgtttaatttatactggtaacttatttaggggggaggggacatgaaggtaggtaaataggtaggcctctaattgaaccacctctctaagttatgtacgtatatataagctgaaattgtgtttgacattctgagggttttctttttctttttccttttttttttttttggtggggggctgggggtcagagtcttgttctgttgcccgggctggagtgcagtggcatgatctcagctcactgcaacctctgccttctggattcaagtgattctcctgcctcagcctcttgagtagctgggactacaggtgcccgccaccacaccagctaatttttgtatttttagtagaggcgaagtttccccatgttggccaggctggtcttgaactcccgacctcaagtgatctgtctacctcggcctcctaaagtgctgagattacaggtgtgagccaccgtgcccggcccattctaagggttttctttgaagacaggtcaaatgctgttagtaagtttcaggagattgttaattcctcagttataccagattttataaaatatttgagaatagatggctaacaagaggttagaaatacttttccttaattttaatccacagtatgttacatgcattctaccactacattttggtgctatttaaggtgtgcaattttctataggtgacttttgcaattcagggaagatttgggcatattaaatgaaagaatatctaattgggggaggtgtgaagggaaagaaattcttttcaaaagctgaccacaaagagtagttaaaagtttttgtcactatcttcacaagtgtgtaaagcacagatttcaacagagtgcttggcatattgtagggtgctcaatggtggtttttattattattactcagattccacagtggcaagaaacatcattctacataatggaaaacatttacatcaaatcccacttactttaatgcgaacttggagataatttatggtattgtattgtaaaccattaatgaaaactttttcacagttgagtgaaattaaaatcactatatctcaa

SEQ ID NO: 247 - skipped

SEQ ID NO: 248 - protein, 642 aa, Unknown
  Description of Unknown: ATB0,+ sequence
MDKLKCPSFFKCREKEKVSASSENFHVGENDENQDRGNWSKKSDYLLSMIGYAVGLGNVWRFPYLTYSNGGGAFLIPYAIMLALAGLPLFFLECSLGQFASLGPVSVWRILPLFQGVGITMVLISIFVTIYYNVIIAYSLYYMFASFQSELPWKNCSSWSDKNCSRSPIVTHCNVSTVNKGIQEIIQMNKSWVDINNFTCINGSEIYQPGQLPSEQYWNKVALQRSSGMNETGVIVWYLALCLLLAWLIVGAALFKGIKSSGKVVYFTALFPYVVLLILLVRGATLEGASKGISYYIGAQSNFTKLKEAEVWKDAATQIFYSLSVAWGGLVALSSYNKFKNNCFSDAIVVCLTNCLTSVFAGFAIFSILGHMAHISGKEVSQVVKSGFDLAFIAYPEALAQLPGGPFWSILFFFMLLTLGLDSQFASIETITTTIQDLFPKVMKKMRVPITLGCCLVLFLLGLVCVTQAGIYWVHLIDHFCAGWGILIAAILELVGIIWIYGGNRFIEDTEMMIGAKRWIFWLWWRACWFVITPILLIAIFIWSLVQFHRPNYGAIPYPDWGVALGWCMIVFCIIWIPIMAIIKIIQAKGNIFQRLISCCRPASNWGPYLEQHRGERYKDMVDPKKEADHEIPTVSGSRKPE

SEQ ID NO: 249 - DNA, 1929 bp, Unknown
  Description of Unknown: ATB0,+ sequence
atggacaagttgaaatgcccgagtttcttcaagtgcagggagaaggagaaagtgtcggcttcatcagagaatttccatgttggtgaaaatgatgagaatcaggaccgtggtaactggtccaaaaaatcggattatcttctatctatgattggatacgcagtgggattaggaaatgtgtggagatttccatatctgacctacagcaatggtggaggcgccttcttgataccttatgcaattatgttagcattggctggtttacctttgttctttctggagtgttcactgggacaatttgctagcttaggtccagtttcagtttggaggattcttccattgtttcaaggtgtgggaattacaatggtcctgatctccatttttgtgacaatctattacaatgtcataattgcctatagtctttactacatgtttgcttcttttcaaagtgaactaccatggaaaaattgttcttcgtggtcagataaaaactgtagcagatcaccaatagtaactcactgtaatgtgagtacagtgaataaaggaatacaagagatcatccaaatgaataaaagctgggtagacatcaacaattttacctgcatcaacggcagtgaaatttatcagccagggcagcttcccagtgaacaatattggaataaagtggcgctccaacggtcaagtggaatgaatgagactggagtaattgtttggtatttagcactttgtcttcttctggcttggctcatagttggagcagcactatttaaaggaatcaaatcgtctggcaaggtggtatattttacagctcttttcccctatgtggtcctactcatcctgttagtacgaggtgcaactctggagggtgcttcaaaaggcatttcatactatattggagcccagtcaaattttacaaaacttaaggaagctgaggtatggaaagatgctgccactcagatattttactccctttcagtggcttggggtggcttagttgctctatcatcttacaataagttcaaaaacaactgcttctctgatgccattgtggtttgtttgacaaactgtctcactagcgtgtttgctggatttgctattttttctatattgggacacatggcccatatatctggaaaggaagtttctcaagttgtaaaatcaggttttgatttggcattcattgcctatccagaggctctagcccaactcccaggtggtccattttggtccatattattttttttcatgcttttaactttgggtctcgattctcagtttgcttcgattgaaacgatcacaacaacaattcaagatttatttcccaaagtgatgaagaaaatgagggttcccataactttgggctgctgcttggttttgtttctccttggtctcgtctgtgtgactcaggctggaatttactgggttcatctgattgaccacttctgtgctggatggggcattttaattgcagctatactggagctagttggaatcatctggatttatggagggaacagattcattgaggatacagaaatgatgattggagcaaagaggtggatattctggctatggtggagagcttgctggtttgtaattacgcctatccttttgattgcaatatttatctggtcattggtgcaatttcatagacctaattatggcgcaattccataccctgactggggagttgctttaggctggtgtatgattgttttctgcattatttggattccaattatggctatcataaaaataattcaggctaaaggaaacatctttcaacgccttataagttgctgcagaccagcttctaactggggtccatacctggaacaacatcgtggggaaagatataaagacatggtagatcctaaaaaagaggctgaccatgaaatacctactgttagtggcagcagaaaaccggaatga

SEQ ID NO: 250 - DNA, 1179 bp, Unknown
  Description of Unknown: EF1alpha promoter sequence
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 251 - DNA, 500 bp, Unknown
  Description of Unknown: PGK promoter sequence
gggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcg

SEQ ID NO: 252 - DNA, 508 bp, Unknown
  Description of Unknown: CMV promoter sequence
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 253 - DNA, 584 bp, Unknown
  Description of Unknown: CAG promoter sequence
gcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 254 - DNA, 225 bp, Unknown
  Description of Unknown: bGH pA transcription termination and polyA signal sequence
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 255 - DNA, 49 bp, Unknown
  Description of Unknown: rbHBB pA transcription termination and polyA signal sequence
aataaaagatctttattttcattagatctgtgtgttggttttttgtgtg

SEQ ID NO: 256 - DNA, 238 bp, Unknown
  Description of Unknown: SV40 pA transcription termination and polyA signal sequence
ctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatatgca

SEQ ID NO: 257 - DNA, 481 bp, Homo sapiens
gacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttt

SEQ ID NO: 258 - DNA, 244 bp, Artificial Sequence
  Description of Artificial Sequence: Synthetic polynucleotide
tacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtgtacatacacgcgcttgactgtgccttt

SEQ ID NO: 259 - DNA, 266 bp, Artificial Sequence
  Description of Artificial Sequence: Synthetic polynucleotide
ttaaacaatttaaaggcaatgctaccaaatactaagcgcgtgtatgtacacttctgacccactgggaatgtgatgaaagaaataaaagctgaaatgaatcattctctctactattattctgatatttcacattcttaaaataaagtggtgatcctaactgaccttaagacagggaatctttactcggattaaatgtcaggaattgtgaaaaagtgagtttaaatgtatttggctaaggtgtatgtaaacttccgacttcaactgta

SEQ ID NO: 260 - DNA, 57 bp, Teschovirus A
gccaccaatttcagcctgctgaaacaggctggcgacgtggaagagaaccctggacct

SEQ ID NO: 261 - DNA, 63 bp, Thosea asigna virus
ggcagcggcgagggcagaggcagcctgctgacctgcggcgacgtggaggagaaccccggcccc

SEQ ID NO: 262 - DNA, 60 bp, Equine rhinitis A virus
ggcagcggccagtgcaccaactacgccctgctgaagctggccggcgacgtggagagcaac

SEQ ID NO: 263 - DNA, 75 bp, Foot-and-mouth disease virus
ggcagcggcgtgaagcagaccctgaacttcgacctgctgaagctggccggcgacgtggagagcaaccccggcccc

SEQ ID NO: 264 - DNA, 714 bp, Artificial Sequence
  Description of Artificial Sequence: Synthetic polynucleotide
gtgtccaagggcgaagaactgtttaccggcgtggtgcccatcctggtggaactggatggggatgtgaacggccacaagttcagcgttagcggagaaggcgaaggcgacgccacatacggaaagctgacactgaagttcatctgcaccaccggcaagctgcctgtgccatggccaacactggtcaccacactgacatacggcgtgcagtgcttcagcagataccccgaccatatgaagcagcatgacttcttcaagagcgccatgcctgagggctacgtgcaagagcggaccatcttctttaaggacgacggcaactacaagaccagggccgaagtgaagttcgagggcgacaccctcgtgaaccggatcgagctgaagggcatcgacttcaaagaggacggcaacatcctgggccacaagctcgagtacaactacaacagccacaacgtgtacatcatggccgacaagcagaaaaacggcatcaaagtgaacttcaagatccggcacaacatcgaggacggctcagtgcagctggccgaccactatcagcagaacacacccatcggagatggccccgttctgctgcccgataaccactacctgagcacacagagcaagctgagcaaggaccccaacgagaagcgggaccacatggtcctgctggaatttgtgacagccgccggaatcaccctcggcatggacgagctttacaaa

SEQ ID NO: 265 - DNA, 714 bp, Artificial Sequence
  Description of Artificial Sequence: Synthetic polynucleotide
gtgagcaagggcgaggagctgttcaccggcgtggtgcccatcctggtggagctggacggcgacgtgaacggccacaagttcagcgtgagcggcgagggcgagggcgacgccacctacggcaagctgaccctgaagttcatctgcaccaccggcaagctgcccgtgccctggcccaccctggtgaccaccctgacctacggcgtgcagtgcttcgccagataccccgaccacatgaagcagcacgacttcttcaagagcgccatgcccgagggctacgtgcaggagagaaccatcttcttcaaggacgacggcaactacaagaccagagccgaggtgaagttcgagggcgacaccctggtgaacagaatcgagctgaagggcatcgacttcaaggaggacggcaacatcctgggccacaagctggagtacaactacaacagccacaaggtgtacatcaccgccgacaagcagaagaacggcatcaaggtgaacttcaagaccagacacaacatcgaggacggcagcgtgcagctggccgaccactaccagcagaacacccccatcggcgacggccccgtgctgctgcccgacaaccactacctgagcacccagagcaagctgagcaaggaccccaacgagaagagagaccacatggtgctgctggagttcgtgaccgccgccggcatcaccctgggcatggacgagctgtacaag

SEQ ID NO: 266 - DNA, 705 bp, Artificial Sequence
  Description of Artificial Sequence: Synthetic polynucleotide
gtgtctaagggcgaagaggacaacatggccatcatcaaagaattcatgcggttcaaggtgcacatggaaggcagcgtgaacggccacgagttcgagattgaaggcgaaggcgagggcagaccttacgagggaacacagaccgccaagctgaaagtcaccaaaggcggccctctgccttttgcctgggacattctgagccctcagtttatgtacggctccaaggcctacgtgaagcaccccgccgatattcccgactatctgaagctgagcttccccgagggcttcaactgggagcgcgtgatgaatttcgaggacggcggcgtggtcaccgtgactcaagatagctctctgcaggacggcgagttcatctacaaagtgaagctgcggggcacaaacttccccagcgacggacctgtgatgcagtgcagaacaatgggctgggaagccagcaccgagagaatgtacccagaagatggcgccctgaagggcgagattaagcagcggctgaaactcaaggatggcggccactacgacgccgaagtgaaaaccacctacaaggccaagaaacccgtgcagctgcctggcgcctacaacgtggacatcaagctggatatcctgagccacaatgaggactacaccatcgtcgagcagtacgagagagccgaggggagacattctaccggcggaatggacgagctgtacaaa

SEQ ID NO: 267 - DNA, 693 bp, Artificial Sequence
  Description of Artificial Sequence: Synthetic polynucleotide
gtgtctaagggcgaagccgtgatcaaagaattcatgcggttcaaggtgcacatggaaggcagcatgaacggccacgagttcgagatcgaaggcgaaggcgagggcagaccttatgagggaacacagaccgccaagctgaaagtgaccaaaggcggccctctgcctttcagctgggacattctgagccctcagtttatgtacggcagccgggccttcatcaagcaccctgccgatattcccgactactacaagcagagcttccccgagggcttcaagtgggagagagtgatgaacttcgaggacggcggagccgtgaccgtgacacaggatacaagcctggaagatggcaccctgatctacaaagtgaagctgcggggcaccaactttccacctgatggccccgtgatgcagaaaaagaccatgggctgggaagccagcaccgagagactgtatcctgaggatggcgtgctgaagggcgacatcaagatggccctgagactgaaggatggcggcagatacctggccgacttcaagaccacctacaaggccaagaaacccgtgcagatgcctggcgcctacaacgtggacagaaagctggacatcaccagccacaacgaggactacaccgtggtggaacagtacgagcggagcgaaggcagacactctacaggcggaatggacgagctgtacaaa

SEQ ID NO: 268 - DNA, 594 bp, Artificial Sequence
  Description of Artificial Sequence: Synthetic polynucleotide
acagagtacaaacctacagtgcgcctggccaccagggacgatgttcctagagccgtcagaactctggccgctgccttcgccgattatccagccacaagacacaccgtggatcccgacagacacatcgagagagtgaccgagctgcaagagctgtttctgaccagagtcggcctggacatcggcaaagtgtgggttgcagatgatggcgccgctgtggctgtgtggacaacacctgaatctgtggaagccggcgcagtgtttgccgagatcggacctagaatggccgagctgagcggatctagactggctgctcaacagcagatggaaggcctgctggctccccacagaccaaaagagcctgcttggtttctggccaccgtgggcgttagccctgaccaccaaggcaaaggactgggatctgctgtggtgctgcctggcgttgaagccgctgaaagagctggcgttccagccttcctggaaacaagcgcccctcggaacctgcctttctacgagagactgggctttaccgtgaccgccgatgtggaagtgccagagggaccaagaacctggtgcatgaccagaaagcctggcgcc

SEQ ID NO: 269 - DNA, 789 bp, Artificial Sequence
  Description of Artificial Sequence: Synthetic polynucleotide
attgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttc

SEQ ID NO: 270 - DNA, 1014 bp, Artificial Sequence
  Description of Artificial Sequence: Synthetic polynucleotide
cctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaa

SEQ ID NO: 271 - DNA, 5448 bp, Artificial Sequence
  Description of Artificial Sequence: Synthetic polynucleotide
gccacctgacgtctaagaaaccattattatcatgacattaacctataaaaataggcgtatcacgaggccctttcgttgtaaaacgacggccagtcgaaccacgcaatgcgtctcgatccgcagtgtcttgcgtctcttacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtgtacatacacgcgcttgactgtgcctttgctcttcaatgggagggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatactgccgccaccatgggctccggcgccaccaactttagcctgctgaaacaggcaggcgacgtggaagagaaccctggacctgtgtccaagggcgaagaactgtttaccggcgtggtgcccatcctggtggaactggatggggatgtgaacggccacaagttcagcgttagcggagaaggcgaaggcgacgccacatacggaaagctgacactgaagttcatctgcaccaccggcaagctgcctgtgccatggccaacactggtcaccacactgacatacggcgtgcagtgcttcagcagataccccgaccatatgaagcagcatgacttcttcaagagcgccatgcctgagggctacgtgcaagagcggaccatcttctttaaggacgacggcaactacaagaccagggccgaagtgaagttcgagggcgacaccctcgtgaaccggatcgagctgaagggcatcgacttcaaagaggacggcaacatcctgggccacaagctcgagtacaactacaacagccacaacgtgtacatcatggccgacaagcagaaaaacggcatcaaagtgaacttcaagatccggcacaacatcgaggacggctcagtgcagctggccgaccactatcagcagaacacacccatcggagatggccccgttctgctgcccgataaccactacctgagcacacagagcaagctgagcaaggaccccaacgagaagcgggaccacatggtcctgctggaatttgtgacagccgccggaatcaccctcggcatggacgagctttacaaaggcggcggaggatctggcggaggtggaagcggaggcggtggaagcacagagtacaaacctacagtgcgcctggccaccagggacgatgttcctagagccgtcagaactctggccgctgccttcgccgattatccagccacaagacacaccgtggatcccgacagacacatcgagagagtgaccgagctgcaagagctgtttctgaccagagtcggcctggacatcggcaaagtgtgggttgcagatgatggcgccgctgtggctgtgtggacaacacctgaatctgtggaagccggcgcagtgtttgccgagatcggacctagaatggccgagctgagcggatctagactggctgctcaacagcagatggaaggcctgctggctccccacagaccaaaagagcctgcttggtttctggccaccgtgggcgttagccctgaccaccaaggcaaaggactgggatctgctgtggtgctgcctggcgttgaagccgctgaaagagctggcgttccagccttcctggaaacaagcgcccctcggaacctgcctttctacgagagactgggctttaccgtgaccgccgatgtggaagtgccagagggaccaagaacctggtgcatgaccagaaagcctggcgcctgagcttctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcgctgcatgaagagcttaaacaatttaaaggcaatgctaccaaatactaagcgcgtgtatgtacacttctgacccactgggaatgtgatgaaagaaataaaagctgaaatgaatcattctctctactattattctgatatttcacattcttaaaataaagtggtgatcctaactgaccttaagacagggaatctttactcggattaaatgtcaggaattgtgaaaaagtgagtttaaatgtatttggctaaggtgtatgtaaacttccgacttcaactgtaagagacggagtcactgccaaccgagacggtcatagctgtttcctgtgtgccgcttcctcgctcactgactcgctgcgctcggtcgttcggctgcggcgagcggtatcagctcactcaaaggcggtaatacggttacccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagtcgaaatacgcgatcgctgttaaaaggacaattacaaacaggaatcgaatgcaaccggcgcaggaacacggccagcgcatcaacaatattttcacctgaatcaggatattcttctaatacctggaatgctgttttcccggggatcgctgtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagt

SEQ ID NO: 272 - DNA, 3802 bp, Artificial Sequence
  Description of Artificial Sequence: Synthetic polynucleotide
ccaatgattacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtgtacatacacgcgcttgactgtgcctttgctcttcaatgggagggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatactgccgccaccatgtaagcttctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcgctgcatgaagagcttaaacaatttaaaggcaatgctaccaaatactaagcgcgtgtatgtacacttctgacccactgggaatgtgatgaaagaaataaaagctgaaatgaatcattctctctactattattctgatatttcacattcttaaaataaagtggtgatcctaactgaccttaagacagggaatctttactcggattaaatgtcaggaattgtgaaaaagtgagtttaaatgtatttggctaaggtgtatgtaaacttccgacttcaactgtaatcggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 273 - DNA, 1236 bp, Artificial Sequence
  Description of Artificial Sequence: Synthetic polynucleotide
atgagcagcaagggatctgtggtgctggcctactctggcggcctggatacctcttgtatcctcgtgtggctgaaagaacagggctacgacgtgatcgcctacctggccaacatcggccagaaagaggacttcgaggaagcccggaagaaggccctgaagctgggagccaagaaggtgttcatcgaggacgtgtcccgcgagttcgtggaagagtttatctggcccgccatccagtctagcgccctgtacgaggatagatacctgctgggcaccagcctggccagaccttgtatcgccagaaagcaggtcgagatcgcccagagagaaggcgccaaatacgtgtcccatggcgccacaggcaagggcaacgatcaagttcgcttcgagctgagctgctactctctggcccctcagatcaaagtgatcgccccttggagaatgcccgagttctacaacagattcaagggccgcaacgacctgatggaatacgccaagcagcacggcatccccattccagtgacacccaagaatccttggagcatggacgagaacctgatgcacatcagctacgaggccggcatcctggaaaaccctaagaatcaggcccctcctggcctgtacaccaagacacaggatccagccaaggctcccaacacacccgacattctggaaatcgagttcaagaaaggcgtgcccgtgaaagtgaccaacgtgaaggatggcaccacacaccagacaagcctggaactgttcatgtacctgaacgaggtggccggcaagcacggcgtgggaagaatcgacatcgtggaaaatcggttcatcggcatgaagtcccggggcatctatgagacaccagccggcaccattctgtatcacgcccacctggacattgaggccttcaccatggaccgggaagtgcggaagatcaagcaaggcctgggcctgaagtttgccgaactggtgtataccggcttctggcactctcctgagtgcgagtttgtgcggcactgcattgccaagagccaagagcgcgtggaaggcaaggtgcaggtttccgtgctgaaaggccaggtgtacattctgggcagagagagccctctgagcctgtataacgaggaactcgtgtccatgaacgtgcagggcgattacgagcctaccgatgccaccggcttcatcaacatcaacagcctgagactgaaagagtaccaccgcctgcagtccaaagtgaccgccaaa

SEQ ID NO: 274 - DNA, 708 bp, Artificial Sequence
  Description of Artificial Sequence: Synthetic polynucleotide
atggtgtccaagggtgaagaggacaacatggcttccttgcctgccacccatgaactccatatcttcgggtctattaacggagtcgactttgatatggtggggcagggtacgggcaaccctaacgacggctacgaagagctgaacctgaagtccactaagggcgacctccagttttctccttggattctggtgccacacatcggttatggttttcatcagtaccttccatacccggacggcatgtccccgttccaggcggctatggtcgacggatctggctaccaggtgcaccgcactatgcagtttgaagacggcgcatctctgaccgtgaactaccgttacacttatgagggctcccatatcaagggtgaggcgcaagtcaagggcaccggtttcccggcggatggaccagtgatgaccaacagtcttaccgcagccgactggtgtcgcagcaaaaagacatatcccaacgacaagaccattatcagcacctttaaatggtcttacacgaccgggaacggtaaacgctataggagcacagcccgcactacctatacctttgcaaaacctatggccgcgaactatctgaaaaaccagccgatgtacgtcttccggaagaccgagctgaagcacagtaagacagagctgaacttcaaagagtggcaaaaagcttttacggacgtgatgggcatggatgaattgtacaag

SEQ ID NO: 275 - protein, 5 aa, Artificial Sequence
  Description of Artificial Sequence: Synthetic peptide
GGGGS

SEQ ID NO: 276 - protein, 10 aa, Artificial Sequence
  Description of Artificial Sequence: Synthetic peptide
GGGGSGGGGS

SEQ ID NO: 277 - protein, 20 aa, Artificial Sequence
  Description of Artificial Sequence: Synthetic peptide
GGGGSGGGGSGGGGSGGGGS

SEQ ID NO: 278 - protein, 6 aa, Artificial Sequence
  Description of Artificial Sequence: Synthetic 6xHis tag
HHHHHH